SEQUENCE LISTING (225 sequences)

SEQ ID NO: 1 - protein, 916 aa, Neisseria gonorrhoeae
  VAR_SEQ 91: note = Xaa is S in wt gyrA gene; Xaa is F in S91F gyrA mutant
MTDATIRHDHKFALETLPVSLEDEMRKSYLDYAMSVIVGRALPDVRDGLKPVHRRVLYAMHELKNNWNAAYKKSARIVGDVIGKYHPHGDXAVYDTIVRMAQNFAMRYVLIDGQGNFGSVDGLAAAAMRYTEIRMAKISHEMLADIEEETVNFGPNYDGSEHEPLVLPTRFPTLLVNGSSGIAVGMATNIPPHNLTDTINACLRLLDEPKTEIDELIDIIQAPDFPTGATIYGLGGVREGYKTGRGRVVMRGKTHIEPIGKNGEREAIVIDEIPYQVNKAKLVEKIGDLVREKTLEGISELRDESDKSGMRVVIELKRNENAEVVLNQLYKLTPLQDSFGINMVVLVDGQPRLLNLKQILSEFLRHRREVVTRRTLFRLKKARHEGHIAEGKAVALSNIDEIIKLIKESPNAAEAKEKLLARPWRSSLVEEMLTRSGLDLEMMRPEGLAANIGLKKQGYYLSEIQADAILRMSLRNLTGLDQKEIIESYKNLMGKIIDFVDILSKPERITQIIRDELEEIKTNYGDERRSEINPFGGDIADEDLIPQREMVVTLTHGGYIKTQPTTDYQAQRRGGRGKQAAATKDEDFIETLFVANTHDYLMCFTNLGKCHWIKVYKLPEGGRNSRGRPINNVIQLEEGEKVSAILAVREFPEDQYVFFATAQGMVKKVQLSAFKNVRAQGIKAIALKEGDYLVGAAQTGGADDIMLFSNLGKAIRFNEYWEKSGNDEAEDADIETEISDDLEDETADNENTLPSGKNGVRPSGRGSGGLRGMRLPADGKIVSLITFAPETEESGLQVLTATANGYGKRTPIADYSRKNKGGQGSIAINTGERNGDLVAATLVGETDDLMLITSGGVLIRTKVEQIRETGRAAAGVKLINLDEGETLVSLERVAEDESELSGASVISNVTEPEAEN

SEQ ID NO: 2 - DNA, 2751 bp, Neisseria gonorrhoeae
  source 1..2751: mol_type = other DNA
  variation 272: note = mutation - n is cytosine (C) in wt gyrA gene; n is thymidine (T) in gyrA mutant
atgaccgacgcaaccatccgccacgaccacaaattcgccctcgaaaccctgcccgtcagccttgaagacgaaatgcgcaaaagctatctcgactacgccatgagcgtcattgtcgggcgcgcgctgccggacgttcgcgacggcctaaagccggtgcaccggcgcgtactgtacgcgatgcacgagctgaaaaataactggaatgccgcctacaaaaaatcggcgcgcatcgtcggcgacgtcatcggtaaataccacccccacggcgattncgcagtttacgacaccatcgtccgtatggcgcaaaatttcgctatgcgttatgtgctgatagacggacagggcaacttcggatcggtggacgggcttgccgccgcagccatgcgctataccgaaatccgcatggcgaaaatctcacatgaaatgctggcagacattgaggaagaaaccgttaatttcggcccgaactacgacggtagcgaacacgagccgcttgtactgccgacccgtttccccacactgctcgtcaacggctcgtccggtatcgccgtcggtatggcgaccaacatcccgccgcacaacctcaccgacaccatcaacgcctgtctgcgtcttttggacgaacccaaaaccgaaatcgacgaactgatcgacattatccaagcccccgacttcccgaccggggcaaccatctacggcttgggcggcgtgcgcgaaggctataaaacaggccgcggccgcgttgttatgcgcggtaagacccatatcgaacccataggcaaaaacggcgaacgcgaacgcatcgttatcgacgaaatcccctatcaggtcaacaaagccaagttggtcgagaaaatcggcgatttggttcgggaaaaaacactggaaggcatttccgagctccgcgacgaatccgacaaatccggtatgcgcgtcgttatcgagctgaaacgcaacgaaaatgccgaagtcgtcttaaaccaactctacaaactgactccgctgcaagacagtttcggcatcaatatggtggttttggtcgacggacaaccgcgcctgttaaacctgaaacagattctctccgaattcctgcgccaccgccgcgaagtcgttacccgacgtacgcttttccggctgaagaaggcacgccatgaagggcatatcgccgaacggaaagccgtcgcactgtccaatatcgatgaaatcatcaagctcatcaaagaatcgcccaacgcggccgaggccaaagaaaaactgcttgcgcgcccttgggccagcagcctcgttgaagaaatgctgacgcgttccggtctggatttggaaatgatgcgtccggaaggattggtcgcaaacattggtctgaaaaaacaaggttattacctgagcgagattcaggcagatgctattttacgcatgagcctgcgaaacctgaccggcctcgatcagaaagaaattatcgaaagctacaaaaacctgatgggtaaaatcatcgactttgtggatatcctctccaaacccgaacgcattacccaaatcatccgtgacgaactggaagaaatcaaaaccaactatggcgacgaacgccgcagcgaaatcaacccgttcggcggcgacattgccgatgaagacctgattccgcaacgcgaaatggtcgtgaccctgacccacggcggctatataaaaacccagccgaccaccgactatcaggctcagcgtcgcggcgggcgcggcaaacaggcggctgccaccaaagacgaagactttatcgaaaccctgtttgttgccaacacgcatgactatttgatgtgttttaccaacctcggcaagtgccactggattaaggtttacaaactgcccgaaggcggacgcaacagccgcggccgtccgattaacaacgtcatccagctggaagaaggcgaaaaagtcagcgcgattctggcagtacgcgagtttcccgaagaccaatacgtcttcttcgccaccgcgcagggaatggtgaaaaaagtccaactttccgcctttaaaaacgtccgcgcccaaggcattaaagccatcgcactcaaagaaggcgactacctcgtcggcgctgcgcaaacaggcggtgcggacgacattatgttgttctccaacttgggcaaagccatccgcttcaacgaatactgggaaaaatccggcaacgacgaagcggaagatgccgacatcgaaaccgagatttcagacgacctcgaagacgaaaccgccgacaacgaaaacaccctgccaagcggcaaaaacggcgtgcgtccgtccggtcgcggcagcggcggtttgcgcggtatgcgcctgcctgccgacggcaaaatcgtcagcctgattaccttcgcccctgaaaccgaagaaagcggtttgcaagttttaaccgccaccgccaacggatacggaaaacgcaccccgattgccgattacagccgcaaaaacaaaggcgggcaaggcagtattgccattaacaccggcgagcgcaacggcgatttggtcgccgcaaccttggtcggcgaaaccgacgatttgatgctgattaccagcggcggcgtgcttatccgtaccaaagtcgaacaaatccgcgaaaccggccgcgccgcagcaggcgtgaaactgattaacttggacgaaggcgaaaccttggtatcgctggaacgtgttgccgaagacgaatccgaactctccggcgcttctgtaatttccaatgtaaccgaaccggaagccgagaactga

SEQ ID NO: 3 - protein, 581 aa, Neisseria gonorrhoeae
  VAR_SEQ 316: note = Xaa is V in wt penA gene; Xaa is T or P in V316T/P penA mutant
  VAR_SEQ 501: note = Xaa is A in wt penA gene; Xaa is V or P or T in A501V/P/T penA mutant
  VAR_SEQ 345: note = insD345 Xaa is D in insD345 penA mutant
  VAR_SEQ 312: note = Xaa is I in wt penA gene; Xaa is M in I312M penA mutant
  VAR_SEQ 311: note = Xaa is A in wt penA gene; Xaa is V in A311V penA mutant
  VAR_SEQ 512: note = Xaa is N in wt penA gene Xaa is Y in N512Y penA mutant
  REGION 375..377: note = MISC_FEATURE - Mosaic region GAE at residues 375-377
  VAR_SEQ 551: note = Xaa is P in wt penA gene; Xaa is L or S in P551L/S penA mutant
  VAR_SEQ 516: note = Xaa is A in wt penA gene; Xaa is G in A516G penA mutant
  VAR_SEQ 34: note = Xaa is A in wt penA gene; Xaa is A in A34 penA mutant
  VAR_SEQ 542: note = Xaa is G in wt penA gene; Xaa is S in G542S penA mutant
  VAR_SEQ 545: note = Xaa is G in wt penA gene; Xaa is S in G545S penA mutant
MLIKSEYKPRMLPKEEQVKKPMTSNGRISFVLMXMAVLFACLIARGLYLQTVTYNFLKEQGDNRIVRTQALPATRGTVSDRNGAVLALSAPTESLFAVPKDMKEMPSAAQLERLSELVDVPVDVLRNKLEQKGKSFIWIKRQLDPKVAEEVKALGLENFVFEKELKRHYPMGNLFAHVIGFTDIDGKGQEGLELSLEDSLYGEDGAEVVLRDRQGNIVDSLDSPRNKAPQNGKDIILSLDQRIQTLAYEELNKAVEYHQAKAGTVVVLDARTGEILALANTPAYDPNRPGRADSEQRRNRAVTDMIEPGSAIKPFVIAKALDAGKTDLNERLNTQPYKIGPSPVRDTHVYPSLDVRGIMQKSSNVGTSKLSARFGAEEMYDFYHELGIGVRMHSGFPGETAGLLRNWRRWRPIEQATMSFGYGLQLSLLQLARAYTALTHDGVLLPLSFEKQAVAPQGKRIFKESTAREVRNLMVSVTEPGGTGTAGAVDGFDVGAKTGTARKFVNGRYADNKHVATFIGFAPAKNPRVIVAVTIDEPTAHGYYGGVVAGPPFKKIMGGSLNILGISPTKPLTAAAVKTPS

SEQ ID NO: 4 - DNA, 1843 bp, Neisseria gonorrhoeae
  variation 1034: note = mutation - N is cytosine (c) in wt penA gene; N is thymine (t) in A311V penA mutant.
  variation 1635: note = mutation - N is adenine (a) in wt penA gene; N is thymine (t) in N512Y penA mutant.
  variation 1603..1604: note = mutation - N1 is guanine (g) and N2 is cytosine (c) in wt penA gene; N1 is guanine (g) and N2 is thymine (t) in A501V penA mutant; or N1 is cytosine (c) and N2 is cytosine (c) in A501P penA mutant; or
  variation 1753..1754: note = mutation - N1 is cytosine (c) and N2 is cytosine (c) in wt penA gene; N1 is cytosine (c) and N2 is thymine (t) in P551L penA mutant; or N1 is thymine (5) and N2 is cytosine (c) in P551S penA mutant.
  variation 1726: note = mutation - N is guanine (g) in wt penA gene; N is adenine (a) in G542S penA mutant.
  variation 1648: note = mutation - N is cytosine (c) in wt penA gene; N is guanine (g) in A516G penA mutant.
  variation 1735..1736: note = mutation - N is guanine (g) in wt penA gene; N is adenine (a) in G545S penA mutant.
  misc_feature 1224..1232: note = Mosaic region at residues 1224-1232
  variation 203: note = mutation - N is adenine (a) in wt penA gene; N is thymine (t) or cytosine (c) or guanine (g) in A34 penA mutant
  variation 1038: note = mutation - N is cytosine (c) in wt penA gene; N is guanine (g) in I312M penA mutant.
  variation 1048..1049: note = mutation - N1 is guanine (g) and N2 is thymine (t) in wt penA gene; N1 is adenine (a) and N2 is cytosine (c) in V316T penA mutant; or N1 is cytosine (c) and N2 is cytosine (c) in V316P penA mutant.
  source 1..1843: mol_type = other DNA
atgttgattaaaagcgaatataagccccggatgctgcccaaagaagagcaggtcaaaaagccgatgaccagtaacggacggattagcttcgtcctgatggcaatggcggtcttgtttgcctgtctgattgcccgcgggctgtatctgcagacggtaacgtataactttttgaaagaacagggcgacaaccggattgtgcggactcaagcattgccggctacacgcggtacggtttcggaccggaacggtgcggttttggcgttgagcgcgccgacggagtccctgtttgccgtgcctaaagatatgaaggaaatgccgtctgccgcccaattggaacgcctgtccgagcttgtcgatgtgccggtcgatgttttgaggaacaaactcgaacagaaaggcaagtcgtttatttggatcaagcggcagctcgatcccaaggttgccgaagaggtcaaagccttgggtttggaaaactttgtatttgaaaaagaattaaaacgccattacccgatgggcaacctgtttgcacacgtcatcggatttaccgatattgacggcaaaggtcaggaaggtttggaactttcgcttgaagacagcctgtatggcgaagacggcgcggaagttgttttgcgggaccggcagggcaatattgtggacagcttggactccccgcgcaataaagcaccgcaaaacggcaaagacatcatcctttccctcgatcagaggattcagaccttggcctatgaagagttgaacaaggcggtcgaataccatcaggcaaaagccggaacggtggtggttttggatgcccgcacgggggaaatcctcgccttggccaatacgcccgcctacgatcccaacagacccggccgggcagacagcgaacagcggcgcaaccgtgccgtaaccgatatgatcgaacctggttcggcaatcaaaccgttcgtgattgcgaaggcattggatgcgggcaaaaccgatttgaacgaacggctgaatacgcagccttataaaatcggaccgtctcccgtgcgcgacgatacccatgtttacccctctttggatgtgcgcggcattatgcagaaatcgtccaacgtcggcacaagcaaactgtctgcgcgtttcggcgccgaagaaatgtatgacttctatcatgaattgggcatcggtgtgcgtatgcactcgggctttccgggggaaactgcaggtttgttgagaaattggcgcaggtggcggcccatcgaacaggcgacgatgtctttcggttacggcctgcaattgagcctgctgcaattggcgcgcgcctataccgcactgacgcacgacggcgttttgctgccgctcagctttgagaagcaggcggttgcgccgcaaggcaaacgcatattcaaagaatcgaccgcgcgcgaggtacgcaatctgatggtttccgtaaccgagccgggcggcaccggtacggcgggtgcggtggacggtttcgatgtcggcgctaaaaccggtacggcgcgcaaactggtcaatggccgctatgtggacaacaaacacgtcggtacgtttatcggttttgcccccgccaaaaacccccgtgtgattgtggcggtaaccatcgacgaaccgactgcccacggctattacggcggcgtagtggcagggtcgcccttcaaaaaaattatgggcggcagcctgaacatcttgggcatttccccgaccaagccactgaccgccgcagccgtcaaaacaccgtcttaatccgagtatcaacgagattgttttatgttcagcaagttaagccctttggctgaaaccggcatcccgaccctgtcgtgtgcaaacgcggcagggc

SEQ ID NO: 5 - protein, 348 aa, Neisseria gonorrhoeae
  VARIANT 120: note = Xaa is G in wt porin protein gene; Xaa is K in G120K porin protein mutant
  VARIANT 121: note = Xaa is A in wt porin protein gene; Xaa is N or D in A121N/D porin protein mutant
MKKSLIALTLAALPVAAMADVTLYGAIKAGVQTYRSVEHTDGKVSKVETGSEIADFGSKIGFKGQEDLGNGLKAVWQLEQGASVAGTNTGWGNKQSFVGLKGGFGTIRAGSLNSPLKNTGANVNAWESGKFTGNVLEISGMAQREHRYLSVRYDSPEFAGFSGSVQYAPKDNSGSNGESYHVGLNYQNSGFFAQYAGLFQRYGEGTKKIEYDDQTYSIPSLFVEKLQVHRLVGGYDNNALYVSVAAQQQDAKLYGAMSGNSHNSQTEVAATAAYRFGNVTPRVSYAHGFKGTVDSANHDNTYDQVVVGAEYDFSKRTSALVSAGWLQGGKGADKIVSTASAVVLRHKF

SEQ ID NO: 6 - DNA, 1047 bp, Neisseria gonorrhoeae
  source 1..1047: mol_type = other DNA
  variation 361..363: note = mutation - GCC mutation at residues 361-363
atgaaaaaatccctgattgccctgactttggcagcccttcctgttgcggcaatggccgatgtcaccctgtacggtgccatcaaagccggcgtacaaacttaccgttctgtagaacatacagacggcaaggtaagtaaagtggaaaccggcagcgaaatcgccgacttcggttcaaaaatcggcttcaaaggccaagaagacctcggcaacggcctgaaggccgtttggcagttggaacaaggtgcctccgtcgccggcactaacaccggctggggcaacaaacaatccttcgtcggcttgaagggcggcttcggtaccatccgcgccggtagcctgaacagccccctgaaaaacaccggcgccaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcaggctcaaacggcgaatcttaccacgttggcttgaactaccaaaacagcggcttcttcgcacaatacgccggcttgttccaaagatacggcgaaggcactaaaaaaatcgaatacgatgatcaaacttatagtatccccagtctgtttgttgaaaaactgcaagttcaccgtttggtaggcggttacgacaataatgccctgtacgtttccgtagccgcacaacaacaagatgccaaattgtatggagcaatgagcggtaattcgcacaactctcaaaccgaagttgccgctaccgcggcataccgtttcggcaatgtaacgccccgcgtttcttacgcccacggcttcaaaggcactgttgatagtgcaaaccacgacaatacttatgaccaagtggttgtcggtgcggaatacgacttctccaaacgcacttctgccttggtttctgccggctggttgcaaggaggcaaaggcgcagacaaaatcgtatcgactgccagcgccgtcgttctgcgccacaaattctaa

SEQ ID NO: 7 - protein, 798 aa, Neisseria gonorrhoeae
  VARIANT 421: note = Xaa is L in wt ponA gene; Xaa is P in L421P ponA mutant
MIKKILTTCFGLFFGFCVFGVGLVAIAILVTYPKLPSLDSLQHYQPKMPLTIYSADGEVIGMYGEQRREFTKIGDFPEVLRNAVIAAEDKRFYRHWGVDVWGVARAAVGNVVSGSVQSGASTITQQVAKNFYLSSEKTFTRKFNEVLLAYKIEQSLSKDKILELYFNQIYLGQRAYGFASAAQIYFNKNVRDLTLAEAAMLAGLPKAPSAYNPIVNPERAKLRQKYILNNMLEEKMITVQQRDQALNEELHYERFVRKIDQSALYVAEMVRRELYEKYGEDAYTQGFKVYTTVRTDHQKAATEALRKALRNFDRGSSYRGAENYIDLSKSEDVEETVSQYLSGLYTVDKMVPAVVLDVTKKKNVVIQLPGGRRVALDRRALGFAARAVDNEKMGEDRIRRGAVIRVKNNGGRWAVVQEPLLQGALVSLDAKTGAVRALVGGYDFHSKTFNRAVQAMRQPGSTFKPFVYSAALSKGMTASTVVNDAPISLPGKGPNGSVWTPKNSDGRYSGYITLRQALTASKNMVSIRILMSIGVGYAQQYIRRFGFRPSELPASLSMALGTGETTPLKVAEAYSVFANGGYRVSSHVIDKIYDRDGRLRAQMQPLVAGQNAPQAIDPRNAYIMYKIMQDVVRVGTARGAAALGRTDIAGKTGTTNDNKDAWFVGFNPDVVTAVYIGFDKPKSMGRAGYGGTIAVPVWVDYMRFALKGKQGKGMKMPEGVVSSNGEYYMKERMVTDPGLMLDNSGIAPQPSRRAKEDDEAAVENEQQGRSDETRQDVQETPVLPSNTDSKQQQLDSLF

SEQ ID NO: 8 - DNA, 2396 bp, Neisseria gonorrhoeae
  source 1..2396: mol_type = other DNA
  variation 1261..1263: note = mutation - CTG mutation at residues 1261-1263
atgattaaaaagattttaactacttgttttggtttgttttttggtttttgtgtatttggagtgggtctggttgccattgctattttggtaacgtatccgaaactgccgtctttggattctttgcagcattaccagcctaaaatgccgttgactatttattcggcggatggagaagtcatcgtatgtatggggagcagcggcgcgaatttacaaaaatcggcgatttccccgaggtgttgcggaatgcggttattgccgccgaggataaacgcttttaccggcattggggggtggatgtttggggtgttgcccgcgctgccgtcggcaatgtcgtgtccggcagcgtgcagtcgggtgcgagtacgattacacagcaggtggcgaaaaatttttatttgagcagtgaaaaaacgttcacacgcaaattcaatgaggtgttgcttgcctataaaatcgagcagtctttaagcaaagacaaaatccttgagttgtatttcaatcagatttacctcggtcagcgcgcctatggttttgcatctgccgcgcaaatctatttcaataagaatgtccgagatttgactttggcggaagccgccatgcttgcgggactgcccaaggctccgtctgcctataatccgattgttaatccggagcgtgccaagttgcgccagaagtatattttgaacaatatgctcgaggagaagatgattaccgtgcaacagcgcgatcaggcattgaatgaggaactgcattatgagcggtttgttcggaaaatcgatcagagtgctttatatgtggcggaaatggtgcgtcgggaactgtatgagaaatatggtgaagatgcctatacgcagggttttaaggtttataccacggtccgcaccgatcatcagaaggcggcaaccgaggcattgcgcaaggctctacggaatttcgatcgcggcagcagctaccgcggtgcggaaaactatatcgatttgagtaagagtgaagatgtcgaggagactgtcagccagtatctgtcgggactctataccgtcgataaaatggttcccgccgttgtgttggatgttactaaaaagaaaaatgtcgtcatacagctgcccggcggcaggcgggttgcgcttgacaggcgcgccttgggttttgcggcccgagcggtcgataatgagaaaatgggggaggaccgtatccgcaggggcgcggtcatccgtgtcaaaaacaacggcgggcgttgggcggtggttcaagagccgttgctgcagggggctttggtttcgctggatgcaaaaaccggagctgtgcgcgcgctggtcggcggttatgattttcacagcaaaacattcaatcgtgccgttcaggcaatgcggcagccgggttcgacctttaagccgtttgtctattcggcggcattatctaaggggatgaccgcgtccacagtggttaacgatgcgccgatttccctgccggggaaagggccgaacggttcggtttggacacctaaaaattcagacggcagatattccggctacattactttgagacaggctctgacggcttccaagaatatggtttccatccgtattttgatgtctatcggtgtcggttacgcgcaacagtatatccggcgtttcggcttcaggccgtccgagctgccggcaagcctgtctatggctttaggtacgggcgagacgacgccgttgaaagtggcggaggcatatagtgtatttgcgaacggcggatatagggtttcttcgcacgtgatcgataagatttatgacagagacggcaggttgcgcgcccaaatgcaacctttggtggcagggcaaaatgcgcctcaggcaatcgatccgcgcaatgcctatattatgtataagattatgcaggatgtggtccgtgtcggtacggcaaggggggcagctgcgttgggaagaacggatattgccggtaaaacgggtacgaccaacgacaataaagatgcgtggtttgtcggttttaaccctgatgtggttactgccgtatatatcggcttcgacaaacctaagagtatggggcgtgccggctacggcggtacgattgcggtgccggtttgggtggactatatgcgttttgcgttgaaaggaaagcagggcaaagggatgaaaatgcctgaaggtgtggtcagcagcaatggcgaatactatatgaaggaacgtatggtaaccgatccgggcttgatgctggacaacagcggtattgcgccgcaaccttcccgacgggcaaaagaagatgatgaagcggcagtagaaaacgaacagcagggaaggtctgacgaaacgcgtcaggacgtacaggaaacgccggtgcttccgagcaatacggattccaaacagcagcagttggattccctgttttaa

SEQ ID NO: 9 - skipped

SEQ ID NO: 10 - DNA, 2117 bp, Neisseria gonorrhoeae
  regulatory 8: regulatory_class = promoter; note = promoter - -35A promoter deletion
  gene 1064..1696: note = MtrR Gene
  source 1..2117: mol_type = other DNA
gatcagcaaacagcaggcggcctttttcaggataaaccgtaccgtcgtcaaatttgatgccgaccgcaatcgcaccatccgccgccagcagcttgccttcggctatctgccggcgcagtttcatcacttcggatgcagactgggtaacgttgggtaacgtcggattggtttggcggatggtggctaaaacagttgtatcgcccgcattcaacagcgtaccttcggaaactttggactgaccgataaagcccgaaatcggcgcggtaatgcgcgaacggttcagattgatgccggcggatttgatcgccgcctgcgccgctttaacgcccgcctcggcagaacgtttcgccgttaccgcagcatcgtactcttgtttactgatggcatcggcggaaaccagcggtttgtaacgcgccaaatccgcatccgctttggcaagcgttgcctgtgccgttgccagttgcgcgcgcgcgctttccagacctgcttcataagtggaactgtcgatctgatacagcggctgcccggcgcggacataactgccttcttggaacaggcgtttttggatgatgccgccgacttgggcgcggacatcggcggtacgcagcgattccaaacgccccggcaactcgacggtcaatgcgacggtttgcggatggacggttacgacgccgacgacgggcgcgggggcttcccgacccgcaggctgcccgccctgcgccgcgtctctgcctttaccgcaagacgacagtgccaatgcaacggcggcagccaacgcggccgcacgcatcgccttagaagcataaaaagccattatttatcctatctgtctggtttgatgtaaagggttttgccaatcaacaggcattcttatttcaggatataaaaaccgcctgctttgatacccgaatgttcgaacgggttgcaaagcaggttatacctgttttcaaagttgagatgcagtctcaattttatgggtttcattatacatacacgattgcacggataaaagtcttttttataatccgccctcgtcaaaccgacccgaaacgaaaacgccattatgagaaaaaccaaaaccgaagccttgaaaaccaaagaacacctgatgcttgccgccttggaaaccttttaccgcaaagggattgcccgcacctcgctcaacgaaatcgcccaagccgccggcgtaacgcgcggcgcgctctattggcatttcaaaaataaggaagacttgtttgacgcgttgttccaacgtatctgcgacgacatcgaaaactgcatcgcgcaagatgccgcagatgccgaaggaggttcttggacggtattccgccacacgctgctgcactttttcgagcggctgcaaagcaacgacatctactacaaattccacaacatcctgtttttaaaatgcgaacacacggagcaaaacgccgccgttatcgccattgcccgcaagcatcaggcaatctggcgcgagaaaattaccgccgttttgaccgaagcggtggaaaatcaggatttggctgacgatttggacaaggaaacggcggtcatcttcatcaaatcgacgttggacgggctgatttggcgttggttctcttccggcgaaagtttcgatttgggcaaaaccgccccgcgcatcatcgggataatgatggacaacttggaaaaccatccctgcctgcgccggaaataatcaagccttggtaacaatgccgtctgaaacaaacaaaccctttcaaacggcatcaaaatgacacaaagcattcttctaaaaatacatattcactaaattgcatttttaatttcccctatcatcggtcttggtcaaaatgtccgttttcttctgaataaacttctaacaaataatgttcaatgaacgttttatctgtcatcaacgatacatctttggcaatgtcttcatacgactcaaaatcatcttcatgccatggatcatatttatccatgattttttgaatttcatttttcatatcatttaccttccaatatttatttacaattaataacaataccattcgaatgtaaactgcatttttctccggcattcttgcaaacaaaaaccgaaaatcccgtcattcccgcgcaggcgggaattc

SEQ ID NO: 11 - protein, 210 aa, Neisseria gonorrhoeae
  VARIANT 39: note = Xaa is A in wt MtrR gene; Xaa is T in +A39T MtrR mutant
  MUTAGEN 45: note = Mutation at residue 45 - G45D
MRKTKTEALKTKEHLMLAALETFYRKGIARTSLNEIAQAAGVTRDALYWHFKNKEDLFDALFQRICDDIENCIAQDAADAEGGSWTVFRHTLLHFFERLQSNDIHYKFHNILFLKCEHTEQNAAVIAIARKHQAIWREKITAVLTEAVENQDLADDLDKETAVIFIKSTLDGLIWRWFSSGESFDLGKTAPRIIGIMMDNLENHPCLRRK

SEQ ID NO: 12 - DNA, 633 bp, Neisseria gonorrhoeae
  source 1..633: mol_type = other DNA
atgagaaaaaccaaaaccgaagccttgaaaaccaaagaacacctgatgcttgccgccttggaaaccttttaccgcaaagggattgcccgcacctcgctcaacgaaatcgcccaagccgccggcgtaacgcgcggcgcgctctattggcatttcaaaaataaggaagacttgtttgacgcgttgttccaacgtatctgcgacgacatcgaaaactgcatcgcgcaagatgccgcagatgccgaaggaggttcttggacggtattccgccacacgctgctgcactttttcgagcggctgcaaagcaacgacatctactacaaattccacaacatcctgtttttaaaatgcgaacacacggagcaaaacgccgccgttatcgccattgcccgcaagcatcaggcaatctggcgcgagaaaattaccgccgttttgaccgaagcggtggaaaatcaggatttggctgacgatttggacaaggaaacggcggtcatcttcatcaaatcgacgttggacgggctgatttggcgttggttctcttccggcgaaagtttcgatttgggcaaaaccgccccgcgcatcatcgggataatgatggacaacttggaaaaccatccctgcctgcgccggaaataa

SEQ ID NO: 13 - DNA, 55 bp, synthetic construct
  misc_feature 1..55: note = Synthetic
  source 1..55: mol_type = other DNA
gaaattaatacgactcactatagggtgtattatgccggtctgaattacaaaaatg

SEQ ID NO: 14 - DNA, 31 bp, synthetic construct
  misc_feature 1..31: note = Synthetic
  source 1..31: mol_type = other DNA
gtacctgatggtttttcaatggatcggtatc

SEQ ID NO: 15 - DNA, 56 bp, synthetic construct
  misc_feature 1..56: note = Synthetic
  source 1..56: mol_type = other DNA
gaaattaatacgactcactataggtgtgtattatgccggtctgaattacaaaaatg

SEQ ID NO: 16 - DNA, 32 bp, synthetic construct
  source 1..32: mol_type = other DNA
  misc_feature 1..32: note = Synthetic
tacctgatggtttttcaatggatcggtatcac

SEQ ID NO: 17 - DNA, 55 bp, synthetic construct
  misc_feature 1..55: note = Synthetic
  source 1..55: mol_type = other DNA
gaaattaatacgactcactataggcatcagctatgcccatggtttcgactttgtc

SEQ ID NO: 18 - DNA, 31 bp, synthetic construct
  source 1..31: mol_type = other DNA
  misc_feature 1..31: note = Synthetic
gaagtgcgcttggaaaaatcgtaatcgacac

SEQ ID NO: 19 - DNA, 55 bp, synthetic construct
  source 1..55: mol_type = other DNA
  misc_feature 1..55: note = Synthetic
gaaattaatacgactcactataggcactgcttcctaccgcttcggtaatacagtc

SEQ ID NO: 20 - DNA, 32 bp, synthetic construct
  source 1..32: mol_type = other DNA
  misc_feature 1..32: note = Synthetic
gaagtgcgcttggaaaaatcgtaatcgacacc

SEQ ID NO: 21 - DNA, 56 bp, synthetic construct
  source 1..56: mol_type = other DNA
  misc_feature 1..56: note = Synthetic
gaaattaatacgactcactataggaataataatgtggcttcgcaattgggtatttt

SEQ ID NO: 22 - DNA, 30 bp, synthetic construct
  source 1..30: mol_type = other DNA
  misc_feature 1..30: note = Synthetic
catatttaagggcataatttccgaaaaagc

SEQ ID NO: 23 - DNA, 56 bp, synthetic construct
  misc_feature 1..56: note = Synthetic
  source 1..56: mol_type = other DNA
gaaattaatacgactcactataggataataatgtggcttcgcaattgggtattttc

SEQ ID NO: 24 - DNA, 30 bp, synthetic construct
  misc_feature 1..30: note = Synthetic
  source 1..30: mol_type = other DNA
gcatatttaagggcataatttccgaaaaag

SEQ ID NO: 25 - RNA, 64 nt, synthetic construct
  misc_feature 1..64: note = Synthetic
  source 1..64: mol_type = other RNA
gatttagactaccccaaaaacgaaggggactaaaactcgcatatttaagggcataatttccgaa

SEQ ID NO: 26 - RNA, 64 nt, synthetic construct
  misc_feature 1..64: note = Synthetic
  source 1..64: mol_type = other RNA
gatttagactaccccaaaaacgaaggggactaaaacgatttgatcatagctggtatgttcgcgt

SEQ ID NO: 27 - RNA, 64 nt, synthetic construct
  source 1..64: mol_type = other RNA
  misc_feature 1..64: note = Synthetic
gatttagactaccccaaaaacgaaggggactaaaacataggcggacttgctgttttgactcgga

SEQ ID NO: 28 - protein, 1152 aa, Leptotrichia wadei
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 29 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRTSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 30 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 31 - protein, 1285 aa, Rhodobacter capsulatus; identical to SEQ ID NO: 30

SEQ ID NO: 32 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 33 - protein, 1285 aa, unidentified
  source 1..1285: note = Herbinix hemicellulosilytica
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 34 - protein, 1344 aa, Eubacterium sp.
MLRRDKEVKKLYNVFNQIQVGTKPKKWNNDEKLSPEENERRAQQKNIKMKNYKWREACSKYVESSQRIINDVIFYSYRKAKNKLRYMRKNEDILKKMQEAEKLSKFSGGKLEDFVAYTLRKSLVVSKYDTQEFDSLAAMVVFLECIGKNNISDHEREIVCKLLELIRKDFSKLDPNVKGSQGANIVRSVRNQNMIVQPQGDRFLFPQVYAKENETVTNKNVEKEGLNEFLLNYANLDDEKRAESLRKLRRILDVYFSAPNHYEKDMDITLSDNIEKEKFNVWEKHECGKKETGLFVDIPDVLMEAEAENIKLDAVVEKRERKVLNDRVRKQNIICYRYTRAVVEKYNSNEPLFFENNAINQYWIHHIENAVERILKNCKAGKLFKLRKGYLAEKVWKDAINLISIKYIALGKAVYNFALDDIWKDKKNKELGIVDERIRNGITSFDYEMIKAHENLQRELAVDIAFSVNNLARAVCDMSNLGNKESDFLLWKRNDIADKLKNKDDMASVSAVLQFFGGKSSWDINIFKDAYKGKKKYNYEVRFIDDLRKAIYCARNENFHFKTALVNDEKWNTELFGKIFERETEFCLNVEKDRFYSNNLYMFYQVSELRNMLDHLYSRSVSRAAQVPSYNSVIVRTAFPEYITNVLGYQKPSYDADTLGKWYSACYYLLKEIYYNSFLQSDRALQLFEKSVKTLSWDDKKQQRAVDNFKDHFSDIKSACTSLAQVCQIYMTEYNQQNNQIKKVRSSNDSIFDQPVYQHYKVLLKKAIANAFADYLKNNKDLFGFIGKPFKANEIREIDKEQFLPDWTSRKYEALCIEVSGSQELQKWYIVGKFLNARSLNLMVGSMRSYIQYVTDIKRRAASIGNELHVSVHDVEKVEKWVQVIEVCSLLASRTSNQFEDYFNDKDDYARYLKSYVDFSNVDMPSEYSALVDFSNEEQSDLYVDPKNPKVNRNIVHSKLFAADHILRDIVEPVSKDNIEEFYSQKAEIAYCKIKGKEITAEEQKAVLKYQKLKNRVELRDIVEYGEIINELLGQLINWSFMRERDLLYFQLGFHYDCLRNDSKKPEGYKNIKVDENSIKDAILYQIIGMYVNGVTVYAPEKDGDKLKEQCVKGGVGVKVSAFHRYSKYLGLNEKTLYNAGLEIFEVVAEHEDIINLRNGIDHFKYYLGDYRSMLSIYSEVFDRFFTYDIKYQKNVLNLLQNILLRHNVIVEPILESGFKTIGEQTKPGAKLSIRSIKSDTFQYKVKGGTLITDAKDERYLETIRKILYYAENEEDNLKKSVVVTNADKYEKNKESDDQNKQKEKKNKDNKGKKNEETKSDAEKNNNERLSYNPFANLNFKLSN

SEQ ID NO: 35 - protein, 600 aa, unidentified
  source 1..600: note = Eubacteriaceae bacterium CHKCI004
MKISKESHKRTAVAVMEDRVGGVVYVPGGSGIDLSNNLKKRSMDTKSLYNVFNQIQAGTAPSEYEWKDYLSEAENKKREAQKMIQKANYELRRECEDYAKKANLAVSRIIFSKKPKKIFSDDDIISHMKKQRLSKFKGRMEDFVLIALRKSLVVSTYNQEVFDSRKAATVFLKNIGKKNISADDERQIKQLMALIREDYDKWNPDKDSSDKKESSGTKVIRSIEHQNMVIQPEKNKLSLSKISNVGKKTKTKQKEKAGLDAFLKEYAQIDENSRMEYLKKLRRLLDTYFAAPSSYIKGAAVSLPENINFSSELNVWERHEAAKKVNINFVEIPESLLNAEQNNNKINKVEQEHSLEQLRTDIRRRNITCYHFANALAADERYHTLFFENMAMNQFWIHHMENAVERILKKCNVGTLFKLRIGYLSEKVWKDMLNLLSIKYIALGKAVYHFALDDIWKADIWKDASDKNSGKINDLTLKGISSFDYEMVKAQEDLQREMAVGVAFSTNNLARVTCKMDDLSDAESDFLLWNKEAIRRHVKYTEKGEILSAILQFFGGRSLWDESLFEKAYSDSNYELKFLDDLKRAIYAARNETFHFKTAA

SEQ ID NO: 36 - protein, 1276 aa, Blautia sp.
  UNSURE 1..1276: note = Blautia sp. Marseille-P2398
MKISKVDHVKSGIDQKLSSQRGMLYKQPQKKYEGKQLEEHVRNLSRKAKALYQVFPVSGNSKMEKELQIINSFIKNILLRLDSGKTSEEIVGYINTYSVASQISGDHIQELVDQHLKESLRKYTCVGDKRIYVPDIIVALLKSKFNSETLQYDNSELKILIDFIREDYLKEKQIKQIVHSIENNSTPLRIAEINGQKRLIPANVDNPKKSYIFEFLKEYAQSDPKGQESLLQHMRYLILLYLYGPDKITDDYCEEIEAWNFGSIVMDNEQLFSEEASMLIQDRIYVNQQIEEGRQSKDTAKVKKNKSKYRMLGDKIEHSINESVVKHYQEACKAVEEKDIPWIKYISDHVMSVYSSKNRVDLDKLSLPYLAKNTWNTWISFIAMKYVDMGKGVYHFAMSDVDKVGKQDNLIIGQIDPKFSDGISSFDYERIKAEDDLHRSMSGYIAFAVNNFARAICSDEFRKKNRKEDVLTVGLDEIPLYDNVKRKLLQYFGGASNWDDSIIDIIDDKDLVACIKENLYVARNVNFHFAGSEKVQKKQDDILEEIVRKETRDIGKHYRKVFYSNNVAVFYCDEDIIKLMNHLYQREKPYQAQIPSYNKVISKTYLPDLIFMLLKGKNRTKISDPSIMNMFRGTFYFLLKEIYYNDFLQASNLKEMFCEGLKNNVKNKKSEKPYQNFMRRFEELENMGMDFGEICQQIMTDYEQQNKQKKKTATAVMSEKDKKIRTLDNDTQKYKHFRTLLYIGLREAFIIYLKDEKNKEWYEFLREPVKREQPEEKEFVNKWKLNQYSDCSELILKDSLAAAWYVVAHFINQAQLNHLIGDIKNYIQFISDIDRRAKSTGNPVSESTEIQIERYRKILRVLEFAKFFCGQITNVLTDYYQDENDFSTHVGHYVKFEKKNMEPAHALQAFSNSLYACGKEKKKAGFYYDGMNPIVNRNITLASMYGNKKLLENAMNPVTEQDIRKYYSLMAELDSVLKNGAVCKSEDEQKNLRHFQNLKNRIELVDVLTLSELVNDLVAQLIGWVYIRERDMMYLQLGLHYIKLYFTDSVAEDSYLRTLDLEEGSIADGAVLYQIASLYSFNLPMYVKPNKSSVYCKKHVNSVATKFDIFEKEYCNGDETVIENGLRLFENINLHKDMVKFRDYLAHFKYFAKLDESILELYSKAYDFFFSYNIKLKKSVSYVLTNVLLSYFINAKLSFSTYKSSGNKTVQHRTTKISVVAQTDYFTYKLRSIVKNKNGVESIENDDRRCEVVNIAARDKEFVDEVCNVINYNSDK

SEQ ID NO: 37 - protein, 1385 aa, unidentified
  source 1..1385: note = Leptotrichia sp. oral taxon 879
MGNLFGHKRWYEVRDKKDFKIKRKVKVKRNYDGNKYILNINENNNKEKIDNNKFIGEFVNYKKNNNVLKEFKRKFHAGNILFKLKGKEEIIRIENNDDFLETEEVVLYIEVYGKSEKLKALEITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDNKIDVILTNFMEIREKIKSNLEIMGFVKFYLNVSGDKKKSENKKMFVEKILNTNVDLTVEDIVDFIVKELKFWNITKRIEKVKKFNNEFLENRRNRTYIKSYVLLDKHEKFKIERENKKDKIVKFFVENIKNNSIKEKIEKILAEFKINELIKKLEKELKKGNCDTEIFGIFKKHYKVNFDSKKFSNKSDEEKELYKIIYRYLKGRIEKILVNEQKVRLKKMEKIEIEKILNESILSEKILKRVKQYTLEHIMYLGKLRHNDIVKMTVNTDDFSRLHAKEELDLELITFFASTNMELNKIFNGKEKVTDFFGFNLNGQKITLKEKVPSFKLNILKKLNFINNENNIDEKLSHFYSFQKEGYLLRNKILHNSYGNIQETKNLKGEYENVEKLIKELKVSDEEISKSLSLDVIFEGKVDIINKINSLKIGEYKDKKYLPSFSKIVLEITRKFREINKDKLFDIESEKIILNAVKYVNKILYEKITSNEENEFLKTLPDKLVKKSNNKKENKNLLSIEEYYKNAQVSSSKGDKKAIKKYQNKVTNAYLEYLENTFTEIIDFSKFNLNYDEIKTKIEERKDNKSKIIIDSISTNINITNDIEYIISIFALLNSNTYINKIRNRFFATSVWLEKQNGTKEYDYENIISILDEVLLINLLRENNITDILDLKNAIIDAKIVENDETYIKNYIFESNEEKLKKRLFCEELVDKEDIRKIFEDENFKFKSFIKKNEIGNFKINFGILSNLECNSEVEAKKIIGKNSKKLESFIQNIIDEYKSNIRTLFSSEFLEKYKEEIDNLVEDTESENKNKFEKIYYPKEHKNELYIYKKNLFLNIGNPNFDKIYGLISKDIKNVDTKILFDDDIKKNKISEIDAILKNLNDKLNGYSNDYKAKYVNKLKENDDFFAKNIQNENYSSFGEFEKDYNKVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFRLIGNNDILERLMKPKKVSVLELESYNSDYIKNLIIELLTKIENTNDTL

SEQ ID NO: 38 - protein, 500 aa, Chloroflexus aggregans
MTDQVRREEVAAGELADTPLAAAQTPAADAAVAATPAPAEAVAPTPEQAVDQPATTGESEAPVTTAQAAAHEAEPAEATGASFTPVSEQQPQKPRRLKDLQPGMELEGKVTSIALYGIFVDVGVGRDGLVHISEMSDRRIDTPSELVQIGDTVKVWVKSVDLDARRISLTMLNPSRGEKPRRSRQSQPAQPQPRRQEVDREKLASLKVGEIVEGVITGFAPFGAFADIGVGKDGLIHISELSEGRVEKPEDAVKVGERYQFKVLEIDGEGTRISLSLRRAQRTQRMQQLEPGQIIEGTVSGIATFGAFVDIGVGRDGLVHISALAPHRVAKVEDVVKVGDKVKVKVLGVDPQSKRISLTMRLEEEQPATTAGDEAAEPAEEVTPTRRGNLERFAAAAQTARERSERGERSERGERRERRERRPAQSSPDTYIVGEDDDESFEGNATIEDLLTKFGGSSSRRDRDRRRRHEDDDDEEMERPSNRRQREAIRRTLQQIGYDE

SEQ ID NO: 39 - protein, 249 aa, Demequina aurantiaca
MDLTWHALLILFIVALLAGFLDTLAGGGGLLTVPALLLTGIPPLQALGTNKLQSSFGTGMATYQVIRKKRVHWRDVRWPMVWAFLGSAAGAVAVQFIDTDALLIIIPVVLALVAAYFLFVPKSHLPPPEPRMSDPAYEATLVPIIGAYDGAFGPGTGSLYALSGVALRAKTLVQSTAIAKTLNFATNFAALLVFAFAGHMLWTVGAVMIAGQLIGAYAGSHMLFRVNPLVLRVLIVVMSLGMLIRVLLD

SEQ ID NO: 40 - protein, 1235 aa, Thalassospira sp.
MRIIKPYGRSHVEGVATQEPRRKLRLNSSPDISRDIPGFAQSHDALIIAQWISAIDKIATKPKPDKKPTQAQINLRTTLGDAAWQHVMAENLLPAATDPAIREKLHLIWQSKIAPWGTARPQAEKDGKPTPKGGWYERFCGVLSPEAITQNVARQIAKDIYDHLHVAAKRKGREPAKQGESSNKPGKFKPDRKRGLIEERAESIAKNALRPGSHAPCPWGPDDQATYEQAGDVAGQIYAAARDCLEEKKRRSGNRNTSSVQYLPRDLAAKILYAQYGRVFGPDTTIKAALDEQPSLFALHKAIKDCYHRLINDARKRDILRILPRNMAALFRLVRAQYDNRDINALIRLGKVIHYHASEQGKSEHHGIRDYWPSQQDIQNSRFWGSDGQADIKRHEAFSRIWRHIIALASRTLHDWADPHSQKFSGENDDILLLAKDAIEDDVFKAGHYERKCDVLFGAQASLFCGAEDFEKAILKQAITGTGNLRNATFHFKGKVRFEKELQELTKDVPVEVQSAIAALWQKDAEGRTRQIAETLQAVLAGHFLTEEQNRHIFAALTAAMAQPGDVPLPRLRRVLARHDSICQRGRILPLSPCPDRAKLEESPALTCQYTVLKMLYDGPFRAWLAQQNSTILNHYIDSTIARTDKAARDMNGRKLAQAEKDLITSRAADLPRLSVDEKMGDFLARLTAATATEMRVQRGYQSDGENAQKQAAFIGQFECDVIGRAFADFLNQSGFDFVLKLKADTPQPDAAQCDVTALIAPDDISVSPPQAWQQVLYFILHLVPVDDASHLLHQIRKWQVLEGKEKPAQIAHDVQSVLMLYLDMHDAKFTGGAALHGIEKFAEFFAHAADFRAVFPPQSLQDQDRSIPRRGLREIVRFGHLPLLQHMSGTVQITHDNVVAWQAARTAGATGMSPIARRQKQREELHALAVERTARFRNADLQNYMHALVDVIKHRQLSAQVTLSDQVRLHRLMMGVLGRLVDYAGLWERDLYFVVLALLYHHGATPDDVFKGQGKKNLADGQVVAALKPKNRKAAAPVGVFDDLDHYGIYQDDRQSIRNGLSHFNMLRGGKAPDLSHWVNQTRSLVAHDRKLKNAVAKSVIEMLAREGFDLDWGIQTDRGQHILSHGKIRTRQAQHFQKSRLHIVKKSAKPDKNDTVKIRENLHGDAMVERVVQLFAAQVQKRYDITVEKRLDHLFLKPQDQKGKNGIHTHNGWSKTEKKRRPSRENRKGNHEN

SEQ ID NO: 41 - protein, 1312 aa, Pseudobutyrivibrio sp.
MKFSKESHRKTAVGVTESNGIIGLLYKDPLNEKEKIEDVVNQRANSTKRLFNLFGTEATSKDISRASKDLAKVVNKAIGNLKGNKKFNKKEQITKGLNTKIIVEELKNVLKDEKKLIVNKDIIDEACSRLLKTSFRTAKTKQAVKMILTAVLIENTNLSKEDEAFVHEYFVKKLVNEYNKTSVKKQIPVALSNQNMVIQPNSVNGTLEISETKKSKETKTTEKDAFRAFLRDYATLDENRRHKMRLCLRNLVNLYFYGETSVSKDDFDEWRDHEDKKQNDELFVKKIVSIKTDRKGNVKEVLDVDATIDAIRTNNIACYRRALAYANENPDVFFSDTMLNKFWIHHVENEVERIYGHINNNTGDYKYQLGYLSEKVWKGIINYLSIKYIAEGKAVYNYAMNALAKDNNSNAFGKLDEKFVNGITSFEYERIKAEETLQRECAVNIAFAANHLANATVDLNEKDSDFLLLKHEDNKDTLGAVARPNILRNILQFFGGKSRWNDFDFSGIDEIQLLDDLRKMIYSLRNSSFHFKTENIDNDSWNTKLIGDMFAYDFNMAGNVQKDKMYSNNVPMFYSTSDIEKMLDRLYAEVHERASQVPSFNSVFVRKNFPDYLKNDLKITSAFGVDDALKWQSAVYYVCKEIYYNDFLQNPETFTMLKDYVQCLPIDIDKSMDQKLKSERNAHKNFKEAFATYCKECDSLSAICQMIMTEYNNQNKGNRKVISARTKDGDKLIYKHYKMILFEALKNVFTIYLEKNINTYGFLKKPKLINNVPAIEEFLPNYNGRQYETLVNRITEETELQKWYIVGRLLNPKQVNQLIGNFRSYVQYVNDVARRAKQTGNNLSNDNIAWDVKNIIQIFDVCTKLNGVTSNILEDYFDDGDDYARYLKNFVDYTNKNNDHSATLLGDFCAKEIDGIKIGIYHDGTNPIVNRNIIQCKLYGATGIISDLTKDGSILSVDYEIIKKYMQMQKEIKVYQQKGICKTKEEQQNLKKYQELKNIVELRNIIDYSEILDELQGQLINWGYLRERDLMYFQLGFHYLCLHNESKKPVGYNNAGDISGAVLYQIVAMYTNGLSLIDANGKSKKNAKASAGAKVGSFCSYSKEIRGVDKDTKEDDDPIYLAGVELFENINEHQQCINLRNYIEHFHYYAKHDRSMLDLYSEVFDRFFTYDMKYTKNVPNMMYNILLQHLVVPAFEFGSSEKRLDDNDEQTKPRAMFTLREKNGLSSEQFTYRLGDGNSTVKLSARGDDYLRAVASLLYYPDRAPEGLIRDAEAEDKFAKINHSNPKSDNRNNRGNFKNPKVQWYNNKTKRK

SEQ ID NO: 42 - protein, 1276 aa, Blautia sp.; identical to SEQ ID NO: 36
  UNSURE 1..1276: note = Blautia sp. Marseille-P2398

SEQ ID NO: 43 - protein, 1168 aa, Leptotrichia sp.
  UNSURE 1..1168: note = Leptotrichia sp. Marseille-P3007
MKITKIDGISHKKYIKEGKLVKSTSEENKTDERLSELLTIRLDTYIKNPDNASEEENRIRRENLKEFFSNKVLYLKDGILYLKDRREKNQLQNKNYSEEDISEYDLKNKNNFLVLKKILLNEDINSEELEIFRNDFEKKLDKINSLKYSLEENKANYQKINENNIKKVEGKSKRNIFYNYYKDSAKRNDYINNIQEAFDKLYKKEDIENLFFLIENSKKHEKYKIRECYHKIIGRKNDKENFATIIYEEIQNVNNMKELIEKVPNVSELKKSQVFYKYYLNKEKLNDENIKYVFCHFVEIEMSKLLKNYVYKKPSNISNDKVKRIFEYQSLKKLIENKLLNKLDTYVRNCGKYSFYLQDGEIATSDFIVGNRQNEAFLRNIIGVSSTAYFSLRNILETENENDITGRMRGKTVKNNKGEEKYISGEIDKLYDNNKQNEVKKNLKMFYSYDFNMNSKKEIEDFFSNIDEAISSIRHGIVHFNLELEGKDIFTFKNIVPSQISKKMFHDEINEKKLKLKIFKQLNSANVFRYLEKYKILNYLNRTRFEFVNKNIPFVPSFTKLYSRIDDLKNSLGIYWKTPKTNDDNKTKEITDAQIYLLKNIYYGEFLNYFMSNNGNFFEITKEIIELNKNDKRNLKTGFYKLQKFENLQEKTPKEYLANIQSLYMINAGNQDEEEKDTYIDFIQKIFLKGFMTYLANNGRLSLIYIGSDEETNTSLAEKKQEFDKFLKKYEQNNNIEIPYEINEFVREIKLGKILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKISDEAKYKISIEELKNYSKKKNEIEENHTTQENLHRKYARPRKDEKFTDEDYKKYEKAIRNIQQYTHLKNKVEFNELNLLQSLLLRILHRLVGYTSIWERDLRFRLKGEFPENQYIEEIFNFDNSKNVKYKNGQIVEKYINFYKELYKDDTEKISIYSDKKVKELKKEKKDLYIRNYIAHFNYIPNAEISLLEMLENLRKLLSYDRKLKNAIMKSIVDILKEYGFVVTFKIEKDKKIRIESLKSEEVVHLKKLKLKDNDKKKEPIKTYRNSKELCKLVKVMFEYKMKEKKSEN

SEQ ID NO: 44 - protein, 1138 aa, unidentified
  source 1..1138: note = Bacteroides ihuae
MRITKVKVKESSDQKDKMVLIHRKVGEGTLVLDENLADLTAPIIDKYKDKSFELSLLKQTLVSEKEMNIPKCDKCTAKERCLSCKQREKRLKEVRGAIEKTIGAVIAGRDIIPRLNIFNEDEICWLIKPKLRNEFTFKDVNKQVVKLNLPKVLVEYSKKNDPTLFLAYQQWIAAYLKNKKGHIKKSILNNRVVIDYSDESKLSKRKQALELWGEEYETNQRIALESYHTSYNIGELVTLLPNPEEYVSDKGEIRPAFHYKLKNVLQMHQSTVFGTNEILCINPIFNENRANIQLSAYNLEVVKYFEHYFPIKKKKKNLSLNQAIYYLKVETLKERLSLQLENALRMNLLQKGKIKKHEFDKNTCSNTLSQIKRDEFFVLNLVEMCAFAANNIRNIVDKEQVNEILSKKDLCNSLSKNTIDKELCTKFYGADFSQIPVAIWAMRGSVQQIRNEIVHYKAEAIDKIFALKTFEYDDMEKDYSDTPFKQYLELSIEKIDSFFIEQLSSNDVLNYYCTEDVNKLLNKCKLSLRRTSIPFAPGFKTIYELGCHLQDSSNTYRIGHYLMLIGGRVANSTVTKASKAYPAYRFMLKLIYNHLFLNKFLDNHNKRFFMKAVAFVLKDNRENARNKFQYAFKEIRMMNNDESIASYMSYIHSLSVQEQEKKGDKNDKVRYNTEKFIEKVFVKGFDDFLSWLGVEFILSPNQEERDKTVTREEYENLMIKDRVEHSINSNQESHIAFFTFCKLLDANHLSDLRNEWIKFRSSGDKEGFSYNFAIDIIELCLLTVDRVEQRRDGYKEQTELKEYLSFFIKGNESENTVWKGFYFQQDNYTPVLYSPIELIRKYGTLELLKLIIVDEDKITQGEFEEWQTLKKVVEDKVTRRNELHQEWEDMKNKSSFSQEKCSIYQKLCRDIDRYNWLDNKLHLVHLRKLHNLVIQILSRMARFIALWDRDFVLLDASRANDDYKLLSFFNFRDFINAKKTKTDDELLAEFGSKIEKKNAPFIKAEDVPLMVECIEAKRSFYQKVFFRNNLQVLADRNFIAHYNYISKTAKCSLFEMIIKLRTLMYYDRKLRNAVVKSIANVFDQNGMVLQLSLDDSHELKVDKVISKRIVHLKNNNIMTDQVPEEYYKICRRLLEMKK

SEQ ID NO: 45 - protein, 204 aa, unidentified
  source 1..204: note = Listeria riparia
MHDAWAENPKKPQSDAFLKEYKACCEAIDTYNWHKNKATLVYVNELHHLLIDILGRLVGYVAIADRDFQCMANQYLKSSGHTERVDSWINTIRKNRPDYIEKLDIFMNKAGLFVSEKNGRNYIAHLNYLSPKHKYSLLYLFEKLREMLKYDRKLKNAVTKSLIDLLDKHGMCVVFANLKNNKHRLVIASLKPKKIETFKWKKIK

SEQ ID NO: 46 - protein, 1082 aa, Insolitispirillum peregrinum
MRIIRPYGSSTVASPSPQDAQPLRSLQRQNGTFDVAEFSRRHPELVLAQWVAMLDKIIRKPAPGKNSTALPRPTAEQRRLRQQVGAALWAEMQRHTPVPPELKAVWDSKVHPYSKDNAPATAKTPSHRGRWYDRFGDPETSAATVAEGVRRHLLDSAQPFRANGGQPKGKGVIEHRALTIQNGTLLHHHQSEKAGPLPEDWSTYRADELVSTIGKDARWIKVAASLYQHYGRIFGPTTPISEAQTRPEFVLHTAVKAYYRRLFKERKLPAERLERLLPRTGEALRHAVTVQHGNRSLADAVRIGKILHYGWLQNGEPDPWPDDAALYSSRYWGSDGQTDIKHSEAVSRVWRRALTAAQRTLTSWLYPAGTDAGDILLIGQKPDSIDRNRLPLLYGDSTRHWTRSPGDVWLFLKQTLENLRNSSFHFKTLSAFTSHLDGTCESEPAEQQAAQALWQDDRQQDHQQVFLSLRALDATTYLPTGPLHRIVNAVQSTDATLPLPRFRRVVTRAANTRLKGFPVEPVNRRTMEDDPLLRCRYGVLKLLYERGFRAWLETRPSIASCLDQSLKRSTKAAQTINGKNSPQGVEILSRATKLLQAEGGGGHGIHDLFDRLYAATAREMRVQVGYHHDAEAARQQAEFIEDLKCEVVARAFCAYLKTLGIQGDTFRRQPEPLPTWPDLPDLPSSTIGTAQAALYSVLHLMPVEDVGSLLHQLRRWLVALQARGGEDGTAITATIPLLELYLNRHDAKFSGGGAGTGLRWDDWQVFFDCQATFDRVFPPGPALDSHRLPLRGLREVLRFGRVNDLAALIGQDKITAAEVDRWHTAEQTIAAQQQRREALHEQLSRKKGTDAEVDEYRALVTAIADHRHLTAHVTLSNVVRLHRLMTTVLGRLVDYGGLWERDLTFVTLYEAHRLGGLRNLLSESRVNKFLDGQTPAALSKKNNAEENGMISKVLGDKARRQIRNDFAHFNMLQQGKKTINLTDEINNARKLMAHDRKLKNAITRSVTTLLQQDGLDIVWTMDASHRLTDAKIDSRNAIHLHKTHNRANIREPLHGKSYCRWVAALFGATSTPSATKKSDKIR

SEQ ID NO: 47 - protein, 1001 aa, synthetic construct
  REGION 320..321: note = misc_feature - Xaa can be any naturally occurring amino acid
  VARIANT 629: note = Xaa can be any naturally occurring amino acid
  VARIANT 75: note = Xaa can be any naturally occurring amino acid
  VARIANT 923: note = Xaa can be any naturally occurring amino acid
  VARIANT 174: note = Xaa can be any naturally occurring amino acid
  REGION 587..588: note = misc_feature - Xaa can be any naturally occurring amino acid
  REGION 1..1001: note = Synthetic bacterial consensus sequence
  VARIANT 59: note = Xaa can be any naturally occurring amino acid
  VARIANT 22: note = Xaa can be any naturally occurring amino acid
  VARIANT 873: note = Xaa can be any naturally occurring amino acid
  VARIANT 287: note = Xaa can be any naturally occurring amino acid
  VARIANT 20: note = Xaa can be any naturally occurring amino acid
  VARIANT 199: note = Xaa can be any naturally occurring amino acid
  REGION 414..415: note = misc_feature - Xaa can be any naturally occurring amino acid
  VARIANT 843: note = Xaa can be any naturally occurring amino acid
  VARIANT 682: note = Xaa can be any naturally occurring amino acid
  REGION 277..278: note = misc_feature - Xaa can be any naturally occurring amino acid
  VARIANT 532: note = Xaa can be any naturally occurring amino acid
  VARIANT 116: note = Xaa can be any naturally occurring amino acid
  VARIANT 446: note = Xaa can be any naturally occurring amino acid
  VARIANT 622: note = Xaa can be any naturally occurring amino acid
  VARIANT 790: note = Xaa can be any naturally occurring amino acid
  VARIANT 706: note = Xaa can be any naturally occurring amino acid
  VARIANT 655: note = Xaa can be any naturally occurring amino acid
  VARIANT 121: note = Xaa can be any naturally occurring amino acid
  VARIANT 936: note = Xaa can be any naturally occurring amino acid
  REGION 547..548: note = misc_feature - Xaa can be any naturally occurring amino acid
  VARIANT 104: note = Xaa can be any naturally occurring amino acid
  VARIANT 378: note = Xaa can be any naturally occurring amino acid
  VARIANT 145: note = Xaa can be any naturally occurring amino acid
  VARIANT 86: note = Xaa can be any naturally occurring amino acid
  REGION 14..16: note = misc_feature - Xaa can be any naturally occurring amino acid
  VARIANT 576: note = Xaa can be any naturally occurring amino acid
  VARIANT 29: note = Xaa can be any naturally occurring amino acid
  VARIANT 192: note = Xaa can be any naturally occurring amino acid
  VARIANT 333: note = Xaa can be any naturally occurring amino acid
  REGION 52..53: note = misc_feature - Xaa can be any naturally occurring amino acid
  VARIANT 688: note = Xaa can be any naturally occurring amino acid
  VARIANT 773: note = Xaa can be any naturally occurring amino acid
  REGION 194..195: note = misc_feature - Xaa can be any naturally occurring amino acid
  REGION 448..449: note = misc_feature - Xaa can be any naturally occurring amino acid
  VARIANT 514: note = Xaa can be any naturally occurring amino acid
  VARIANT 786: note = Xaa can be any naturally occurring amino acid
  VARIANT 206: note = Xaa can be any naturally occurring amino acid
  REGION 217..219: note = misc_feature - Xaa can be any naturally occurring amino acid
  VARIANT 866: note = Xaa can be any naturally occurring amino acid
  VARIANT 402: note = Xaa can be any naturally occurring amino acid
  VARIANT 627: note = Xaa can be any naturally occurring amino acid
  VARIANT 282: note = Xaa can be any naturally occurring amino acid
  VARIANT 418: note = Xaa can be any naturally occurring amino acid
  REGION 553..554: note = misc_feature - Xaa can be any naturally occurring amino acid
  VARIANT 367: note = Xaa can be any naturally occurring amino acid
  VARIANT 565: note = Xaa can be any naturally occurring amino acid
  VARIANT 167: note = Xaa can be any naturally occurring amino acid
  VARIANT 834: note = Xaa can be any naturally occurring amino acid
  VARIANT 889: note = Xaa can be any naturally occurring amino acid
  VARIANT 245: note = Xaa can be any naturally occurring amino acid
  VARIANT 451: note = Xaa can be any naturally occurring amino acid
  REGION 851..852: note = misc_feature - Xaa can be any naturally occurring amino acid
  VARIANT 962: note = Xaa can be any naturally occurring amino acid
  VARIANT 698: note = Xaa can be any naturally occurring amino acid
  VARIANT 343: note = Xaa can be any naturally occurring amino acid
  REGION 760..761: note = misc_feature - Xaa can be any naturally occurring amino acid
  VARIANT 996: note = Xaa can be any naturally occurring amino acid
  REGION 374..375: note = misc_feature - Xaa can be any naturally occurring amino acid
  VARIANT 73: note = Xaa can be any naturally occurring amino acid
  VARIANT 233: note = Xaa can be any naturally occurring amino acid
  VARIANT 111: note = Xaa can be any naturally occurring amino acid
  VARIANT 236: note = Xaa can be any naturally occurring amino acid
  VARIANT 371: note = Xaa can be any naturally occurring amino acid
  REGION 78..79: note = misc_feature - Xaa can be any naturally occurring amino acid
  VARIANT 985: note = Xaa can be any naturally occurring amino acid
  VARIANT 467: note = Xaa can be any naturally occurring amino acid
  VARIANT 392: note = Xaa can be any naturally occurring amino acid
  REGION 69..71: note = misc_feature - Xaa can be any naturally occurring amino acid
  VARIANT 36: note = Xaa can be any naturally occurring amino acid
  REGION 89..90: note = misc_feature - Xaa can be any naturally occurring amino acid
  VARIANT 48: note = Xaa can be any naturally occurring amino acid
  VARIANT 133: note = Xaa can be any naturally occurring amino acid
  VARIANT 101: note = Xaa can be any naturally occurring amino acid
  VARIANT 243: note = Xaa can be any naturally occurring amino acid
  VARIANT 723: note = Xaa can be any naturally occurring amino acid
  VARIANT 771: note = Xaa can be any naturally occurring amino acid
  VARIANT 870: note = Xaa can be any naturally occurring amino acid
  VARIANT 896: note = Xaa can be any naturally occurring amino acid
  VARIANT 970: note = Xaa can be any naturally occurring amino acid
  REGION 202..203: note = misc_feature - Xaa can be any naturally occurring amino acid
  VARIANT 779: note = Xaa can be any naturally occurring amino acid
  VARIANT 798: note = Xaa can be any naturally occurring amino acid
  VARIANT 691: note = Xaa can be any naturally occurring amino acid
  VARIANT 11: note = Xaa can be any naturally occurring amino acid
  VARIANT 966: note = Xaa can be any naturally occurring amino acid
  REGION 737..738: note = misc_feature - Xaa can be any naturally occurring amino acid
  VARIANT 262: note = Xaa can be any naturally occurring amino acid
  VARIANT 600: note = Xaa can be any naturally occurring amino acid
  VARIANT 916: note = Xaa can be any naturally occurring amino acid
  VARIANT 662: note = Xaa can be any naturally occurring amino acid
  VARIANT 314: note = Xaa can be any naturally occurring amino acid
  VARIANT 228: note = Xaa can be any naturally occurring amino acid
  VARIANT 800: note = Xaa can be any naturally occurring amino acid
  REGION 7..9: note = misc_feature - Xaa can be any naturally occurring amino acid
  VARIANT 551: note = Xaa can be any naturally occurring amino acid
  VARIANT 57: note = Xaa can be any naturally occurring amino acid
  VARIANT 539: note = Xaa can be any naturally occurring amino acid
  REGION 42..43: note = misc_feature - Xaa can be any naturally occurring amino acid
  REGION 528..529: note = misc_feature - Xaa can be any naturally occurring amino acid
  VARIANT 357: note = Xaa can be any naturally occurring amino acid
  VARIANT 877: note = Xaa can be any naturally occurring amino acid
  REGION 604..605: note = misc_feature - Xaa can be any naturally occurring amino acid
  VARIANT 267: note = Xaa can be any naturally occurring amino acid
  VARIANT 307: note = Xaa can be any naturally occurring amino acid
  VARIANT 148: note = Xaa can be any naturally occurring amino acid
  VARIANT 489: note = Xaa can be any naturally occurring amino acid
  VARIANT 510: note = Xaa can be any naturally occurring amino acid
  VARIANT 500: note = Xaa can be any naturally occurring amino acid
  VARIANT 686: note = Xaa can be any naturally occurring amino acid
  REGION 128..130: note = misc_feature - Xaa can be any naturally occurring amino acid
MKISKVXXXVXKKXXXGKLXKXVNERNRXAKRLSNXLBKYIXXIDKIXKKEXXKKFXAXEEITLKLNQXXXBXLXKAXXDLRKDNXYSXXKKILHNEDINXEEXELLINDXLEKLXKIESXKYSYQKXXXNYXMSVQEHSKKSIXRIXESAKRNKEALDKFLKEYAXLDPRMEXLAKLRKLLELYFYFKNDXIXXEEEXNVXXHKXLKENHPDFVEXXXNKENAELNXYAIEXKKXLKYYFPXKXAKNSNDKIFEKQELKKXWIHQXENAVERILLXXGKVXYKLQXGYLAELWKIRINEIFIKYIXVGKAVAXFALRNXXKBENDILGGKIXKKLNGITSFXYEKIKAEEILQREXAVEVAFAANXLYAXDLXXIRXSILQFFGGASNWDXFLFFHFATSXISDKKWNAELIXXKKXGLVIREKLYSNNVAMFYSKDDLEKLLNXLXXFXLRASQVPSFKKVYVRXBFPQNLLKKFNDEKDDEAYSAXYYLLKEIYYNXFLPYFSANNXFFFXVKNLVLKANKDKFXXAFXDIREMNXGSPIEYLXXTQXNXXNEGRKKEEKEXDFIKFLLQIFXKGFDDYLKNNXXFILKFIPEPTEXIEIXXELQAWYIVGKFLNARKXNLLGXFXSYLKLLDDIELRALRNENIKYQSSNXEKEVLEXCLELIGLLSLDLNDYFBDEXDFAXYXGKXLDFEKKXMKDLAELXPYDQNDGENPIVNRNIXLAKKYGTLNLLEKXXDKVSEKEIKEYYELKKEIEEYXXKGEELHEEWXQXKNRVEXRDILEYXEELXGQIINYNXLXNKVLLYFQLGLHYLLLDILGRLVGYTGIWERDAXLYQIAAMYXNGLPEYIXXKKNDKYKDGQIVGXKINXFKXDKKXLYNAGLELFENXNEHKNIXIRNYIAHFNYLSKAESSLLXYSENLRXLFSYDRKLKNAVXKSLINILLRHGMVLKFKFGTDKKSVXIRSXKKIXHLKSIAKKLYYPEVXVSKEYCKLVKXLLKYK

SEQ ID NO: 48 - protein, 1224 aa, Bergeyella zoohelcum
MENKTSLGNNIYYNPFKPQDKSYFAGYFNAAMENTDSVFRELGKRLKGKEYTSENFFDAIFKENISLVEYERYVKLLSDYFPMARLLDKKEVPIKERKENFKKNFKGIIKAVRDLRNFYTHKEHGEVEITDEIFGVLDEMLKSTVLTVKKKKVKTDKTKEILKKSIEKQLDILCQKKLEYLRDTARKIEEKRRNQRERGEKELVAPFKYSDKRDDLIAAIYNDAFDVYIDKKKDSLKESSKAKYNTKSDPQQEEGDLKIPISKNGVVFLLSLFLTKQEIHAFKSKIAGFKATVIDEATVSEATVSHGKNSICFMATHEIFSHLAYKKLKRKVRTAEINYGEAENAEQLSVYAKETLMMQMLDELSKVPDVVYQNLSEDVQKTFIEDWNEYLKENNGDVGTMEEEQVIHPVIRKRYEDKFNYFAIRFLDEFAQFPTLRFQVHLGNYLHDSRPKENLISDRRIKEKITVFGRLSELEHKKALFIKNTETNEDREHYWEIFPNPNYDFPKENISVNDKDFPIAGSILDREKQPVAGKIGIKVKLLNQQYVSEVDKAVKAHQLKQRKASKPSIQNIIEEIVPINESNPKEAIVFGGQPTAYLSMNDIHSILYEFFDKWEKKKEKLEKKGEKELRKEIGKELEKKIVGKIQAQIQQIIDKDTNAKILKPYQDGNSTAIDKEKLIKDLKQEQNILQKLKDEQTVREKEYNDFIAYQDKNREINKVRDRNHKQYLKDNLKRKYPEAPARKEVLYYREKGKVAVWLANDIKRFMPTDFKNEWKGEQHSLLQKSLAYYEQCKEELKNLLPEKVFQHLPFKLGGYFQQKYLYQFYTCYLDKRLEYISGLVQQAENFKSENKVFKKVENECFKFLKKQNYTHKELDARVQSILGYPIFLERGFMDEKPTIIKGKTFKGNEALFADWFRYYKEYQNFQTFYDTENYPLVELEKKQADRKRKTKIYQQKKNDVFTLLMAKHIFKSVFKQDSIDQFSLEDLYQSREERLGNQERARQTGERNTNYIWNKTVDLKLCDGKITVENVKLKNVGDFIKYEYDQRVQAFLKYEENIEWQAFLIKESKEEENYPYVVEREIEQYEKVRREELLKEVHLIEEYILEKVKDKEILKKGDNQNFKYYILNGLLKQLKNEDVESYKVFNLNTEPEDVNINQLKQEATDLEQKAFVLTYIRNKFAHNQLPKKEFWDYCQEKYGKIEKEKTYAEYFAEVFKKEKEALIK

SEQ ID NO: 49 - protein, 1126 aa, Prevotella intermedia
MEDDKKTTDSIRYELKDKHFWAAFLNLARHNVYITVNHINKILEEGEINRDGYETTLKNTWNEIKDINKKDRLSKLIIKHFPFLEAATYRLNPTDTTKQKEEKQAEAQSLESLRKSFFVFIYKLRDLRNHYSHYKHSKSLERPKFEEGLLEKMYNIFNASIRLVKEDYQYNKDINPDEDFKHLDRTEEEFNYYFTKDNEGNITESGLLFFVSLFLEKKDAIWMQQKLRGFKDNRENKKKMTNEVFCRSRMLLPKLRLQSTQTQDWILLDMLNELIRCPKSLYERLREEDREKFRVPIEIADEDYDAEQEPFKNTLVRHQDRFPYFALRYFDYNEIFTNLRFQIDLGTYHFSIYKKQIGDYKESHHLTHKLYGFERIQEFTKQNRPDEWRKFVKTFNSFETSKEPYIPETTPHYHLENQKIGIRFRNDNDKIWPSLKTNSEKNEKSKYKLDKSFQAEAFLSVHELLPMMFYYLLLKTENTDNDNEIETKKKENKNDKQEKHKIEEIIENKITEIYALYDTFANGEIKSIDELEEYCKGKDIEIGHLPKQMIAILKDEHKVMATEAERKQEEMLVDVQKSLESLDNQINEEIENVERKNSSLKSGKIASWLVNDMMRFQPVQKDNEGKPLNNSKANSTEYQLLQRTLAFFGSEHERLAPYFKQTKLIESSNPHPFLKDTEWEKCNNILSFYRSYLEAKKNFLESLKPEDWEKNQYFLKLKEPKTKPKTLVQGWKNGFNLPRGIFTEPIRKWFMKHRENITVAELKRVGLVAKVIPLFFSEEYKDSVQPFYNYHFNVGNINKPDEKNFLNCEERRELLRKKKDEFKKMTDKEKEENPSYLEFKSWNKFERELRLVRNQDIVTWLLCMELFNKKKIKELNVEKIYLKNINTNTTKKEKNTEEKNGEEKNIKEKNNILNRIMPMRLPIKVYGRENFSKNKKKKIRRNTFFTVYIEEKGTKLLKQGNFKALERDRRLGGLFSFVKTPSKAESKSNTISKLRVEYELGEYQKARIEIIKDMLALEKTLIDKYNSLDTDNFNKMLTDWLELKGEPDKASFQNDVDLLIAVRNAFSHNQYPMRNRIAFANINPFSLSSANTSEEKGLGIANQLKDKTHKTIEKIIEIEKPIETKE

SEQ ID NO: 50 - protein, 1127 aa, Prevotella buccae
MQKQDKLFVDRKKNAIFAFPKYITIMENKEKPEPIYYELTDKHFWAAFLNLARHNVYTTINHINRRLEIAELKDDGYMMGIKGSWNEQAKKLDKKVRLRDLIMKHFPFLEAAAYEMTNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYNAEEEPFKNTLVRHQDRFPYFVLRYFDLNEIFEQLRFQIDLGTYHFSIYNKRIGDEDEVRHLTHHLYGFARIQDFAPQNQPEEWRKLVKDLDHFETSQEPYISKTAPHYHLENEKIGIKFCSAHNNLFPSLQTDKTCNGRSKFNLGTQFTAEAFLSVHELLPMMFYYLLLTKDYSRKESADKVEGIIRKEISNIYAIYDAFANNEINSIADLTRRLQNTNILQGHLPKQMISILKGRQKDMGKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNRLKPKKRQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKALVKDRRLNGLFSFAETTDLNLEEHPISKLSVDLELIKYQTTRISIFEMTLGLEKKLIDKYSTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 51 - protein, 1135 aa, Porphyromonas gingivalis
MNTVPASENKGQSRTVEDDPQYFGLYLNLARENLIEVESHVRIKFGKKKLNEESLKQSLLCDHLLSVDRWTKVYGHSRRYLPFLHYFDPDSQIEKDHDSKTGVDPDSAQRLIRELYSLLDFLRNDFSHNRLDGTTFEHLEVSPDISSFITGTYSLACGRAQSRFAVFFKPDDFVLAKNRKEQLISVADGKECLTVSGFAFFICLFLDREQASGMLSRIRGFKRTDENWARAVHETFCDLCIRHPHDRLESSNTKEALLLDMLNELNRCPRILYDMLPEEERAQFLPALDENSMNNLSENSLDEESRLLWDGSSDWAEALTKRIRHQDRFPYLMLRFIEEMDLLKGIRFRVDLGEIELDSYSKKVGRNGEYDRTITDHALAFGKLSDFQNEEEVSRMISGEASYPVRFSLFAPRYAIYDNKIGYCHTSDPVYPKSKTGEKRALSNPQSMGFISVHDLRKLLLMELLCEGSFSRMQSDFLRKANRILDETAEGKLQFSALFPEMRHRFIPPQNPKSKDRREKAETTLEKYKQEIKGRKDKLNSQLLSAFDMDQRQLPSRLLDEWMNIRPASHSVKLRTYVKQLNEDCRLRLRKFRKDGDGKARAIPLVGEMATFLSQDIVRMIISEETKKLITSAYYNEMQRSLAQYAGEENRRQFRAIVAELRLLDPSSGHPFLSATMETAHRYTEGFYKCYLEKKREWLAKIFYRPEQDENTKRRISVFFVPDGEARKLLPLLIRRRMKEQNDLQDWIRNKQAHPIDLPSHLFDSKVMELLKVKDGKKKWNEAFKDWWSTKYPDGMQPFYGLRRELNIHGKSVSYIPSDGKKFADCYTHLMEKTVRDKKRELRTAGKPVPPDLAADIKRSFHRAVNEREFMLRLVQEDDRLMLMAINKMMTDREEDILPGLKNIDSILDEENQFSLAVHAKVLEKEGEGGDNSLSLVPATIEIKSKRKDWSKYIRYRYDRRVPGLMSHFPEHKATLDEVKTLLGEYDRCRIKIFDWAFALEGAIMSDRDLKPYLHESSSREGKSGEHSTLVKMLVEKKGCLTPDESQYLILIRNKAAHNQFPCAAEMPLIYRDVSAKVGSIEGSSAKDLPEGSSLVDSLWKKYEMIIRKILPILDPENRFFGKLLNNMSQPINDL

SEQ ID NO: 52 - protein, 1115 aa, Bacteroides pyogenes
MESIKNSQKSTGKTLQKDPPYFGLYLNMALLNVRKVENHIRKWLGDVALLPEKSGFHSLLTTDNLSSAKWTRFYYKSRKFLPFLEMFDSDKKSYENRRETAECLDTIDRQKISSLLKEVYGKLQDIRNAFSHYHIDDQSVKHTALIISSEMHRFIENAYSFALQKTRARFTGVFVETDFLQAEEKGDNKKFFAIGGNEGIKLKDNALIFLICLFLDREEAFKFLSRATGFKSTKEKGFLAVRETFCALCCRQPHERLLSVNPREALLMDMLNELNRCPDILFEMLDEKDQKSFLPLLGEEEQAHILENSLNDELCEAIDDPFEMIASLSKRVRYKNRFPYLMLRYIEEKNLLPFIRFRIDLGCLELASYPKKMGEENNYERSVTDHAMAFGRLTDFHNEDAVLQQITKGITDEVRFSLYAPRYAIYNNKIGFVRTSGSDKISFPTLKKKGGEGHCVAYTLQNTKSFGFISIYDLRKILLLSFLDKDKAKNIVSGLLEQCEKHWKDLSENLFDAIRTELQKEFPVPLIRYTLPRSKGGKLVSSKLADKQEKYESEFERRKEKLTEILSEKDFDLSQIPRRMIDEWLNVLPTSREKKLKGYVETLKLDCRERLRVFEKREKGEHPLPPRIGEMATDLAKDIIRMVIDQGVKQRITSAYYSEIQRCLAQYAGDDNRRHLDSIIRELRLKDTKNGHPFLGKVLRPGLGHTEKLYQRYFEEKKEWLEATFYPAASPKRVPRFVNPPTGKQKELPLIIRNLMKERPEWRDWKQRKNSHPIDLPSQLFENEICRLLKDKIGKEPSGKLKWNEMFKLYWDKEFPNGMQRFYRCKRRVEVFDKVVEYEYSEEGGNYKKYYEALIDEVVRQKISSSKEKSKLQVEDLTLSVRRVFKRAINEKEYQLRLLCEDDRLLFMAVRDLYDWKEAQLDLDKIDNMLGEPVSVSQVIQLEGGQPDAVIKAECKLKDVSKLMRYCYDGRVKGLMPYFANHEATQEQVEMELRHYEDHRRRVFNWVFALEKSVLKNEKLRRFYEESQGGCEHRRCIDALRKASLVSEEEYEFLVHIRNKSAHNQFPDLEIGKLPPNVTSGFCECIWSKYKAIICRIIPFIDPERRFFGKLLEQK

SEQ ID NO: 53 - protein, 953 aa, Alistipes sp.
MSNEIGAFREHQFAYAPGNEKQEEATFATYFNLALSNVEGMMFGEVESNPDKIEKSLDTLPPAILRQIASFIWLSKEDHPDKAYSTEEVKVIVTDLVRRLCFYRNYFSHCFYLDTQYFYSDELVDTTAIGEKLPYNFHHFITNRLFRYSLPEITLFRWNEGERKYEILRDGLIFFCCLFLKRGQAERFLNELRFFKRTDEEGRIKRTIFTKYCTRESHKHIGIEEQDFLIFQDIIGDLNRVPKVCDGVVDLSKENERYIKNRETSNESDENKARYRLLIREKDKFPYYLMRYIVDFGVLPCITFKQNDYSTKEGRGQFHYQDAAVAQEERCYNFVVRNGNVYYSYMPQAQNVVRISELQGTISVEELRNMVYASINGKDVNKSVEQYLYHLHLLYEKILTISGQTIKEGRVDVEDYRPLLDKLLLRPASNGEELRRELRKLLPKRVCDLLSNRFDCSEGVSAVEKRLKAILLRHEQLLLSQNPALHIDKIKSVIDYLYLFFSDDEKFRQQPTEKAHRGLKDEEFQMYHYLVGDYDSHPLALWKELEASGRLKPEMRKLTSATSLHGLYMLCLKGTVEWCRKQLMSIGKGTAKVEAIADRVGLKLYDKLKEYTPEQLEREVKLVVMHGYAAAATPKPKAQAAIPSKLTELRFYSFLGKREMSFAAFIRQDKKAQKLWLRNFYTVENIKTLQKRQAAADAACKKLYNLVGEVERVHTNDKVLVLVAQRYRERLLNVGSKCAVTLDNPERQQKLADVYEVQNAWLSIRFDDLDFTLTHVNLSNLRKAYNLIPRKHILAFKEYLDNRVKQKLCEECRNVRRKEDLCTCCSPRYSNLTSWLKENHSESSIEREAATMMLLDVERKLLSFLLDERRKAIIEYGKFIPFSALVKECRLADAGLCGIRNDVLHDNVISYADAIGKLSAYFPKEASEAVEYIRRTKEVREQRREELMANSSQ

SEQ ID NO: 54 - protein, 1322 aa, Prevotella sp.
MSKECKKQRQEKKRRLQKANFSISLTGKHVFGAYFNMARTNFVKTINYILPIAGVRGNYSENQINKMLHALFLIQAGRNEELTTEQKQWEKKLRLNPEQQTKFQKLLFKHFPVLGPMMADVADHKAYLNKKKSTVQTEDETFAMLKGVSLADCLDIICLMADTLTECRNFYTHKDPYNKPSQLADQYLHQEMIAKKLDKVVVASRRILKDREGLSVNEVEFLTGIDHLHQEVLKDEFGNAKVKDGKVMKTFVEYDDFYFKISGKRLVNGYTVTTKDDKPVNVNTMLPALSDFGLLYFCVLFLSKPYAKLFIDEVRLFEYSPFDDKENMIMSEMLSIYRIRTPRLHKIDSHDSKATLAMDIFGELRRCPMELYNLLDKNAGQPFFHDEVKHPNSHTPDVSKRLRYDDRFPTLALRYIDETELFKRIRFQLQLGSFRYKFYDKENCIDGRVRVRRIQKEINGYGRMQEVADKRMDKWGDLIQKREERSVKLEHEELYINLDQFLEDTADSTPYVTDRRPAYNIHANRIGLYWEDSQNPKQYKVFDENGMYIPELVVTEDKKAPIKMPAPRCALSVYDLPAMLFYEYLREQQDNEFPSAEQVIIEYEDDYRKFFKAVAEGKLKPFKRPKEFRDFLKKEYPKLRMADIPKKLQLFLCSHGLCYNNKPETVYERLDRLTLQHLEERELHIQNRLEHYQKDRDMIGNKDNQYGKKSFSDVRHGALARYLAQSMMEWQPTKLKDKEKGHDKLTGLNYNVLTAYLATYGHPQVPEEGFTPRTLEQVLINAHLIGGSNPHPFINKVLALGNRNIEELYLHYLEEELKHIRSRIQSLSSNPSDKALSALPFIHHDRMRYHERTSEEMMALAARYTTIQLPDGLFTPYILEILQKHYTENSDLQNALSQDVPVKLNPTCNAAYLITLFYQTVLKDNAQPFYLSDKTYTRNKDGEKAESFSFKRAYELFSVLNNNKKDTFPFEMIPLFLTSDEIQERLSAKLLDGDGNPVPEVGEKGKPATDSQGNTIWKRRIYSEVDDYAEKLTDRDMKISFKGEWEKLPRWKQDKIIKRRDETRRQMRDELLQRMPRYIRDIKDNERTLRRYKTQDMVLFLLAEKMFTNIISEQSSEFNWKQMRLSKVCNEAFLRQTLTFRVPVTVGETTIYVEQENMSLKNYGEFYRFLTDDRLMSLLNNIVETLKPNENGDLVIRHTDLMSELAAYDQYRSTIFMLIQSIENLIITNNAVLDDPDADGFWVREDLPKRNNFASLLELINQLNNVELTDDERKLLVAIRNAFSHNSYNIDFSLIKDVKHLPEVAKGILQHLQSMLGVEITK

SEQ ID NO: 55 - protein, 1322 aa, Prevotella sp.; identical to SEQ ID NO: 54

SEQ ID NO: 56 - protein, 1095 aa, Riemerella anatipestifer
MEKPLLPNVYTLKHKFFWGAFLNIARHNAFITICHINEQLGLKTPSNDDKIVDVVCETWNNILNNDHDLLKKSQLTELILKHFPFLTAMCYHPPKKEGKKKGHQKEQQKEKESEAQSQAEALNPSKLIEALEILVNQLHSLRNYYSHYKHKKPDAEKDIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSRILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLMLSKPIRKEIKKHGRVGFISRAITLYFKEKYQDKHQSFYNLSYKLEAKAPLLKREEHYEYWQQNKPQSPTESQRLELHTSDRWKDYLLYKRWQHLEKKLRLYRNQDVMLWLMTLELTKNHFKELNLNYHQLKLENLAVNVQEADAKLNPLNQTLPMVLPVKVYPATAFGEVQYHKTPIRTVYIREEHTKALKMGNFKALVKDRRLNGLFSFIKEENDTQKHPISQLRLRRELEIYQSLRVDAFKETLSLEEKLLNKHTSLSSLENEFRALLEEWKKEYAASSMVTDEHIAFIASVRNAFCHNQYPFYKEALHAPIPLFTVAQPTTEEKDGLGIAEALLKVLREYCEIVKSQI

SEQ ID NO: 57 - protein, 1124 aa, Prevotella aurantiaca
MEDDKKTTGSISYELKDKHFWAAFLNLARHNVYITINHINKLLEIREIDNDEKVLDIKTLWQKGNKDLNQKARLRELMTKHFPFLETAIYTKNKEDKKEVKQEKQAEAQSLESLKDCLFLFLDKLQEARNYYSHYKYSEFSKEPEFEEGLLEKMYNIFGNNIQLVINDYQHNKDINPDEDFKHLDRKGQFKYSFADNEGNITESGLLFFVSLFLEKKDAIWMQQKLNGFKDNLENKKKMTHEVFCRSRILMPKLRLESTQTQDWILLDMLNELIRCPKSLYERLQGDDREKFKVPFDPADEDYNAEQEPFKNTLIRHQDRFPYFVLRYFDYNEIFKNLRFQIDLGTYHFSIYKKLIGGQKEDRHLTHKLYGFERIQEFAKQNRPDEWKAIVKDLDTYETSNKRYISETTPHYHLENQKIGIRFRNGNKEIWPSLKTNDENNEKSKYKLDKQYQAEAFLSVHELLPMMFYYLLLKKEKPNNDEINASIVEGFIKREIRNIFKLYDAFANGEINNIDDLEKYCADKGIPKRHLPKQMVAILYDEHKDMVKEAKRKQKEMVKDTKKLLATLEKQTQKEKEDDGRNVKLLKSGEIARWLVNDMMRFQPVQKDNEGKPLNNSKANSTEYQMLQRSLALYNNEEKPTRYFRQVNLIESNNPHPFLKWTKWEECNNILTFYYSYLTKKIEFLNKLKPEDWKKNQYFLKLKEPKTNRETLVQGWKNGFNLPRGIFTEPIREWFKRHQNNSKEYEKVEALDRVGLVTKVIPLFFKEEYFKDKEENFKEDTQKEINDCVQPFYNFPYNVGNIHKPKEKDFLHREERIELWDKKKDKFKGYKEKIKSKKLTEKDKEEFRSYLEFQSWNKFERELRLVRNQDIVTWLLCKELIDKLKIDELNIEELKKLRLNNIDTDTAKKEKNNILNRVMPMELPVTVYEIDDSHKIVKDKPLHTIYIKEAETKLLKQGNFKALVKDRRLNGLFSFVKTNSEAESKRNPISKLRVEYELGEYQEARIEIIQDMLALEEKLINKYKDLPTNKFSEMLNSWLEGKDEADKARFQNDVDFLIAVRNAFSHNQYPMHNKIEFANIKPFSLYTANNSEEKGLGIANQLKDKTKETTDKIKKIEKPIETKE

SEQ ID NO: 58 - protein, 1151 aa, Prevotella saccharolytica
MEDKPFWAAFFNLARHNVYLTVNHINKLLDLEKLYDEGKHKEIFEREDIFNISDDVMNDANSNGKKRKLDIKKIWDDLDTDLTRKYQLRELILKHFPFIQPAIIGAQTKERTTIDKDKRSTSTSNDSLKQTGEGDINDLLSLSNVKSMFFRLLQILEQLRNYYSHVKHSKSATMPNFDEDLLNWMRYIFIDSVNKVKEDYSSNSVIDPNTSFSHLIYKDEQGKIKPCRYPFTSKDGSINAFGLLFFVSLFLEKQDSIWMQKKIPGFKKASENYMKMTNEVFCRNHILLPKIRLETVYDKDWMLLDMLNEVVRCPLSLYKRLTPAAQNKFKVPEKSSDNANRQEDDNPFSRILVRHQNRFPYFVLRFFDLNEVFTTLRFQINLGCYHFAICKKQIGDKKEVHHLIRTLYGFSRLQNFTQNTRPEEWNTLVKTTEPSSGNDGKTVQGVPLPYISYTIPHYQIENEKIGIKIFDGDTAVDTDIWPSVSTEKQLNKPDKYTLTPGFKADVFLSVHELLPMMFYYQLLLCEGMLKTDAGNAVEKVLIDTRNAIFNLYDAFVQEKINTITDLENYLQDKPILIGHLPKQMIDLLKGHQRDMLKAVEQKKAMLIKDTERRLKLLDKQLKQETDVAAKNTGTLLKNGQIADWLVNDMMRFQPVKRDKEGNPINCSKANSTEYQMLQRAFAFYATDSCRLSRYFTQLHLIHSDNSHLFLSRFEYDKQPNLIAFYAAYLKAKLEFLNELQPQNWASDNYFLLLRAPKNDRQKLAEGWKNGFNLPRGLFTEKIKTWFNEHKTIVDISDCDIFKNRVGQVARLIPVFFDKKFKDHSQPFYRYDFNVGNVSKPTEANYLSKGKREELFKSYQNKFKNNIPAEKTKEYREYKNFSLWKKFERELRLIKNQDILIWLMCKNLFDEKIKPKKDILEPRIAVSYIKLDSLQTNTSTAGSLNALAKVVPMTLAIHIDSPKPKGKAGNNEKENKEFTVYIKEEGTKLLKWGNFKTLLADRRIKGLFSYIEHDDIDLKQHPLTKRRVDLELDLYQTCRIDIFQQTLGLEAQLLDKYSDLNTDNFYQMLIGWRKKEGIPRNIKEDTDFLKDVRNAFSHNQYPDSKKIAFRRIRKFNPKELILEEEEGLGIATQMYKEVEKVVNRIKRIELFD

SEQ ID NO: 59 - protein, 1159 aa, Myroides odoratimimus
MKDILTTDTTEKQNRFYSHKIADKYFFGGYFNLASNNIYEVFEEVNKRNTFGKLAKRDNGNLKNYIIHVFKDELSISDFEKRVAIFASYFPILETVDKKSIKERNRTIDLTLSQRIRQFREMLISLVTAVDQLRNFYTHYHHSDIVIENKVLDFLNSSFVSTALHVKDKYLKTDKTKEFLKETIAAELDILIEAYKKKQIEKKNTRFKANKREDILNAIYNEAFWSFINDKDKDKDKETVVAKGADAYFEKNHHKSNDPDFALNISEKGIVYLLSFFLTNKEMDSLKANLTGFKGKVDRESGNSIKYMATQRIYSFHTYRGLKQKIRTSEEGVKETLLMQMIDELSKVPNVVYQHLSTTQQNSFIEDWNEYYKDYEDDVETDDLSRVIHPVIRKRYEDRFNYFAIRFLDEFFDFPTLRFQVHLGDYVHDRRTKQLGKVESDRIIKEKVTVFARLKDINSAKASYFHSLEEQDKEELDNKWTLFPNPSYDFPKEHTLQHQGEQKNAGKIGIYVKLRDTQYKEKAALEEARKSLNPKERSATKASKYDIITQIIEANDNVKSEKPLVFTGQPIAYLSMNDIHSMLFSLLTDNAELKKTPEEVEAKLIDQIGKQINEILSKDTDTKILKKYKDNDLKETDTDKITRDLARDKEEIEKLILEQKQRADDYNYTSSTKFNIDKSRKRKHLLFNAEKGKIGVWLANDIKRFMFKESKSKWKGYQHTELQKLFAYFDTSKSDLELILSNMVMVKDYPIELIDLVKKSRTLVDFLNKYLEARLEYIENVITRVKNSIGTPQFKTVRKECFTFLKKSNYTVVSLDKQVERILSMPLFIERGFMDDKPTMLEGKSYKQHKEKFADWFVHYKENSNYQNFYDTEVYEITTEDKREKAKVTKKIKQQQKNDVFTLMMVNYMLEEVLKLSSNDRLSLNELYQTKEERIVNKQVAKDTQERNKNYIWNKVVDLQLCDGLVHIDNVKLKDIGNFRKYENDSRVKEFLTYQSDIVWSAYLSNEVDSNKLYVIERQLDNYESIRSKELLKEVQEIECSVYNQVANKESLKQSGNENFKQYVLQGLLPIGMDVREMLILSTDVKFKKEEIIQLGQAGEVEQDLYSLIYIRNKFAHNQLPIKEFFDFCENNYRSISDNEYYAEYYMEIFRSIKEKYAN

SEQ ID NO: 60 - protein, 1120 aa, Prevotella intermedia
MEDDKKTTDSIRYELKDKHFWAAFLNLARHNVYITVNHINKILEEDEINRDGYENTLENSWNEIKDINKKDRLSKLIIKHFPFLEATTYRQNPTDTTKQKEEKQAEAQSLESLKKSFFVFIYKLRDLRNHYSHYKHSKSLERPKFEEDLQNKMYNIFDVSIQFVKEDYKHNTDINPKKDFKHLDRKRKGKFHYSFADNEGNITESGLLFFVSLFLEKKDAIWVQKKLEGFKCSNKSYQKMTNEVFCRSRMLLPKLRLESTQTQDWILLDMLNELIRCPKSLYERLQGVNRKKFYVSFDPADEDYDAEQEPFKNTLVRHQDRFPYFALRYFDYNEVFANLRFQIDLGTYHFSIYKKLIGGQKEDRHLTHKLYGFERIQEFDKQNRPDEWKAIVKDSDTFKKKEEKEEEKPYISETTPHYHLENKKIGIAFKNHNIWPSTQTELTNNKRKKYNLGTSIKAEAFLSVHELLPMMFYYLLLKTENTKNDNKVGGKKETKKQGKHKIEAIIESKIKDIYALYDAFANGEINSEDELKEYLKGKDIKIVHLPKQMIAILKNEHKDMAEKAEAKQEKMKLATENRLKTLDKQLKGKIQNGKRYNSAPKSGEIASWLVNDMMRFQPVQKDENGESLNNSKANSTEYQLLQRTLAFFGSEHERLAPYFKQTKLIESSNPHPFLNDTEWEKCSNILSFYRSYLKARKNFLESLKPEDWEKNQYFLMLKEPKTNRETLVQGWKNGFNLPRGFFTEPIRKWFMEHWKSIKVDDLKRVGLVAKVTPLFFSEKYKDSVQPFYNYPFNVGDVNKPKEEDFLHREERIELWDKKKDKFKGYKAKKKFKEMTDKEKEEHRSYLEFQSWNKFERELRLVRNQDIVTWLLCTELIDKLKIDELNIKELKKLRLKDINTDTAKKEKNNILNRVMPMELPVTVYKVNKGGYIIKNKPLHTIYIKEAETKLLKQGNFKALVKDRRLNGLFSFVKTPSEAESESNPISKLRVEYELGKYQNARLDIIEDMLALEKKLIDKYNSLDTDNFHNMLTGWLELKGEAKKARFQNDVKLLTAVRNAFSHNQYPMYDENLFGNIERFSLSSSNIIESKGLDIAAKLKEEVSKAAKKIQNEEDNKKEKET

SEQ ID NO: 61 - protein, 1199 aa, Capnocytophaga canimorsus
MKNIQRLGKGNEFSPFKKEDKFYFGGFLNLANNNIEDFFKEIITRFGIVITDENKKPKETFGEKILNEIFKKDISIVDYEKWVNIFADYFPFTKYLSLYLEEMQFKNRVICFRDVMKELLKTVEALRNFYTHYDHEPIKIEDRVFYFLDKVLLDVSLTVKNKYLKTDKTKEFLNQHIGEELKELCKQRKDYLVGKGKRIDKESEIINGIYNNAFKDFICKREKQDDKENHNSVEKILCNKEPQNKKQKSSATVWELCSKSSSKYTEKSFPNRENDKHCLEVPISQKGIVFLLSFFLNKGEIYALTSNIKGFKAKITKEEPVTYDKNSIRYMATHRMFSFLAYKGLKRKIRTSEINYNEDGQASSTYEKETLMLQMLDELNKVPDVVYQNLSEDVQKTFIEDWNEYLKENNGDVGTMEEEQVIHPVIRKRYEDKFNYFAIRFLDEFAQFPTLRFQVHLGNYLCDKRTKQICDTTTEREVKKKITVFGRLSELENKKAIFLNEREEIKGWEVFPNPSYDFPKENISVNYKDFPIVGSILDREKQPVSNKIGIRVKIADELQREIDKAIKEKKLRNPKNRKANQDEKQKERLVNEIVSTNSNEQGEPVVFIGQPTAYLSMNDIHSVLYEFLINKISGEALETKIVEKIETQIKQIIGKDATTKILKPYTNANSNSINREKLLRDLEQEQQILKTLLEEQQQREKDKKDKKSKRKHELYPSEKGKVAVWLANDIKRFMPKAFKEQWRGYHHSLLQKYLAYYEQSKEELKNLLPKEVFKHFPFKLKGYFQQQYLNQFYTDYLKRRLSYVNELLLNIQNFKNDKDALKATEKECFKFFRKQNYIINPINIQIQSILVYPIFLKRGFLDEKPTMIDREKFKENKDTELADWFMHYKNYKEDNYQKFYAYPLEKVEEKEKFKRNKQINKQKKNDVYTLMMVEYIIQKIFGDKFVEENPLVLKGIFQSKAERQQNNTHAATTQERNLNGILNQPKDIKIQGKITVKGVKLKDIGNFRKYEIDQRVNTFLDYEPRKEWMAYLPNDWKEKEKQGQLPPNNVIDRQISKYETVRSKILLKDVQELEKIISDEIKEEHRHDLKQGKYYNFKYYILNGLLRQLKNENVENYKVFKLNTNPEKVNITQLKQEATDLEQKAFVLTYIRNKFAHNQLPKKEFWDYCQEKYGKIEKEKTYAEYFAEVFKREKEALIK

SEQ ID NO: 62 - protein, 1175 aa, Porphyromonas gulae
MTEQSERPYNGTYYTLEDKHFWAAFLNLARHNAYITLTHIDRQLAYSKADITNDQDVLSFKALWKNFDNDLERKSRLRSLILKHFSFLEGAAYGKKLFESKSSGNKSSKNKELTKKEKEELQANALSLDNLKSILFDFLQKLKDFRNYYSHYRHSGSSELPLFDGNMLQRLYNVFDVSVQRVKIDHEHNDEVDPHYHFNHLVRKGKKDRYGHNDNPSFKHHFVDGEGMVTEAGLLFFVSLFLEKRDAIWMQKKIRGFKGGTETYQQMTNEVFCRSRISLPKLKLESLRMDDWMLLDMLNELVRCPKPLYDRLREDDRACFRVPVDILPDEDDTDGGGEDPFKNTLVRHQDRFPYFALRYFDLKKVFTSLRFHIDLGTYHFAIYKKMIGEQPEDRHLTRNLYGFGRIQDFAEEHRPEEWKRLVRDLDYFETGDKPYISQTSPHYHIEKGKIGLRFMPEGQHLWPSPEVGTTRTGRSKYAQDKRLTAEAFLSVHELMPMMFYYFLLREKYSEEVSAERVQGRIKRVIEDVYAVYDAFARDEINTRDELDACLADKGIRRGHLPRQMIAILSQEHKDMEEKIRKKLQEMMADTDHRLDMLDRQTDRKIRIGRKNAGLPKSGVIADWLVRDMMRFQPVAKDASGKPLNNSKANSTEYRMLQRALALFGGEKERLTPYFRQMNLTGGNNPHPFLHETRWESHTNILSFYRSYLRARKAFLERIGRSDRVENRPFLLLKEPKTDRQTLVAGWKGEFHLPRGIFTEAVRDCLIEMGHDEVASYKEVGFMAKAVPLYFERACEDRVQPFYDSPFNVGNSLKPKKGRFLSKEERAEEWERGKERFRDLEAWSYSAARRIEDAFAGIEYASPGNKKKIEQLLRDLSLWEAFESKLKVRADRINLAKLKKEILEAQEHPYHDFKSWQKFERELRLVKNQDIITWMMCRDLMEENKVEGLDTGTLYLKDIRPNVQEQGSLNVLNRVKPMRLPVVVYRADSRGHVHKEEAPLATVYIEERDTKLLKQGNFKSFVKDRRLNGLFSFVDTGGLAMEQYPISKLRVEYELAKYQTARVCVFELTLRLEESLLTRYPHLPDESFREMLESWSDPLLAKWPELHGKVRLLIAVRNAFSHNQYPMYDEAVFSSIRKYDPSSPDAIEERMGLNIAHRLSEEVKQAKETVERIIQA

SEQ ID NO: 63 - protein, 1090 aa, Prevotella sp.
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIKKAFGEYAIMK

SEQ ID NO: 64 - protein, 1150 aa, Flavobacterium branchiophilum
MENLNKILDKENEICISKIFNTKGIAAPITEKALDNIKSKQKNDLNKEARLHYFSIGHSFKQIDTKKVFDYVLIEELKDEKPLKFITLQKDFFTKEFSIKLQKLINSIRNINNHYVHNFNDINLNKIDSNVFHFLKESFELAIIEKYYKVNKKYPLDNEIVLFLKELFIKDENTALLNYFTNLSKDEAIEYILTFTITENKIWNINNEHNILNIEKGKYLTFEAMLFLITIFLYKNEANHLLPKLYDFKNNKSKQELFTFFSKKFTSQDIDAEEGHLIKFRDMIQYLNHYPTAWNNDLKLESENKNKIMTTKLIDSIIEFELNSNYPSFATDIQFKKEAKAFLFASNKKRNQTSFSNKSYNEEIRHNPHIKQYRDEIASALTPISFNVKEDKFKIFVKKHVLEEYFPNSIGYEKFLEYNDFTEKEKEDFGLKLYSNPKTNKLIERIDNHKLVKSHGRNQDRFMDFSMRFLAENNYFGKDAFFKCYKFYDTQEQDEFLQSNENNDDVKFHKGKVTTYIKYEEHLKNYSYWDCPFVEENNSMSVKISIGSEEKILKIQRNLMIYFLENALYNENVENQGYKLVNNYYRELKKDVEESIASLDLIKSNPDFKSKYKKILPKRLLHNYAPAKQDKAPENAFETLLKKADFREEQYKKLLKKAEHEKNKEDFVKRNKGKQFKLHFIRKACQMMYFKEKYNTLKEGNAAFEKKDPVIEKRKNKEHEFGHHKNLNITREEFNDYCKWMFAFNGNDSYKKYLRDLFSEKHFFDNQEYKNLFESSVNLEAFYAKTKELFKKWIETNKPTNNENRYTLENYKNLILQKQVFINVYHFSKYLIDKNLLNSENNVIQYKSLENVEYLISDFYFQSKLSIDQYKTCGKLFNKLKSNKLEDCLLYEIAYNYIDKKNVHKIDIQKILTSKIILTINDANTPYKISVPFNKLERYTEMIAIKNQNNLKARFLIDLPLYLSKNKIKKGKDSAGYEIIIKNDLEIEDINTINNKIINDSVKFTEVLMELEKYFILKDKCILSKNYIDNSEIPSLKQFSKVWIKENENEIINYRNIACHFHLPLLETFDNLLLNVEQKFIKEELQNVSTINDLSKPQEYLILLFIKFKHNNFYLNLFNKNESKTIKNDKEVKKNRVLQKFINQVILKKK

SEQ ID NO: 65 - protein, 1159 aa, Myroides odoratimimus
MKDILTTDTTEKQNRFYSHKIADKYFFGGYFNLASNNIYEVFEEVNKRNTFGKLAKRDNGNLKNYIIHVFKDELSISDFEKRVAIFASYFPILETVDKKSIKERNRTIDLTLSQRIRQFREMLISLVTAVDQLRNFYTHYHHSDIVIENKVLDFLNSSFVSTALHVKDKYLKTDKTKEFLKETIAAELDILIEAYKKKQIEKKNTRFKANKREDILNAIYNEAFWSFINDKDKDKDKETVVAKGADAYFEKNHHKSNDPDFALNISEKGIVYLLSFFLTNKEMDSLKANLTGFKGKVDRESGNSIKYMATQRIYSFHTYRGLKQKIRTSEEGVKETLLMQMIDELSKVPNVVYQHLSTTQQNSFIEDWNEYYKDYEDDVETDDLSRVTHPVIRKRYEDRFNYFAIRFLDEFFDFPTLRFQVHLGDYVHDRRTKQLGKVESDRIIKEKVTVFARLKDINSAKASYFHSLEEQDKEELDNKWTLFPNPSYDFPKEHTLQHQGEQKNAGKIGIYVKLRDTQYKEKAALEEARKSLNPKERSATKASKYDIITQIIEANDNVKSEKPLVFTGQPIAYLSMNDIHSMLFSLLTDNAELKKTPEEVEAKLIDQIGKQINEILSKDTDTKILKKYKDNDLKETDTDKITRDLARDKEEIEKLILEQKQRADDYNYTSSTKFNIDKSRKRKHLLFNAEKGKIGVWLANDIKRFMFKESKSKWKGYQHIELQKLFAYFDTSKSDLELILSNMVMVKDYPIELIDLVKKSRTLVDFLNKYLEARLEYIENVITRVKNSIGTPQFKTVRKECFTFLKKSNYTVVSLDKQVERILSMPLFIERGFMDDKPTMLEGKSYKQHKEKFADWFVHYKENSNYQNFYDTEVYEITTEDKREKAKVTKKIKQQQKNDVFTLMMVNYMLEEVLKLSSNDRLSLNELYQTKEERIVNKQVAKDTQERNKNYIWNKVVDLQLCDGLVHIDNVKLKDIGNFRKYENDSRVKEFLTYQSDIVWSAYLSNEVDSNKLYVIERQLDNYESIRSKELLKEVQEIECSVYNQVANKESLKQSGNENFKQYVLQGLLPIGMDVREMLILSTDVKFKKEEIIQLGQAGEVEQDLYSLIYIRNKFAHNQLPIKEFFDFCENNYRSISDNEYYAEYYMEIFRSIKEKYAN

SEQ ID NO: 66 - protein, 1214 aa, Flavobacterium columnare
MSSKNESYNKQKTFNHYKQEDKYFFGGFLNNADDNLRQVGKEFKTRINFNHNNNELASVFKDYFNKEKSVAKREHALNLLSNYFPVLERIQKHTNHNFEQTREIFELLLDTIKKLRDYYTHHYHKPITINPKIYDFLDDTLLDVLITIKKKKVKNDTSRELLKEKLRPELTQLKNQKREELIKKGKKLLEENLENAVFNHCLIPFLEENKTDDKQNKTVSLRKYRKSKPNEETSITLTQSGLVFLMSFFLHRKEFQVFTSGLERFKAKVNTIKEEEISLNKNNIVYMITHWSYSYYNFKGLKHRIKTDQGVSTLEQNNTTHSLTNTNTKEALLTQIVDYLSKVPNEIYETLSEKQQKEFEEDINEYMRENPENEDSTFSSIVSHKVIRKRYENKFNYFAMRFLDEYAELPTLRFMVNFGDYIKDRQKKILESIQFDSERIIKKEIHLFEKLSLVTEYKKNVYLKETSNIDLSRFPLFPNPSYVMANNNIPFYIDSRSNNLDEYLNQKKKAQSQNKKRNLTFEKYNKEQSKDAIIAMLQKEIGVKDLQQRSTIGLLSCNELPSMLYEVIVKDIKGAELENKIAQKIREQYQSIRDFTLDSPQKDNIPTTLIKTINTDSSVTFENQPIDIPRLKNALQKELTLTQEKLLNVKEHEIEVDNYNRNKNTYKFKNQPKNKVDDKKLQRKYVFYRNEIRQEANWLASDLIHFMKNKSLWKGYMHNELQSFLAFFEDKKNDCIALLETVFNLKEDCILTKGLKNLFLKHGNFIDFYKEYLKLKEDFLSTESTFLENGFIGLPPKILKKELSKRLKYIFIVFQKRQFIIKELEEKKNNLYADAINLSRGIFDEKPTMIPFKKPNPDEFASWFVASYQYNNYQSFYELTPDIVERDKKKKYKNLRAINKVKIQDYYLKLMVDTLYQDLFNQPLDKSLSDFYVSKAEREKIKADAKAYQKLNDSSLWNKVIHLSLQNNRITANPKLKDIGKYKRALQDEKIATLLTYDARTWTYALQKPEKENENDYKELHYTALNMELQEYEKVRSKELLKQVQELEKKILDKFYDFSNNASHPEDLEIEDKKGKRHPNFKLYITKALLKNESEIINLENIDIEILLKYYDYNTEELKEKIKNMDEDEKAKIINTKENYNKITNVLIKKALVLIIIRNKMAHNQYPPKFIYDLANRFVPKKEEEYFATYFNRVFETITKELWENKEKKDKTQV

SEQ ID NO: 67 - protein, 1119 aa, Porphyromonas gingivalis
MTEQNEKPYNGTYYTLEDKHFWAAFLNLARHNAYITLAHIDRQLAYSKADITNDEDILFFKGQWKNLDNDLERKARLRSLILKHFSFLEGAAYGKKLFESQSSGNKSSKKKELSKKEKEELQANALSLDNLKSILFDFLQKLKDFRNYYSHYRHPESSELPLFDGNMLQRLYNVFDVSVQRVKRDHEHNDKVDPHRHFNHLVRKGKKDKYGNNDNPFFKHHFVDREGTVTEAGLLFFVSLFLEKRDAIWMQKKIRGFKGGTEAYQQMTNEVFCRSRISLPKLKLESLRTDDWMLLDMLNELVRCPKSLYDRLREEDRARFRVPVDILSDEDDTDGTEEDPFKNTLVRHQDRFPYFALRYFDLKKVFTSLRFHIDLGTYHFAIYKKNIGEQPEDRHLTRNLYGFGRIQDFAEEHRPEEWKRLVRDLDYFETGDKPYITQTTPHYHIEKGKIGLRFVPEGQHLWPSPEVGATRTGRSKYAQDKRLTAEAFLSVHELMPMMFYYFLLREKYSEEVSAEKVQGRIKRVIEDVYAVYDAFARDEINTRDELDACLADKGIRRGHLPRQMIAILSQEHKDMEEKVRKKLQEMIADTDHRLDMLDRQTDRKIRIGRKNAGLPKSGVVADWLVRDMMRFQPVAKDTSGKPLNNSKANSTEYRMLQRALALFGGEKERLTPYFRQMNLTGGNNPHPFLHETRWESHTNILSFYRSYLEARKAFLQSIGRSDRVENHRFLLLKEPKTDRQTLVAGWKGEFHLPRGIFTEAVRDCLIEMGYDEVGSYKEVGFMAKAVPLYFERASKDRVQPFYDYPFNVGNSLKPKKGRFLSKEKRAEEWESGKERFRLAKLKKEILEAKEHPYHDFKSWQKFERELRLVKNQDIITWMMCRDLMEENKVEGLDTGTLYLKDIRTDVQEQGSLNVLNRVKPMRLPVVVYRADSRGHVHKEQAPLATVYIEERDTKLLKQGNFKSFVKDRRLNGLFSFVDTGALAMEQYPISKLRVEYELAKYQTARVCAFEQTLELEESLLTRYPHLPDKNFRKMLESWSDPLLDKWPDLHGNVRLLIAVRNAFSHNQYPMYDETLFSSIRKYDPSSPDAIEERMGLNIAHRLSEEVKQAKEMVERIIQA

SEQ ID NO: 68 - protein, 1020 aa, Porphyromonas sp.
MTEQSERPYNGTYYTLEDKHFWAAFLNLARHNAYITLTHIDRQLAYSKADITNDQDVLSFKALWKNFDNDLERKSRLRSLILKHFSFLEGAAYGKKLFESKSSGNKSSKNKELTKKEKEELQANALSLDNLKSILFDFLQKLKDFRNYYSHYRHSESSELPLFDGNMLQRLYNVFDVSVQRVKRDHEHNDKVDPHRHFNHLVRKGKKDRYGHNDNPSFKHHFVDSEGMVTEAGLLFFVSLFLEKRDAIWMQKKIRGFKGGTETYQQMTNEVFCRSRISLPKLKLESLRTDDWMLLDMLNELVRCPKPLYDRLREDDRACFRVPVDILPDEDDTDGGGEDPFKNTLVRHQDRFPYFALRYFDLKKVFTSLRFHIDLGTYHFAIYKKMIGEQPEDRHLTRNLYGFGRIQDFAEEHRPEEWKRLVRDLDYFETGDKPYISQTTPHYHIEKGKIGLRFVPEGQHLWPSPEVGTTRTGRSKYAQDKRLTAEAFLSVHELMPMMFYYFLLREKYSEEVSAEKVQGRIKRVIEDVYAIYDAFARDEINTLKELDACLADKGIRRGHLPKQMIGILSQERKDMEEKVRKKLQEMIADTDHRLDMLDRQTDRKIRIGRKNAGLPKSGVIADWLVRDMMRFQPVAKDTSGKPLNNSKANSTEYRMLQRALALFGGEKERLTPYFRQMNLTGGNNPHPFLHETRWESHTNILSFYRSYLRARKAFLERIGRSDRVENCPFLLLKEPKTDRQTLVAGWKGEFHLPRGIFTEAVRDCLIEMGYDEVGSYREVGFMAKAVPLYFERACEDRVQPFYDSPFNVGNSLKPKKGRFLSKEDRAEEWERGKERFRDLEAWSHSAARRIKDAFAGIEYASPGNKKKIEQLLRDLSLWEAFESKLKVRADKINLAKLKKEILEAQEHPYHDFKSWQKFERELRLVKNQDIITWMMCRDLMEENKVEGLDTGTLYLKDIRPNVQEQGSLNVLNRVKPMRLPVVVYRADSRGHVHKEEAPLATVYIEERDTKLLKQGNFKSF

SEQ ID NO: 69 - protein, 1132 aa, Prevotella intermedia
MEDDKKTKESTNMLDNKHFWAAFLNLARHNVYITVNHINKVLELKNKKDQDIIIDNDQDILAIKTHWEKVNGDLNKTERLRELMTKHFPFLETAIYTKNKEDKEEVKQEKQAKAQSFDSLKHCLFLFLEKLQEARNYYSHYKYSESTKEPMLEKELLKKMYNIFDDNIQLVIKDYQHNKDINPDEDFKHLDRTEEEFNYYFTTNKKGNITASGLLFFVSLFLEKKDAIWMQQKLRGFKDNRESKKKMTHEVFCRSRMLLPKLRLESTQTQDWILLDMLNELIRCPKSLYERLQGEYRKKFNVPFDSADEDYDAEQEPFKNTLVRHQDRFPYFALRYFDYNEIFTNLRFQIDLGTYHFSIYKKLIGGQKEDRHLTHKLYGFERIQEFAKQNRTDEWKAIVKDFDTYETSEEPYISETAPHYHLENQKIGIRFRNDNDEIWPSLKTNGENNEKRKYKLDKQYQAEAFLSVHELLPMMFYYLLLKKEEPNNDKKNASIVEGFIKREIRDIYKLYDAFANGEINNIDDLEKYCEDKGIPKRHLPKQMVAILYDEHKDMAEEAKRKQKEMVKDTKKLLATLEKQTQGEIEDGGRNIRLLKSGEIARWLVNDMMRFQPVQKDNEGNPLNNSKANSTEYQMLQRSLALYNKEEKPTRYFRQVNLINSSNPHPFLKWTKWEECNNILSFYRSYLTKKIEFLNKLKPEDWEKNQYFLKLKEPKTNRETLVQGWKNGFNLPRGIFTEPIREWFKRHQNDSEEYEKVETLDRVGLVTKVIPLFFKKEDSKDKEEYLKKDAQKEINNCVQPFYGFPYNVGNIHKPDEKDFLPSEERKKLWGDKKYKFKGYKAKVKSKKLTDKEKEEYRSYLEFQSWNKFERELRLVRNQDIVTWLLCTELIDKLKVEGLNVEELKKLRLKDIDTDTAKQEKNNILNRVMPMQLPVTVYEIDDSHNIVKDRPLHTVYIEETKTKLLKQGNFKALVKDRRLNGLFSFVDTSSETELKSNPISKSLVEYELGEYQNARIETIKDMLLLEETLIEKYKTLPTDNFSDMLNGWLEGKDEADKARFQNDVKLLVAVRNAFSHNQYPMRNRIAFANINPFSLSSADTSEEKKLDIANQLKDKTHKIIKRIIEIEKPIETKE

SEQ ID NO: 70 - protein, 1134 aa, Prevotella intermedia
MKMEDDKKTKESTNMLDNKHFWAAFLNLARHNVYITVNHINKVLELKNKKDQDIIIDNDQDILAIKTHWEKVNGDLNKTERLRELMTKHFPFLETAIYTKNKEDKEEVKQEKQAKAQSFDSLKHCLFLFLEKLQEARNYYSHYKYSESTKEPMLEKELLKKMYNIFDDNIQLVIKDYQHNKDINPDEDFKHLDRTEEEFNYYFTTNKKGNITASGLLFFVSLFLEKKDAIWMQQKLRGFKDNRESKKKMTHEVFCRSRMLLPKLRLESTQTQDWILLDMLNELIRCPKSLYERLQGEYRKKFNVPFDSADEDYDAEQEPFKNTLVRHQDRFPYFALRYFDYNEIFTNLRFQIDLGTYHFSIYKKLIGGQKEDRHLTHKLYGFERIQEFAKQNRTDEWKAIVKDFDTYETSEEPYISETAPHYHLENQKIGIRFRNDNDEIWPSLKTNGENNEKRKYKLDKQYQAEAFLSVHELLPMMFYYLLLKKEEPNNDKKNASIVEGFIKREIRDIYKLYDAFANGEINNIDDLEKYCEDKGIPKRHLPKQMVAILYDEHKDMAEEAKRKQKEMVKDTKKLLATLEKQTQGEIEDGGRNIRLLKSGEIARWLVNDMMRFQPVQKDNEGNPLNNSKANSTEYQMLQRSLALYNKEEKPTRYFRQVNLINSSNPHPFLKWTKWEECNNILSFYRSYLTKKIEFLNKLKPEDWEKNQYFLKLKEPKTNRETLVQGWKNGFNLPRGIFTEPIREWFKRHQNDSEEYEKVETLDRVGLVTKVIPLFFKKEDSKDKEEYLKKDAQKEINNCVQPFYGFPYNVGNIHKPDEKDFLPSEERKKLWGDKKYKFKGYKAKVKSKKLTDKEKEEYRSYLEFQSWNKFERELRLVRNQDIVTWLLCTELIDKLKVEGLNVEELKKLRLKDIDTDTAKQEKNNILNRVMPMQLPVTVYEIDDSHNIVKDRPLHTVYIEETKTKLLKQGNFKALVKDRRLNGLFSFVDTSSETELKSNPISKSLVEYELGEYQNARIETIKDMLLLEETLIEKYKTLPTDNFSDMLNGWLEGKDEADKARFQNDVKLLVAVRNAFSHNQYPMRNRIAFANINPFSLSSADTSEEKKLDIANQLKDKTHKIIKRIIEIEKPIETKE

SEQ ID NO: 71 - protein, 1133 aa, Prevotella intermedia
MEDDKKTTDSISYELKDKHFWAAFLNLARHNVYITVNHINKVLELKNKKDQDIIIDNDQDILAIKTHWEKVNGDLNKTERLRELMTKHFPFLETAIYSKNKEDKEEVKQEKQAKAQSFDSLKHCLFLFLEKLQETRNYYSHYKYSESTKEPMLEKELLKKMYNIFDDNIQLVIKDYQHNKDINPDEDFKHLDRTEEDFNYYFTRNKKGNITESGLLFFVSLFLEKKDAIWMQQKLRGFKDNRESKKKMTHEVFCRSRMLLPKLRLESTQTQDWILLDMLNELIRCPKSLYERLQGEDREKFKVPFDPADEDYDAEQEPFKNTLVRHQDRFPYFALRYFDYNEIFTNLRFQIDLGTFHFSIYKKLIGGQKEDRHLTHKLYGFERIQEFAKQNRPDEWKAIVKDLDTYETSNERYISETTPHYHLENQKIGIRFRNDNDEIWPSLKTNGENNEKSKYKLDKQYQAEAFLSVHELLPMMFYYLLLKKEEPNNDKKNASIVEGFIKREIRDMYKLYDAFANGEINNIDDLEKYCEDKGIPKRHLPKQMVAILYDEHKDMVKEAKRKQRKMVKDTEKLLAALEKQTQEKTEDGGRNIRLLKSGEIARWLVNDMMRFQPVQKDNEGNPLNNSKANSTEYQMLQRSLALYNKEEKPTRYFRQVNLINSSNPHPFLKWTKWEECNNILSFYRSYLTKKIEFLNKLKPEDWEKNQYFLKLKEPKTNRETLVQGWKNGFNLPRGIFTEPIREWFKRHQNDSKEYEKVEALDRVGLVTKVIPLFFKKEDSKDKEEDLKKDAQKEINNCVQPFYSFPYNVGNIHKPDEKDFLHREERIELWDKKKDKFKGYKAKVKSKKLTDKEKEEYRSYLEFQSWNKFERELRLVRNQDIVTWLLCTELIDKLKVEGLNVEELKKLRLKDIDTDTAKQEKNNILNRVMPMQLPVTVYEIDDSHNIVKDRPLHTVYIEETKTKLLKQGNFKALVKDRRLNGLFSFVDTSSEAELKSNPISKSLVEYELGEYQNARIETIKDMLLLEETLIEKYKNLPTDNFSDMLNGWLEGKDEADKARFQNDVKLLVAVRNAFSHNQYPMRNRIAFANINPFSLSSADTSEEKKLDIANQLKDKTHKIIKRIIEIEKPIETKE

SEQ ID NO: 72 - protein, 1127 aa, Prevotella buccae; identical to SEQ ID NO: 50

SEQ ID NO: 73 - protein, 1125 aa, Prevotella pallens
MKEEEKGKTPVVSTYNKDDKHFWAAFLNLARHNVYITVNHINKILGEGEINRDGYENTLEKSWNEIKDINKKDRLSKLIIKHFPFLEVTTYQRNSADTTKQKEEKQAEAQSLESLKKSFFVFIYKLRDLRNHYSHYKHSKSLERPKFEEDLQEKMYNIFDASIQLVKEDYKHNTDIKTEEDFKHLDRKGQFKYSFADNEGNITESGLLFFVSLFLEKKDAIWVQKKLEGFKCSNESYQKMTNEVFCRSRMLLPKLRLQSTQTQDWILLDMLNELIRCPKSLYERLREEDRKKFRVPIEIADEDYDAEQEPFKNALVRHQDRFPYFALRYFDYNEIFTNLRFQIDLGTYHFSIYKKQIGDYKESHHLTHKLYGFERIQEFTKQNRPDEWRKFVKTFNSFETSKEPYIPETTPHYHLENQKIGIRFRNDNDKIWPSLKTNSEKNEKSKYKLDKSFQAEAFLSVHELLPMMFYYLLLKTENTDNDNEIETKKKENKNDKQEKHKIEEIIENKITEIYALYDAFANGKINSIDKLEEYCKGKDIEIGHLPKQMIAILKSEHKDMATEAKRKQEEMLADVQKSLESLDNQINEEIENVERKNSSLKSGEIASWLVNDMMRFQPVQKDNEGNPLNNSKANSTEYQMLQRSLALYNKEEKPTRYFRQVNLIESSNPHPFLNNTEWEKCNNILSFYRSYLEAKKNFLESLKPEDWEKNQYFLMLKEPKTNCETLVQGWKNGFNLPRGIFTEPIRKWFMEHRKNITVAELKRVGLVAKVIPLFFSEEYKDSVQPFYNYLFNVGNINKPDEKNFLNCEERRELLRKKKDEFKKMTDKEKEENPSYLEFQSWNKFERELRLVRNQDIVTWLLCMELFNKKKIKELNVEKIYLKNINTNTTKKEKNTEEKNGEEKIIKEKNNILNRIMPMRLPIKVYGRENFSKNKKKKIRRNTFFTVYIEEKGTKLLKQGNFKALERDRRLGGLFSFVKTHSKAESKSNTISKSRVEYELGEYQKARIEIIKDMLALEETLIDKYNSLDTDNFHNMLTGWLKLKDEPDKASFQNDVDLLIAVRNAFSHNQYPMRNRIAFANINPFSLSSANTSEEKGLGIANQLKDKTHKTIEKIIEIEKPIETKE

SEQ ID NO: 74 - protein, 1157 aa, Myroides odoratimimus
MKDILTTDTTEKQNRFYSHKIADKYFFGGYFNLASNNIYEVFEEVNKRNTFGKLAKRDNGNLKNYIIHVFKDELSISDFEKRVAIFASYFPILETVDKKSIKERNRTIDLTLSQRIRQFREMLISLVTAVDQLRNFYTHYHHSEIVIENKVLDFLNSSLVSTALHVKDKYLKTDKTKEFLKETIAAELDILIEAYKKKQIEKKNTRFKANKREDILNAIYNEAFWSFINDKDKDKETVVAKGADAYFEKNHHKSNDPDFALNISEKGIVYLLSFFLTNKEMDSLKANLTGFKGKVDRESGNSIKYMATQRIYSFHTYRGLKQKIRTSEEGVKETLLMQMIDELSKVPNVVYQHLSTTQQNSFIEDWNEYYKDYEDDVETDDLSRVIHPVIRKRYEDRFNYFAIRFLDEFFDFPTLRFQVHLGDYVHDRRTKQLGKVESDRIIKEKVTVFARLKDINSAKANYFHSLEEQDKEELDNKWTLFPNPSYDFPKEHTLQHQGEQKNAGKIGIYVKLRDTQYKEKAALEEARKSLNPKERSATKASKYDIITQIIEANDNVKSEKPLVFTGQPIAYLSMNDIHSMLFSLLTDNAELKKTPEEVEAKLIDQIGKQINEILSKDTDTKILKKYKDNDLKETDTDKITRDLARDKEEIEKLILEQKQRADDYNYTSSTKFNIDKSRKRKHLLFNAEKGKIGVWLANDIKRFMTEEFKSKWKGYQHTELQKLFAYYDTSKSDLDLILSDMVMVKDYPIELIALVKKSRTLVDFLNKYLEARLGYMENVITRVKNSIGTPQFKTVRKECFTFLKKSNYTVVSLDKQVERILSMPLFIERGFMDDKPTMLEGKSYQQHKEKFADWFVHYKENSNYQNFYDTEVYEITTEDKREKAKVTKKIKQQQKNDVFTLMMVNYMLEEVLKLSSNDRLSLNELYQTKEERIVNKQVAKDTQERNKNYIWNKVVDLQLCEGLVRIDKVKLKDIGNFRKYENDSRVKEFLTYQSDIVWSAYLSNEVDSNKLYVIERQLDNYESIRSKELLKEVQEIECSVYNQVANKESLKQSGNENFKQYVLQGLVPIGMDVREMLILSTDVKFIKEEIIQLGQAGEVEQDLYSLIYIRNKFAHNQLPIKEFFDFCENNYRSISDNEYYAEYYMEIFRSIKEKYTS

SEQ ID NO: 75 - protein, 1157 aa, Myroides odoratimimus
MKDILTTDTTEKQNRFYSHKIADKYFFGGYFNLASNNIYEVFEEVNKRNTFGKLAKRDNGNLKNYIIHVFKDELSISDFEKRVAIFASYFPILETVDKKSIKERNRTIDLTLSQRIRQFREMLISLVTAVDQLRNFYTHYHHSEIVIENKVLDFLNSSLVSTALHVKDKYLKTDKTKEFLKETIAAELDILIEAYKKKQIEKKNTRFKANKREDILNAIYNEAFWSFINDKDKDKETVVAKGADAYFEKNHHKSNDPDFALNISEKGIVYLLSFFLTNKEMDSLKANLTGFKGKVDRESGNSIKYMATQRIYSFHTYRGLKQKIRTSEEGVKETLLMQMIDELSKVPNVVYQHLSTTQQNSFIEDWNEYYKDYEDDVETDDLSRVIHPVIRKRYEDRFNYFAIRFLDEFFDFPTLRFQVHLGDYVHDRRTKQLGKVESDRIIKEKVTVFARLKDINSAKASYFHSLEEQDKEELDNKWTLFPNPSYDFPKEHTLQHQGEQKNAGKIGIYVKLRDTQYKEKAALEEARKSLNPKERSATKASKYDIITQIIEANDNVKSEKPLVFTGQPIAYLSMNDIHSMLFSLLTDNAELKKTPEEVEAKLIDQIGKQINEILSKDTDTKILKKYKDNDLKETDTDKITRDLARDKEEIEKLILEQKQRADDYNYTSSTKFNIDKSRKRKHLLFNAEKGKIGVWLANDIKRFMFKESKSKWKGYQHTELQKLFAYFDTSKSDLELILSDMVMVKDYPIELIDLVRKSRTLVDFLNKYLEARLGYIENVITRVKNSIGTPQFKTVRKECFAFLKESNYTVASLDKQIERILSMPLFIERGFMDSKPTMLEGKSYQQHKEDFADWFVHYKENSNYQNFYDTEVYEIITEDKREQAKVTKKIKQQQKNDVFTLMMVNYMLEEVLKLPSNDRLSLNELYQTKEERIVNKQVAKDTQERNKNYIWNKVVDLQLCEGLVRIDKVKLKDIGNFRKYENDSRVKEFLTYQSDIVWSGYLSNEVDSNKLYVIERQLDNYESIRSKELLKEVQEIECIVYNQVANKESLKQSGNENFKQYVLQGLLPRGTDVREMLILSTDVKFKKEEIMQLGQVREVEQDLYSLIYIRNKFAHNQLPIKEFFDFCENNYRPISDNEYYAEYYMEIFRSIKEKYAS

SEQ ID NO: 76 - protein, 1224 aa, Bergeyella zoohelcum; identical to SEQ ID NO: 48

SEQ ID NO: 77 - protein, 1150 aa, Prevotella saccharolytica
MMEKENVQGSHIYYEPTDKCFWAAFYNLARHNAYLTIAHINSFVNSKKGINNDDKVLDIIDDWSKFDNDLLMGARLNKLILKHFPFLKAPLYQLAKRKTRKQQGKEQQDYEKKGDEDPEVIQEAIANAFKMANVRKTLHAFLKQLEDLRNHFSHYNYNSPAKKMEVKFDDGFCNKLYYVFDAALQMVKDDNRMNPEINMQTDFEHLVRLGRNRKIPNTFKYNFTNSDGTINNNGLLFFVSLFLEKRDAIWMQKKIKGFKGGTENYMRMTNEVFCRNRMVIPKLRLETDYDNHQLMFDMLNELVRCPLSLYKRLKQEDQDKFRVPIEFLDEDNEADNPYQENANSDENPTEETDPLKNTLVRHQHRFPYFVLRYFDLNEVFKQLRFQINLGCYHFSIYDKTIGERTEKRHLTRTLFGFDRLQNFSVKLQPEHWKNMVKHLDTEESSDKPYLSDAMPHYQIENEKIGIHFLKTDTEKKETVWPSLEVEEVSSNRNKYKSEKNLTADAFLSTHELLPMMFYYQLLSSEEKTRAAAGDKVQGVLQSYRKKIFDIYDDFANGTINSMQKLDERLAKDNLLRGNMPQQMLAILEHQEPDMEQKAKEKLDRLITETKKRIGKLEDQFKQKVRIGKRRADLPKVGSIADWLVNDMMRFQPAKRNADNTGVPDSKANSTEYRLLQEALAFYSAYKDRLEPYFRQVNLIGGTNPHPFLHRVDWKKCNHLLSFYHDYLEAKEQYLSHLSPADWQKHQHFLLLKVRKDIQNEKKDWKKSLVAGWKNGFNLPRGLFTESIKTWFSTDADKVQITDTKLFENRVGLIAKLIPLYYDKVYNDKPQPFYQYPFNINDRYKPEDTRKRFTAASSKLWNEKKMLYKNAQPDSSDKIEYPQYLDFLSWKKLERELRMLRNQDMMVWLMCKDLFAQCTVEGVEFADLKLSQLEVDVNVQDNLNVLNNVSSMILPLSVYPSDAQGNVLRNSKPLHTVYVQENNTKLLKQGNFKSLLKDRRLNGLFSFIAAEGEDLQQHPLTKNRLEYELSIYQTMRISVFEQTLQLEKAILTRNKTLCGNNFNNLLNSWSEHRTDKKTLQPDIDFLIAVRNAFSHNQYPMSTNTVMQGIEKFNIQTPKLEEKDGLGIASQLAKKTKDAASRLQNIINGGTN

SEQ ID NO: 78 - protein, 1175 aa, Porphyromonas gingivalis
MTEQNEKPYNGTYYTLEDKHFWAAFFNLARHNAYITLTHIDRQLAYSKADITNDEDILFFKGQWKNLDNDLERKARLRSLILKHFSFLEGAAYGKKLFESQSSGNKSSKKKELTKKEKEELQANALSLDNLKSILFDFLQKLKDFRNYYSHYRHPESSELPLFDGNMLQRLYNVFDVSVQRVKRDHEHNDKVDPHRHFNHLVRKGKKDRCGNNDNPFFKHHFVDREEKVTEAGLLFFVSLFLEKRDAIWMQKKIRGFKGGTETYQQMTNEVFCRSRISLPKLKLESLRTDDWMLLDMLNELVRCPKSLYDRLREEDRARFRVPVDILSDEDDTDGTEEDPFKNTLVRHQDRFPYFALRYFDLKKVFTSLRFHIDLGTYHFAIYKKNIGEQPEDRHLTRNLYGFGRIQDFAEEHRPEEWKRLVRDLDYFETGDKPYITQTTPHYHIEKGKIGLRFVPEGQLLWPSPEVGATRTGRSKYAQDKRFTAEAFLSVHELMPMMFYYFLLREKYSEEASAERVQGRIKRVIEDVYAVYDAFARGEIDTLDRLDACLADKGIRRGHLPRQMIAILSQEHKDMEEKVRKKLQEMIADTDHRLDMLDRQTDRKIRIGRKNAGLPKSGVIADWLVRDMMRFQPVAKDTSGKPLNNSKANSTEYRMLQRALALFGGEKERLTPYFRQMNLTGGNNPHPFLHETRWESHTNILSFYRSYLKARKAFLQSIGRSDRVENHRFLLLKEPKTDRQTLVAGWKGEFHLPRGIFTEAVRDCLIEMGLDEVGSYKEVGFMAKAVPLYFERACKDRVQPFYDYPFNVGNSLKPKKGRFLSKEKRAEEWESGKERFRDLEAWSHSAARRIEDAFAGIENASRENKKKIEQLLQDLSLWETFESKLKVKADKINIAKLKKEILEAKEHPYLDFKSWQKFERELRLVKNQDIITWMMCRDLMEENKVEGLDTGTLYLKDIRTDVHEQGSLNVLNRVKPMRLPVVVYRADSRGHVHKEQAPLATVYIEERDTKLLKQGNFKSFVKDRRLNGLFSFVDTGALAMEQYPISKLRVEYELAKYQTARVCAFEQTLELEESLLTRYPHLPDKNFRKMLESWSDPLLDKWPDLHGNVRLLIAVRNAFSHNQYPMYDETLFSSIRKYDPSSPDAIEERMGLNIAHRLSEEVKQAKEMVERIIQA

SEQ ID NO: 79 - protein, 1115 aa, Bacteroides pyogenes
MESIKNSQKSTGKTLQKDPPYFGLYLNMALLNVRKVENHIRKWLGDVALLPEKSGFHSLLTTDNLSSAKWTRFYYKSRKFLPFLEMFDSDKKSYENRRETTECLDTIDRQKISSLLKEVYGKLQDIRNAFSHYHIDDQSVKHTALIISSEMHRFIENAYSFALQKTRARFTGVFVETDFLQAEEKGDNKKFFAIGGNEGIKLKDNALIFLICLFLDREEAFKFLSRATGFKSTKEKGFLAVRETFCALCCRQPHERLLSVNPREALLMDMLNELNRCPDILFEMLDEKDQKSFLPLLGEEEQAHILENSLNDELCEAIDDPFEMIASLSKRVRYKNRFPYLMLRYIEEKNLLPFIRFRIDLGCLELASYPKKMGEENNYERSVTDHAMAFGRLTDFHNEDAVLQQITKGITDEVRFSLYAPRYAIYNNKIGFVRTGGSDKISFPTLKKKGGEGHCVAYTLQNTKSFGFISIYDLRKILLLSFLDKDKAKNIVSGLLEQCEKHWKDLSENLFDAIRTELQKEFPVPLIRYTLPRSKGGKLVSSKLADKQEKYESEFERRKEKLTEILSEKDFDLSQIPRRMIDEWLNVLPTSREKKLKGYVETLKLDCRERLRVFEKREKGEHPVPPRIGEMATDLAKDIIRMVIDQGVKQRITSAYYSEIQRCLAQYAGDDNRRHLDSIIRELRLKDTKNGHPFLGKVLRPGLGHTEKLYQRYFEEKKEWLEATFYPAASPKRVPRFVNPPTGKQKELPLIIRNLMKERPEWRDWKQRKNSHPIDLPSQLFENEICRLLKDKIGKEPSGKLKWNEMFKLYWDKEFPNGMQRFYRCKRRVEVFDKVVEYEYSEEGGNYKKYYEALIDEVVRQKISSSKEKSKLQVEDLTLSVRRVFKRAINEKEYQLRLLCEDDRLLFMAVRDLYDWKEAQLDLDKIDNMLGEPVSVSQVIQLEGGQPDAVIKAECKLKDVSKLMRYCYDGRVKGLMPYFANHEATQEQVEMELRHYEDHRRRVFNWVFALEKSVLKNEKLRRFYEESQGGCEHRRCIDALRKASLVSEEEYEFLVHIRNKSAHNQFPDLEIGKLPPNVTSGFCECIWSKYKAIICRIIPFIDPERRFFGKLLEQK

SEQ ID NO: 80 - protein, 1135 aa, Porphyromonas gingivalis
MNTVPASENKGQSRTVEDDPQYFGLYLNLARENLIEVESHVRIKFGKKKLNEESLKQSLLCDHLLSVDRWTKVYGHSRRYLPFLHYFDPDSQIEKDHDSKTGVDPDSAQRLIRELYSLLDFLRNDFSHNRLDGTTFEHLEVSPDISSFITGTYSLACGRAQSRFADFFKPDDFVLAKNRKEQLISVADGKECLTVSGLAFFICLFLDREQASGMLSRIRGFKRTDENWARAVHETFCDLCIRHPHDRLESSNTKEALLLDMLNELNRCPRILYDMLPEEERAQFLPALDENSMNNLSENSLNEESRLLWDGSSDWAEALTKRIRHQDRFPYLMLRFIEEMDLLKGIRFRVDLGEIELDSYSKKVGRNGEYDRTITDHALAFGKLSDFQNEEEVSRMISGEASYPVRFSLFAPRYAIYDNKIGYCHTSDPVYPKSKTGEKRALSNPRSMGFISVHDLRKLLLMELLCEGSFSRMQSDFLRKANRILDETAEGKLQFSALFPEMRHRFIPPQNPKSKDRREKAETTLEKYKQEIKGRKDKLNSQLLSAFDMDQRQLPSRLLDEWMNIRPASHSVKLRTYVKQLNEDCRLRLQKFRKDGDGKARAIPLVGEMATFLSQDIVRMIISEETKKLITSAYYNEMQRSLAQYAGEENRHQFRAIVAELRLLDPSSGHPFLSATMETAHRYTEDFYKCYLEKKREWLAKTFYRPEQDENTKRRISVFFVPDGEARKLLPLLIRRRMKEQNDLQDWIRNKQAHPIDLPSHLFDSKIMELLKVKDGKKKWNEAFKDWWSTKYPDGMQPFYGLRRELNIHGKSVSYIPSDGKKFADCYTHLMEKTVQDKKRELRTAGKPVPPDLAADIKRSFHRAVNEREFMLRLVQEDDRLMLMAINKMMTDREEDILPGLKNIDSILDEENQFSLAVHAKVLEKEGEGGDNSLSLVPATIEIKSKRKDWSKYIRYRYDRRVPGLMSHFPEHKATLDEVKTLLGEYDRCRIKIFDWAFALEGAIMSDRDLKPYLHESSSREGKSGEHSTLVKMLVEKKGCLTPDESQYLILIRNKAAHNQFPCAAEMPLIYRDVSAKVGSIEGSSAKDLPEGSSLVDSLWKKYEMIIRKILPILDPENRFFGKLLNNMSQPINDL

SEQ ID NO: 81 - protein, 1135 aa, Porphyromonas gingivalis
MNTVPASENKGQSRTVEDDPQYFGLYLNLARENLIEVESHVRIKFGKKKLNEESLKQSLLCDHLLSVDRWTKVYGHSRRYLPFLHYFDPDSQIEKDHDSKTGVDPDSAQRLIRELYSLLDFLRNDFSHNRLDGTTFEHLEVSPDISSFITGTYSLACGRAQSRFADFFKPDDFVLAKNRKEQLISVADGKECLTVSGLAFFICLFLDREQASGMLSRIRGFKRTDENWARAVHETFCDLCIRHPHDRLESSNTKEALLLDMLNELNRCPRILYDMLPEEERAQFLPALDENSMNNLSENSLNEESRLLWDGSSDWAEALTKRIRHQDRFPYLMLRFIEEMDLLKGIRFRVDLGEIELDSYSKKVGRNGEYDRTITDHALAFGKLSDFQNEEEVSRMISGEASYPVRFSLFAPRYAIYDNKIGYCHTSDPVYPKSKTGEKRALSNPQSMGFISVHDLRKLLLMELLCEGSFSRMQSGFLRKANRILDETAEGKLQFSALFPEMRHRFIPPQNPKSKDRREKAETTLEKYKQEIKGRKDKLNSQLLSAFDMNQRQLPSRLLDEWMNIRPASHSVKLRTYVKQLNEDCRLRLRKFRKDGDGKARAIPLVGEMATFLSQDIVRMIISEETKKLITSAYYNEMQRSLAQYAGEENRRQFRAIVAELHLLDPSSGHPFLSATMETAHRYTEDFYKCYLEKKREWLAKTFYRPEQDENTKRRISVFFVPDGEARKLLPLLIRRRMKEQNDLQDWIRNKQAHPIDLPSHLFDSKIMELLKVKDGKKKWNEAFKDWWSTKYPDGMQPFYGLRRELNIHGKSVSYIPSDGKKFADCYTHLMEKTVQDKKRELRTAGKPVPPDLAADIKRSFHRAVNEREFMLRLVQEDDRLMLMAINKMMTDREEDILPGLKNIDSILDEENQFSLAVHAKVLEKEGEGGDNSLSLVPATIEIKSKRKDWSKYIRYRYDRRVPGLMSHFPEHKATLDEVKTLLGEYDRCRIKIFDWAFALEGAIMSDRDLKPYLHESSSREGKSGEHSTLVKMLVEKKGCLTPDESQYLILIRNKAAHNQFPCAAEMPLIYRDVSAKVGSIEGSSAKDLPEGSSLVDSLWKKYEMIIRKILPILDHENRFFGKLLNNMSQPINDL

SEQ ID NO: 82 - protein, 1135 aa, Porphyromonas gingivalis
MNTVPASENKGQSRTVEDDPQYFGLYLNLARENLIEVESHVRIKFGKKKLNEESLKQSLLCDHLLSVDRWTKVYGHSRRYLPFLHYFDPDSQIEKDHDSKTGVDPDSAQRLIRELYSLLDFLRNDFSHNRLDGTTFEHLEVSPDISSFITGTYSLACGRAQSRFADFFKPDDFVLAKNRKEQLISVADGKECLTVSGLAFFICLFLDREQASGMLSRIRGFKRTDENWARAVHETFCDLCIRHPHDRLESSNTKEALLLDMLNELNRCPRILYDMLPEEERAQFLPALDENSMNNLSENSLNEESRLLWDGSSDWAEALTKRIRHQDRFPYLMLRFIEEMDLLKGIRFRVDLGEIELDSYSKKVGRNGEYDRTITDHALAFGKLSDFQNEEEVSRMISGEASYPVRFSLFAPRYAIYDNKIGYCHTSDPVYPKSKTGEKRALSNPRSMGFISVHDLRKLLLMELLCEGSFSRMQSDFLRKANRILDETAEGKLQFSALFPEMRHRFIPPQNPKSKDRREKAETTLEKYKQEIKGRKDKLNSQLLSAFDMDQRQLPSRLLDEWMNIRPASHSVKLRTYVKQLNEDCRLRLQKFRKDGDGKARAIPLVGEMATFLSQDIVRMIISEETKKLITSAYYNEMQRSLAQYAGEENRHQFRAIVAELRLLDPSSGHPFLSATMETAHRYTEDFYKCYLEKKREWLAKTFYRPEQDENTKRRISVFFVPDGEARKLLPLLIRRRMKEQNDLQDWIRNKQAHPIDLPSHLFDSKVMELLKVKDGKKKWNEAFKDWWSTKYPDGMQPFYGLRRELNIHGKSVSYIPSDGKKFADCYTHLMEKTVRDKKRELRTAGKPVPPDLAAYIKRSFHRAVNEREFMLRLVQEDDRLMLMAINKIMTDREEDILPGLKNIDSILDKENQFSLAVHAKVLEKEGEGGDNSLSLVPATIEIKSKRKDWSKYIRYRYDRRVPGLMSHFPEHKATLDEVKTLLGEYDRCRIKIFDWAFALEGAIMSDRDLKPYLHESSSREGKSGEHSTLVKMLVEKKGCLTPDESQYLILIRNKAAHNQFPCAAEIPLIYRDVSAKVGSIEGSSAKDLPEGSSLVDSLWKKYEMIIRKILPILDPENRFFGKLLNNMSQPINDL

SEQ ID NO: 83 - protein, 1125 aa, Prevotella intermedia
MKMEDDKKTTESTNMLDNKHFWAAFLNLARHNVYITVNHINKVLELKNKKDQDIIIDNDQDILAIKTHWEKVNGDLNKTERLRELMTKHFPFLETAIYTKNKEDKEEVKQEKQAEAQSLESLKDCLFLFLEKLQEARNYYSHYKYSESTKEPMLEEGLLEKMYNIFDDNIQLVIKDYQHNKDINPDEDFKHLDRKGQFKYSFADNEGNITESGLLFFVSLFLEKKDAIWMQQKLTGFKDNRESKKKMTHEVFCRRRMLLPKLRLESTQTQDWILLDMLNELIRCPKSLYERLQGEYRKKFNVPFDSADEDYDAEQEPFKNTLVRHQDRFPYFALRYFDYNEIFTNLRFQIDLGTYHFSIYKKLIGGQKEDRHLTHKLYGFERIQEFAKQNRPDEWKALVKDLDTYETSNERYISETTPHYHLENQKIGIRFRNGNKEIWPSLKTNGENNEKSKYKLDKPYQAEAFLSVHELLPMMFYYLLLKKEEPNNDKKNASIVEGFIKREIRDMYKLYDAFANGEINNIGDLEKYCEDKGIPKRHLPKQMVAILYDEPKDMVKEAKRKQKEMVKDTKKLLATLEKQTQEEIEDGGRNIRLLKSGEIARWLVNDMMRFQPVQKDNEGNPLNNSKANSTEYQMLQRSLALYNKEEKPTRYFRQVNLINSSNPHPFLKWTKWEECNNILSFYRNYLTKKIEFLNKLKPEDWEKNQYFLKLKEPKTNRETLVQGWKNGFNLPRGIFTEPIREWFKRHQNDSKEYEKVEALKRVGLVTKVIPLFFKEEYFKEDAQKEINNCVQPFYSFPYNVGNIHKPDEKDFLPSEERKKLWGDKKDKFKGYKAKVKSKKLTDKEKEEYRSYLEFQSWNKFERELRLVRNQDIVTWLLCTELIDKMKVEGLNVEELQKLRLKDIDTDTAKQEKNNILNRIMPMQLPVTVYEIDDSHNIVKDRPLHTVYIEETKTKLLKQGNFKALVKDRRLNGLFSFVDTSSKAELKDKPISKSVVEYELGEYQNARIETIKDMLLLEKTLIKKYEKLPTDNFSDMLNGWLEGKDESDKARFQNDVKLLVAVRNAFSHNQYPMRNRIAFANINPFSLSSADISEEKKLDIANQLKDKTHKIIKKIIEIEKPIETKE

SEQ ID NO: 84 - protein, 1161 aa, unidentified
  source 1..1161: note = Bacteroidota bacterium
MENQTQKGKGIYYYYTKNEDKHYFGSFLNLANNNIEQIIEEFRIRLSLKDEKNIKEIINNYFTDKKSYTDWERGINILKEYLPVIDYLDLAITDKEFEKIDLKQKETAKRKYFRTNFSLLIDTIIDLRNFYTHYFHKPISINPDVAKFLDKNLLNVCLDIKKQKMKTDKTKQALKDGLDKELKKLIELKKAELKEKKIKTWNITENVEGAVYNDAFNHMVYKNNAGVTILKDYHKSILPDDKIDSELKLNFSISGLVFLLSMFLSKKEIEQFKSNLEGFKGKVIGENGEYEISKFNNSLKYMATHWIFSYLTFKGLKQRVKNTFDKETLLMQMIDELNKVPHEVYQTLSKEQQNEFLEDINEYVQDNEENKKSMENSIVVHPVIRKRYDDKFNYFAIRFLDEFANFPTLKFFVTAGNFVHDKREKQIQGSMLTSDRMIKEKINVFGKLTEIAKYKSDYFSNENTLETSEWELFPNPSYLLIQNNIPVHIDLIHNTEEAKQCQIAIDRIKCTTNPAKKRNTRKSKEEIIKIIYQKNKNIKYGDPTALLSSNELPALIYELLVNKKSGKELENIIVEKIVNQYKTIAGFEKGQNLSNSLITKKLKKSEPNEDKINAEKIILAINRELEITENKLNIIKNNRAEFRTGAKRKHIFYSKELGQEATWIAYDLKRFMPEASRKEWKGFHHSELQKFLAFYDRNKNDAKALLNMFWNFDNDQLIGNDLNSAFREFHFDKFYEKYLIKRDEILEGFKSFISNFKDEPKLLKKGIKDIYRVFDKRYYIIKSTNAQKEQLLSKPICLPRGIFDNKPTYIEGVKVESNSALFADWYQYTYSDKHEFQSFYDMPRDYKEQFEKFELNNIKSIQNKKNLNKSDKFIYFRYKQDLKIKQIKSQDLFIKLMVDELFNVVFKNNIELNLKKLYQTSDERFKNQLIADVQKNREKGDTSDNKMNENFIWNMTIPLSLCNGQIEEPKVKLKDIGKFRKLETDDKVIQLLEYDKSKVWKKLEIEDELENMPNSYERIRREKLLKGIQEFEHFLLEKEKFDGINHPKHFEQDLNPNFKTYVINGVLRKNSKLNYTEIDKLLDLEHISIKDIETSAKEIHLAYFLIHVRNKFGHNQLPKLEAFELMKKYYKKNNEETYAEYFHKVSSQIVNEFKNSLEKHS

SEQ ID NO: 85 - protein, 1165 aa, Chryseobacterium jejuense
MEKTQTGLGIYYDHTKLQDKYFFGGFFNLAQNNIDNVIKAFIIKFFPERKDKDINIAQFLDICFKDNDADSDFQKKNKFLRIHFPVIGFLTSDNDKAGFKKKFALLLKTISELRNFYTHYYHKSIEFPSELFELLDDIFVKTTSEIKKLKKKDDKTQQLLNKNLSEEYDIRYQQQIERLKELKAQGKRVSLTDETAIRNGVFNAAFNHLIYRDGENVKPSRLYQSSYSEPDPAENGISLSQNSILFLLSMFLERKETEDLKSRVKGFKAKIIKQGEEQISGLKFMATHWVFSYLCFKGIKQKLSTEFHEETLLIQIIDELSKVPDEVYSAFDSKTKEKFLEDINEYMKEGNADLSLEDSKVIHPVIRKRYENKFNYFAIRFLDEYLSSTSLKFQVHVGNYVHDRRVKHINGTGFQTERIVKDRIKVFGRLSNISNLKADYIKEQLELPNDSNGWEIFPNPSYIFIDNNVPIHVLADEATKKGIELFKDKRRKEQPEELQKRKGKISKYNIVSMIYKEAKGKDKLRIDEPLALLSLNEIPALLYQILEKGATPKDIELIIKNKLTERFEKIKNYDPETPAPASQISKRLRNNTTAKGQEALNAEKLSLLIEREIENTETKLSSIEEKRLKAKKEQRRNTPQRSIFSNSDLGRIAAWLADDIKRFMPAEQRKNWKGYQHSQLQQSLAYFEKRPQEAFLLLKEGWDTSDGSSYWNNWVMNSFLENNHFEKFYKNYLMKRVKYFSELAGNIKQHTHNTKFLRKFIKQQMPADLFPKRHYILKDLETEKNKVLSKPLVFSRGLFDNNPTFIKGVKVTENPELFAEWYSYGYKTEHVFQHFYGWERDYNELLDSELQKGNSFAKNSIYYNRESQLDLIKLKQDLKIKKIKIQDLFLKRIAEKLFENVFNYPTTLSLDEFYLTQEERAEKERIALAQSLREEGDNSPNIIKDDFIWSKTIAFRSKQIYEPAIKLKDIGKFNRFVLDDEESKASKLLSYDKNKIWNKEQLERELSIGENSYEVIRREKLFKEIQNLELQILSNWSWDGINHPREFEMEDQKNTRHPNFKMYLVNGILRKNINLYKEDEDFWLESLKENDFKTLPSEVLETKSEMVQLLFLVILIRNQFAHNQLPEIQFYNFIRKNYPEIQNNTVAELYLNLIKLAVQKLKDNS

SEQ ID NO: 86 - protein, 1156 aa, unidentified
  source 1..1156: note = Chryseobacterium carnipullorum
MNTRVTGMGVSYDHTKKEDKHFFGGFLNLAQDNITAVIKAFCIKFDKNPMSSVQFAESCFTDKDSDTDFQNKVRYVRTHLPVIGYLNYGGDRNTFRQKLSTLLKAVDSLRNFYTHYYHSPLALSTELFELLDTVFASVAVEVKQHKMKDDKTRQLLSKSLAEELDIRYKQQLERLKELKEQGKNIDLRDEAGIRNGVLNAAFNHLIYKEGEIAKPTLSYSSFYYGADSAENGITISQSGLLFLLSMFLGKKEIEDLKSRIRGFKAKIVRDGEENISGLKFMATHWIFSYLSFKGMKQRLSTDFHEETLLIQIIDELSKVPDEVYHDFDTATREKFVEDINEYIREGNEDFSLGDSTIIHPVIRKRYENKFNYFAVRFLDEFIKFPSLRFQVHLGNFVHDRRIKDIHGTGFQTERVVKDRIKVFGKLSEISSLKTEYIEKELDLDSDTGWEIFPNPSYVFIDNNIPIYISTNKTFKNGSSEFIKLRRKEKPEEMKMRGEDKKEKRDIASMIGNAGSLNSKTPLAMLSLNEMPALLYEILVKKTTPEEIELIIKEKLDSHFENIKNYDPEKPLPASQISKRLRNNTTDKGKKVINPEKLIHLINKEIDATEAKFALLAKNRKELKEKFRGKPLRQTIFSNMELGREATWLADDIKRFMPDILRKNWKGYQHNQLQQSLAFFNSRPKEAFTILQDGWDFADGSSFWNGWIINSFVKNRSFEYFYEAYFEGRKEYFSSLAENIKQHTSNHRNLRRFIDQQMPKGLFENRHYLLENLETEKNKILSKPLVFPRGLFDTKPTFIKGIKVDEQPELFAEWYQYGYSTEHVFQNFYGWERDYNDLLESELEKDNDFSKNSIHYSRTSQLELIKLKQDLKIKKIKIQDLFLKLIAGHIFENIFKYPASFSLDELYLTQEERLNKEQEALIQSQRKEGDHSDNIIKDNFIGSKTVTYESKQISEPNVKLKDIGKFNRFLLDDKVKTLLSYNEDKVWNKNDLDLELSIGENSYEVIRREKLFKKIQNFELQTLTDWPWNGTDHPEEFGTTDNKGVNHPNFKMYVVNGILRKHTDWFKEGEDNWLENLNETHFKNLSFQELETKSKSIQTAFLIIMIRNQFAHNQLPAVQFFEFIQKKYPEIQGSTTSELYLNFINLAVVELLELLEK

SEQ ID NO: 87 - protein, 1036 aa, Chryseobacterium ureilyticum
METQILGNGISYDHTKTEDKHFFGGFLNTAQNNIDLLIKAYISKFESSPRKLNSVQFPDVCFKKNDSDADFQHKLQFIRKHLPVIQYLKYGGNREVLKEKFRLLLQAVDSLRNFYTHFYHKPIQLPNELLTLLDTIFGEIGNEVRQNKMKDDKTRHLLKKNLSEELDFRYQEQLERLRKLKSEGKKVDLRDTEAIRNGVLNAAFNHLIFKDAEDFKPTVSYSSYYYDSDTAENGISISQSGLLFLLSMFLGRREMEDLKSRVRGFKARIIKHEEQHVSGLKFMATHWVFSEFCFKGIKTRLNADYHEETLLIQLIDELSKVPDELYRSFDVATRERFIEDINEYIRDGKEDKSLIESKIVHPVIRKRYESKFNYFAIRFLDEFVNFPTLRFQVHAGNYVHDRRIKSIEGTGFKTERLVKDRIKVFGKLSTISSLKAEYLAKAVNITDDTGWELLPHPSYVFIDNNIPIHLTVDPSFKNGVKEYQEKRKLQKPEEMKNRQGGDKMHKPAISSKIGKSKDINPESPVALLSMNEIPALLYEILVKKASPEEVEAKIRQKLTAVFERIRDYDPKVPLPASQVSKRLRNNTDTLSYNKEKLVELANKEVEQTERKLALITKNRRECREKVKGKFKRQKVFKNAELGTEATWLANDIKRFMPEEQKKNWKGYQHSQLQQSLAFFESRPGEARSLLQAGWDFSDGSSFWNGWVMNSFARDNTFDGFYESYLNGRMKYFLRLADNIAQQSSTNKLISNFIKQQMPKGLFDRRLYMLEDLATEKNKILSKPLIFPRGIFDDKPTFKKGVQVSEEPEAFADWYSYGYDVKHKFQEFYAWDRDYEELLREELEKDTAFTKNSIHYSRESQIELLAKKQDLKVKKVRIQDLYLKLMAEFLFENVFGHELALPLDQFYLTQEERLKQEQEAIVQSQRPKGDDSPNIVKENFIWSKTIPFKSGRVFEPNVKLKDIGKFRNLLTDEKVDILLSYNNTEIGKQVIENELIIGAGSYEFIRREQLFKEIQQMKRLSLRSVRGMGVPIRLNLK

SEQ ID NO: 88 - protein, 1127 aa, unidentified
  source 1..1127: note = Segatella buccae
MQKQDKLFVDRKKNAIFAFPKYITIMENQEKPEPIYYELTDKHFWAAFLNLARHNVYTTINHINRRLEIAELKDDGYMMDIKGSWNEQAKKLDKKVRLRDLIMKHFPFLEAAAYEITNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYDAEEEPFKNTLVRHQDRFPYFVLRYFDLNEIFEQLRFQIDLGTYHFSIYNKRIGDEDEVRHLTHHLYGFARIQDFAQQNQPEVWRKLVKDLDYFEASQEPYIPKTAPHYHLENEKIGIKFCSTHNNLFPSLKTEKTCNGRSKFNLGTQFTAEAFLSVHELLPMMFYYLLLTKDYSRKESADKVEGIIRKEISNIYAIYDAFANGEINSIADLTCRLQKTNILQGHLPKQMISILEGRQKDMEKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNKLKPQKGQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKVLAKDRRLNGLLSFAETTDIDLEKNPITKLSVDHELIKYQTTRISIFEMTLGLEKKLINKYPTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 89 - protein, 1135 aa, Porphyromonas gingivalis
MNTVPASENKGQSRTVEDDPQYFGLYLNLARENLIEVESHVRIKFGKKKLNEESLKQSLLCDHLLSVDRWTKVYGHSRRYLPFLHYFDPDSQIEKDHDSKTGVDPDSAQRLIRELYSLLDFLRNDFSHNRLDGTTFEHLEVSPDISSFITGTYSLACGRAQSRFADFFKPDDFVLAKNRKEQLISVADGKECLTVSGLAFFICLFLDREQASGMLSRIRGFKRTDENWARAVHETFCDLCIRHPHDRLESSNTKEALLLDMLNELNRCPRILYDMLPEEERAQFLPALDENSMNNLSENSLNEESRLLWDGSSDWAEALTKRIRHQDRFPYLMLRFIEEMDLLKGIRFRVDLGEIELDSYSKKVGRNGEYDRTITDHALAFGKLSDFQNEEEVSRMISGEASYPVRFSLFAPRYAIYDNKIGYCHTSDPVYPKSKTGEKRALSNPQSMGFISVHNLRKLLLMELLCEGSFSRMQSDFLRKANRILDETAEGKLQFSALFPEMRHRFIPPQNPKSKDRREKAETTLEKYKQEIKGRKDKLNSQLLSAFDMNQRQLPSRLLDEWMNIRPASHSVKLRTYVKQLNEDCRLRLRKFRKDGDGKARAIPLVGEMATFLSQDIVRMIISEETKKLITSAYYNEMQRSLAQYAGEENRRQFRAIVAELHLLDPSSGHPFLSATMETAHRYTEDFYKCYLEKKREWLAKTFYRPEQDENTKRRISVFFVPDGEARKLLPLLIRRRMKEQNDLQDWIRNKQAHPIDLPSHLFDSKIMELLKVKDGKKKWNEAFKDWWSTKYPDGMQPFYGLRRELNIHGKSVSYIPSDGKKFADCYTHLMEKTVQDKKRELRTAGKPVPPDLAADIKRSFHRAVNEREFMLRLVQEDDRLMLMAINKMMTDREEDILPGLKNIDSILDEENQFSLAVHAKVLEKEGEGGDNSLSLVPATIEIKSKRKDWSKYIRYRYDRRVPGLMSHFPEHKATLDEVKTLLGEYDRCRIKIFDWAFALEGAIMSDRDLKPYLHESSSREGKSGEHSTLVKMLVEKKGCLTPDESQYLILIRNKAAHNQFPCAAEMPLIYRDVSAKVGSIEGSSAKDLPEGSSLVDSLWKKYEMIIRKILPILDPENRFFGKLLNNMSQPINDL

SEQ ID NO: 90 - protein, 1175 aa, Porphyromonas gingivalis
MTEQNEKPYNGTYYTLEDKHFWAAFFNLARHNAYITLAHIDRQLAYSKADITNDEDILFFKGQWKNLDNDLERKARLRSLILKHFSFLEGAAYGKKLFESQSSGNKSSKKKELTKKEKEELQANALSLDNLKSILFDFLQKLKDFRNYYSHYRHPESSELPLFDGNMLQRLYNVFDVSVQRVKRDHEHNDKVDPHRHFNHLVRKGKKDKYGNNDNPFFKHHFVDREEKVTEAGLLFFVSLFLEKRDAIWMQKKIRGFKGGTEAYQQMTNEVFCRSRISLPKLKLESLRTDDWMLLDMLNELVRCPKSLYDRLREEDRARFRVPVDILSDEDDTDGTEEDPFKNTLVRHQDRFPYFALRYFDLKKVFTSLRFHIDLGTYHFAIYKKNIGEQPEDRHLTRNLYGFGRIQDFAEEHRPEEWKRLVRDLDYFETGDKPYITQTTPHYHIEKGKIGLRFVPEGQLLWPSPEVGATRTGRSKYAQDKRFTAEAFLSVHELMPMMFYYFLLREKYSEEASAEKVQGRIKRVIEDVYAVYDAFARDEINTRDELDACLADKGIRRGHLPRQMIAILSQEHKDMEEKVRKKLQEMIADTDHRLDMLDRQTDRKIRIGRKNAGLPKSGVIADWLVRDMMRFQPVAKDTSGKPLNNSKANSTEYRMLQRALALFGGEKERLTPYFRQMNLTGGNNPHPFLHETRWESHTNILSFYRSYLKARKAFLQSIGRSDREENHRFLLLKEPKTDRQTLVAGWKSEFHLPRGIFTEAVRDCLIEMGYDEVGSYKEVGFMAKAVPLYFERACKDRVQPFYDYPFNVGNSLKPKKGRFLSKEKRAEEWESGKERFRDLEAWSHSAARRIEDAFVGIEYASWENKKKIEQLLQDLSLWETFESKLKVKADKINIAKLKKEILEAKEHPYHDFKSWQKFERELRLVKNQDIITWMMCRDLMEENKVEGLDTGTLYLKDIRTDVQEQGSLNVLNHVKPMRLPVVVYRADSRGHVHKEEAPLATVYIEERDTKLLKQGNFKSFVKDRRLNGLFSFVDTGALAMEQYPISKLRVEYELAKYQTARVCAFEQTLELEESLLTRYPHLPDESFREMLESWSDPLLDKWPDLQREVRLLIAVRNAFSHNQYPMYDETIFSSIRKYDPSSLDAIEERMGLNIAHRLSEEVKLAKEMVERIIQA

SEQ ID NO: 91 - protein, 1125 aa, Prevotella pallens; identical to SEQ ID NO: 73

SEQ ID NO: 92 - protein, 1157 aa, Myroides odoratimimus; identical to SEQ ID NO: 74

SEQ ID NO: 93 - protein, 1157 aa, Myroides odoratimimus; identical to SEQ ID NO: 75

SEQ ID NO: 94 - protein, 1127 aa, Prevotella sp.
MQKQDKLFVDRKKNAIFAFPKYITIMENQEKPEPIYYELTDKHFWAAFLNLARHNVYTTINHINRRLEIAELKDDGYMMGIKGSWNEQAKKLDKKVRLRDLIMKHFPFLEAAAYEITNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYDAEEEPFKNTLVRHQDRFPYFVLRYFDLNEIFEQLRFQIDLGTYHFSIYNKRIGDEDEVRHLTHHLYGFARIQDFAPQNQPEEWRKLVKDLDHFETSQEPYISKTAPHYHLENEKIGIKFCSTHNNLFPSLKREKTCNGRSKFNLGTQFTAEAFLSVHELLPMMFYYLLLTKDYSRKESADKVEGIIRKEISNIYAIYDAFANNEINSIADLTCRLQKTNILQGHLPKQMISILEGRQKDMEKEAERKIGEMIDDTQRRLDLLCKQTNQKIRIGKRNAGLLKSGKIADWLVSDMMRFQPVQKDTNNAPINNSKANSTEYRMLQHALALFGSESSRLKAYFRQMNLVGNANPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNKLKPQKGQFLDKKERVELWQKNKELFKNYPSEKNKTDLAYLDFLSWKKFERELRLIKNQDIVTWLMFKELFKTTTVEGLKIGEIHLRDIDTNTANEESNNILNRIMPMKLPVKTYETDNKGNILKERPLATFYIEETETKVLKQGNFKVLAKDRRLNGLLSFAETTDIDLEKNPITKLSVDYELIKYQTTRISIFEMTLGLEKKLIDKYSTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 95 - protein, 1119 aa, Porphyromonas gingivalis
MTEQNERPYNGTYYTLEDKHFWAAFFNLARHNAYITLAHIDRQLAYSKADITNDEDILFFKGQWKNLDNDLERKARLRSLILKHFSFLEGAAYGKKLFESQSSGNKSSKKKELTKKEKEELQANALSLDNLKSILFDFLQKLKDFRNYYSHYRHPESSELPLFDGNMLQRLYNVFDVSVQRVKRDHEHNDKVDPHRHFNHLVRKGKKDRYGNNDNPFFKHHFVDREEKVTEAGLLFFVSLFLEKRDAIWMQKKIRGFKGGTETYQQMTNEVFCRSRISLPKLKLESLRTDDWMLLDMLNELVRCPKSLYDRLREEDRARFRVPVDILSDEDDTDGTEEDPFKNTLVRHQDRFPYFALRYFDLKKVFTSLRFHIDLGTYHFAIYKKNIGEQPEDRHLTRNLYGFGRIQDFAEEHRPEEWKRLVRDLDYFETGDKPYITQTTPHYHIEKGKIGLRFVPEGQHLWPSPEVGATRTGRSKYAQDKRLTAEAFLSVHELMPMMFYYFLLREKYSDEASAERVQGRIKRVIEDVYAVYDAFARGEINTRDELDACLADKGIRRGHLPRQMIGILSQEHKDMEEKVRKKLQEMIVDTDHRLDMLDRQTDRKIRIGRKNAGLPKSGVIADWLVRDMMRFQPVAKDTSGKPLNNSKANSTEYRMLQRALALFGGEKERLTPYFRQMNLTGGNNPHPFLHETRWESHTNILSFYRSYLKARKAFLQSIGRSDRVENHRFLLLKEPKTDRQTLVAGWKGEFHLPRGIFTEAVRDCLIEMGLDEVGSYKEVGFMAKAVPLYFERACKDRVQPFYDYPFNVGNSLKPKKGRFLSKEKRAEEWESGKERFRLAKLKKEILEAKEHPYLDFKSWQKFERELRLVKNQDIITWMICRDLMEENKVEGLDTGTLYLKDIRTDVQEQGNLNVLNRVKPMRLPVVVYRADSRGHVHKEQAPLATVYIEERDTKLLKQGNFKSFVKDRRLNGLFSFVDTGALAMEQYPISKLRVEYELAKYQTARVCAFEQTLELEESLLTRYPHLPDKNFRKMLESWSDPLLDKWPDLHGNVRLLIAVRNAFSHNQYPMYDEAVFSSIRKYDPSSPDAIEERMGLNIAHRLSEEVKQAKEMAERIIQA

SEQ ID NO: 96 - protein, 1154 aa, Paludibacter propionicigenes
MKTSANNIYFNGINSFKKIFDSKGAIAPIAEKSCRNFDIKAQNDVNKEQRIHYFAVGHTFKQLDTENLFEYVLDENLRAKRPTRFISLQQFDKEFIENIKRLISDIRNINSHYIHRFDPLKIDAVPTNIIDFLKESFELAVIQIYLKEKGINYLQFSENPHADQKLVAFLHDKFLPLDEKKTSMLQNETPQLKEYKEYRKYFKTLSKQAAIDQLLFAEKETDYIWNLFDSHPVLTISAGKYLSFYSCLFLLSMFLYKSEANQLISKIKGFKKNTTEEEKSKREIFTFFSKRFNSMDIDSEENQLVKFRDLILYLNHYPVAWNKDLELDSSNPAMTDKLKSKIIELEINRSFPLYEGNERFATFAKYQIWGKKHLGKSIEKEYINASFTDEEITAYTYETDTCPELKDAHKKLADLKAAKGLFGKRKEKNESDIKKTETSIRELQHEPNPIKDKLIQRIEKNLLTVSYGRNQDRFMDFSARFLAEINYFGQDASFKMYHFYATDEQNSELEKYELPKDKKKYDSLKFHQGKLVHFISYKEHLKRYESWDDAFVIENNAIQLKLSFDGVENTVTIQRALLIYLLEDALRNIQNNTAENAGKQLLQEYYSHNKADLSAFKQILTQQDSIEPQQKTEFKKLLPRRLLNNYSPAINHLQTPHSSLPLILEKALLAEKRYCSLVVKAKAEGNYDDFIKRNKGKQFKLQFIRKAWNLMYFRNSYLQNVQAAGHHKSFHIERDEFNDFSRYMFAFEELSQYKYYLNEMFEKKGFFENNEFKILFQSGTSLENLYEKTKQKFEIWLASNTAKTNKPDNYHLNNYEQQFSNQLFFINLSHFINYLKSTGKLQTDANGQIIYEALNNVQYLIPEYYYTDKPERSESKSGNKLYNKLKATKLEDALLYEMAMCYLKADKQIADKAKHPITKLLTSDVEFNITNKEGIQLYHLLVPFKKIDAFIGLKMHKEQQDKKHPTSFLANIVNYLELVKNDKDIRKTYEAFSTNPVKRTLTYDDLAKIDGHLISKSIKFTNVTLELERYFIFKESLIVKKGNNIDFKYIKGLRNYYNNEKKKNEGIRNKAFHFGIPDSKSYDQLIRDAEVMFIANEVKPTHATKYTDLNKQLHTVCDKLMETVHNDYFSKEGDGKKKREAAGQKYFENIISAK

SEQ ID NO: 97 - protein, 1119 aa, Porphyromonas gingivalis
MTEQNEKPYNGTYYTLEDKHFWAAFFNLARHNAYITLAHIDRQLAYSKADITNDEDILFFKGQWKNLDNDLERKARLRSLILKHFSFLEGAAYGKKLFESQSSGNKSSKNKELTKKEKEELQANALSLDNLKSILFDFLQKLKDFRNYYSHYRHPESSELPLFDGNMLQRLYNVFDVSVQRVKRDHEHNDKVDPHRHFNHLVRKGKKDRYGNNDNPFFKHHFVDREGTVTEAGLLFFVSLFLEKRDAIWMQKKIRGFKGGTETYQQMTNEVFCRSRISLPKLKLESLRTDDWMLLDMLNELVRCPKSLYDRLREEDRARFRVPVDILSDEEDTDGAEEDPFKNTLVRHQDRFPYFALRYFDLKKVFTSLRFQIDLGTYHFAIYKKNIGEQPEDRHLTRNLYGFGRIQDFAEEHRPEEWKRLVRDLDYFETGDKPYITQTTPHYHIEKGKIGLRFVPEGQHLWPSPEVGATRTGRSKYAQDKRFTAEAFLSAHELMPMMFYYFLLREKYSEEASAERVQGRIKRVIEDVYAVYDAFARDEINTRDELDACLADKGIRRGHLPRQMIGILSQEHKDMEEKIRKKLQEMMADTDHRLDMLDRQTDRKIRIGRKNAGLPKSGVIADWLVRDMMRFQPVAKDTSGKPLNNSKANSTEYRMLQRALALFGGEKERLTPYFRQMNLTGGNNPHPFLHETRWESHTNILSFYRSYLKARKAFLQSIGRSDRVENHRFLLLKEPKTDRQTLVAGWKGEFHLPRGIFTEAVRDCLIEMGLDEVGSYKEVGFMAKAVPLYFERACKDWVQPFYNYPFNVGNSLKPKKGRFLSKEKRAEEWESGKERFRLAKLKKEILEAKEHPYLDFKSWQKFERELRLVKNQDIITWMICGDLMEENKVEGLDTGTLYLKDIRTDVQEQGSLNVLNRVKPMRLPVVVYRADSRGHVHKEQAPLATVYIEERDTKLLKQGNFKSFVKDRRLNGLFSFVDTGALAMEQYPISKLRVEYELAKYQTARVCAFEQTLELEESLLTRCPHLPDKNFRKMLESWSDPLLDKWPDLHRKVRLLIAVRNAFSHNQYPMYDEAVFSSIRKYDPSFPDAIEERMGLNIAHRLSEEVKQAKETVERIIQA

SEQ ID NO: 98 - protein, 1179 aa, Flavobacterium columnare
MSSKNESYNKQKTFNHYKQEDKYFFGGFLNNADDNLRQVGKEFKTRINFNHNNNELASVFKDYFNKEKSVAKREHALNLLSNYFPVLERIQKHTNHNFEQTREIFELLLDTIKKLRDYYTHHYHKPITINPKIYDFLDDTLLDVLITIKKKKVKNDTSRELLKEKLRPELTQLKNQKREELIKKGKKLLEENLENAVFNHCLRPFLEENKTDDKQNKTVSLRKYRKSKPNEETSITLTQSGLVFLMSFFLHRKEFQVFTSGLEGFKAKVNTIKEEEISLNKNNIVYMITHWSYSYYNFKGLKHRIKTDQGVSTLEQNNTTHSLTNTNTKEALLTQIVDYLSKVPNEIYETLSEKQQKEFEEDINEYMRENPENEDSTFSSIVSHKVIRKRYENKFNYFAMRFLDEYAELPTLRFMVNFGDYIKDRQKKILESIQFDSERIIKKEIHLFEKLSLVTEYKKNVYLKETSNIDLSRFPLFPNPSYVMANNNIPFYIDSRSNNLDEYLNQKKKAQSQNKKRNLTFEKYNKEQSKDAIIAMLQKEIGVKDLQQRSTIGLLSCNELPSMLYEVIVKDIKGAELENKIAQKIREQYQSIRDFTLDSPQKDNIPTTLIKTINTDSSVTFENQPIDIPRLKNAIQKELTLTQEKLLNVKEHEIEVDNYNRNKNTYKFKNQPKNKVDDKKLQRKYVFYRNEIRQEANWLASDLIHFMKNKSLWKGYMHNELQSFLAFFEDKKNDCIALLETVFNLKEDCILTKGLKNLFLKHGNFIDFYKEYLKLKEDFLNTESTFLENGLIGLPPKILKKELSKRFKYIFIVFQKRQFIIKELEEKKNNLYADAINLSRGIFDEKPTMIPFKKPNPDEFASWFVASYQYNNYQSFYELTPDIVERDKKKKYKNLRAINKVKIQDYYLKLMVDTLYQDLFNQPLDKSLSDFYVSKAEREKIKADAKAYQKRNDSSLWNKVIHLSLQNNRITANPKLKDIGKYKRALQDEKIATLLTYDDRTWTYALQKPEKENENDYKELHYTALNMELQEYEKVRSKELLKQVQELEKQILEEYTDFLSTQIHPADFEREGNPNFKKYLAHSILENEDDLDKLPEKVEAMRELDETITNPIIKKAIVLIIIRNKMAHNQYPPKFIYDLANRFVPKKEEEYFATYFNRVFETITKELWENKEKKDKTQV

SEQ ID NO: 99 - protein, 1145 aa, Psychroflexus torquis
MESIIGLGLSFNPYKTADKHYFGSFLNLVENNLNAVFAEFKERISYKAKDENISSLIEKHFIDNMSIVDYEKKISILNGYLPIIDFLDDELENNLNTRVKNFKKNFIILAEAIEKLRDYYTHFYHDPITFEDNKEPLLELLDEVLLKTILDVKKKYLKTDKTKEILKDSLREEMDLLVIRKTDELREKKKTNPKIQHTDSSQIKNSIFNDAFQGLLYEDKGNNKKTQVSHRAKTRLNPKDIHKQEERDFEIPLSTSGLVFLMSLFLSKKEIEDFKSNIKGFKGKVVKDENHNSLKYMATHRVYSILAFKGLKYRIKTDTFSKETLMMQMIDELSKVPDCVYQNLSETKQKDFIEDWNEYFKDNEENTENLENSRVVHPVIRKRYEDKFNYFAIRFLDEFANFKTLKFQVFMGYYIHDQRTKTIGTTNITTERTVKEKINVFGKLSKMDNLKKHFFSQLSDDENTDWEFFPNPSYNFLTQADNSPANNIPIYLELKNQQIIKEKDAIKAEVNQTQNRNPNKPSKRDLLNKILKTYEDFHQGDPTAILSLNEIPALLHLFLVKPNNKTGQQIENIIRIKIEKQFKAINHPSKNNKGIPKSLFADTNVRVNAIKLKKDLEAELDMLNKKHIAFKENQKASSNYDKLLKEHQFTPKNKRPELRKYVFYKSEKGEEATWLANDIKRFMPKDFKTKWKGCQHSELQRKLAFYDRHTKQDIKELLSGCEFDHSLLDINAYFQKDNFEDFFSKYLENRIETLEGVLKKLHDFKNEPTPLKGVFKNCFKFLKRQNYVTESPEIIKKRILAKPTFLPRGVFDERPTMKKGKNPLKDKNEFAEWFVEYLENKDYQKFYNAEEYRMRDADFKKNAVIKKQKLKDFYTLQMVNYLLKEVFGKDEMNLQLSELFQTRQERLKLQGIAKKQMNKETGDSSENTRNQTYIWNKDVPVSFFNGKVTIDKVKLKNIGKYKRYERDERVKTFIGYEVDEKWMMYLPHNWKDRYSVKPINVIDLQIQEYEEIRSHELLKEIQNLEQYIYDHTTDKNILLQDGNPNFKMYVLNGLLIGIKQVNIPDFIVLKQNTNFDKIDFTGIASCSELEKKTIILIAIRNKFAHNQLPNKMIYDLANEFLKIEKNETYANYYLKVLKKMISDLA

SEQ ID NO: 100 - protein, 948 aa, Riemerella anatipestifer
MFFSFHNAQRVIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSRILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKHQPWEPYQYCLLLKVPKENRKNLVKGWEQGGISLPRGLFTEAIRETLSKDLTLSKPIRKEIKKHGRVGFISRAITLYFKEKYQDKHQSFYNLSYKLEAKAPLLKKEEHYEYWQQNKPQSPTESQRLELHTSDRWKDYLLYKRWQHLEKKLRLYRNQDIMLWLMTLELTKNHFKELNLNYHQLKLENLAVNVQEADAKLNPLNQTLPMVLPVKVYPTTAFGEVQYHETPIRTVYIREEQTKALKMGNFKALVKDRRLNGLFSFIKEENDTQKHPISQLRLRRELEIYQSLRVDAFKETLSLEEKLLNKHASLSSLENEFRTLLEEWKKKYAASSMVTDKHIAFIASVRNAFCHNQYPFYKETLHAPILLFTVAQPTTEEKDGLGIAEALLKVLREYCEIVKSQI

SEQ ID NO: 101 - protein, 1139 aa, unidentified
  source 1..1139: note = Hoylesella pleuritidis
MENDKRLEESACYTLNDKHFWAAFLNLARHNVYITVNHINKTLELKNKKNQEIIIDNDQDILAIKTHWAKVNGDLNKTDRLRELMIKHFPFLEAAIYSNNKEDKEEVKEEKQAKAQSFKSLKDCLFLFLEKLQEARNYYSHYKYSESSKEPEFEEGLLEKMYNTFDASIRLVKEDYQYNKDIDPEKDFKHLERKEDFNYLFTDKDNKGKITKNGLLFFVSLFLEKKDAIWMQQKFRGFKDNRGNKEKMTHEVFCRSRMLLPKIRLESTQTQDWILLDMLNELIRCPKSLYERLQGAYREKFKVPFDSIDEDYDAEQEPFRNTLVRHQDRFPYFALRYFDYNEIFKNLRFQIDLGTYHFSIYKKLIGGKKEDRHLTHKLYGFERIQEFTKQNRPDKWQAIIKDLDTYETSNERYISETTPHYHLENQKIGIRFRNDNNDIWPSLKTNGEKNEKSKYNLDKPYQAEAFLSVHELLPMMFYYLLLKMENTDNDKEDNEVGTKKKGNKNNKQEKHKIEEIIENKIKDIYALYDAFTNGEINSIDELAEQREGKDIEIGHLPKQLIVILKNKSKDMAEKANRKQKEMIKDTKKRLATLDKQVKGEIEDGGRNIRLLKSGEIARWLVNDMMRFQPVQKDNEGKPLNNSKANSTEYQMLQRSLALYNKEEKPTRYFRQVNLIKSSNPHPFLEDTKWEECYNILSFYRNYLKAKIKFLNKLKPEDWKKNQYFLMLKEPKTNRKTLVQGWKNGFNLPRGIFTEPIKEWFKRHQNDSEEYKKVEALDRVGLVAKVIPLFFKEEYFKEDAQKEINNCVQPFYSFPYNVGNIHKPEEKNFLHCEERRKLWDKKKDKFKGYKAKEKSKKMTDKEKEEHRSYLEFQSWNKFERELRLVRNQDILTWLLCTKLIDKLKIDELNIEELQKLRLKDIDTDTAKKEKNNILNRVMPMRLPVTVYEIDKSFNIVKDKPLHTVYIEETGTKLLKQGNFKALVKDRRLNGLFSFVKTSSEAESKSKPISKLRVEYELGAYQKARIDIIKDMLALEKTLIDNDENLPTNKFSDMLKSWLKGKGEANKARLQNDVGLLVAVRNAFSHNQYPMYNSEVFKGMKLLSLSSDIPEKEGLGIAKQLKDKIKETIERIIEIEKEIRN

SEQ ID NO: 102 - protein, 1135 aa, Porphyromonas gingivalis; identical to SEQ ID NO: 80

SEQ ID NO: 103 - protein, 1135 aa, Porphyromonas gingivalis
MNTVPASENKGQSRTVEDDPQYFGLYLNLARENLIEVESHVRIKFGKKKLNEESLKQSLLCDHLLSVDRWTKVYGHSRRYLPFLHYFDPDSQIEKDHDSKTGVDPDSAQRLIRELYSLLDFLRNDFSHNRLDGTTFEHLEVSPDISSFITGTYSLACGRAQSRFADFFKPDDFVLAKNRKEQLISVADGKECLTVSGLAFFICLFLDREQASGMLSRIRGFKRTNENWARAVHETFCDLCIRHPHDRLESSNTKEALLLDMLNELNRCPRILYDMLPEEERAQFLPALDENSMNNLSENSLNEESRLLWDGSSDWAEALTKRIRHQDRFPYLMLRFIEEMDLLKGIRFRVDLGEIELDSYSKKVGRNGEYDRTITDHALAFGKLSDFQNEEEVSRMISGEASYPVRFSLFAPRYAIYDNKIGYCHTSDPVYPKSKTGEKRALSNPQSMGFISVHDLRKLLLMELLCEGSFSRMQSGFLRKANRILDETAEGKLQFSALFPEMRHRFIPPQNPKSKDRREKAETTLEKYKQEIKGRKDKLNSQLLSAFDMNQRQLPSRLLDEWMNIRPASHSVKLRTYVKQLNEDCRLRLRKFRKDGDGKARAIPLVGEMATFLSQDIVRMIISEETKKLITSAYYNEMQRSLAQYAGEENRRQFRAIVAELHLLDPSSGHPFLSATMETAHRYTEDFYKCYLEKKREWLAKTFYRPEQDENTKRRISVFFVPDGEARKLLPLLIRRRMKEQNDLQDWIRNKQAHPIDLPSHLFDSKIMELLKVKDGKKKWNEAFKDWWSTKYPDGMQPFYGLRRELNIHGKSVSYIPSDGKKFADCYTHLMEKTVQDKKRELRTAGKPVPPDLAADIKRSFHRAVNEREFMLRLVQEDDRLMLMAINKMMTDREEDILPGLKNIDSILDKENQFSLAVHAKVLEKEGEGGDNSLSLVPATIEIKSKRKDWSKYIRYRYDRRVPGLMSHFPEHKATLDEVKTLLGEYDRCRIKIFDWAFALEGAIMSDRDLKPYLHESSSREGKSGEHSTLVKMLVEKKGCLTPDESQYLILIRNKAAHNQFPCAAEMPLIYRDVSAKVGSIEGSSAKDLPEGSSLVDSLWKKYEMIIRKILPILDHENRFFGKLLNNMSQPINDL

SEQ ID NO: 104 - protein, 1135 aa, Porphyromonas gingivalis; identical to SEQ ID NO: 81

SEQ ID NO: 105 - protein, 1135 aa, Porphyromonas gingivalis; identical to SEQ ID NO: 82

SEQ ID NO: 106 - protein, 1135 aa, Porphyromonas gingivalis
MNTVPASENKGQSRTVEDDPQYFGLYLNLARENLIEVESHVRIKFGKKKLNEESLKQSLLCDHLLSVDRWTKVYGHSRRYLPFLHYFDPDSQIEKDHDSKTGVDPDSAQRLIRELYSLLDFLRNDFSHNRLDGTTFEHLEVSPDISSFITGTYSLACGRAQSRFADFFKPDDFVLAKNRKEQLISVADGKECLTVSGLAFFICLFLDREQASGMLSRIRGFKRTDENWARAVHETFCDLCIRHPHDRLESSNTKEALLLDMLNELNRCPRILYDMLPEEERAQFLPALDENSMNNLSENSLNEESRLLWDGSSDWAEALTKRIRHQDRFPYLMLRFIEEMDLLKGIRFRVDLGEIELDSYSKKVGRNGEYDRTITDHALAFGKLSDFQNEEEVSRMISGEASYPVRFSLFAPRYAIYDNKIGYCHTSDPVYPKSKTGEKRALSNPRSMGFISVHDLRKLLLMELLCEGSFSRMQSDFLRKANRILDETAEGKLQFSALFPEMRHRFIPPQNPKSKDRREKAETTLEKYKQEIKGRKDKLNSQLLSAFDMNQRQLPSRLLDEWMNIRPASHSVKLRTYVKQLNEDCRLRLRKFRKDGDGKARAIPLVGEMATFLSQDIVRMIISEETKKLITSAYYNEMQRSLAQYAGEENRRQFRAIVAELHLLDPSSGHPFLSATMETAHRYTEDFYKCYLEKKREWLAKTFYRPEQDENTKRRISVFFVPDGEARKLLPLLIRRRMKEQNDLQDWIRNKQAHPIDLPSHLFDSKIMELLKVKDGKKKWNEAFKDWWSTKYPDGMQPFYGLRRELNIHGKSVSYIPSDGKKFADCYTHLMEKTVQDKKRELRTAGKPVPPDLAADIKRSFHRAVNEREFMLRLVQEDDRLMLMAINKMMTDREEDILPGLKNIDSILDEENQFSLAVHAKVLEKEGEGGDNSLSLVPATIEIKSKRKDWSKYIRYRYDRRVPGLMSHFPEHKATLDEVKTLLGEYDRCRIKIFDWAFALEGAIMSDRDLKPYLHESSSREGKSGEHSTLVKMLVEKKGCLTPDESQYLILIRNKAAHNQFPCAAEMPLIYRDVSAKVGSIEGSSAKDLPEGSSLVDSLWKKYEMIIRKILPILDPENRFFGKLLNNMSQPINDL

SEQ ID NO: 107 - protein, 1135 aa, Porphyromonas gingivalis; identical to SEQ ID NO: 106

SEQ ID NO: 108 - protein, 1135 aa, Porphyromonas gingivalis; identical to SEQ ID NO: 106

SEQ ID NO: 109 - protein, 1119 aa, Porphyromonas gingivalis
MTEQNERPYNGTYYTLEDKHFWAAFFNLARHNAYITLAHIDRQLAYSKADITNDEDILFFKGQWKNLDNDLERKARLRSLILKHFSFLEGAAYGKKLFESQSSGNKSSKKKELTKKEKEELQANALSLDNLKSILFDFLQKLKDFRNYYSHYRHPESSELPLFDGNMLQRLYNVFDVSVQRVKRDHEHNDKVDPHRHFNHLVRKGKKDRYGNNDNPFFKHHFVDREGTVTEAGLLFFVSLFLEKRDAIWMQKKIRGFKGGTEAYQQMTNEVFCRSRISLPKLKLESLRTDDWMLLDMLNELVRCPKSLYDRLREEDRARFRVPIDILSDEDDTDGTEEDPFKNTLVRHQDRFPYFALRYFDLKKVFTSLRFHIDLGTYHFAIYKKNIGEQPEDRHLTRNLYGFGRIQDFAEEHRPEEWKRLVRDLDYFETGDKPYITQTTPHYHIEKGKIGLRFVPEGQHLWPSPEVGATRTGRSKYAQDKRLTAEAFLSVHELMPMMFYYFLLREKYSEEVSAEKVQGRIKRVIEDVYAVYDAFARGEIDTLDRLDACLADKGIRRGHLPRQMIAILSQEHKDMEEKVRKKLQEMIADTDHRLDMLDRQTDRKIRIGRKNAGLPKSGVIADWLVRDMMRFQPVAKDTSGKPLNNSKANSTEYRMLQRALALFGGEKERLTPYFRQMNLTGGNNPHPFLHETRWESHTNILSFYRSYLKARKAFLQSIGRSDREENHRFLLLKEPKTDRQTLVAGWKSEFHLPRGIFTEAVRDCLIEMGYDEVGSYKEVGFMAKAVPLYFERACKDRVQPFYDYPFNVGNSLKPKKGRFLSKEKRAEEWESGKERFRLAKLKKEILEAKEHPYLDFKSWQKFERELRLVKNQDIITWMMCRDLMEENKVEGLDTGTLYLKDIRTDVHEQGSLNVLNRVKPMRLPVVVYRADSRGHVHKEQAPLATVYIEERDTKLLKQGNFKSFVKDRRLNGLFSFVDTGALAMEQYPISKLRVEYELAKYQTARVCAFEQTLELEESLLTRYPHLPDKNFRKMLESWSDPLLDKWPDLHRKVRLLIAVRNAFSHNQYPMYDEAVFSSIRKYDPSSPDAIEERMGLNIAHRLSEEVKQAKEMAERIIQV

SEQ ID NO: 110 - protein, 1175 aa, Porphyromonas gulae
MTEQSERPYNGTYYTLEDKHFWAAFLNLARHNAYITLTHIDRQLAYSKADITNDQDVLSFKALWKNLDNDLERKSRLRSLILKHFSFLEGAAYGKKLFESKSSGNKSSKNKELTKKEKEELQANALSLDNLKSILFDFLQKLKDFRNYYSHYRHSGSSELPLFDGNMLQRLYNVFDVSVQRVKRDHEHNDKVDPHRHFNHLVRKGKKDRYGHNDNPSFKHHFVDSEGMVTEAGLLFFVSLFLEKRDAIWMQKKIRGFKGGTETYQQMTNEVFCRSRISLPKLKLESLRMDDWMLLDMLNELVRCPKPLYDRLREDDRACFRVPVDILPDEDDTDGGGEDPFKNTLVRHQDRFPYFALRYFDLKKVFTSLRFHIDLGTYHFAIYKKMIGEQPEDRHLTRNLYGFGRIQDFAEEHRPEEWKRLVRDLDYFETGDKPYISQTSPHYHIEKGKIGLRFMPEGQHLWPSPEVGTTRTGRSKYAQDKRLTAEAFLSVHELMPMMFYYFLLREKYSEEVSAEKVQGRIKRVIEDVYAIYDAFARDEINTLKELDACLADKGIRRGHLPKQMIAILSQEHKNMEEKVRKKLQEMIADTDHRLDMLDRQTDRKIRIGRKNAGLPKSGVIADWLVRDMMRFQPVAKDASGKPLNNSKANSTEYRMLQRALALFGGEKERLTPYFRQMNLTGGNNPHPFLHDTRWESHTNILSFYRSYLRARKAFLERIGRSDRMENRPFLLLKEPKTDRQTLVAGWKSEFHLPRGIFTEAVRDCLIEMGYDEVGSYREVGFMAKAVPLYFERACEDRVQPFYDSPFNVGNSLKPKKGRFLSKEERAEEWERGKERFRDLEAWSHSAARRIEDAFAGIEYASPGNKKKIEQLLRDLSLWEAFESKLKVRADKINLAKLKKEILEAQEHPYHDFKSWQKFERELRLVKNQDIITWMMCRDLMEENKVEGLDTGTLYLKDIRTNVQEQGSLNVLNHVKPMRLPVVVYRADSRGHVHKEEAPLATVYIEERDTKLLKQGNFKSFVKDRRLNGLFSFVDTGGLAMEQYPISKLRVEYELAKYQTARVCAFEQTLELEESLLTRYPHLPDKNFRKMLESWSDPLLAKWPELHGKVRLLIAVRNAFSHNQYPMYDEAVFSSIRKYDPSSPDAIEERMGLNIAHRLSEEVKQAKETVERIIQA

SEQ ID NO: 111 - protein, 1119 aa, Porphyromonas gulae
MTEQSERPYNGTYYTLEDKHFWAAFLNLARHNAYITLTHIDRQLAYSKADITNDQDVLSFKALWKNLDNDLERKSRLRSLILKHFSFLEGAAYGKKLFESKSSGNKSSKNKELTKKEKEELQANALSLDNLKSILFDFLQKLKDFRNYYSHYRHSGSSELPLFDGNMLQRLYNVFDVSVQRVKRDHEHNDKVDPHRHFNHLVRKGKKDRYGHNDNPSFKHHFVDGEGMVTEAGLLFFVSLFLEKRDAIWMQKKIRGFKGGTETYQQMTNEVFCRSRISLPKLKLESLRTDDWMLLDMLNELVRCPKPLYDRLREKDRARFRVPVDILPDEDDTDGGGEDPFKNTLVRHQDRFPYFALRYFDLKKVFTSLRFHIDLGTYHFAIYKKVIGEQPEDRHLTRNLYGFGRIQDFAEEHRPEEWKRLVRDLDYFETGDKPYISQTTPHYHIEKGKIGLRFVPEGQHLWPSPEVGTTRTGRSKYAQDKRLTAEAFLSVHELMPMMFYYFLLREKYSEEVSAEKVQGRIKRVIEDVYAIYDAFARDEINTRDELDACLADKGIRRGHLPKQMIGILSQEHKNMEEKVRKKLQEMIADTDHRLDMLDRQTDRKIRIGRKNAGLPKSGVIADWLVRDMMRFQPVAKDTSGKPLNNSKANSTEYRMLQRALALFGGEKERLTPYFRQMNLTGGNNPHPFLDETRWESHTNILSFYRSYLRARKAFLERIGRSDRVENRPFLLLKEPKTDRQTLVAGWKSEFHLPRGIFTEAVRDCLIEMGYDEVGSYKEVGFMAKAVPLYFERACKDRVQPFYDSPFNVGNSLKPKKGRFLSKEKRAEEWESGKERFRLAKLKKEILEAQEHPYHDFKSWQKFERELRLVKNQDIITWMMCRDLMEENKVEGLDTGTLYLKDIRPNVQEQGSLNVLNRVKPMRLPVVVYRADSRGHVHKEEAPLATVYIEERDTKLLKQGNFKSFVKDRRLNGLFSFVDTGGLAMEQYPISKLRVEYELAKYQTARVCVFELTLRLEESLLSRYPHLPDESFREMLESWSDPLLAKWPELHGKVRLLIAVRNAFSHNQYPMYDEAVFSSIRKYDPSSPDAIEERMGLNIAHRLSEEVKQAKETVERIIQA

SEQ ID NO: 112 - protein, 1119 aa, Porphyromonas gulae; identical to SEQ ID NO: 111

SEQ ID NO: 113 - protein, 1175 aa, Porphyromonas gulae
MTEQSERPYNGTYYTLEDKHFWAAFLNLARHNAYITLTHIDRQLAYSKADITNDQDVLSFKALWKNFDNDLERKSRLRSLILKHFSFLEGAAYGKKLFESKSSGNKSSKNKELTKKEKEELQANALSLDNLKSILFDFLQKLKDFRNYYSHYRHSESSELPLFDGNMLQRLYNVFDVSVQRVKRDHEHNDKVDPHRHFNHLVRKGKKDRYGHNDNPSFKHHFVDGEGMVTEAGLLFFVSLFLEKRDAIWMQKKIRGFKGGTETYQQMTNEVFCRSRISLPKLKLESLRTDDWMLLDMLNELVRCPKPLYDRLREDDRACFRVPVDILPDEDDTDGGGEDPFKNTLVRHQDRFPYFALRYFDLKKVFTSLRFHIDLGTYHFAIYKKMIGEQPEDRHLTRNLYGFGRIQDFAEEHRPEEWKRLVRDLDYFETGDKPYISQTSPHYHIEKGKIGLRFMPEGQHLWPSPEVGTTRTGRSKYAQDKRLTAEAFLSVHELMPMMFYYFLLREKYSEEVSAEKVQGRIKRVIEDVYAIYDAFARDEINTLKELDACLADKGIRRGHLPKQMIAILSQEHKDMEEKIRKKLQEMIADTDHRLDMLDRQTDRKIRIGRKNAGLPKSGVIADWLVRDMMRFQPVAKDTSGKPLNNSKANSTEYRMLQRALALFGGEKKRLTPYFRQMNLTGGNNPHPFLHETRWESHTNILSFYRSYLRARKAFLERIGRSDRMENRPFLLLKEPKTDRQTLVAGWKSEFHLPRGIFTEAVRDCLIEMGYDEVGSYREVGFMAKAVPLYFERACEDRVQPFYDSPFNVGNSLKPKKGRFLSKEERAEEWERGKERFRDLEAWSHSAARRIEDAFAGIEYASPGNKKKIEQLLRDLSLWEAFESKLKVRADKINLAKLKKEILEAQEHPYHDFKSWQKFERELRLVKNQDIITWMMCRDLMEENKVEGLDTGTLYLKDIRPNVQEQGSLNVLNRVKPMRLPVVVYRADSRGHVHKEEAPLATVYIEERDTKLLKQGNFKSFVKDRRLNGLFSFVDTGGLAMEQYPISKLRVEYELAKYQTARVCVFELTLRLEESLLTRYPHLPDESFRKMLESWSDPLLAKWPELHGKVRLLIAVRNAFSHNQYPMYDEAVFSSIRKYDPSSPDAIEERMGLNIAHRLSEEVKQAKETVERIIQV

SEQ ID NO: 114 - protein, 1119 aa, Porphyromonas gulae
MTEQSERPYNGTYYTLEDKHFWAAFLNLARHNAYITLTHIDRQLAYSKADITNDEDILFFKGQWKNLDNDLERKSRLRSLILKHFSFLEGAAYGKKFFESKSSGNKSSKNKELTKKEKEELQANALSLDNLKSILFDFLQKLKDFRNYYSHYRHSGSSELPLFDGNMLQRLYNVFDVSVQRVKRDHEHNDEVDPHYHFNHLVRKGKKDRYGHNDNPSFKHHFVDGEGMVTEAGLLFFVSLFLEKRDAIWMQKKIRGFKGGTEPYEQMTNEVFCRSRISLPKLKLESLRTDDWMLLDMLNELVRCPKPLYDRLREKDRACFRVPVDILPDEDDTDGGGEDPFKNTLVRHQDRFPYFALRYFDLKKVFTSLRFHIDLGTYHFAIYKKMIGEQPEDRHLTRNLYGFGRIQDFAEEHRPEEWKRLVRDLDYFETGDKPYISQTTPHYHIEKGKIGLRFVPEGQHLWPSPEVGTTRTGRSKYAQDKRLTAEAFLSVHELMPMMFYYFLLREKYSEEVSAEKVQGRIKRVIEDVYAIYDAFARDEINTLKELDACLADKGIRRGHLPKQMIGILSQERKDMEEKVRKKLQEMIADTDHRLDMLDRQTDRKIRIGRKNAGLPKSGVIADWLVRDMMRFQPVAKDTSGKPLNNSKANSTEYRMLQRALALFGGEKERLTPYFRQMNLTGGNNPHPFLHETRWESHTNILSFYRSYLRARKAFLERIGRSDRVENCPFLLLKEPKTDRQTLVAGWKGEFHLPRGIFTEAVRDCLIEMGYDEVGSYREVGFMAKAVPLYFERACEDRVQPFYDSPFNVGNSLKPKKGRFLSKEKRAEEWESGKERFRLAKLKKEILEAQEHPYHDFKSWQKFERELRLVKNQDIITWMMCRDLMEENKVEGLDTGTLYLKDIRPNVQEQGSLNVLNRVKPMRLPVVVYRADSRGHVHKEEAPLATVYIEERDTKLLKQGNFKSFVKDRRLNGLFSFVDTGALAMEQYPISKLRVEYELAKYQTARVCAFEQTLELEESLLTRYPHLPDESFREMLESWSDPLLTKWPELHGKVRLLIAVRNAFSHNQYPMYDEAVFSSIWKYDPSSPDAIEERMGLNIAHRLSEEVKQAKETIERIIQA

SEQ ID NO: 115 - protein, 1174 aa, Porphyromonas gulae
MTEQSERPYNGTYYTLEDKHFWAAFLNLARHNAYITLTHIDRQLAYSKADITNDQDVLSFKALWKNLDNDLERKSRLRSLILKHFSFLEGAAYGKKLFESKSSGNKSSKNKELTKKEKEELQANALSLDNLKSILFDFLQKLKDFRNYYSHYRHSGSSELPLFDGNMLQRLYNVFDVSVQRVKRDHEHNDKVDPHYHFNHLVRKGKKDRYGHNDNPSFKHHFVDSEGMVTEAGLLFFVSLFLEKRDAIWMQKKIRGFKGGTGPYEQMTNEVFCRSRISLPKLKLESLRTDDWMLLDMLNELVRCPKPLYDRLREKDRACFRVPVDILPDEDDTDGGGEDPFKNTLVRHQDRFPYFALRYFDLKKVFTSLRFHIDLGTYHFAIYKKMIGEQPEDRHLTRNLYGFGRIQDFAEEHRPEEWKRLVRDLDYLETGDKPYISQTTPHYHIEKGKIGLRFVPEGQHLWPSPEVGTTRTGRSKCAQDKRLTAEAFLSVHELMPMMFYYFLLREKYSEEVSAEKVQGRIKRVIEDVYAIYDAFARDEINTLKELDTCLADKGIRRGHLPKQMITILSQERKDMKEKIRKKLQEMIADTDHRLDMLDRQTDRKIRIGRKNAGLPKSGVIADWLVRDMMRFQPVAKDASGKPLNNSKANSTEYRMLQRALALFGGEKERLTPYFRQMNLTGGNNPHPFLHETRWESHTNILSFYRSYLRARKAFLERIGRSDRVENCPFLLLKEPKTDRQTLVAGWKDEFHLPRGIFTEAVRDCLIEMGYDEVGSYREVGFMAKAVPLYFERACEDRVQPFYDSPFNVGNSLKPKKGRFLSKEDRAEEWERGMERFRDLEAWSHSAARRIKDAFAGIEYASPGNKKKIEQLLRDLSLWEAFESKLKVRADKINLAKLKKEILEAQEHPYHDFKSWQKFERELRLVKNQDIITWMMCRDLMEENKVEGLDTGTLYLKDIRPNVQEQGSLNVLNRVKPMRLPVVVYRADSRGHVHKEAPLATVYIEERNTKLLKQGNFKSFVKDRRLNGLFSFVDTGGLAMEQYPISKLRVEYELAKYQTARVCVFELTLRLEESLLSRYPHLPDESFREMLESWSDPLLAKWPELHGKVRLLIAVRNAFSHNQYPMYDEAVFSSIRKYDPSSPDAIEERMGLNIAHRLSEEVKQAKETVERIIQA

SEQ ID NO: 116 - protein, 1135 aa, Porphyromonas gulae
MNTVPATENKGQSRTVEDDPQYFGLYLNLARENLIEVESHVRIKFGKKKLNEESLKQSLLCDHLLSIDRWTKVYGHSRRYLPFLHCFDPDSGIEKDHDSKTGVDPDSAQRLIRELYSLLDFLRNDFSHNRLDGTTFEHLKVSPDISSFITGAYTFACERAQSRFADFFKPDDFLLAKNRKEQLISVADGKECLTVSGFAFFICLFLDREQASGMLSRIRGFKRTDENWARAVHETFCDLCIRHPHDRLESSNTKEALLLDMLNELNRCPRILYDMLPEEERAQFLPALDENSMNNLSENSLNEESRLLWDGSSDWAEALTKRIRHQDRFPYLMLRFIEEMDLLKGIRFRVDLGEIELDSYSKKVGRNGEYDRTITDHALAFGKLSDFQNEEEVSRMISGEASYPVRFSLFAPRYAIYDNKIGYCHTSDPVYPKSKTGEKRALSNPQSMGFISVHDLRKLLLMELLCEGSFSRMQSDFLRKANRILDETAEGKLQFSALFPEMRHRFIPPQNPKSKDRREKAETTLEKYKQEIKGRKDKLNSQLLSAFDMNQRQLPSRLLDEWMNIRPASHSVKLRTYVKQLNEDCRLRLRKFRKDGDGKARAIPLVGEMATFLSQDIVRMIISEETKKLITSAYYNEMQRSLAQYAGEENRRQFRAIVAELHLLDPSSGHPFLSATMETAHRYTEDFYKCYLEKKREWLAKTFYRPEQDENTKRRISVFFVPDGEARKLLPLLIRRRMKEQNDLQDWIRNKQAHPIDLPSHLFDSKIMELLKVKDGKKKWNEAFKDWWSTKYPDGMQPFYGLRRELNIHGKSVSYIPSDGKKFADCYTHLMEKTVRDKKRELRTAGKPVPPDLAAYIKRSFHRAVNEREFMLRLVQEDDRLMLMAINKMMTDREEDILPGLKNIDSILDEENQFSLAVHAKVLEKEGEGGDNSLSLVPATIEIKSKRKDWSKYIRYRYDRRVPGLMSHFPEHKATLDEVKTLLGEYDRCRIKIFDWAFALEGAIMSDRDLKPYLHESSSREGKSGEHSTLVKMLVEKKGCLTPDESQYLILIRNKAAHNQFPCAAEMPLIYRDVSAKVGSIEGSSAKDLPEGSSLVDSLWKKYEMIIRKILPILDHENRFFGKLLNNMSQPINDL

SEQ ID NO: 117 - protein, 1218 aa, Capnocytophaga cynodegmi
MENKTSLGNNIYYNPFKPQDKSYFAGYLNAAMENIDSVFRELGKRLKGKEYTSENFFDAIFKENISLVEYERYVKLLSDYFPMARLLDKKEVPIKERKENFKKNFRGIIKAVRDLRNFYTHKEHGEVEITDEIFGVLDEMLKSTVLTVKKKKIKTDKTKEILKKSIEKQLDILCQKKLEYLKDTARKIEEKRRNQRERGEKKLVPRFEYSDRRDDLIAAIYNDAFDVYIDKKKDSLKESSKTKYNTESYPQQEEGDLKIPISKNGVVFLLSLFLSKQEVHAFKSKIAGFKATVIDEATVSHRKNSICFMATHEIFSHLAYKKLKRKVRTAEINYSEAENAEQLSIYAKETLMMQMLDELSKVPDVVYQNLSEDVQKTFIEDWNEYLKENNGDVGTMEEEQVIHPVIRKRYEDKFNYFAIRFLDEFAQFPTLRFQVHLGNYLHDSRPKEHLISDRRIKEKITVFGRLSELEHKKALFIKNTETNEDRKHYWEVFPNPNYDFPKENISVNDKDFPIAGSILDREKQPTAGKIGIKVNLLNQKYISEVDKAVKAHQLKQRNNKPSIQNIIEEIVPINGSNPKEIIVFGGQPTAYLSMNDIHSILYEFFDKWEKKKEKLEKKGEKELRKEIGKELEEKIVGKIQTQIQQIIDKDINAKILKPYQDDDSTAIDKEKLIKDLKQEQKILQKLKNEQTAREKEYQECIAYQEESRKIKRSDKSRQKYLRNQLKRKYPEVPTRKEILYYQEKGKVAVWLANDIKRFMPTDFKNEWKGEQHSLLQKSLAYYEQCKEELKNLLPQQKVFKHLPFELGGHFQQKYLYQFYTRYLDKRLEHISGLVQQAENFKNENKVFKKVENECFKFLKKQNYTHKGLDAQAQSVLGYPIFLERGFMDEKPTIIKGKTFKGNESLFTDWFRYYKEYQNFQTFYDTENYPLVELEKKQADRKRETKIYQQKKNDVFTLLMAKHIFKSVFKQDSIDRFSLEDLYQSREERLENQEKAKQTGERNTNYIWNKTVDLNLCDGKVTVENVKLKNVGNFIKYEYDQRVQTFLKYEENIKWQAFLIKESKEEENYPYIVEREIEQYEKVRREELLKEVHLIEEYILEKVKDKEILKKGDNQNFKYYILNGLLKQLKNEDVESYKVFNLNTKPEDVNINQLKQEATDLEQKAFVLTYIRNKFAHNQLPKKEFWDYCQEKYGKIEKEKTYAEYFAEVFKREKEALMK

SEQ ID NO: 118 - protein, 1090 aa, unidentified
  source 1..1090: note = Prevotella pectinovora
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLIDGCEFLTSTEQPLSGMISKYYTVALRNTKERYGYKTEDLAFIQDNIKKITKDAYGKRKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSTDRFVPLLLQYIDYGKLFDHIRFHVNMGKLRYLLKADKTCIDGQTRVRVIEQPLNGFGRLEEAETMRKQENGTFGNSGIRIRDFENVKRDDANPANYPYIVDTYTHYILENNKVEMFISDKGSSAPLLPLIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVDFYERYLIERKFYLTGLCNEIKRGNRVDVPFIRRDQNKWKTPAMKTLGRIYSEDLPVELPRQMFDNEIKSHLKSLPQMEGIDFNNANVTYLIAEYMKRVLNDDFQTFYQWKRNYHYMDMLKGEYDRKGSLQHCFTSVEEREGLWKERASRTERYRKLASNKIRSNRQMRNASSEEIETILDKRLSNCRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGIVEIKALPEIAMSIKKAFGEYAIMK

SEQ ID NO: 119 - protein, 1090 aa, unidentified
  source 1..1090: note = Prevotella pectinovora
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDQASHYKTYDEKLIDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKQPKDRIHSEKSNKSVAMDMLNEIKRCPNELFETLSAEKQSRFRIISNDHNEVLMKRSSDRFVPLLLQYIDYGKLFDHIRFHVNMGKLRYLLKADKTCIDGQTRVRVIEQPLNGFGRLEEVETMRKQENGTFGNSGIRIRDFENMKRDDANPANYPYIVDTYTHYILENNKVEMFISDEETPAPLLPVIEDDRYVVKTIPSCRMSTLEIPAMAFHMFLFGSKKTEKLIVDVHNRYKRLFKAMQKEEVTAENIASFGIAESDLPQKIIDLISGNAHGKDVDAFIRLTVDDMLADTERRIKRFKDDRKSIRSADNKMGKRGFKQISTGKLADFLAKDIVLFQPSVNDGENKITGLNYRIMQSAIAVYNSGDDYEAKQQFKLMFEKARLIGKGTTEPHPFLYKVFVRSIPANAVDFYERYLIERKFYLIGLSNEIKKGNRVDVPFIRRDQNKWKTPAMKTLGRIYDEDLPVELPRQMFDNEIKSHLKSLPQMEGIDFNNANVTYLIAEYMKRVLNDDFQTFYQWKRNYRYMDMLRGEYDRKGSLQSCFTSVEEREGLWKERASRTERYRKLASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDTVSKEDIMEEFKKYDQCRPEISSIVFNLEKWAFDTYPELSARVDREEKVDFKSILKILLNNKNINKEQSDILRKIRNAFDHNNYPDKGVVEIRALPEIAMSIKKAFGEYAIMK

SEQ ID NO: 120 - protein, 1090 aa, unidentified
  source 1..1090: note = Prevotella pectinovora
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLIDGCEFLTSTEQPFSGMISKYYTVALRNTKERYGYKAEDLAFIQDNRYKFTKDAYGKRKSQVNTGSFLSLQDYNGDTTKKLHLSGVGIALLICLFLDKQYINLFLSRLPIFSSYNAQSEERRIIIRSFGINSIKQPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEVETMRKQENGTFGNSGIRIRDFENMKRDDANPANYPYIVETYTHYILENNKVEMFISDEENPTPLLPVIEDDRYVVKTIPSCRMSTLEIPAMAFHMFLFGSEKTEKLIIDVHDRYKRLFQAMQKEEVTAENIASFGIAESDLPQKIMDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFVRSIPANAVDFYERYLIERKFYLIGLSNEIKKGNRVDVPFIRRDQNKWKTPAMKTLGRIYSEDLPVELPRQMFDNEIKSHLKSLPQMEGIDFNNANVTYLIAEYMKRVLNDDFQTFYQWKRNYRYMDMLRGEYDRKGSLQHCFTSIEEREGLWKERASRTERYRKLASNKIRSNRQMRNASSEEIETILDKRLSNCRNEYQKSEKIIRRYRVQDALLFLLAKKTLTELADFDGERFKLKEIMPDAEKGILSEIMPMSFTFEKGGKIYTITSGGMKLKNYGDFFVLASDKRIGNLLELVGSNTVSKEDIMEEFKKYDQCRPEISSIVFNLEKWAFDTYPELPARVDRKEKVDFWSILDVLSNNKDINNEQSYILRKIRNAFDHNNYPDKGIVEIKALPEIAMSIKKAFGEYAIMK

SEQ ID NO: 121 - protein, 1131 aa, unidentified
  source 1..1131: note = Phaeodactylibacter xiamenensis
MTNTPKRRTLHRHPSYFGAFLNIARHNAFMIMEHLSTKYDMEDKNTLDEAQLPNAKLFGCLKKRYGKPDVTEGVSRDLRRYFPFLNYPLFLHLEKQQNAEQAATYDINPEDIEFTLKGFFRLLNQMRNNYSHYISNTDYGKFDKLPVQDIYEAAIFRLLDRGKHTKRFDVFESKHTRHLESNNSEYRPRSLANSPDHENTVAFVTCLFLERKYAFPFLSRLDCFRSTNDAAEGDPLIRKASHECYTMFCCRLPQPKLESSDILLDMVNELGRCPSALYNLLSEEDQARFHIKREEITGFEEDPDEELEQEIVLKRHSDRFPYFALRYFDDTEAFQTLRFDVYLGRWRTKPVYKKRIYGQERDRVLTQSIRTFTRLSRLLPIYENVKHDAVRQNEEDGKLVNPDVTSQFHKSWIQIESDDRAFLSDRIEHFSPHYNFGDQVIGLKFINPDRYAAIQNVFPKLPGEEKKDKDAKLVNETADAIISTHEIRSLFLYHYLSKKPISAGDERRFIQVDTETFIKQYIDTIKLFFEDIKSGELQPIADPPNYQKNEPLPYVRGDKEKTQEERAQYRERQKEIKERRKELNTLLQNRYGLSIQYIPSRLREYLLGYKKVPYEKLALQKLRAQRKEVKKRIKDIEKMRTPRVGEQATWLAEDIVFLTPPKMHTPERKTTKHPQKLNNDQFRIMQSSLAYFSVNKKAIKKFFQKETGIGLSNRETSHPFLYRIDVGRCRGILDFYTGYLKYKMDWLDDAIKKVDNRKHGKKEAKKYEKYLPSSIQHKTPLELDYTRLPVYLPRGLFKKAIVKALAAHADFQVEPEEDNVIFCLDQLLDGDTQDFYNWQRYYRSALTEKETDNQLVLAHPYAEQILGTIKTLEGKQKNNKLGNKAKQKIKDELIDLKRAKRRLLDREQYLRAVQAEDRALWLMIQERQKQKAEHEEIAFDQLDLKNITKILTESIDARLRIPDTKVDITDKLPLRRYGDLRRVAKDRRLVNLASYYHVAGLSEIPYDLVKKELEEYDRRRVAFFEHVYQFEKEVYDRYAAELRNENPKGESTYFSHWEYVAVAVKHSADTHFNELFKEKVMQLRNKFHHNEFPYFDWLLPEVEKASAALYADRVFDVAEGYYQKMRKLMRQ

SEQ ID NO: 122 - protein, 1155 aa, Flavobacterium sp.
MDNNITVEKTELGLGITYNHDKVEDKHYFGGFFNLAQNNIDLVAQEFKKRLLIQGKDSINIFANYFSDQCSITNLERGIKILAEYFPVVSYIDLDEKNKSKSIREHLILLLETINNLRNYYTHYYHKKIIIDGSLFPLLDTILLKVVLEIKKKKLKEDKTKQLLKKGLEKEMTILFNLMKAEQKEKKIKGWNIDENIKGAVLNRAFSHLLYNDELSDYRKSKYNTEDETLKDTLTESGILFLLSFFLNKKEQEQLKANIKGYKGKIASIPDEEITLKNNSLRNMATHWTYSHLTYKGLKHRIKTDHEKETLLVNMVDYLSKVPHEIYQNLSEQNKSLFLEDINEYMRDNEENHDSSEASRVIHPVIRKRYENKFAYFAIRFLDEFAEFPTLRFMVNVGNYIHDNRKKDIGGTSLITNRTIKQQINVFGNLTEIHKKKNDYFEKEENKEKTLEWELFPNPSYHFQKENIPIFIDLEKSKETNDLAKEYAKEKKKIFGSSRKKQQNTAKKNRETIINLVFDKYKTSDRKTVTFEQPTALLSFNELNSFLYAFLVENKTGKELEKIIIEKIANQYQILKNCSSTVDKTNDNIPKSIKKIVNTTTDSFYFEGKKIDIEKLEKDITIEIEKTNEKLETIKENEESAQNYKRNERNTQKRKLYRKYVFFTNEIGIEATWITNDILRFLDNKENWKGYQHSELQKFISQYDNYKKEALGLLESEWNLESDAFFGQNLKRMFQSNSTFETFYKKYLDNRKNTLETYLSAIENLKTMTDVRPKVLKKKWTELFRFFDKKIYLLSTIETKINELITKPINLSRGIFEEKPTFINGKNPNKENNQHLFANWFIYAKKQTILQDFYNLPLEQPKAITNLKKHKYKLERSINNLKIEDIYIKQMVDFLYQKLFEQSFIGSLQDLYTSKEKREIEKGKAKNEQTPDESFIWKKQVEINTHNGRIIAKTKIKDIGKFKNLLTDNKIAHLISYDDRIWDFSLNNDGDITKKLYSINTELESYETIRREKLLKQIQQFEQFLLEQETEYSAERKHPEKFEKDCNPNFKKYIIEGVLNKIIPNHEIEEIEILKSKEDVFKINFSDILILNNDNIKKGYLLIMIRNKFAHNQLIDKNLFNFSLQLYSKNENENFSEYLNKVCQNIIQEFKEKLK

SEQ ID NO: 123 - protein, 1175 aa, Porphyromonas gulae
MTEQSERPYNGTYYTLEDKHFWAAFLNLARHNAYITLTHIDRQLAYSKADITNDQDVLSFKALWKNFDNDLERKSRLRSLILKHFSFLEGAAYGKKLFESKSSGNKSSKNKELTKKEKEELQANALSLDNLKSILFDFLQKLKDFRNYYSHYRHSESSELPLFDGNMLQRLYNVFDVSVQRVKRDHEHNDKVDPHRHFNHLVRKGKKDRYGHNDNPSFKHHFVDSEGMVTEAGLLFFVSLFLEKRDAIWMQKKIRGFKGGTETYQQMTNEVFCRSRISLPKLKLESLRTDDWMLLDMLNELVRCPKPLYDRLREKDRARFRVPVDILPDEDDTDGGGEDPFKNTLVRHQDRFPYFALRYFDLKKVFTSLRFHIDLGTYHFAIYKKMIGEQPEDRHLTRNLYGFGRIQDFAEEHRPEEWKRLVRDLDYFETGDKPYISQTTPHYHIEKGKIGLRFMPEGQHLWPSPEVGTTRTGRSKYAQDKRLTAEAFLSVHELMPMMFYYFLLREKYSEEVSAEKVQGRIKRVIEDVYAIYDAFARDEINTLKELDACLADKGIRRGHLPKQMIAILSQEHKDMEEKIRKKLQEMIADTDHRLDMLDRQTDRKIRIGRKNAGLPKSGVIADWLVRDMMRFQPVAKDTSGKPLNNSKANSTEYRMLQRALALFGGEKKRLTPYFRQMNLTGGNNPHPFLHETRWESHTNILSFYRSYLRARKAFLERIGRSDRMENRPFLLLKEPKTDRQTLVAGWKSEFHLPRGIFTEAVRDCLIEMGYDEVGSYREVGFMAKAVPLYFERACEDRVQPFYDSPFNVGNSLKPKKGRFLSKEERAEEWERGKERFRDLEAWSHSAARRIEDAFAGIEYASPGNKKKIEQLLRDLSLWEAFESKLKVRADKINLAKLKKEILEAQEHPYHDFKSWQKFERELRLVKNQDIITWMMCRDLMEENKVEGLDTGTLYLKDIRPNVQEQGSLNVLNRVKPMRLPVVVYRADSRGHVHKEEAPLATVYIEERDTKLLKQGNFKSFVKDRRLNGLFSFVDTGGLAMEQYPISKLRVEYELAKYQTARVCVFELTLRLEESLLTRYPHLPDESFRKMLESWSDPLLAKWPELHGKVRLLIAVRNAFSHNQYPMYDEAVFSSIRKYDPSSPDAIEERMGLNIAHRLSEEVKQAKETVERIIQV

SEQ ID NO: 124 - protein, 1140 aa, Chryseobacterium sp.
METQTIGHGIAYDHSKIQDKHFFGGFLNLAENNIKAVLKAFSEKFNVGNVDVKQFADVSLKDNLPDNDFQKRVSFLKMYFPVVDFINIPNNRAKFRSDLTTLFKSVDQLRNFYTHYYHKPLDFDASLFILLDDIFARTAKEVRDQKMKDDKTRQLLSKSLSEELQKGYELQLERLKELNRLGKKVNIHDQLGIKNGVLNNAFNHLIYKDGESFKTKLTYSSALTSFESAENGIEISQSGLLFLLSMFLKRKEIEDLKNRNKGFKAKVVIDEDGKVNGLKFMATHWVFSYLCFKGLKSKLSTEFHEETLLIQIIDELSKVPDELYCAFDKETRDKFIEDINEYVKEGHQDFSLEDAKVIHPVIRKRYENKFNYFAIRFLDEFVKFPSLRFQVHVGNYVHDRRIKNIDGTTFETERVVKDRIKVFGRLSEISSYKAQYLSSVSDKHDETGWEIFPNPSYVFINNNIPIHISVDTSFKKEIADFKKLRRAQVPDELKIRGAEKKRKFEITQMIGSKSVLNQEEPIALLSLNEIPALLYEILINGKEPAEIERIIKDKLNERQDVIKNYNPENWLPASQISRRLRSNKGERIINTDKLLQLVTKELLVTEQKLKIISDNREALKQKKEGKYIRKFIFTNSELGREAIWLADDIKRFMPADVRKEWKGYQHSQLQQSLAFYNSRPKEALAILESSWNLKDEKIIWNEWILKSFTQNKFFDAFYNEYLKGRKKYFAFLSEHIVQYTSNAKNLQKFIKQQMPKDLFEKRHYIIEDLQTEKNKILSKPFIFPRGIFDKKPTFIKGVKVEDSPESFANWYQYGYQKDHQFQKFYDWKRDYSDVFLEHLGKPFINNGDRRTLGMEELKERIIIKQDLKIKKIKIQDLFLRLIAENLFQKVFKYSAKLPLSDFYLTQEERMEKENMAALQNVREEGDKSPNIIKDNFIWSKMIPYKKGQIIENAVKLKDIGKLNVLSLDDKVQTLLSYDDAKPWSKIALENEFSIGENSYEVIRREKLFKEIQQFESEILFRSGWDGINHPAQLEDNRNPKFKMYIVNGILRKSAGLYSQGEDIWFEYNADFNNLDADVLETKSELVQLAFLVTAIRNKFAHNQLPAKEFYFYIRAKYGFADEPSVALVYLNFTKYAINEF

SEQ ID NO: 125 - protein, 948 aa, Riemerella anatipestifer
MFFSFHNAQRVIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSRILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLMLSKPIRKEIKKHGRVGFISRAITLYFKEKYQDKHQSFYNLSYKLEAKAPLLKREEHYEYWQQNKPQSPTESQRLELHTSDRWKDYLLYKRWQHLEKKLRLYRNQDVMLWLMTLELTKNHFKELNLNYHQLKLENLAVNVQEADAKLNPLNQTLPMVLPVKVYPATAFGEVQYHKTPIRTVYIREEHTKALKMGNFKALVKDRRLNGLFSFIKEENDTQKHPISQLRLRRELEIYQSLRVDAFKETLSLEEKLLNKHTSLSSLENEFRALLEEWKKEYAASSMVTDEHIAFIASVRNAFCHNQYPFYKEALHAPIPLFTVAQPTTEEKDGLGIAEALLKVLREYCEIVKSQI

SEQ ID NO: 126 - protein, 1175 aa, Porphyromonas gingivalis
MTEQNEKPYNGTYYTLEDKHFWAAFFNLARHNAYITLAHIDRQLAYSKADITNDEDILFFKGQWKNLDNDLERKARLRSLILKHFSFLEGAAYGKKLFESQSSGNKSSKKKELTKKEKEELQANALSLDNLKSILFDFLQKLKDFRNYYSHYRHPESSELPLFDGNMLQRLYNVFDVSVQRVKRDHEHNDKVDPHRHFNHLVRKGKKDKYGNNDNPFFKHHFVDREEKVTEAGLLFFVSLFLEKRDAIWMQKKIRGFKGGTEAYQQMTNEVFCRSRISLPKLKLESLRTDDWMLLDMLNELVRCPKLLYDRLREEDRARFRVPVDILSDEDDTDGTEEDPFKNTLVRHQDRFPYFALRYFDLKKVFTSLRFHIDLGTYHFAIYKKNIGEQPEDRHLTRNLYGFGRIQDFAEEHRPEEWKRLVRDLDYFETGDKPYITQTTPHYHIEKGKIGLRFVPEGQLLWPSPEVGATRTGRSKYAQDKRFTAEAFLSVHELMPMMFYYFLLREKYSEEASAEKVQGRIKRVIEDVYAVYDAFARDEINTRDELDACLADKGIRRGHLPRQMIAILSQEHKDMEEKVRKKLQEMIADTDHRLDMLDRQTDRKIRIGRKNAGLPKSGVIADWLVRDMMRFQPVAKDTSGKPLNNSKANSTEYRMLQRALALFGGEKERLTPYFRQMNLTGGNNPHPFLHETRWESHTNILSFYRSYLKARKAFLQSIGRSDREENHRFLLLKEPKTDRQTLVAGWKSEFHLPRGIFTEAVRDCLIEMGYDEVGSYKEVGFMAKAVPLYFERACKDRVQPFYDYPFNVGNSLKPKKGRFLSKEKRAEEWESGKERFRDLEAWSHSAARRIEDAFVGIEYASWENKKKIEQLLQDLSLWETFESKLKVKADKINIAKLKKEILEAKEHPYHDFKSWQKFERELRLVKNQDIITWMMCRDLMEENKVEGLDTGTLYLKDIRTDVQEQGSLNVLNHVKPMRLPVVVYRADSRGHVHKEEAPLATVYIEERDTKLLKQGNFKSFVKDRRLNGLFSFVDTGALAMEQYPISKLRVEYELAKYQTARVCAFEQTLELEESLLTRYPHLPDESFREMLESWSDPLLDKWPDLQREVRLLIAVRNAFSHNQYPMYDETIFSSIRKYDPSSLDAIEERMGLNIAHRLSEEVKLAKEMVERIIQA

SEQ ID NO: 127 - protein, 1175 aa, Porphyromonas gingivalis
MTEQNEKPYNGTYYTLKDKHFWAAFFNLARHNAYITLTHIDRQLAYSKADITNDEDILFFKGQWKNLDNDLERKARLRSLILKHFSFLEGAAYGKKLFESQSSGNKSSKKKELTKKEKEELQANALSLDNLKSILFDFLQKLKDFRNYYSHYRHPESSELPMFDGNMLQRLYNVFDVSVQRVKRDHEHNDKVDPHRHFNHLVRKGKKDRCGNNDNPFFKHHFVDREGKVTEAGLLFFVSLFLEKRDAIWMQKKIRGFKGGTETYQQMTNEVFCRSRISLPKLKLESLRTDDWMLLDMLNELVRCPKSLYDRLREEDRACFRVPVDILSDEDDTDGAEEDPFKNTLVRHQDRFPYFALRYFDLKKVFTSLRFHIDLGTYHFAIYKKNIGEQPEDRHLTRNLYGFGRIQDFAEEHRPEEWKRLVRDLDCFETGDKPYITQTTPHYHIEKGKIGLRFVPEGQHLWPSPEVGATRTGRSKYAQDKRFTAEAFLSVHELMPMMFYYFLLREKYSEEVSAERVQGRIKRVIEDVYAVYDAFARDEINTRDELDACLADKGIRRGHLPRQMIAILSQKHKDMEEKVRKKLQEMIADTDHRLDMLDRQTDRKIRIGRKNAGLPKSGVIADWLVRDMMRFQPVAKDTSGKPLNNSKANSTEYRMLQRALALFGGEKERLTPYFRQMNLTGGNNPHPFLHETRWESHTNILSFYRSYLKARKAFLQSIGRSDRVENHRFLLLKEPKTDRQTLVAGWKGEFHLPRGIFTEAVRDCLIEMGLDEVGSYKEVGFMAKAVPLYFERACKDRVQPFYDYPFNVGNSLKPKKGRFLSKEKRAEEWESGKERFRDLEAWSHSAARRIEDAFAGIENASRENKKKIEQLLQDLSLWETFESKLKVKADKINIAKLKKEILEAKEHPYLDFKSWQKFERELRLVKNQDIITWMMCRDLMEENKVEGLDTGTLYLKDIRTDVQEQGSLNVLNHVKPMRLPVVVYRADSRGHVHKEQAPLATVYIEERDTKLLKQGNFKSFVKDRRLNGLFSFVDTGALAMEQYPISKLRVEYELAKYQTARVCAFEQTLELEESLLTRYPHLPDENFRKMLESWSDPLLDKWPDLHRKVRLLIAVRNAFSHNQYPMYDEAVFSSIRKYDPSSPDAIEERMGLNIAHRLSEEVKQAKEMAERIIQA

SEQ ID NO: 128 - protein, 1213 aa, unidentified
  source 1..1213: note = Flavobacterium covae
MSSKNESYNKQKTFNHYKQEDKYFFGGFLNNADDNLRQVGKEFKTRINFNHNNNELASVFKDYFNKEKSVAKREHALNLLSNYFPVLERIQKHTNHNFEQTREIFELLLDTIKKLRDYYTHHYHKPITINPKVYDFLDDTLLDVLITIKKKKVKNDTSRELLKEKFRPELTQLKNQKREELIKKGKKLLEENLENAVFNHCLRPFLEENKTDDKQNKTVSLRKYRKSKPNEETSITLTQSGLVFLISFFLHRKEFQVFTSGLEGFKAKVNTIKEEEISLNKNNIVYMITHWSYSYYNFKGLKHRIKTDQGVSTLEQNNTTHSLTNTNTKEALLTQIVDYLSKVPNEIYETLSEKQQKEFEEDINEYMRENPENEDSTFSSIVSHKVIRKRYENKFNYFAMRFLDEYAELPTLRFMVNFGDYIKDRQKKILESIQFDSERIIKKEIHLFEKLGLVTEYKKNVYLKETSNIDLSRFPLFPSPSYVMANNNIPFYIDSRSNNLDEYLNQKKKAQSQNRKRNLTFEKYNKEQSKDAIIAMLQKEIGVKDLQQRSTIGLLSCNELPSMLYEVIVKDIKGAELENKIAQKIREQYQSIRDFTLDSPQKDNIPTTLTKTISTDTSVTFENQPIDIPRLKNALQKELTLTQEKLLNVKQHEIEVDNYNRNKNTYKFKNQPKDKVDDNKLQRKYVFYRNEIGQEANWLASDLIHFMKNKSLWKGYMHNELQSFLAFFEDKKNDCIALLETVFNLKEDCILTKDLKNLFLKHGNFIDFYKEYLKLKEDFLNTESTFLENGFIGLPPKILKKELSKRLNYIFIVFQKRQFIIKELEEKKNNLYADAINLSRGIFDEKPTMIPFKKPNPDEFASWFVASYQYNNYQSFYELTPDKIENDKKKKYKNLRAINKVKIQDYYLKLMVDTLYQDLFNQPLDKSLSDFYVSKTDREKIKADAKAYQKRNDSFLWNKVIHLSLQNNRITANPKLKDIGKYKRALQDEKIATLLTYDDRTWTYALQKPEKENENDYKELHYTALNMELQEYEKVRSKKLLKQVQELEKQILDKFYDFSNNATHPEDLEIEDKKGKRHPNFKLYITKALLKNESEIINLENIDIEILIKYYDYNTEKLKEKIKNMDEDEKAKIVNTKENYNKITNVLIKKALVLIIIRNKMAHNQYPPKFIYDLATRFVPKKEEEYFACYFNRVFETITTELWENKKKAKEIV

SEQ ID NO: 129 - protein, 1119 aa, Porphyromonas gingivalis
MTEQNERPYNGTYYTLEDKHFWAAFFNLARHNAYITLTHIDRQLAYSKADITNDEDILFFKGQWKNLDNDLERKARLRSLILKHFSFLEGAAYGKKLFENKSSGNKSSKKKELTKKEKEELQANALSLDNLKSILFDFLQKLKDFRNYYSHYRHPESSELPLFDGNMLQRLYNVFDVSVQRVKRDHEHNDKVDPHRHFNHLVRKGKKDRCGNNDNPFFKHHFVDREGKVTEAGLLFFVSLFLEKRDAIWMQKKIRGFKGGTEAYQQMTNEVFCRSRISLPKLKLESLRTDDWMLLDMLNELVRCPKSLYDRLREEDRARFRVPVDILSDEDDTDGTEEDPFKNTLVRHQDRFPYFALRYFDLKKVFTSLRFHIDLGTYHFAIYKKNIGEQPEDRHLTRNLYGFGRIQDFAEEHRPEEWKRLVRDLDYFETGDKPYITQTTPHYHIEKGKIGLRFVPEGQHLWPSPEVGATRTGRSKYAQDKRLTAEAFLSVHELMPMMFYYFLLREKYSEEVSAEKVQGRIKRVIEDVYAVYDAFARGEIDTLDRLDACLADKGIRRGHLPRQMIAILSQEHKDMEEKVRKKLQEMIADTDHRLDMLDRQTDRKIRIGRKNAGLPKSGVIADWLVRDMMRFQPVAKDTSGKPLNNSKANSTEYRMLQRALALFGGEKERLTPYFRQMNLTGGNNPHPFLHETRWESHTNILSFYRSYLKARKAFLQSIGRSDREENHRFLLLKEPKTDRQTLVAGWKSEFHLPRGIFTEAVRDCLIEMGYDEVGSYKEVGFMAKAVPLYFERACKDRVQPFYDYPFNVGNSLKPKKGRFLSKEKRAEEWESGKERFRLAKLKKEILEAKEHPYLDFKSWQKFERELRLVKNQDIITWMMCRDLMEENKVEGLDTGTLYLKDIRTEVQEQGSLNVLNRVKPMRLPVVVYRADSRGHVHKEQAPLATVYIEERDTKLLKQGNFKSFVKDRRLNGLFSFVDTGGLAMEQYPISKLRVEYELAKYQTARVCAFEQTLELEESLLTRCPHLPDKNFRKMLESWSDPLLDKWPDLQREVWLLIAVRNAFSHNQYPMYDEAVFSSIRKYDPSSPDAIEERMGLNIAHRLSEEVKQAKEMAERIIQA

SEQ ID NO: 130 - protein, 1135 aa, Porphyromonas gingivalis
MNTVPASENKGQSRTVEDDPQYFGLYLNLARENLIEVESHVRIKFGKKKLNEESLKQSLLCDHLLSVDRWTKVYGHSRRYLPFLHYFDPDSQIEKDHDSKTGVDPDSAQRLIRELYSLLDFLRNDFSHNRLDGTTFEHLEVSPDISSFITGTYSLACGRAQSRFADFFKPDDFVLAKNRKEQLISVADGKECLTVSGLAFFICLFLDREQASGMLSRIRGFKRTDENWARAVHETFCDLCIRHPHDRLESSNTKEALLLDMLNELNRCPRILYDMLPEEERAQFLPALDENSMNNLSENSLNEESRLLWDGSSDWAEALTKRIRHQDRFPYLMLRFIEEMDLLKGIRFRVDLGEIELDSYSKKVGRNGEYDRTITDHALAFGKLSDFQNEEEVSRMISGEASYPVRFSLFAPRYAIYDNKIGYCHTSDPVYPKSKTGEKRALSNPQSMGFISVHDLRKLLLMELLCEGSFSRMQSGFLRKANRILDETAEGKLQFSALFPEMRHRFIPPQNPKSKDRREKAETTLEKYKQEIKGRKDKLNSQLLSAFDMNQRQLPSRLLDEWMNIRPASHSVKLRTYVKQLNEDCRLRLRKFRKDGDGKARAIPLVGEMATFLSQDIVRMIISEETKKLITSAYYNEMQRSLAQYAGEENRRQFRAIVAELHLLDPSSGHPFLSATMETAHRYTEDFYKCYLEKKREWLAKTFYRPEQDENTKRRISVFFVPDGEARKLLPLLIRRRMKEQNDLQDWIRNKQAHPIDLPSHLFDSKIMELLKVKDGKKKWNEAFKDWWSTKYPDGMQPFYGLRRELNIHGKSVSYIPSDGKKFADCYTHLMEKTVQDKKRELRTAGKPVPPDLAADIKRSFHRAVNEREFMLRLVQEDDRLMLMAINKMMTDREEDILPGLKNIDSILDKENQFSLAVHAKVLEKEGEGGDNSLSLVPATIEIKSKRKDWSKYIRYRYDRRVPGLMSHFPEHKATLDEVKTLLGEYDRCRIKIFDWAFALEGAIMSDRDLKPYLHESSSREGKSGEHSTLVKMLVEKKGCLTPDESQYLILIRNKAAHNQFPCAAEMPLIYRDVSAKVGSIEGSSAKDLPEGSSLVDSLWKKYEMIIRKILPILDPENRFFGKLLNNMSQPINDL

SEQ ID NO: 131 - protein, 950 aa, Riemerella anatipestifer
MFFSFHNAQRVIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQSEKMTTEVFCRSRILLPKLRLESRYDHNQMLLDMLSELSRCPKLLYEKLSEKDKKCFQVEADGFLDEIEEEQNPFKDTLIRHQDRFPYFALRYLDLNESFKSIRFQVDLGTYHYCIYDKKIGYEQEKRHLTRTLLNFGRLQDFTEINRPQEWKALTKDLDYNETSNQPFISKTTPHYHITDNKIGFRLRTSKELYPSLEVKDGANRIAKYPYNSDFVAHAFISISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVVYDAQNQPIKSSKANSTESRLIRRALALYGGEKNRLEGYFKQTNLIGNTNPHPFLNKFNWKACRNLVDFYQQYLEQREKFLEAIKHQPWEPYQYCLLLKVPKENRKNLVKGWEQGGISLPRGLFTEAIRETLSKDLTLSKPIRKEIKKHGRVGFISRAITLYFKEKYQDKHQSFYNLSYKLEAKAPLLKKEEHYEYWQQNKPQSPTESQRLELHTSDRWKDYLLYKRWQHLEKKLRLYRNQDIMLWLMTLELTKNHFKELNLNYHQLKLENLAVNVQEADAKLNPLNQTLPMVLPVKVYPTTAFGEVQYHETPIRTVYIREEQTKALKMGNFKALVKDRHLNGLFSFIKEENDTQKHPISQLRLRRELEIYQSLRVDAFKETLSLEEKLLNKHASLSSLENEFRTLLEEWKKKYAASSMVTDKHIAFIASVRNAFCHNQYPFYKETLHAPILLFTVAQPTTEEKDGLGIAEALLRVLREYCEIVKSQI

SEQ ID NO: 132 - protein, 1213 aa, unidentified
  source 1..1213: note = Flavobacterium covae
MSSKNESYNKQKTFNHYKQEDKYFFGGFLNNADDNLRQVGKEFKTRINFNHNNNELASVFKDYFNKEKSVAKREHALNLLSNYFPVLERIQKHTNHNFEQTREIFELLLDTIKKLRDYYTHHYHKPITINPKIYDFLDDTLLDVLITIKKKKVKNDTSRELLKEKLRPELTQLKNQKREELIKKGKKLLEENLENAVFNHCLRPFLEENKTDDKQNKTVSLRKYRKSKPNEETSITLTQSGLVFLMSFFLHRKEFQVFTSGLEGFKAKVNTIKEEKISLNKNNIVYMITHWSYSYYNFKGLKHRIKTDQGVSTLEQNNTTHSLTNTNTKEALLTQIVDYLSKVPNEIYETLSEKQQKEFEEDINEYMRENPENEDSTFSSIVSHKVIRKRYENKFNYFAMRFLDEYAELPTLRFMVNFGDYIKDRQKKILESIQFDSERIIKKEIHLFEKLGLVTEYKKNVYLKETSNIDLSRFPLFPSPSYVMANNNIPFYIDSRSNNLDEYLNQKKKAQSQNRKRNLTFEKYNKEQSKDAIIAMLQKEIGVKDLQQRSTIGLLSCNELPSMLYEVIVKDIKGAELENKIAQKIREQYQSIRDFTLNSPQKDNIPTTLIKTISTDTSVTFENQPIDIPRLKNAIQKELALTQEKLLNVKQHEIEVNNYNRNKNTYKFKNQPKDKVDDNKLQRKYVFYRNEIGQEANWLASDLIHFMKNKSLWKGYMHNELQSFLAFFEDKKNDCIALLETVFNLKEDCILTKDLKNLFLKHGNFIDFYKEYLKLKEDFLNTESTFLENGFIGLPPKILKKELSKRLNYIFIVFQKRQFIIKELEEKKNNLYADAINLSRGIFDEKPTMIPFKKPNPDEFASWFVASYQYNNYQSFYELTPDKIENDKKKKYKNLRAINKVKIQDYYLKLMVDTLYQDLFNQPLDKSLSDFYVSKTDREKIKADAKAYQKRNDSFLWNKVIHLSLQNNRITANPKLKDIGKYKRALQDEKIATLLTYDDRTWTYALQKPEKENENDYKELHYTALNMELQEYEKVRSKKLLKQVQELEKQILDKFYDFSNNATHPEDLEIEDKKGKRHPNFKLYITKALLKNESEIINLENIDIEILIKYYDYNTEKLKEKIKNMDEDEKAKIVNTKENYNKITNVLIKKALVLIIIRNKMAHNQYPPKFIYDLATRFVPKKEEEYFACYFNRVFETITTELWENKKKAKEIV

SEQ ID NO: 133 - protein, 1095 aa, Riemerella anatipestifer
MEKPLPPNVYTLKHKFFWGAFLNIARHNAFITICHINEQLGLTTPPNDDKIADVVCGTWNNILNNDHDLLKKSQLTELILKHFPFLAAMCYHPPKKEGKKKGSQKEQQKEKENEAQSQAEALNPSELIKVLKTLVKQLRTLRNYYSHHSHKKPDAEKDIFKHLYKAFDASLRMVKEDYKAHFTVNLTQDFAHLNRKGKNKQDNPDFDRYRFEKDGFFTESGLLFFTNLFLDKRDAYWMLKKVSGFKASHKQSEKMTTEVFCRSRILLPKLRLESRYDHNQMLLDMLSELSRYPKLLYEKLSEEDKKRFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSKQPFISKTTPHYHITDNKIGFRLGTSKELYPSLEVKDGANRIAQYPYNSDFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIESSKANSTEFQLIQRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSKDLTLSKPIRKEIKKHGRVGFISRAITLYFREKYQDDHQSFYDLPYKLEAKASPLPKKEHYEYWQQNKPQSPTELQRLELHTSDRWKDYLLYKRWQHLEKKLRLYRNQDVMLWLMTLELTKNHFKELNLNYHQLKLENLAVNVQEADAKLNPLNQTLPMVLPVKVYPATAFGEVQYQETPIRTVYIREEQTKALKMGNFKALVKDRRLNGLFSFIKEENDTQKHPISQLRLRRELEIYQSLRVDAFKETLNLEEKLLKKHTSLSSVENKFRILLEEWKKEYAASSMVTDEHIAFIASVRNAFCHNQYPFYEEALHAPIPLFTVAQQTTEEKDGLGIAEALLRVLREYCEIVKSQI

SEQ ID NO: 134 - protein, 656 aa, unidentified
  source 1..656: note = Sinomicrobium oceani
PMERRDGKPTKIEISNQIDRNIKDNNFKDIYPGEPLAMLSLNELPALLFELLRRPSITPQDIEDRMVEKLYERFQIIRDYKPGDGLSTSKISKKLRKADNSTRLDGKKLLRAIQTETRNAREKLHTLEENKALQKNRKRRTVYTTREQGREASWLAQDLKRFMPIASRKEWRGYHHSQLQQILAFYDQNPKQPLELLEQFWDLKEDTYVWNSWIHKSLSQHNGFVPMYEGYLKGRLGYYKKLESDIIGFLEEHKVLKRYYTQQHLNVIFRERLYFIKTETKQKLELLARPLVFPRGIFDDKPTFVQDKKVVDHPELFADWYVYSYKDDHSFQEFYHYKRDYNEIFETELSWDIDFKDNKRQLNPSEQMDLFRMKWDLKIKKIKIQDIFLKIVAEDIYLKIFGHKIPLSLSDFYISRQERLTLDEQAVAQSMRLPGDTSENQIKESNLWQTTVPYEKEQIREPKIKLKDIGKFKYFLQQQKVLNLLKYDPQHVWTKAELEEELYIGKHSYEVVRREMLLQKCHQLEKHILEQFRFDGSNHPRELEQGNHPNFKMYIVNGILTKRGELEIEAENWWLELGNSKNSLDKVEVELLTMKTIPEQKAFLLILIRNKFAHNQLPADNYFHYASNLMNLKKSDTYSLFWFTVADTIVQEFMSL

SEQ ID NO: 135 - protein, 1133 aa, Reichenbachiella agariperforans
MKTNPLIASSGEKPNYKKFNTESDKSFKKIFQNKGSIAPIAEKACKNFEIKSKSPVNRDGRLHYFSVGHAFKNIDSKNVFRYELDESQMDMKPTQFLALQKEFFDFQGALNGLLKHIRNVNSHYVHTFEKLEIQSINQKLITFLIEAFELAVIHSYLNEEELSYEAYKDDPQSGQKLVQFLCDKFYPNKEHEVEERKTILAKNKRQALEHLLFIEVTSDIDWKLFEKHKVFTISNGKYLSFHACLFLLSLFLYKSEANQLISKIKGFKRNDDNQYRSKRQIFTFFSKKFTSQDVNSEEQHLVKFRDVIQYLNHYPSAWNKHLELKSGYPQMTDKLMRYIVEAEIYRSFPDQTDNHRFLLFAIREFFGQSCLDTWTGNTPINFSNQEQKGFSYEINTSAEIKDIETKLKALVLKGPLNFKEKKEQNRLEKDLRREKKEQPTNRVKEKLLTRIQHNMLYVSYGRNQDRFMDFAARFLAETDYFGKDAKFKMYQFYTSDEQRDHLKEQKKELPKKEFEKLKYHQSKLVDYFTYAEQQARYPDWDTPFVVENNAIQIKVTLFNGAKKIVSVQRNLMLYLLEDALYSEKRENAGKGLISGYFVHHQKELKDQLDILEKETEISREQKREFKKLLPKRLLHRYSPAQINDTTEWNPMEVILEEAKAQEQRYQLLLEKAILHQTEEDFLKRNKGKQFKLRFVRKAWHLMYLKELYMNKVAEHGHHKSFHITKEEFNDFCRWMFAFDEVPKYKEYLCDYFSQKGFFNNAEFKDLIESSTSLNDLYEKTKQRFEGWSKDLTKQSDENKYLLANYESMLKDDMLYVNISHFISYLESKGKINRNAHGHIAYKALNNVPHLIEEYYYKDRLAPEEYKSHGKLYNKLKTVKLEDALLYEMAMHYLSLEPALVPKVKTKVKDILSSNIAFDIKDAAGHHLYHLLIPFHKIDSFVALINHQSQQEKDPDKTSFLAKIQPYLEKVKNSKDLKAVYHYYKDTPHTLRYEDLNMIHSHIVSQSVQFTKVALKLEEYFIAKKSITLQIARQISYSEIADLSNYFTDEVRNTAFHFDVPETAYSMILQGIESEFLDREIKPQKPKSLSELSTQQVSVCTAFLETLHNNLFDRKDDKKERLSKARERYFEQIN

SEQ ID NO: 136 - protein, 668 aa, Fusobacterium necrophorum
MTEKKSIIFKNKSSVEIVKKDIFSQTPDNMIRNYKITLKISEKNPRVVEAEIEDLMNSTILKDGRRSARREKSMTERKLIEEKVAENYSLLANCPMEEVDSIKIYKIKRFLTYRSNMLLYFASINSFLCEGIKGKDNETEEIWHLKDNDVRKEKVKENFKNKLIQSTENYNSSLKNQIEEKEKLLRKESKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKFDSMKAHFHNINSEDTKEAYFWDIHSSSNYKTKYNERKNLVNEYTELLGSSKEKKLLREEITQINRKLLKLKQEMEEITKKNSLFRLEYKMKIAFGFLFCEFDGNISKFKDEFDASNQEKIIQYHKNGEKYLTYFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKIKKFL

SEQ ID NO: 137 - protein, 1110 aa, Fusobacterium necrophorum
MKVRYRKQAQLDTFIIKTEIVNNDIFIKSIIEKAREKYRYSFLFDGEEKYHFKNKSSVEIVKNDIFSQTPDNMIRNYKITLKISEKNPRVVEAEIEDLMNSTILKDGRRSARREKSMTERKLIEEKVAENYSLLANCPIEEVDSIKIYKIKRFLTYRSNMLLYFASINSFLCEGIKGKDNETEEIWHLKDNDVRKEKVKENFKNKLIQSTENYNSSLKNQIEEKEKLSSKEFKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKLDSMKAHFRNINSEDTKEAYFWDIHSSRNYKTKYNERKNLVNEYTKLLGSSKEKKLLREEITKINRQLLKLKQEMEEITKKNSLFRLEYKMKIAFGFLFCEFDGNISKFKDEFDASNQEKIIQYHKNGEKYLTSFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKNKKVSLKNNGMFNKTILLFVFKYYQIAFKLFNDIELYSLFFLREKSEKPFEVFLEELKDKMIGKQLNFGQLLYVVYEVLVKNKDLDKILSKKIDYRKDKSFSPEIAYLRNFLSHLNYSKFLDNFMKINTNKSDENKEVLIPSIKIQKMIQFIEKCNLQNQIDFDFNFVNDFYMRKEKMFFIQLKQIFPDINSTEKQKKSEKEEILRKRYHLINKKNEQIKDEHEAQSQLYEKILSLQKIFSCDKNNFYRRLKEEKLLFLEKQGKKKISMKEIKDKIASDISDLLGILKKEITRDIKDKLTEKFRYCEEKLLNISFYNHQDKKKEEGIRVFLIRDKNSDNFKFESILDDGSNKIFISKNGKEITIQCCDKVLETLMIEKNTLKISSNGKIISLIPHYSYSIDVKY

SEQ ID NO: 138 - protein, 1115 aa, Fusobacterium necrophorum
MEKFRRQNRNSIIKIIISNYDTKGIKELKVRYRKQAQLDTFIIKTEIVNNDIFIKSIIEKAREKYRYSFLFDGEEKYHFKNKSSVEIVKKDIFSQTPDNMIRNYKITLKISEKNPRVVEAEIEDLMNSTILKDGRRSARREKSMTERKLIEEKVAKNYSLLANCPMEEVDSIKIYKIKRFLTYRSNMLLYFASINSFLCEGIKGKDNETEEIWHLKDNDVRKEKVRENFKNKLIQSTENYNSSLKNQIEEKEKLLRKEFKKGAFYRTIIKKLQQERIKELSEKSLTEDCEKIIKLYSKLRHSLMHYDYQYFENLFENKKNDDLMKDLNLDLFKSLPLIRKMKLNNKVNYLEDGDTLFVLQKTKKAKTLYQIYDALCEQKNGFNKFINDFFVSDGEENTVFKQIINEKFQSEMEFLEKRISESEKKNEKLKKKLDSMKAHFRNINSEDTKEAYFWDIHSSRNYKTKYNERKNLVNEYTELLGSSKEKKLLREEITKINRQLLKLKQEMEEITKKNSLFRLEYKMKIAFGFLFCEFDGNISKFKDEFDASNQEKIIQYHKNGEKYLTSFLKEEEKEKFNLEKMQKIIQKTEEEDWLLPETKNNLFKFYLLTYLLLPYELKGDFLGFVKKHYYDIKNVDFIDENQNNIQVSQTVEKQEDYFYHKIRLFEKNTKKYEIVKYSIVPNEKLKQYFEDLGIDIKYLTVEQKSEVSEEKNKKVSLKNNGMFNKTILLFVFKYYQIAFKLFNDIELYSLFFLREKSGKPLEIFRKELESKMKDGYLNFGQLLYVVYEVLVKNKDLDKILSKKIDYRKDKSFSPEIAYLRNFLSHLNYSKFLDNFMKINTNKSDENKEVLIPSIKIQKMIQFIEKCNLQNQIDFDFNFVNDFYMRKEKMFFIQLKQIFPDINSTEKQKMNEKEEILRNRYHLTDKKNEQIKDEHEAQSQLYEKILSLQKIYSSDKNNFYGRLKEEKLLFLEKQGKKKLSMEEIKDKIAGDISDLLGILKKEITRDIKDKLTEKFRYCEEKLLNLSFYNHQDKKKEESIRVFLIRDKNSDNFKFESILDDGSNKIFISKNGKEITIQCCDKVLETLIIEKNTLKISSNGKIISLIPHYSYSIDVKY

SEQ ID NO: 139 - protein, 796 aa, Tissierella sp.
MEKDKKGEKIDISQEMIEEDLRKILILFSRLRHSMVHYDYEFYQALYSGKDFVISDKNNLENRMISQLLDLNIFKELSKVKLIKDKAISNYLDKNTTIHVLGQDIKAIRLLDIYRDICGSKNGFNKFINTMITISGEEDREYKEKVIEHFNKKMENLSTYLEKLEKQDNAKRNNKRVYNLLKQKLIEQQKLKEWFGGPYVYDIHSSKRYKELYIERKKLVDRHSKLFEEGLDEKNKKELTKINDELSKLNSEMKEMTKLNSKYRLQYKLQLAFGFILEEFDLNIDTFINNFDKDKDLIISNFMKKRDIYLNRVLDRGDNRLKNIIKEYKFRDTEDIFCNDRDNNLVKLYILMYILLPVEIRGDFLGFVKKNYYDMKHVDFIDKKDKEDKDTFFHDLRLFEKNIRKLEITDYSLSSGFLSKEHKVDIEKKINDFINRNGAMKLPEDITIEEFNKSLILPIMKNYQINFKLLNDIEISALFKIAKDRSITFKQAIDEIKNEDIKKNSKKNDKNNHKDKNINFTQLMKRALHEKIPYKAGMYQIRNNISHIDMEQLYIDPLNSYMNSNKNNITISEQIEKIIDVCVTGGVTGKELNNNIINDYYMKKEKLVFNLKLRKQNDIVSIESQEKNKREEFVFKKYGLDYKDGEINIIEVIQKVNSLQEELRNIKETSKEKLKNKETLFRDISLINGTIRKNINFKIKEMVLDIVRMDEIRHINIHIYYKGENYTRSNIIKFKYAIDGENKKYYLKQHEINDINLELKDKFVTLICNMDKHPNKNKQTINLESNYIQNVKFIIP

SEQ ID NO: 140 - protein, 1111 aa, Fusobacterium necrophorum
MEKFRRQNRSSIIKIIISNYDTKGIKELKVRYRKQAQLDTFIIKTEIVNNDIFIKSIIEKAREKYRYSFLFDGEEKYHFKNKSSVEIVKKDIFSQTPDNMIRNYKITLKISEKNPRVVEAEIEDLMNSTILKDGRRSARREKSMTERKLIEEKVAENYSLLANCPMEEVDSIKIYKIKRFLTYRSNMLLYFASINSFLCEGIKGKDNETEEIWHLKDNDVRKEKVKENFKNKLIQSTENYNSSLKNQIEEKEKLLRKESKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEIEFLEKRISESEKKNEKLKKKLDSMKAHFRNINSEDTKEAYFWDIHSSRNYKTKYNERKNLVNEYTELLGSSKEKKLLREEITKINRQLLKLKQEMEEITKKNSLFRLEYKMKMAFGFLFCEFDGNISRFKDEFDASNQEKIIQYHKNGEKYLTYFLKEEEKEKFNLKKLQETIQKTGEENWLLPQNKNNLFKFYLLTYLLLPYELKGDFLGFVKKHYYDIKNVDFMDENQSSKIIESKEDDFYHKIRLFEKNTKKYEIVKYSIVPDKKLKQYFKDLGIDTKYLILDQKSEVSGEKNKKVSLKNNGMFNKTILLFVFKYYQIAFKLFNDIELYSLFFLREKSGKPFEVFLKELKDKMIGKQLNFGQLLYVVYEVLVKNKDLSEILSERIDYRKDMCFSAEIADLRNFLSHLNYSKFLDNFMKINTNKSDENKEVLIPSIKIQKMIKFIEECNLQSQIDFDFNFVNDFYMRKEKMFFIQLKQIFPDINSTEKQKMNEKEEILRNRYHLTDKKNEQIKDEHEAQSQLYEKILSLQKIYSSDKNNFYGRLKEEKLLFLEKQEKKKLSMEEIKDKIAGDISDLLGILKKEITRDIKDKLTEKFRYCEEKLLNLSFYNHQDKKKEESIRVFLIRDKNSDNFKFESILDDGSNKIFISKNGKEITIQCCDKVLETLIIEKNTLKISSNGKIISLIPHYSYSIDVKY

SEQ ID NO: 141 - protein, 1121 aa, Fusobacterium perfoetens
MGKPNRSSIIKIIISNYDNKGIKEVKVRYNKQAQLDTFLIKSELKDGKFILYSIVDKAREKYRYSFEIDKTNINKNEILIIKKDIYSNKEDKVIRKYILSFEVSEKNDRTIVTKIKDCLETQKKEKFERENTRRLISETERKLLSEETQKTYSKIACCSPEDIDSVKIYKIKRYLAYRSNMLLFFSLINDIFVKGVVKDNGEEVGEIWRIIDSKEIDEKKTYDLLVENFKKRMSQEFINYKQSIENKIEKNTNKIKEIEQKLKKEKYKKEINRLKKQLIELNRENDLLEKDKIELSDEEIREDIEKILKIYSDLRHKLMHYNYQYFENLFENKKISKEKNEDVNLTELLDLNLFRYLPLVRQLKLENKTNYLEKEDKITVLGVSDSAIKYYSYYNFLCEQKNGFNNFINSFFSNDGEENKSFKEKINLSLEKEIEIMEKETNEKIKEINKNELQLMKEQKELGTAYVLDIHSLNDYKISHNERNKNVKLQNDIMNGNRDKNALDKINKKLVELKIKMDKITKRNSILRLKYKLQVAYGFLMEEYKGNIKKFKDEFDISKEKIKSYKSKGEKYLEVKSEKKYITKILNSIEDIHNITWLKNQEENNLFKFYVLTYILLPFEFRGDFLGFVKKHYYDIKNVEFLDENNDRLTPEQLEKMKNDSFFNKIRLFEKNSKKYDILKESILTSERIGKYFSLLNTGAKYFEYGGEENRGIFNKNIIIPIFKYYQIVLKLYNDVELAMLLTLSESDEKDINKIKELVTLKEKVSPKKIDYEKKYKFSVLLDCFNRIINLGKKDFLASEEVKEVAKTFTNLAYLRNKICHLNYSKFIDDLLTIDTNKSTTDSEGKLLINDRIRKLIKFIRENNQKMNISIDYNYINDYYMKKEKFIFGQRKQAKTIIDSGKKANKRNKAEELLKMYRVKKENINLIYELSKKLNELTKSELFLLDKKLLKDIDFTDVKIKNKSFFELKNDVKEVANIKQALQKHSSELIGIYKKEVIMAIKRSIVSKLIYDEEKVLSIIIYDKTNKKYEDFLLEIRRERDINKFQFLIDEKKEKLGYEKIIETKEKKKVVVKIQNNSELVSEPRIIKNKDKKKAKTPEEISKLGILDLTNHYCFNLKITL

SEQ ID NO: 142 - protein, 1121 aa, Fusobacterium perfoetens; identical to SEQ ID NO: 141

SEQ ID NO: 143 - protein, 1111 aa, unidentified
  source 1..1111: note = Anaerosalibacter massiliensis
MKSGRREKAKSNKSSIVRVIISNFDDKQVKEIKVLYTKQGGIDVIKFKSTEKDEKGRMKFNFDCAYNRLEEEEFNSFGGKGKQSFFVTTNEDLTELHVTKRHKTTGEIIKDYTIQGKYTPIKQDRTKVTVSITDNKDHFDSNDLGDKIRLSRSLTQYTNRILLDADVMKNYREIVCSDSEKVDETINIDSQEIYKINRFLSYRSNMIIYYQMINNFLLHYDGEEDKGGNDSINLINEIWKYENKKNDEKEKIIERSYKSIEKSINQYILNHNTEVESGDKEKKIDISEERIKEDLKKTFILFSRLRHYMVHYNYKFYENLYSGKNFIIYNKDKSKSRRFSELLDLNIFKELSKIKLVKNRAVSNYLDKKTTIHVLNKNINAIKLLDIYRDICETKNGFNNFINNMMTISGEEDKEYKEMVTKHFNENMNKLSIYLENFKKHSDFKTNNKKKETYNLLKQELDEQKKLRLWFNAPYVYDIHSSKKYKELYVERKKYVDIHSKLIEAGINNDNKKKLNEINVKLCELNTEMKEMTKLNSKYRLQYKLQLAFGFILEEFNLDIDKFVSAFDKDNNLTISKFMEKRETYLSKSLDRRDNRFKKLIKDYKFRDTEDIFCSDRENNLVKLYILMYILLPVEIRGDFLGFVKKNYYDLKHVDFIDKRNNDNKDTFFHDLRLFEKNVKRLEVTSYSLSDGFLGKKSREKFGKELEKFIYKNVSIALPTNIDIKEFNKSLVLPMMKNYQIIFKLLNDIEISALFLIAKKEGNEGSITFKKVIDKVRKEDMNGNINFSQVMKMALNEKVNCQIRNSIAHINMKQLYIEPLNIYINNNQNKKTISEQMEEIIDICITKGLTGKELNKNIINDYYMKKEKLVFNLKLRKRNNLVSIDAQQKNMKEKSILNKYDLNYKDENLNIKEIILKVNDLNNKQKLLKETTEGESNYKNALSKDILLLNGIIRKNINFKIKEMILGIIQQNEYRYVNINIYDKIRKEDHNIDLKINNKYIEISCYENKSNESTDERINFKIKYMDLKVKNELLVPSCYEDIYIKKKIDLEIRYIENCKVVYIDIYYKKYNINLEFDGKTLFVKFNKDVKKNNQKVNLESNYIQNIKFIVS

SEQ ID NO: 144 - protein, 1137 aa, unidentified
  UNSURE 1..1137: note = Fusobacterium necrophrom
MEKFRRQNRNSIIKIIISNYDTKGIKELKVRYRKQAQLDTFIIKTEIVNNDIFIKSIIEKAREKYRYSFLFDGEEKYHFKNKSSVEIVKKDIFSQTPDNMIRNYKITLKISEKNPRVVEAEIEDLMNSTILKDGRRSARREKSMTERKLIEEKVAKNYSLLANCPMEEVDSIKIYKIKRFLTYRSNMLLYFASINSFLCEGIKGKDNETEEIWHLKDNDVRKEKVRENFKNKLIQSTENYNSSLKNQIEEKEKLLRKEFKKGAFYRTIIKKLQQERIKELSEKSLTEDCEKIIKLYSKLRHSLMHYDYQYFENLFENKKNDDLMKDLNLDLFKSLPLIRKMKLNNKVNYLEDGDTLFVLQKTKKAKTLYQIYDALCEQKNGFNKFINDFFVSDGEENTVFKQIINEKFQSEMEFLEKRISESEKKNEKLKKKLDSMKAHFRNINSEDTKEAYFWDIHSSRNYKTKYNERKNLVNEYTELLGSSKEKKLLREEITKINRQLLKLKQEMEEITKKNSLFRLEYKMKIAFGFLFCEFDGNISKFKDEFDASNQEKIIQYHKNGEKYLTSFLKEEEKEKFNLEKMQKIIQKTEEEDWLLPETKNNLFKFYLLTYLLLPYELKGDFLGFVKKHYYDIKNVDFIDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKNKKVSLKNNGMFNKTILLFVFKYYQIAFKLFNDIELYSLFFLREKSGKPLEIFRKELESKMKDGYLNFGQLLYVVYEVLVKNKDLDKILSKKIDYRKDKSFSPEIAYLRNFLSHLNYSKFLDNFMKINTNKSDENKEVLIPSIKIQKMIQFIEKCNLQNQIDFDFNFVNDFYMRKEKMFFIQLKQIFPDINSTEKQKMNEKEEILRNRYHLTDKKNEQIKDEHEAQSQLYEKILSLQKIYSSDKNNFYGRLKEEKLLFLEKQGKKKLSMEEIKDKIAGDISDLLGILKKEITRDIKDKLTEKFRYCEEKLLNLSFYNHQDKKKEESIRVFLIRDKNSDNFKFESILDDGSNKIFISKNGKEITIQCCDKVLETLIIEKNTLKISSNGKIISLIPHYSYSIDVKY

SEQ ID NO: 145 - protein, 919 aa, Cetobacterium sp.
MEEIKHKKNKSSIIRVIVSNYDMTGIKEIKVLYQKQGGVDTFNLKTIINLESGNLEIISCKPKEREKYRYEFNCKTEINTISITKKDKVLKKEIRKYSLELYFKNEKKDTVVAKVTDLLKAPDKIEGERNHLRKLSSSTERKLLSKTLCKNYSEISKTPIEEIDSIKIYKIKRFLNYRSNFLIYFALINDFLCAGVKEDDINEVWLIQDKEHTAFLENRIEKITDYIFDKLSKDIENKKNQFEKRIKKYKTSLEELKTETLEKNKTFYIDSIKTKITNLENKITELSLYNSKESLKEDLIKIISIFTNLRHSLMHYDYKSFENLFENIENEELKNLLDLNLFKSIRMSDEFKTKNRTNYLDGTESFTIVKKHQNLKKLYTYYNNLCDKKNGFNTFINSFFVTDGIENTDFKNLIILHFEKEMEEYKKSIEYYKIKISNEKNKSKKEKLKEKIDLLQSELINMREHKNLLKQIYFFDIHNSIKYKELYSERKNLIEQYNLQINGVKDVTAINHINTKLLSLKNKMDKITKQNSLYRLKYKLKIAYSFLMIEFDGDVSKFKNNFDPTNLEKRVEYLDKKEEYLNYTAPKNKFNFAKLEEELQKIQSTSEMGADYLNVSPENNLFKFYILTYIMLPVEFKGDFLGFVKNHYYNIKNVDFMDESLLDENEVDSNKLNEKIENLKDSSFFNKIRLFEKNIKKYEIVKYSVSTQENMKEYFKQLNLDIPYLDYKSTDEIGIFNKNMILPIFKYYQNVFKLCNDIEIHALLALANKKQQNLEYAIYCCSKKNSLNYNELLKTFNRKTYQNLSFIRNKIAHLNYKELFSDLFNNELDLNTKVRCLIEFSQNNKFDQIDLGMNFINDYYMKKTRFIFNQRRLRDLNVPSKEKIIDGKRKQQNDSNNELLKKYGLSRTNIKDIFNKAWY

SEQ ID NO: 146 - protein, 1111 aa, Fusobacterium necrophorum
MEKFRRQNRNSIIKIIISNYDTKGIKELKVRYRKQAQLDTFIIKTEIVNNDIFIKSIIEKAREKYRYSFLFDGEEKYHFKNKSSVEIVKKDIFSQTPDNMIRNYKITLKISEKNPRVVEAEIEDLMNSTILKDGRRSARREKSVTERKLIEEKVAENYSLLANCPMEEVDSIKIYKIKRFLTYRSNMLLYFASINSFLCEGIKGKENETEEIWHLKDNDVRKEKVKENFKNKLIQSTENYNSSLKNQIEEKEKLLRKESKKGAFYRTIIKKLQQERIKELSEKSLTEDCEKIIKLYSKLRHSLMHYDYQYFENLFENKETPELKDKLDLHLFKSLPLIRKMKLNNKVNYLEDGDTLFVLQKTKKAKTLYQIYDALCEQKNGFNKFINDFFVSDGEENTVFKQIINEKFQSEMEFLGKRISESEEKNPKLKKKFDSMKAHFHNINSEDTKEAYFWDIHSSSNYKTKYNERKNLVNEYTELLGSSKEKKLLREEITQINRKLLKLKQEMEEITKKNSLFRLEYKMKMAFGFLFCEFDGNISRFKDEFDASNQEKIIQYHKNGEKYLTYFLKEEEKEKFNLKKLQETIQKTGKENWLLPQNKNNLFKFYLLTYLLLPYELKGDFLGFVKKHYYDIKNVDFMDENQSSKIIESKEDDFYHKIRLFEKNTKKYEIVKYSIVPDEKLKQYFKDLGIDTKYLILEQKSEVSGEKNKKVSLKNNGMFNKTILLFVFKYYQIAFKLFNDIELYSLFFLREKSGKPFEVFLKELKDKMIGKQLNFGQLLYVIYEVLVKNKDLSEILSERIDYRKDMCFSAEIADLRNFLSHLNYSKFLDNFMKINTNKSDENKEVLIPSIKIQKMIKFIEECNLQSQIDFDFNFVNDFYMRKEKMFFIQLKQIFPDINSTEKQKMNEKEEILRNRYHLTDKKNEQIKDEHEAQSQLYEKILSLQKIYSSDKNNFYGRLKEEKLLFLGKQGKKKLSMEEIKDKIAGDISDLLGILKKEITRDIKDKLTEKFRYCEEKLLNLSFYNHQDKKKEESIRVFLIRDKNSDNFKFESILDDGSNKIFISKNGKEITIQCCDKVLETLMIEKNTLKISSNGKIISLVPHYSYSIDVKY

SEQ ID NO: 147 - protein, 1158 aa, Fusobacterium varium
MENKNKPNRGSIVRIIISNYDMKGIKELKVRYRKQAQLDTFILQTTLDKSNNSILINDFRVKAREKYRYSFTYDGKEKFSVPSNSIIVTKIDNAAPEKSKEIRKYKITLGIDEKCKTGSMITAAIEDLLEDDRVREGIRNPRRKASKTERKLITESICHNYAQITQCPVEEIDAVKIYKVKRFLSYRSNMLLFFALINDFLCKNLKNEKGEKINEIWEMENKGNNKKIDFDENYNILVAQIKEYFTKEIENYNNRIDNIIDKKELLKYSEEKEESEKNKKLEELNKLESQKLKILTDEEIKADVIKIIKIFSDLRHSLMHYEYKYFENLFENKKNEELAELLNLNLFKNLTLLRQMKIENKTNYLEGDEKFNILGKDVRAKNALGHYDLLVEQKNGFNNFINSFFVQDGTENLEFKKFIDENFIKAQKELEEDIKNCKESVKKLEKKLKENPKKSEDLEKKLEKKQKKLKELKKELEKMKQHYKRLNCAYVWDIHSSTVYKKLYNERKNLIEKYNKQLNGLQDKNAITGINAQLLRIKKEMEEITKSNSLFRLKYKMQIAYAFLEMEYEGNIAKFKNEFDCSKTEKIQEWLEKSEEYLNYCMEKEEDGKNYKFHFKEISEIKDTHNEEWLENTSENNLFKFYILTYLLLPMEFKGDFLGVVKKHYYDIKNVDFTDESEKELSQEQIDKMIGDSFFHKIRLFEKNTKRYEIIKYSILTSDEIKKYFELLELKVPYLEYKGIDEIGIFNKNIILPIFKYYQIIFRLYNDLEIHGLFNVSFDINKILSDLKSYGNENINFREFLYVIKQNNNSSTEEEYQKIWEKLESKYLKEPLLTPEKKEINKKTEKELKKLDGISFLRNKISHLEYEKIIEGVLKTAVNGENKKTSETNADKVFLNEKIKKIINFIKENELDKIELGFNFINDFFMKKEQFMFGQIKQVKEGNSDSITTERKRKEENNKRLKITYGLNYNNLSKIYEFSNTLREIVNSPLFLKDSTLLKKVDLSKVMLKEKPICSLQYENNTKLEDDIKRILLKDSSDIMGIYKAEVVKKLKEKLVLIFKYDEEKKIYVTVYDTSKAVPENISKEILVKRNNSKEEYFFEDNKKKYTTQYYTLEITKENELKVIPAKKLEGKEFKTEKKEENKLMLNNHYCFNVKIIY

SEQ ID NO: 148 - protein, 1197 aa, Leptotrichia shahii
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALEKRPAATKKAGQAKKKKGSYPYDVPDYAYPYDVPDYAYPYDVPDYA

SEQ ID NO: 149 - protein, 1197 aa, Leptotrichia wadei; identical to SEQ ID NO: 148

SEQ ID NO: 150 - protein, 1340 aa, Listeria seeligeri
MQISKVNHKHVAVGQKDRERITGFIYNDPVGDEKSLEDVVAKRANDTKVLFNVFNTKDLYDSQESDKSEKDKEIISKGAKFVAKSFNSAITILKKQNKIYSTLTSQQVIKELKDKFGGARIYDDDIEEALTETLKKSFRKENVRNSIKVLIENAAGIRSSLSKDEEELIQEYFVKQLVEEYTKTKLQKNVVKSIKNQNMVIQPDSDSQVLSLSESRREKQSSAVSSDTLVNCKEKDVLKAFLTDYAVLDEDERNSLLWKLRNLVNLYFYGSESIRDYSYTKEKSVWKEHDEQKANKTLFIDEICHITKIGKNGKEQKVLDYEENRSRCRKQNINYYRSALNYAKNNTSGIFENEDSNHFWIHLIENEVERLYNGIENGEEFKFETGYISEKVWKAVINHLSIKYIALGKAVYNYAMKELSSPGDIEPGKIDDSYINGITSFDYEIIKAEESLQRDISMNVVFATNYLACATVDTDKDFLLFSKEDIRSCTKKDGNLCKNIMQFWGGYSTWKNFCEEYLKDDKDALELLYSLKSMLYSMRNSSFHFSTENVDNGSWDTELIGKLFEEDCNRAARIEKEKFYNNNLHMFYSSSLLEKVLERLYSSHHERASQVPSFNRVFVRKNFPSSLSEQRITPKFTDSKDEQIWQSAVYYLCKEIYYNDFLQSKEAYKLFREGVKNLDKNDINNQKAADSFKQAVVYYGKAIGNATLSQVCQAIMTEYNRQNNDGLKKKSAYAEKQNSNKYKHYPLFLKQVLQSAFWEYLDENKEIYGFISAQIHKSNVEIKAEDFIANYSSQQYKKLVDKVKKTPELQKWYTLGRLINPRQANQFLGSIRNYVQFVKDIQRRAKENGNPIRNYYEVLESDSIIKILEMCTKLNGTTSNDIHDYFRDEDEYAEYISQFVNFGDVHSGAALNAFCNSESEGKKNGIYYDGINPIVNRNWVLCKLYGSPDLISKIISRVNENMIHDFHKQEDLIREYQIKGICSNKKEQQDLRTFQVLKNRVELRDIVEYSEIINELYGQLIKWCYLRERDLMYFQLGFHYLCLNNASSKEADYIKINVDDRNISGAILYQIAAMYINGLPVYYKKDDMYVALKSGKKASDELNSNEQTSKKINYFLKYGNNILGDKKDQLYLAGLELFENVAEHENIIIFRNEIDHFHYFYDRDRSMLDLYSEVFDRFFTYDMKLRKNVVNMLYNILLDHNIVSSFVFETGEKKVGRGDSEVIKPSAKIRLRANNGVSSDVFTYKVGSKDELKIATLPAKNEEFLLNVARLIYYPDMEAVSENMVREGVVKVEKSNDKKGKISRGSNTRSSNQSKYNNKSKNRMNYSMGSIFEKMDLKFD

SEQ ID NO: 151 - protein, 1437 aa, unidentified
  source 1..1437: note = Lachnospiraceae bacterium
MKISKVREENRGAKLTVNAKTAVVSENRSQEGILYNDPSRYGKSRKNDEDRDRYIESRLKSSGKLYRIFNEDKNKRETDELQWFLSEIVKKINRRNGLVLSDMLSVDDRAFEKAFEKYAELSYTNRRNKVSGSPAFETCGVDAATAERLKGIISETNFINRIKNNIDNKVSEDIIDRIIAKYLKKSLCRERVKRGLKKLLMNAFDLPYSDPDIDVQRDFIDYVLEDFYHVRAKSQVSRSIKNMNMPVQPEGDGKFAITVSKGGTESGNKRSAEKEAFKKFLSDYASLDERVRDDMLRRMRRLVVLYFYGSDDSKLSDVNEKFDVWEDHAARRVDNREFIKLPLENKLANGKTDKDAERIRKNTVKELYRNQNIGCYRQAVKAVEEDNNGRYFDDKMLNMFFIHRIEYGVEKIYANLKQVTEFKARTGYLSEKIWKDLINYISIKYIAMGKAVYNYAMDELNASDKKEIELGKISEEYLSGISSFDYELIKAEEMLQRETAVYVAFAARHLSSQTVELDSENSDFLLLKPKGTMDKNDKNKLASNNILNFLKDKETLRDTILQYFGGHSLWTDFPFDKYLAGGKDDVDFLTDLKDVIYSMRNDSFHYATENHNNGKWNKELISAMFEHETERMTVVMKDKFYSNNLPMFYKNDDLKKLLIDLYKDNVERASQVPSFNKVFVRKNFPALVRDKDNLGIELDLKADADKGENELKFYNALYYMFKEIYYNAFLNDKNVRERFITKATKVADNYDRNKERNLKDRIKSAGSDEKKKLREQLQNYIAENDFGQRIKNIVQVNPDYTLAQICQLIMTEYNQQNNGCMQKKSAARKDINKDSYQHYKMLLLVNLRKAFLEFIKENYAFVLKPYKHDLCDKADFVPDFAKYVKPYAGLISRVAGSSELQKWYIVSRFLSPAQANHMLGFLHSYKQYVWDIYRRASETGTEINHSIAEDKIAGVDITDVDAVIDLSVKLCGTISSEISDYFKDDEVYAEYISSYLDFEYDGGNYKDSLNRFCNSDAVNDQKVALYYDGEHPKLNRNIILSKLYGERRFLEKITDRVSRSDIVEYYKLKKETSQYQTKGIFDSEDEQKNIKKFQEMKNIVEFRDLMDYSEIADELQGQLINWIYLRERDLMNFQLGYHYACLNNDSNKQATYVTLDYQGKKNRKINGAILYQICAMYINGLPLYYVDKDSSEWTVSDGKESTGAKIGEFYRYAKSFENTSDCYASGLEIFENISEHDNITELRNYIEHFRYYSSFDRSFLGIYSEVFDRFFTYDLKYRKNVPTILYNILLQHFVNVRFEFVSGKKMIGIDKKDRKIAKEKECARITIREKNGVYSEQFTYKLKNGTVYVDARDKRYLQSIIRLLFYPEKVNMDEMIEVKEKKKPSDNNTGKGYSKRDRQQDRKEYDKYKEKKKKEGNFLSGMGGNINWDEINAQLKN

SEQ ID NO: 152 - protein, 1385 aa, Clostridium aminophilum
MKFSKVDHTRSAVGIQKATDSVHGMLYTDPKKQEVNDLDKRFDQLNVKAKRLYNVFNQSKAEEDDDEKRFGKVVKKLNRELKDLLFHREVSRYNSIGNAKYNYYGIKSNPEEIVSNLGMVESLKGERDPQKVISKLLLYYLRKGLKPGTDGLRMILEASCGLRKLSGDEKELKVFLQTLDEDFEKKTFKKNLIRSIENQNMAVQPSNEGDPIIGITQGRFNSQKNEEKSAIERMMSMYADLNEDHREDVLRKLRRLNVLYFNVDTEKTEEPTLPGEVDTNPVFEVWHDHEKGKENDRQFATFAKILTEDRETRKKEKLAVKEALNDLKSAIRDHNIMAYRCSIKVTEQDKDGLFFEDQRINRFWIHHIESAVERILASINPEKLYKLRIGYLGEKVWKDLLNYLSIKYIAVGKAVFHFAMEDLGKTGQDIELGKLSNSVSGGLTSFDYEQIRADETLQRQLSVEVAFAANNLFRAVVGQTGKKIEQSKSEENEEDFLLWKAEKIAESIKKEGEGNTLKSILQFFGGASSWDLNHFCAAYGNESSALGYETKFADDLRKAIYSLRNETFHFTTLNKGSFDWNAKLIGDMFSHEAATGIAVERTRFYSNNLPMFYRESDLKRIMDHLYNTYHPRASQVPSFNSVFVRKNFRLFLSNTLNTNTSFDTEVYQKWESGVYYLFKEIYYNSFLPSGDAHHLFFEGLRRIRKEADNLPIVGKEAKKRNAVQDFGRRCDELKNLSLSAICQMIMTEYNEQNNGNRKVKSTREDKRKPDIFQHYKMLLLRTLQEAFAIYIRREEFKFIFDLPKTLYVMKPVEEFLPNWKSGMFDSLVERVKQSPDLQRWYVLCKFLNGRLLNQLSGVIRSYIQFAGDIQRRAKANHNRLYMDNTQRVEYYSNVLEVVDFCIKGTSRFSNVFSDYFRDEDAYADYLDNYLQFKDEKIAEVSSFAALKTFCNEEEVKAGIYMDGENPVMQRNIVMAKLFGPDEVLKNVVPKVTREEIEEYYQLEKQIAPYRQNGYCKSEEDQKKLLRFQRIKNRVEFQTITEFSEIINELLGQLISWSFLRERDLLYFQLGFHYLCLHNDTEKPAEYKEISREDGTVIRNAILHQVAAMYVGGLPVYTLADKKLAAFEKGEADCKLSISKDTAGAGKKIKDFFRYSKYVLIKDRMLTDQNQKYTIYLAGLELFENTDEHDNITDVRKYVDHFKYYATSDENAMSILDLYSEIHDRFFTYDMKYQKNVANMLENILLRHFVLIRPEFFTGSKKVGEGKKITCKARAQIEIAENGMRSEDFTYKLSDGKKNISTCMIAARDQKYLNTVARLLYYPHEAKKSIVDTREKKNNKKTNRGDGTFNKQKGTARKEKDNGPREFNDTGFSNTPFAGFDPFRNS

SEQ ID NO: 153 - protein, 1175 aa, Carnobacterium gallinarum
MRITKVKIKLDNKLYQVTMQKEEKYGTLKLNEESRKSTAEILRLKKASFNKSFHSKTINSQKENKNATIKKNGDYISQIFEKLVGVDTNKNIRKPKMSLTDLKDLPKKDLALFIKRKFKNDDIVEIKNLDLISLFYNALQKVPGEHFTDESWADFCQEMMPYREYKNKFIERKIILLANSIEQNKGFSINPETFSKRKRVLHQWAIEVQERGDFSILDEKLSKLAEIYNFKKMCKRVQDELNDLEKSMKKGKNPEKEKEAYKKQKNFKIKTIWKDYPYKTHIGLIEKIKENEELNQFNIEIGKYFEHYFPIKKERCTEDEPYYLNSETIATTVNYQLKNALISYLMQIGKYKQFGLENQVLDSKKLQEIGIYEGFQTKFMDACVFATSSLKNIIEPMRSGDILGKREFKEAIATSSFVNYHHFFPYFPFELKGMKDRESELIPFGEQTEAKQMQNIWALRGSVQQIRNEIFHSFDKNQKFNLPQLDKSNFEFDASENSTGKSQSYIETDYKFLFEAEKNQLEQFFIERIKSSGALEYYPLKSLEKLFAKKEMKFSLGSQVVAFAPSYKKLVKKGHSYQTATEGTANYLGLSYYNRYELKEESFQAQYYLLKLIYQYVFLPNFSQGNSPAFRETVKAILRINKDEARKKMKKNKKFLRKYAFEQVREMEFKETPDQYMSYLQSEMREEKVRKAEKNDKGFEKNITMNFEKLLMQIFVKGFDVFLTTFAGKELLLSSEEKVIKETEISLSKKINEREKTLKASIQVEHQLVATNSAISYWLFCKLLDSRHLNELRNEMIKFKQSRIKFNHTQHAELIQNLLPIVELTILSNDYDEKNDSQNVDVSAYFEDKSLYETAPYVQTDDRTRVSFRPILKLEKYHTKSLIEALLKDNPQFRVAATDIQEWMHKREEIGELVEKRKNLHTEWAEGQQTLGAEKREEYRDYCKKIDRFNWKANKVTLTYLSQLHYLITDLLGRMVGFSALFERDLVYFSRSFSELGGETYHISDYKNLSGVLRLNAEVKPIKIKNIKVIDNEENPYKGNEPEVKPFLDRLHAYLENVIGIKAVHGKIRNQTAHLSVLQLELSMIESMNNLRDLMAYDRKLKNAVTKSMIKILDKHGMILKLKIDENHKNFEIESLIPKEIIHLKDKAIKTNQVSEEYCQLVLALLTTNPGNQLN

SEQ ID NO: 154 - protein, 1164 aa, Carnobacterium gallinarum
MRMTKVKINGSPVSMNRSKLNGHLVWNGTTNTVNILTKKEQSFAASFLNKTLVKADQVKGYKVLAENIFIIFEQLEKSNSEKPSVYLNNIRRLKEAGLKRFFKSKYHEEIKYTSEKNQSVPTKLNLIPLFFNAVDRIQEDKFDEKNWSYFCKEMSPYLDYKKSYLNRKKEILANSIQQNRGFSMPTAEEPNLLSKRKQLFQQWAMKFQESPLIQQNNFAVEQFNKEFANKINELAAVYNVDELCTAITEKLMNFDKDKSNKTRNFEIKKLWKQHPHNKDKALIKLFNQEGNEALNQFNIELGKYFEHYFPKTGKKESAESYYLNPQTIIKTVGYQLRNAFVQYLLQVGKLHQYNKGVLDSQTLQEIGMYEGFQTKFMDACVFASSSLRNIIQATTNEDILTREKFKKELEKNVELKHDLFFKTEIVEERDENPAKKIAMTPNELDLWAIRGAVQRVRNQIFHQQINKRHEPNQLKVGSFENGDLGNVSYQKTIYQKLFDAEIKDIEIYFAEKIKSSGALEQYSMKDLEKLFSNKELTLSLGGQVVAFAPSYKKLYKQGYFYQNEKTIELEQFTDYDFSNDVFKANYYLIKLIYHYVFLPQFSQANNKLFKDTVHYVIQQNKELNTTEKDKKNNKKIRKYAFEQVKLMKNESPEKYMQYLQREMQEERTIKEAKKTNEEKPNYNFEKLLIQIFIKGFDTFLRNFDLNLNPAEELVGTVKEKAEGLRKRKERIAKILNVDEQIKTGDEEIAFWIFAKLLDARHLSELRNEMIKFKQSSVKKGLIKNGDLIEQMQPILELCILSNDSESMEKESFDKIEVFLEKVELAKNEPYMQEDKLTPVKFRFMKQLEKYQTRNFIENLVIENPEFKVSEKIVLNWHEEKEKIADLVDKRTKLHEEWASKAREIEEYNEKIKKNKSKKLDKPAEFAKFAEYKIICEAIENFNRLDHKVRLTYLKNLHYLMIDLMGRMVGFSVLFERDFVYMGRSYSALKKQSIYLNDYDTFANIRDWEVNENKHLFGTSSSDLTFQETAEFKNLKKPMENQLKALLGVTNHSFEIRNNIAHLHVLRNDGKGEGVSLLSCMNDLRKLMSYDRKLKNAVTKAIIKILDKHGMILKLTNNDHTKPFEIESLKPKKIIHLEKSNHSFPMDQVSQEYCDLVKKMLVFTN

SEQ ID NO: 155 - protein, 1154 aa, Paludibacter propionicigenes
MRVSKVKVKDGGKDKMVLVHRKTTGAQLVYSGQPVSNETSNILPEKKRQSFDLSTLNKTIIKFDTAKKQKLNVDQYKIVEKIFKYPKQELPKQIKAEEILPFLNHKFQEPVKYWKNGKEESFNLTLLIVEAVQAQDKRKLQPYYDWKTWYIQTKSDLLKKSIENNRIDLTENLSKRKKALLAWETEFTASGSIDLTHYHKVYMTDVLCKMLQDVKPLTDDKGKINTNAYHRGLKKALQNHQPAIFGTREVPNEANRADNQLSIYHLEVVKYLEHYFPIKTSKRRNTADDIAHYLKAQTLKTTIEKQLVNAIRANIIQQGKTNHHELKADTTSNDLIRIKTNEAFVLNLTGTCAFAANNIRNMVDNEQTNDILGKGDFIKSLLKDNTNSQLYSFFFGEGLSTNKAEKETQLWGIRGAVQQIRNNVNHYKKDALKTVFNISNFENPTITDPKQQTNYADTIYKARFINELEKIPEAFAQQLKTGGAVSYYTIENLKSLLTTFQFSLCRSTIPFAPGFKKVFNGGINYQNAKQDESFYELMLEQYLRKENFAEESYNARYFMLKLIYNNLFLPGFTTDRKAFADSVGFVQMQNKKQAEKVNPRKKEAYAFEAVRPMTAADSIADYMAYVQSELMQEQNKKEEKVAEETRINFEKFVLQVFIKGFDSFLRAKEFDFVQMPQPQLTATASNQQKADKLNQLEASITADCKLTPQYAKADDATHIAFYVFCKLLDAAHLSNLRNELIKFRESVNEFKFHHLLEIIEICLLSADVVPTDYRDLYSSEADCLARLRPFIEQGADITNWSDLFVQSDKHSPVIHANIELSVKYGTTKLLEQIINKDTQFKTTEANFTAWNTAQKSIEQLIKQREDHHEQWVKAKNADDKEKQERKREKSNFAQKFIEKHGDDYLDICDYINTYNWLDNKMHFVHLNRLHGLTIELLGRMAGFVALFDRDFQFFDEQQIADEFKLHGFVNLHSIDKKLNEVPTKKIKEIYDIRNKIIQINGNKINESVRANLIQFISSKRNYYNNAFLHVSNDEIKEKQMYDIRNHIAHFNYLTKDAADFSLIDLINELRELLHYDRKLKNAVSKAFIDLFDKHGMILKLKLNADHKLKVESLEPKKIYHLGSSAKDKPEYQYCTNQVMMAYCNMCRSLLEMKK

SEQ ID NO: 156 - protein, 970 aa, unidentified
  source 1..970: note = Listeria weihenstephanensis
MLALLHQEVPSQKLHNLKSLNTESLTKLFKPKFQNMISYPPSKGAEHVQFCLTDIAVPAIRDLDEIKPDWGIFFEKLKPYTDWAESYIHYKQTTIQKSIEQNKIQSPDSPRKLVLQKYVTAFLNGEPLGLDLVAKKYKLADLAESFKVVDLNEDKSANYKIKACLQQHQRNILDELKEDPELNQYGIEVKKYIQRYFPIKRAPNRSKHARADFLKKELIESTVEQQFKNAVYHYVLEQGKMEAYELTDPKTKDLQDIRSGEAFSFKFINACAFASNNLKMILNPECEKDILGKGDFKKNLPNSTTQSDVVKKMIPFFSDEIQNVNFDEAIWAIRGSIQQIRNEVYHCKKHSWKSILKIKGFEFEPNNMKYTDSDMQKLMDKDIAKIPDFIEEKLKSSGIIRFYSHDKLQSIWEMKQGFSLLTTNAPFVPSFKRVYAKGHDYQTSKNRYYDLGLTTFDILEYGEEDFRARYFLTKLVYYQQFMPWFTADNNAFRDAANFVLRLNKNRQQDAKAFINIREVEEGEMPRDYMGYVQGQIAIHEDSTEDTPNHFEKFISQVFIKGFDSHMRSADLKFIKNPRNQGLEQSEIEEMSFDIKVEPSFLKNKDDYIAFWTFCKMLDARHLSELRNEMIKYDGHLTGEQEIIGLALLGVDSRENDWKQFFSSEREYEKIMKGYVGEELYQREPYRQSDGKTPILFRGVEQARKYGTETVIQRLFDASPEFKVSKCNITEWERQKETIEETIERRKELHNEWEKNPKKPQNNAFFKEYKECCDAIDAYNWHKNKTTLVYVNELHHLLIEILGRYVGYVAIADRDFQCMANQYFKHSGITERVEYWGDNRLKSIKKLDTFLKKEGLFVSEKNARNHIAHLNYLSLKSECTLLYLSERLREIFKYDRKLKNAVSKSLIDILDRHGMSVVFANLKENKHRLVIKSLEPKKLRHLGEKKIDNGYIETNQVSEEYCGIVKRLLEI

SEQ ID NO: 157 - protein, 1051 aa, Listeria sp.
MKITKMRVDGRTIVMERTSKEGQLGYEGIDGNKTTEIIFDKKKESFYKSILNKTVRKPDEKEKNRRKQAINKAINKEITELMLAVLHQEVPSQKLHNLKSLNTESLTKLFKPKFQNMISYPPSKGAEHVQFCLTDIAVPAIRDLDEIKPDWGIFFEKLKPYTDWAESYIHYKQTTIQKSIEQNKIQSPDSPRKLVLQKYVTAFLNGEPLGLDLVAKKYKLADLAESFKLVDLNEDKSANYKIKACLQQHQRNILDELKEDPELNQYGIEVKKYIQRYFPIKRAPNRSKHARADFLKKELIESTVEQQFKNAVYHYVLEQGKMEAYELTDPKTKDLQDIRSGEAFSFKFINACAFASNNLKMILNPECEKDILGKGNFKKNLPNSTTRSDVVKKMIPFFSDELQNVNFDEAIWAIRGSIQQIRNEVYHCKKHSWKSILKIKGFEFEPNNMKYADSDMQKLMDKDIAKIPEFIEEKLKSSGVVRFYRHDELQSIWEMKQGFSLLTTNAPFVPSFKRVYAKGHDYQTSKNRYYNLDLTTFDILEYGEEDFRARYFLTKLVYYQQFMPWFTADNNAFRDAANFVLRLNKNRQQDAKAFINIREVEEGEMPRDYMGYVQGQIAIHEDSIEDTPNHFEKFISQVFIKGFDRHMRSANLKFIKNPRNQGLEQSEIEEMSFDIKVEPSFLKNKDDYIAFWIFCKMLDARHLSELRNEMIKYDGHLTGEQEIIGLALLGVDSRENDWKQFFSSEREYEKIMKGYVVEELYQREPYRQSDGKTPILFRGVEQARKYGTETVIQRLFDANPEFKVSKCNLAEWERQKETIEETIKRRKELHNEWAKNPKKPQNNAFFKEYKECCDAIDAYNWHKNKTTLAYVNELHHLLIEILGRYVGYVAIADRDFQCMANQYFKHSGITERVEYWGDNRLKSIKKLDTFLKKEGLFVSEKNARNHIAHLNYLSLKSECTLLYLSERLREIFKYDRKLKNAVSKSLIDILDRHGMSVVFANLKENKHRLVIKSLEPKKLRHLGGKKIDGGYIETNQVSEEYCGIVKRLLEM

SEQ ID NO: 158 - protein, 1152 aa, Leptotrichia wadei; identical to SEQ ID NO: 28

SEQ ID NO: 159 - protein, 1285 aa, Rhodobacter capsulatus; identical to SEQ ID NO: 29

SEQ ID NO: 160 - protein, 1285 aa, Rhodobacter capsulatus; identical to SEQ ID NO: 30

SEQ ID NO: 161 - protein, 1285 aa, Rhodobacter capsulatus; identical to SEQ ID NO: 30

SEQ ID NO: 162 - protein, 1159 aa, Leptotrichia buccalis; identical to SEQ ID NO: 32

SEQ ID NO: 163 - protein, 1284 aa, unidentified
  source 1..1284: note = Herbinix hemicellulosilytica
KLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 164 - protein, 1344 aa, Eubacterium rectale; identical to SEQ ID NO: 34

SEQ ID NO: 165 - protein, 1373 aa, unidentified
  source 1..1373: note = Eubacteriaceae bacterium
MKISKESHKRTAVAVMEDRVGGVVYVPGGSGIDLSNNLKKRSMDTKSLYNVFNQIQAGTAPSEYEWKDYLSEAENKKREAQKMIQKANYELRRECEDYAKKANLAVSRIIFSKKPKKIFSDDDIISHMKKQRLSKFKGRMEDFVLIALRKSLVVSTYNQEVFDSRKAATVFLKNIGKKNISADDERQIKQLMALIREDYDKWNPDKDSSDKKESSGTKVIRSIEHQNMVIQPEKNKLSLSKISNVGKKTKTKQKEKAGLDAFLKEYAQIDENSRMEYLKKLRRLLDTYFAAPSSYIKGAAVSLPENINFSSELNVWERHEAAKKVNINFVEIPESLLNAEQNNNKINKVEQEHSLEQLRTDIRRRNITCYHFANALAADERYHTLFFENMAMNQFWIHHMENAVERILKKCNVGTLFKLRIGYLSEKVWKDMLNLLSIKYIALGKAVYHFALDDIWKADIWKDASDKNSGKINDLTLKGISSFDYEMVKAQEDLQREMAVGVAFSTNNLARVTCKMDDLSDAESDFLLWNKEAIRRHVKYTEKGEILSAILQFFGGRSLWDESLFEKAYSDSNYELKFLDDLKRAIYAARNETFHFKTAAIDGGSWNTRLFGSLFEKEAGLCLNVEKNKFYSNNLVLFYKQEDLRVFLDKLYGKECSRAAQIPSYNTILPRKSFSDFMKQLLGLKEPVYGSAILDQWYSACYYLFKEVYYNLFLQDSSAKALFEKAVKALKGADKKQEKAVESFRKRYWEISKNASLAEICQSYITEYNQQNNKERKVRSANDGMFNEPIYQHYKMLLKEALKMAFASYIKNDKELKFVYKPTEKLFEVSQDNFLPNWNSEKYNTLISEVKNSPDLQKWYIVGKFMNARMLNLLLGSMRSYLQYVSDIQKRAAGLGENQLHLSAENVGQVKKWIQVLEVCLLLSVRISDKFTDYFKDEEEYASYLKEYVDFEDSAMPSDYSALLAFSNEGKIDLYVDASNPKVNRNIIQAKLYAPDMVLKKVVKKISQDECKEFNEKKEQIMQFKNKGDEVSWEEQQKILEYQKLKNRVELRDLSEYGELINELLGQLINWSYLRERDLLYFQLGFHYSCLMNESKKPDAYKTIRRGTVSIENAVLYQIIAMYINGFPVYAPEKGELKPQCKTGSAGQKIRAFCQWASMVEKKKYELYNAGLELFEVVKEHDNIIDLRNKIDHFKYYQGNDSILALYGEIFDRFFTYDMKYRNNVLNHLQNILLRHNVIIKPIISKDKKEVGRGKMKDRAAFLLEEVSSDRFTYKVKEGERKIDAKNRLYLETVRDILYFPNRAVNDKGEDVIICSKKAQDLNEKKADRDKNHDKSKDTNQKKEGKNQEEKSENKEPYSDRMTWKPFAGIKLE

SEQ ID NO: 166 - protein, 1276 aa, Blautia sp.; identical to SEQ ID NO: 36

SEQ ID NO: 167 - protein, 1385 aa, Leptotrichia sp.; identical to SEQ ID NO: 37

SEQ ID NO: 168 - protein, 500 aa, Chloroflexus aggregans; identical to SEQ ID NO: 38

SEQ ID NO: 169 - protein, 249 aa, Demequina aurantiaca; identical to SEQ ID NO: 39

SEQ ID NO: 170 - protein, 1235 aa, Thalassospira sp.; identical to SEQ ID NO: 40

SEQ ID NO: 171 - protein, 1080 aa, Prevotella buccae
MQKQDKLFVDRKKNAIFAFPKYITIMENKEKPEPIYYELTDKHFWAAFLNLARHNVYTTINHINRRLEIAELKDDGYMMGIKGSWNEQAKKLDKKVRLRDLIMKHFPFLEAAAYEMTNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYNAEEEPFKNTLVRHQDRFPYFVLRYFDLNEIFEQLRFQIDLGTYHFSIYNKRIGDEDEVRHLTHHLYGFARIQDFAPQNQPEEWRKLVKDLDHFETSQEPYISKTAPHYHLENEKIGIKFCSAHNNLFPSLQTDKTCNGRSKFNLGTQFTAEAFLSVHELLPMMFYYLLLTKDYSRKESADKVEGIIRKEISNIYAIYDAFANNEINSIADLTRRLQNTNILQGHLPKQMISILKGRQKDMGKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNRLKPKKRQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKALVKDRRLNGLFSFAETTDLNLEEHPISKLSVDLELIKYQTTRISIFEMTLGLEKKLIDKYSTLPTDSFRNMLERWLQCKANRPELKNYVNSLIAVRNAFSHNQYPMYD

SEQ ID NO: 172 - protein, 953 aa, Alistipes sp.; identical to SEQ ID NO: 53

SEQ ID NO: 173 - protein, 1320 aa, Prevotella sp.
MSKECKKQRQEKKRRLQKANFSISLTGKHVFGAYFNMARTNFVKTINYILPIAGVRGNYSENQINKMLHALFLIQAGRNEELTTEQKQWEKKLRLNPEQQTKFQKLLFKHFPVLGPMMADVADHKAYLNKKKSTVQTEDETFAMLKGVSLADCLDIICLMADTLTECRNFYTHKDPYNKPSQLADQYLHQEMIAKKLDKVVVASRRILKDREGLSVNEVEFLTGIDHLHQEVLKDEFGNAKVKDGKVMKTFVEYDDFYFKISGKRLVNGYTVTTKDDKPVNVNTMLPALSDFGLLYFCVLFLSKPYAKLFIDEVRLFEYSPFDDKENMIMSEMLSIYRIRTPRLHKIDSHDSKATLAMDIFGELRRCPMELYNLLDKNAGQPFFHDEVKHPNSHTPDVSKRLRYDDRFPTLALRYIDETELFKRIRFQLQLGSFRYKFYDKENCIDGRVRVRRIQKEINGYGRMQEVADKRMDKWGDLIQKREERSVKLEHEELYINLDQFLEDTADSTPYVTDRRPAYNIHANRIGLYWEDSQNPKQYKVFDENGMYIPELVVTEDKKAPIKMPAPRCALSVYDLPAMLFYEYLREQQDNEFPSAEQVIIEYEDDYRKFFKAVAEGKLKPFKRPKEFRDFLKKEYPKLRMADIPKKLQLFLCSHGLCYNNKPETVYERLDRLTLQHLEERELHIQNRLEHYQKDRDMIGNKDNQYGKKSFSDVRHGALARYLAQSMMEWQPTKLKDKEKGHDKLTGLNYNVLTAYLATYGHPQVPEEGFTPRTLEQVLINAHLIGGSNPHPFINKVLALGNRNIEELYLHYLEEELKHIRSRIQSLSSNPSDKALSALPFIHHDRMRYHERTSEEMMALAARYTTIQLPDGLFTPYILEILQKHYTENSDLQNALSQDVPVKLNPTCNAAYLITLFYQTVLKDNAQPFYLSDKTYTRNKDGEKAESFSFKRAYELFSVLNNNKKDTFPFEMIPLFLTSDEIQERLSAKLLDGDGNPVPEVGEKGKPATDSQGNTIWKRRIYSEVDDYAEKLTDRDMKISFKGEWEKLPRWKQDKIIKRRDETRRQMRDELLQRMPRYIRDIKDNERTLRRYKTQDMVLFLLAEKMFTNIISEQSSEFNWKQMRLSKVCNEAFLRQTLTFRVPVTVGETTIYVEQENMSLKNYGEFYRFLTDDRLMSLLNNIVETLKPNENGDLVIRHTDLMSELAAYDQYRSTIFMLIQSIENLIITNNAVLDDPDADGFWVREDLPKRNNFASLLELINQLNNVELTDDERKLLVAIRNAFSHNSYNIDFSLIKDVKHLPEVAKGILQHLQSMLGVEI

SEQ ID NO: 174 - protein, 960 aa, Riemerella anatipestifer
ALNPSKLIEALEILVNQLHSLRNYYSHYKHKKPDAEKDIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSRILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLMLSKPIRKEIKKHGRVGFISRAITLYFKEKYQDKHQSFYNLSYKLEAKAPLLKREEHYEYWQQNKPQSPTESQRLELHTSDRWKDYLLYKRWQHLEKKLRLYRNQDVMLWLMTLELTKNHFKELNLNYHQLKLENLAVNVQEADAKLNPLNQTLPMVLPVKVYPATAFGEVQYHKTPIRTVYIREEHTKALKMGNFKALVKDRRLNGLFSFIKEENDTQKHPISQLRLRRELEIYQSLRVDAFKETLSLEEKLLNKHTSLSSLENEFRALLEEWKKEYAASSMVTDEHIAFIASVRNAFCHNQYPFYKEALHAPIPLFTVAQPTTEEKDGLGIAEAL

SEQ ID NO: 175 - protein, 1080 aa, Prevotella aurantiaca
MEDDKKTTGSISYELKDKHFWAAFLNLARHNVYITINHINKLLEIREIDNDEKVLDIKTLWQKGNKDLNQKARLRELMTKHFPFLETAIYTKNKEDKKEVKQEKQAEAQSLESLKDCLFLFLDKLQEARNYYSHYKYSEFSKEPEFEEGLLEKMYNIFGNNIQLVINDYQHNKDINPDEDFKHLDRKGQFKYSFADNEGNITESGLLFFVSLFLEKKDAIWMQQKLNGFKDNLENKKKMTHEVFCRSRILMPKLRLESTQTQDWILLDMLNELIRCPKSLYERLQGDDREKFKVPFDPADEDYNAEQEPFKNTLIRHQDRFPYFVLRYFDYNEIFKNLRFQIDLGTYHFSIYKKLIGGQKEDRHLTHKLYGFERIQEFAKQNRPDEWKAIVKDLDTYETSNKRYISETTPHYHLENQKIGIRFRNGNKEIWPSLKTNDENNEKSKYKLDKQYQAEAFLSVHELLPMMFYYLLLKKEKPNNDEINASIVEGFIKREIRNIFKLYDAFANGEINNIDDLEKYCADKGIPKRHLPKQMVAILYDEHKDMVKEAKRKQKEMVKDTKKLLATLEKQTQKEKEDDGRNVKLLKSGEIARWLVNDMMRFQPVQKDNEGKPLNNSKANSTEYQMLQRSLALYNNEEKPTRYFRQVNLIESNNPHPFLKWTKWEECNNILTFYYSYLTKKIEFLNKLKPEDWKKNQYFLKLKEPKTNRETLVQGWKNGFNLPRGIFTEPIREWFKRHQNNSKEYEKVEALDRVGLVTKVIPLFFKEEYFKDKEENFKEDTQKEINDCVQPFYNFPYNVGNIHKPKEKDFLHREERIELWDKKKDKFKGYKEKIKSKKLTEKDKEEFRSYLEFQSWNKFERELRLVRNQDIVTWLLCKELIDKLKIDELNIEELKKLRLNNIDTDTAKKEKNNILNRVMPMELPVTVYEIDDSHKIVKDKPLHTIYIKEAETKLLKQGNFKALVKDRRLNGLFSFVKTNSEAESKRNPISKLRVEYELGEYQEARIEIIQDMLALEEKLINKYKDLPTNKFSEMLNSWLEGKDEADKARFQNDVDFLIAVRNAFSHNQYPMHNKIEFANI

SEQ ID NO: 176 - protein, 1140 aa, Prevotella saccharolytica
MEDKPFWAAFFNLARHNVYLTVNHINKLLDLEKLYDEGKHKEIFEREDIFNISDDVMNDANSNGKKRKLDIKKIWDDLDTDLTRKYQLRELILKHFPFIQPAIIGAQTKERTTIDKDKRSTSTSNDSLKQTGEGDINDLLSLSNVKSMFFRLLQILEQLRNYYSHVKHSKSATMPNFDEDLLNWMRYIFIDSVNKVKEDYSSNSVIDPNTSFSHLIYKDEQGKIKPCRYPFTSKDGSINAFGLLFFVSLFLEKQDSIWMQKKIPGFKKASENYMKMTNEVFCRNHILLPKIRLETVYDKDWMLLDMLNEVVRCPLSLYKRLTPAAQNKFKVPEKSSDNANRQEDDNPFSRILVRHQNRFPYFVLRFFDLNEVFTTLRFQINLGCYHFAICKKQIGDKKEVHHLIRTLYGFSRLQNFTQNTRPEEWNTLVKTTEPSSGNDGKTVQGVPLPYISYTIPHYQIENEKIGIKIFDGDTAVDTDIWPSVSTEKQLNKPDKYTLTPGFKADVFLSVHELLPMMFYYQLLLCEGMLKTDAGNAVEKVLIDTRNAIFNLYDAFVQEKINTITDLENYLQDKPILIGHLPKQMIDLLKGHQRDMLKAVEQKKAMLIKDTERRLKLLDKQLKQETDVAAKNTGTLLKNGQIADWLVNDMMRFQPVKRDKEGNPINCSKANSTEYQMLQRAFAFYATDSCRLSRYFTQLHLIHSDNSHLFLSRFEYDKQPNLIAFYAAYLKAKLEFLNELQPQNWASDNYFLLLRAPKNDRQKLAEGWKNGFNLPRGLFTEKIKTWFNEHKTIVDISDCDIFKNRVGQVARLIPVFFDKKFKDHSQPFYRYDFNVGNVSKPTEANYLSKGKREELFKSYQNKFKNNIPAEKTKEYREYKNFSLWKKFERELRLIKNQDILIWLMCKNLFDEKIKPKKDILEPRIAVSYIKLDSLQTNTSTAGSLNALAKVVPMTLAIHIDSPKPKGKAGNNEKENKEFTVYIKEEGTKLLKWGNFKTLLADRRIKGLFSYIEHDDIDLKQHPLTKRRVDLELDLYQTCRIDIFQQTLGLEAQLLDKYSDLNTDNFYQMLIGWRKKEGIPRNIKEDTDFLKDVRNAFSHNQYPDSKKIAFRRIRKFNPKELILEEEEGLGIATQMYKEVEKV

SEQ ID NO: 177 - protein, 1080 aa, Prevotella intermedia
MEDDKKTTDSIRYELKDKHFWAAFLNLARHNVYITVNHINKILEEDEINRDGYENTLENSWNEIKDINKKDRLSKLIIKHFPFLEATTYRQNPTDTTKQKEEKQAEAQSLESLKKSFFVFIYKLRDLRNHYSHYKHSKSLERPKFEEDLQNKMYNIFDVSIQFVKEDYKHNTDINPKKDFKHLDRKRKGKFHYSFADNEGNITESGLLFFVSLFLEKKDAIWVQKKLEGFKCSNKSYQKMTNEVFCRSRMLLPKLRLESTQTQDWILLDMLNELIRCPKSLYERLQGVNRKKFYVSFDPADEDYDAEQEPFKNTLVRHQDRFPYFALRYFDYNEVFANLRFQIDLGTYHFSIYKKLIGGQKEDRHLTHKLYGFERIQEFDKQNRPDEWKAIVKDSDTFKKKEEKEEEKPYISETTPHYHLENKKIGIAFKNHNIWPSTQTELTNNKRKKYNLGTSIKAEAFLSVHELLPMMFYYLLLKTENTKNDNKVGGKKETKKQGKHKIEAIIESKIKDIYALYDAFANGEINSEDELKEYLKGKDIKIVHLPKQMIAILKNEHKDMAEKAEAKQEKMKLATENRLKTLDKQLKGKIQNGKRYNSAPKSGEIASWLVNDMMRFQPVQKDENGESLNNSKANSTEYQLLQRTLAFFGSEHERLAPYFKQTKLIESSNPHPFLNDTEWEKCSNILSFYRSYLKARKNFLESLKPEDWEKNQYFLMLKEPKTNRETLVQGWKNGFNLPRGFFTEPIRKWFMEHWKSIKVDDLKRVGLVAKVTPLFFSEKYKDSVQPFYNYPFNVGDVNKPKEEDFLHREERIELWDKKKDKFKGYKAKKKFKEMTDKEKEEHRSYLEFQSWNKFERELRLVRNQDIVTWLLCTELIDKLKIDELNIKELKKLRLKDINTDTAKKEKNNILNRVMPMELPVTVYKVNKGGYIIKNKPLHTIYIKEAETKLLKQGNFKALVKDRRLNGLFSFVKTPSEAESESNPISKLRVEYELGKYQNARLDIIEDMLALEKKLIDKYNSLDTDNFHNMLTGWLELKGEAKKARFQNDVKLLTAVRNAFSHNQYPMYDENLFGNIERFSL

SEQ ID NO: 178 - protein, 1199 aa, Capnocytophaga canimorsus; identical to SEQ ID NO: 61

SEQ ID NO: 179 - protein, 1140 aa, Porphyromonas gulae
MTEQSERPYNGTYYTLEDKHFWAAFLNLARHNAYITLTHIDRQLAYSKADITNDQDVLSFKALWKNFDNDLERKSRLRSLILKHFSFLEGAAYGKKLFESKSSGNKSSKNKELTKKEKEELQANALSLDNLKSILFDFLQKLKDFRNYYSHYRHSGSSELPLFDGNMLQRLYNVFDVSVQRVKIDHEHNDEVDPHYHFNHLVRKGKKDRYGHNDNPSFKHHFVDGEGMVTEAGLLFFVSLFLEKRDAIWMQKKIRGFKGGTETYQQMTNEVFCRSRISLPKLKLESLRMDDWMLLDMLNELVRCPKPLYDRLREDDRACFRVPVDILPDEDDTDGGGEDPFKNTLVRHQDRFPYFALRYFDLKKVFTSLRFHIDLGTYHFAIYKKMIGEQPEDRHLTRNLYGFGRIQDFAEEHRPEEWKRLVRDLDYFETGDKPYISQTSPHYHIEKGKIGLRFMPEGQHLWPSPEVGTTRTGRSKYAQDKRLTAEAFLSVHELMPMMFYYFLLREKYSEEVSAERVQGRIKRVIEDVYAVYDAFARDEINTRDELDACLADKGIRRGHLPRQMIAILSQEHKDMEEKIRKKLQEMMADTDHRLDMLDRQTDRKIRIGRKNAGLPKSGVIADWLVRDMMRFQPVAKDASGKPLNNSKANSTEYRMLQRALALFGGEKERLTPYFRQMNLTGGNNPHPFLHETRWESHTNILSFYRSYLRARKAFLERIGRSDRVENRPFLLLKEPKTDRQTLVAGWKGEFHLPRGIFTEAVRDCLIEMGHDEVASYKEVGFMAKAVPLYFERACEDRVQPFYDSPFNVGNSLKPKKGRFLSKEERAEEWERGKERFRDLEAWSYSAARRIEDAFAGIEYASPGNKKKIEQLLRDLSLWEAFESKLKVRADRINLAKLKKEILEAQEHPYHDFKSWQKFERELRLVKNQDIITWMMCRDLMEENKVEGLDTGTLYLKDIRPNVQEQGSLNVLNRVKPMRLPVVVYRADSRGHVHKEEAPLATVYIEERDTKLLKQGNFKSFVKDRRLNGLFSFVDTGGLAMEQYPISKLRVEYELAKYQTARVCVFELTLRLEESLLTRYPHLPDESFREMLESWSDPLLAKWPELHGKVRLLIAVRNAFSHNQYPMYDEAVFSSIRKYDP

SEQ ID NO: 180 - protein, 1080 aa, Prevotella sp.
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIK

SEQ ID NO: 181 - protein, 1140 aa, Flavobacterium branchiophilum
MENLNKILDKENEICISKIFNTKGIAAPITEKALDNIKSKQKNDLNKEARLHYFSIGHSFKQIDTKKVFDYVLIEELKDEKPLKFITLQKDFFTKEFSIKLQKLINSIRNINNHYVHNFNDINLNKIDSNVFHFLKESFELAIIEKYYKVNKKYPLDNEIVLFLKELFIKDENTALLNYFTNLSKDEAIEYILTFTITENKIWNINNEHNILNIEKGKYLTFEAMLFLITIFLYKNEANHLLPKLYDFKNNKSKQELFTFFSKKFTSQDIDAEEGHLIKFRDMIQYLNHYPTAWNNDLKLESENKNKIMTTKLIDSIIEFELNSNYPSFATDIQFKKEAKAFLFASNKKRNQTSFSNKSYNEEIRHNPHIKQYRDEIASALTPISFNVKEDKFKIFVKKHVLEEYFPNSIGYEKFLEYNDFTEKEKEDFGLKLYSNPKTNKLIERIDNHKLVKSHGRNQDRFMDFSMRFLAENNYFGKDAFFKCYKFYDTQEQDEFLQSNENNDDVKFHKGKVTTYIKYEEHLKNYSYWDCPFVEENNSMSVKISIGSEEKILKIQRNLMIYFLENALYNENVENQGYKLVNNYYRELKKDVEESIASLDLIKSNPDFKSKYKKILPKRLLHNYAPAKQDKAPENAFETLLKKADFREEQYKKLLKKAEHEKNKEDFVKRNKGKQFKLHFIRKACQMMYFKEKYNTLKEGNAAFEKKDPVIEKRKNKEHEFGHHKNLNITREEFNDYCKWMFAFNGNDSYKKYLRDLFSEKHFFDNQEYKNLFESSVNLEAFYAKTKELFKKWIETNKPTNNENRYTLENYKNLILQKQVFINVYHFSKYLIDKNLLNSENNVIQYKSLENVEYLISDFYFQSKLSIDQYKTCGKLFNKLKSNKLEDCLLYEIAYNYIDKKNVHKIDIQKILTSKIILTINDANTPYKISVPFNKLERYTEMIAIKNQNNLKARFLIDLPLYLSKNKIKKGKDSAGYEIIIKNDLEIEDINTINNKIINDSVKFTEVLMELEKYFILKDKCILSKNYIDNSEIPSLKQFSKVWIKENENEIINYRNIACHFHLPLLETFDNLLLNVEQKFIKEELQNVSTINDLSKPQEYLILLFIKFKHNNFYLNLFNKNESKTIKNDKEVKKNRVLQK

SEQ ID NO: 182 - protein, 1080 aa, Porphyromonas gingivalis
MTEQNEKPYNGTYYTLEDKHFWAAFLNLARHNAYITLAHIDRQLAYSKADITNDEDILFFKGQWKNLDNDLERKARLRSLILKHFSFLEGAAYGKKLFESQSSGNKSSKKKELSKKEKEELQANALSLDNLKSILFDFLQKLKDFRNYYSHYRHPESSELPLFDGNMLQRLYNVFDVSVQRVKRDHEHNDKVDPHRHFNHLVRKGKKDKYGNNDNPFFKHHFVDREGTVTEAGLLFFVSLFLEKRDAIWMQKKIRGFKGGTEAYQQMTNEVFCRSRISLPKLKLESLRTDDWMLLDMLNELVRCPKSLYDRLREEDRARFRVPVDILSDEDDTDGTEEDPFKNTLVRHQDRFPYFALRYFDLKKVFTSLRFHIDLGTYHFAIYKKNIGEQPEDRHLTRNLYGFGRIQDFAEEHRPEEWKRLVRDLDYFETGDKPYITQTTPHYHIEKGKIGLRFVPEGQHLWPSPEVGATRTGRSKYAQDKRLTAEAFLSVHELMPMMFYYFLLREKYSEEVSAEKVQGRIKRVIEDVYAVYDAFARDEINTRDELDACLADKGIRRGHLPRQMIAILSQEHKDMEEKVRKKLQEMIADTDHRLDMLDRQTDRKIRIGRKNAGLPKSGVVADWLVRDMMRFQPVAKDTSGKPLNNSKANSTEYRMLQRALALFGGEKERLTPYFRQMNLTGGNNPHPFLHETRWESHTNILSFYRSYLEARKAFLQSIGRSDRVENHRFLLLKEPKTDRQTLVAGWKGEFHLPRGIFTEAVRDCLIEMGYDEVGSYKEVGFMAKAVPLYFERASKDRVQPFYDYPFNVGNSLKPKKGRFLSKEKRAEEWESGKERFRLAKLKKEILEAKEHPYHDFKSWQKFERELRLVKNQDIITWMMCRDLMEENKVEGLDTGTLYLKDIRTDVQEQGSLNVLNRVKPMRLPVVVYRADSRGHVHKEQAPLATVYIEERDTKLLKQGNFKSFVKDRRLNGLFSFVDTGALAMEQYPISKLRVEYELAKYQTARVCAFEQTLELEESLLTRYPHLPDKNFRKMLESWSDPLLDKWPDLHGNVRLLIAVRNAFSHNQYPMYDETLFSSIR

SEQ ID NO: 183 - protein, 780 aa, Prevotella intermedia
MEDDKKTKESTNMLDNKHFWAAFLNLARHNVYITVNHINKVLELKNKKDQDIIIDNDQDILAIKTHWEKVNGDLNKTERLRELMTKHFPFLETAIYTKNKEDKEEVKQEKQAKAQSFDSLKHCLFLFLEKLQEARNYYSHYKYSESTKEPMLEKELLKKMYNIFDDNIQLVIKDYQHNKDINPDEDFKHLDRTEEEFNYYFTTNKKGNITASGLLFFVSLFLEKKDAIWMQQKLRGFKDNRESKKKMTHEVFCRSRMLLPKLRLESTQTQDWILLDMLNELIRCPKSLYERLQGEYRKKFNVPFDSADEDYDAEQEPFKNTLVRHQDRFPYFALRYFDYNEIFTNLRFQIDLGTYHFSIYKKLIGGQKEDRHLTHKLYGFERIQEFAKQNRTDEWKAIVKDFDTYETSEEPYISETAPHYHLENQKIGIRFRNDNDEIWPSLKTNGENNEKRKYKLDKQYQAEAFLSVHELLPMMFYYLLLKKEEPNNDKKNASIVEGFIKREIRDIYKLYDAFANGEINNIDDLEKYCEDKGIPKRHLPKQMVAILYDEHKDMAEEAKRKQKEMVKDTKKLLATLEKQTQGEIEDGGRNIRLLKSGEIARWLVNDMMRFQPVQKDNEGNPLNNSKANSTEYQMLQRSLALYNKEEKPTRYFRQVNLINSSNPHPFLKWTKWEECNNILSFYRSYLTKKIEFLNKLKPEDWEKNQYFLKLKEPKTNRETLVQGWKNGFNLPRGIFTEPIREWFKRHQNDSEEYEKVETLDRVGLVTKVIPLFFKKEDSKD

SEQ ID NO: 184 - protein, 1080 aa, Fusobacterium necrophorum
MEKFRRQNRNSIIKIIISNYDTKGIKELKVRYRKQAQLDTFIIKTEIVNNDIFIKSIIEKAREKYRYSFLFDGEEKYHFKNKSSVEIVKKDIFSQTPDNMIRNYKITLKISEKNPRVVEAEIEDLMNSTILKDGRRSARREKSMTERKLIEEKVAKNYSLLANCPMEEVDSIKIYKIKRFLTYRSNMLLYFASINSFLCEGIKGKDNETEEIWHLKDNDVRKEKVRENFKNKLIQSTENYNSSLKNQIEEKEKLLRKEFKKGAFYRTIIKKLQQERIKELSEKSLTEDCEKIIKLYSKLRHSLMHYDYQYFENLFENKKNDDLMKDLNLDLFKSLPLIRKMKLNNKVNYLEDGDTLFVLQKTKKAKTLYQIYDALCEQKNGFNKFINDFFVSDGEENTVFKQIINEKFQSEMEFLEKRISESEKKNEKLKKKLDSMKAHFRNINSEDTKEAYFWDIHSSRNYKTKYNERKNLVNEYTELLGSSKEKKLLREEITKINRQLLKLKQEMEEITKKNSLFRLEYKMKIAFGFLFCEFDGNISKFKDEFDASNQEKIIQYHKNGEKYLTSFLKEEEKEKFNLEKMQKIIQKTEEEDWLLPETKNNLFKFYLLTYLLLPYELKGDFLGFVKKHYYDIKNVDFIDENQNNIQVSQTVEKQEDYFYHKIRLFEKNTKKYEIVKYSIVPNEKLKQYFEDLGIDIKYLTVEQKSEVSEEKNKKVSLKNNGMFNKTILLFVFKYYQIAFKLFNDIELYSLFFLREKSGKPLEIFRKELESKMKDGYLNFGQLLYVVYEVLVKNKDLDKILSKKIDYRKDKSFSPEIAYLRNFLSHLNYSKFLDNFMKINTNKSDENKEVLIPSIKIQKMIQFIEKCNLQNQIDFDFNFVNDFYMRKEKMFFIQLKQIFPDINSTEKQKMNEKEEILRNRYHLTDKKNEQIKDEHEAQSQLYEKILSLQKIYSSDKNNFYGRLKEEKLLFLEKQGKKKLSMEEIKDKIAGDISDLLGILKKEITRDIKDKLTEKFRYCEEKLLNLSFYNHQDKKKEESIRVFLIRDKNSDNFKFESILDDGSNKIFISKNGKEITIQCCDK

SEQ ID NO: 185 - protein, 1080 aa, Fusobacterium necrophorum
MEKFRRQNRSSIIKIIISNYDTKGIKELKVRYRKQAQLDTFIIKTEIVNNDIFIKSIIEKAREKYRYSFLFDGEEKYHFKNKSSVEIVKKDIFSQTPDNMIRNYKITLKISEKNPRVVEAEIEDLMNSTILKDGRRSARREKSMTERKLIEEKVAENYSLLANCPMEEVDSIKIYKIKRFLTYRSNMLLYFASINSFLCEGIKGKDNETEEIWHLKDNDVRKEKVKENFKNKLIQSTENYNSSLKNQIEEKEKLLRKESKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEIEFLEKRISESEKKNEKLKKKLDSMKAHFRNINSEDTKEAYFWDIHSSRNYKTKYNERKNLVNEYTELLGSSKEKKLLREEITKINRQLLKLKQEMEEITKKNSLFRLEYKMKMAFGFLFCEFDGNISRFKDEFDASNQEKIIQYHKNGEKYLTYFLKEEEKEKFNLKKLQETIQKTGEENWLLPQNKNNLFKFYLLTYLLLPYELKGDFLGFVKKHYYDIKNVDFMDENQSSKIIESKEDDFYHKIRLFEKNTKKYEIVKYSIVPDKKLKQYFKDLGIDTKYLILDQKSEVSGEKNKKVSLKNNGMFNKTILLFVFKYYQIAFKLFNDIELYSLFFLREKSGKPFEVFLKELKDKMIGKQLNFGQLLYVVYEVLVKNKDLSEILSERIDYRKDMCFSAEIADLRNFLSHLNYSKFLDNFMKINTNKSDENKEVLIPSIKIQKMIKFIEECNLQSQIDFDFNFVNDFYMRKEKMFFIQLKQIFPDINSTEKQKMNEKEEILRNRYHLTDKKNEQIKDEHEAQSQLYEKILSLQKIYSSDKNNFYGRLKEEKLLFLEKQEKKKLSMEEIKDKIAGDISDLLGILKKEITRDIKDKLTEKFRYCEEKLLNLSFYNHQDKKKEESIRVFLIRDKNSDNFKFESILDDGSNKIFISKNGKEITIQCCDKVLET

SEQ ID NO: 186 - protein, 1080 aa, Fusobacterium necrophorum
MKVRYRKQAQLDTFIIKTEIVNNDIFIKSIIEKAREKYRYSFLFDGEEKYHFKNKSSVEIVKNDIFSQTPDNMIRNYKITLKISEKNPRVVEAEIEDLMNSTILKDGRRSARREKSMTERKLIEEKVAENYSLLANCPIEEVDSIKIYKIKRFLTYRSNMLLYFASINSFLCEGIKGKDNETEEIWHLKDNDVRKEKVKENFKNKLIQSTENYNSSLKNQIEEKEKLSSKEFKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKLDSMKAHFRNINSEDTKEAYFWDIHSSRNYKTKYNERKNLVNEYTKLLGSSKEKKLLREEITKINRQLLKLKQEMEEITKKNSLFRLEYKMKIAFGFLFCEFDGNISKFKDEFDASNQEKIIQYHKNGEKYLTSFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKNKKVSLKNNGMFNKTILLFVFKYYQIAFKLFNDIELYSLFFLREKSEKPFEVFLEELKDKMIGKQLNFGQLLYVVYEVLVKNKDLDKILSKKIDYRKDKSFSPEIAYLRNFLSHLNYSKFLDNFMKINTNKSDENKEVLIPSIKIQKMIQFIEKCNLQNQIDFDFNFVNDFYMRKEKMFFIQLKQIFPDINSTEKQKKSEKEEILRKRYHLINKKNEQIKDEHEAQSQLYEKILSLQKIFSCDKNNFYRRLKEEKLLFLEKQGKKKISMKEIKDKIASDISDLLGILKKEITRDIKDKLTEKFRYCEEKLLNISFYNHQDKKKEEGIRVFLIRDKNSDNFKFESILDDGSNKIFISKNGKEITIQCCDKVLETL

SEQ ID NO: 187 - protein, 660 aa, Fusobacterium necrophorum
MTEKKSIIFKNKSSVEIVKKDIFSQTPDNMIRNYKITLKISEKNPRVVEAEIEDLMNSTILKDGRRSARREKSMTERKLIEEKVAENYSLLANCPMEEVDSIKIYKIKRFLTYRSNMLLYFASINSFLCEGIKGKDNETEEIWHLKDNDVRKEKVKENFKNKLIQSTENYNSSLKNQIEEKEKLLRKESKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKFDSMKAHFHNINSEDTKEAYFWDIHSSSNYKTKYNERKNLVNEYTELLGSSKEKKLLREEITQINRKLLKLKQEMEEITKKNSLFRLEYKMKIAFGFLFCEFDGNISKFKDEFDASNQEKIIQYHKNGEKYLTYFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVS

SEQ ID NO: 188 - protein, 1080 aa, Fusobacterium perfoetens
MGKPNRSSIIKIIISNYDNKGIKEVKVRYNKQAQLDTFLIKSELKDGKFILYSIVDKAREKYRYSFEIDKTNINKNEILIIKKDIYSNKEDKVIRKYILSFEVSEKNDRTIVTKIKDCLETQKKEKFERENTRRLISETERKLLSEETQKTYSKIACCSPEDIDSVKIYKIKRYLAYRSNMLLFFSLINDIFVKGVVKDNGEEVGEIWRIIDSKEIDEKKTYDLLVENFKKRMSQEFINYKQSIENKIEKNTNKIKEIEQKLKKEKYKKEINRLKKQLIELNRENDLLEKDKIELSDEEIREDIEKILKIYSDLRHKLMHYNYQYFENLFENKKISKEKNEDVNLTELLDLNLFRYLPLVRQLKLENKTNYLEKEDKITVLGVSDSAIKYYSYYNFLCEQKNGFNNFINSFFSNDGEENKSFKEKINLSLEKEIEIMEKETNEKIKEINKNELQLMKEQKELGTAYVLDIHSLNDYKISHNERNKNVKLQNDIMNGNRDKNALDKINKKLVELKIKMDKITKRNSILRLKYKLQVAYGFLMEEYKGNIKKFKDEFDISKEKIKSYKSKGEKYLEVKSEKKYITKILNSIEDIHNITWLKNQEENNLFKFYVLTYILLPFEFRGDFLGFVKKHYYDIKNVEFLDENNDRLTPEQLEKMKNDSFFNKIRLFEKNSKKYDILKESILTSERIGKYFSLLNTGAKYFEYGGEENRGIFNKNIIIPIFKYYQIVLKLYNDVELAMLLTLSESDEKDINKIKELVTLKEKVSPKKIDYEKKYKFSVLLDCFNRIINLGKKDFLASEEVKEVAKTFTNLAYLRNKICHLNYSKFIDDLLTIDTNKSTTDSEGKLLINDRIRKLIKFIRENNQKMNISIDYNYINDYYMKKEKFIFGQRKQAKTIIDSGKKANKRNKAEELLKMYRVKKENINLIYELSKKLNELTKSELFLLDKKLLKDIDFTDVKIKNKSFFELKNDVKEVANIKQALQKHSSELIGIYKKEVIMAIKRSIVSKLIYDEEKVLSIIIYDKTNKKYEDFLLEIRRERDINKFQFLIDEKKEKLGYEKIIETKEKKKVVVKIQNNSEL

SEQ ID NO: 189 - protein, 897 aa, Fusobacterium ulcerans
MENKGNNKKIDFDENYNILVAQIKEYFTKEIENYNNRIDNIIDKKELLKYSEKKEESEKNKKLEELNKLKSQKLKILTDEEIKADVIKIIKIFSDLRHSLMHYEYKYFENLFENKKNEELAELLNLNLFKNLTLLRQMKIENKTNYLEGREEFNIIGKNIKAKEVLGHYNLLAEQKNGFNNFINSFFVQDGTENLEFKKLIDEHFVNAKKRLERNIKKSKKLEKELEKMEQHYQRLNCAYVWDIHTSTTYKKLYNKRKSLIEEYNKQINEIKDKEVITAINVELLRIKKEMEEITKSNSLFRLKYKMQIAYAFLEIEFGGNIAKFKDEFDCSKMEEVQKYLKKGVKYLKYYKDKEAQKNYEFPFEEIFENKDTHNEEWLENTSENNLFKFYILTYLLLPMEFKGDFLGVVKKHYYDIKNVDFTDESEKELSQVQLDKMIGDSFFHKIRLFEKNTKRYEIIKYSILTSDEIKRYFRLLELDVPYFEYEKGTDEIGIFNKNIILTIFKYYQIIFRLYNDLEIHGLFNISSDLDKILRDLKSYGNKNINFREFLYVIKQNNNSSTEEEYRKIWENLEAKYLRLHLLTPEKEEIKTKTKEELEKLNEISNLRNGICHLNYKEIIEEILKTEISEKNKEATLNEKIRKVINFIKENELDKVELGFNFINDFFMKKEQFMFGQIKQVKEGNSDSITTERERKEKNNKKLKETYELNCDNLSEFYETSNNLRERANSSSLLEDSAFLKKIGLYKVKNNKVNSKVKDEEKRIENIKRKLLKDSSDIMGMYKAEVVKKLKEKLILIFKHDEEKRIYVTVYDTSKAVPENISKEILVKRNNSKEEYFFEDNNKKYVTEYYTLEITETNELKVIPAKKLEGKEFKTEKNKENKLMLNNHYCFNVKIIY

SEQ ID NO: 190 - protein, 1080 aa, unidentified
  source 1..1080: note = Anaerosalibacter sp.
MKSGRREKAKSNKSSIVRVIISNFDDKQVKEIKVLYTKQGGIDVIKFKSTEKDEKGRMKFNFDCAYNRLEEEEFNSFGGKGKQSFFVTTNEDLTELHVTKRHKTTGEIIKDYTIQGKYTPIKQDRTKVTVSITDNKDHFDSNDLGDKIRLSRSLTQYTNRILLDADVMKNYREIVCSDSEKVDETINIDSQEIYKINRFLSYRSNMIIYYQMINNFLLHYDGEEDKGGNDSINLINEIWKYENKKNDEKEKIIERSYKSIEKSINQYILNHNTEVESGDKEKKIDISEERIKEDLKKTFILFSRLRHYMVHYNYKFYENLYSGKNFIIYNKDKSKSRRFSELLDLNIFKELSKIKLVKNRAVSNYLDKKTTIHVLNKNINAIKLLDIYRDICETKNGFNNFINNMMTISGEEDKEYKEMVTKHFNENMNKLSIYLENFKKHSDFKTNNKKKETYNLLKQELDEQKKLRLWFNAPYVYDIHSSKKYKELYVERKKYVDIHSKLIEAGINNDNKKKLNEINVKLCELNTEMKEMTKLNSKYRLQYKLQLAFGFILEEFNLDIDKFVSAFDKDNNLTISKFMEKRETYLSKSLDRRDNRFKKLIKDYKFRDTEDIFCSDRENNLVKLYILMYILLPVEIRGDFLGFVKKNYYDLKHVDFIDKRNNDNKDTFFHDLRLFEKNVKRLEVTSYSLSDGFLGKKSREKFGKELEKFIYKNVSIALPTNIDIKEFNKSLVLPMMKNYQIIFKLLNDIEISALFLIAKKEGNEGSITFKKVIDKVRKEDMNGNINFSQVMKMALNEKVNCQIRNSIAHINMKQLYIEPLNIYINNNQNKKTISEQMEEIIDICITKGLTGKELNKNIINDYYMKKEKLVFNLKLRKRNNLVSIDAQQKNMKEKSILNKYDLNYKDENLNIKEIILKVNDLNNKQKLLKETTEGESNYKNALSKDILLLNGIIRKNINFKIKEMILGIIQQNEYRYVNINIYDKIRKEDHNIDLKINNKYIEISCYENKSNESTDERINFKIKYMDLKVKNELLVPSCYEDIYIKKKIDLEIRYIENCKVVYIDIYYKKYNINLEFDGKT

SEQ ID NO: 191 - protein, 1145 aa, Alicyclobacillus macrosporangiidus
MVAVKSIKVKLMLGHLPEIREGLWHLHEAVNLGVRYYTEWLALLRQGNLYRRGKDGAQECYMTAEQCRQELLVRLRDRQKRNGHTGDPGTDEELLGVARRLYELLVPQSVGKKGQAQMLASGFLSPLADPKSEGGKGTSKSGRKPAWMGMKEAGDSRWVEAKARYEANKAKDPTKQVIASLEMYGLRPLFDVFTETYKTIRWMPLGKHQGVRAWDRDMFQQSLERLMSWESWNERVGAEFARLVDRRDRFREKHFTGQEHLVALAQRLEQEMKEASPGFESKSSQAHRITKRALRGADGIIDDWLKLSEGEPVDRFDEILRKRQAQNPRRFGSHDLFLKLAEPVFQPLWREDPSFLSRWASYNEVLNKLEDAKQFATFTLPSPCSNPVWARFENAEGTNIFKYDFLFDHFGKGRHGVRFQRMIVMRDGVPTEVEGIVVPIAPSRQLDALAPNDAASPIDVFVGDPAAPGAFRGQFGGAKIQYRRSALVRKGRREEKAYLCGFRLPSQRRTGTPADDAGEVFLNLSLRVESQSEQAGRRNPPYAAVFHISDQTRRVIVRYGEIERYLAEHPDTGIPGSRGLTSGLRVMSVDLGLRTSAAISVFRVAHRDELTPDAHGRQPFFFPIHGMDHLVALHERSHLIRLPGETESKKVRSIREQRLDRLNRLRSQMASLRLLVRTGVLDEQKRDRNWERLQSSMERGGERMPSDWWDLFQAQVRYLAQHRDASGEAWGRMVQAAVRTLWRQLAKQVRDWRKEVRRNADKVKIRGIARDVPGGHSLAQLDYLERQYRFLRSWSAFSVQAGQVVRAERDSRFAVALREHIDNGKKDRLKKLADRILMEALGYVYVTDGRRAGQWQAVYPPCQLVLLEELSEYRFSNDRPPSENSQLMVWSHRGVLEELIHQAQVHDVLVGTIPAAFSSRFDARTGAPGIRCRRVPSIPLKDAPSIPIWLSHYLKQTERDAAALRPGELIPTGDGEFLVTPAGRGASGVRVVHADINAAHNLQRRLWENFDLSDIRVRCDRREGKDGTVVLIPRLTNQRVKERYSGVIFTSEDGVSFTVGDAKTRRRSSASQGEGDDLSDEEQELLAEADDARERSVVLFRDPSGFVNGGRWTAQRAFWGMVHNRIETLLAERFSVSGAAEKVRG

SEQ ID NO: 192 - protein, 1108 aa, unidentified
  source 1..1108: note = Bacillus hisashii
MATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSM

SEQ ID NO: 193 - protein, 1364 aa, unidentified
  source 1..1364: note = Candidatus Lindowbacteria bacterium
MPRDDLDLLTNLNSTAKGIRERGKTKEGTDKKKSGRKSSWPMDKAAWETAKTSDSSAHFLEKLKQHPDLKDAFGNLSSGGSKKLEYYKKLAGSAPWKESQSVILEKAARWKEAKQEREEKEQDSSEHGSKAAYRRLFDAGCLPMPEFAKYIDENQIEFGDLKLSDCGAEWKRGMWNQAGQRVRSHMGWQRRREKENAVYSLRKELFEKGGAIRRKKSEELTPEDILPGKAAPDQNDWQERPAYGNQMWFIGLRSYEENEMAKYAEEAGMGSRSAPRIRRGTIKGWSKLRERWLQILKRNPQATRDDLIGELNALRSQDPRAYGDARLFDWLSKTDQRFLWDGFDADGKILCGRDDRDCVSAFVAYNEEFADEPSSITLTETDERLHPVWPFFGESSAVPYEIEYDLETACPTAIRLPLLVGKENGGYAERQGTRLPLAEYADLASSFQLPTPVRLDVLVEIREVTRAGRKVTCPFSYFKQNGVWYVREGEIPSGESIQIKQTDRKIENGKIFISSKLRMAYRDDLMVSPATGDFGSIKILWERIELASHVDQKKLPETAPARSRVFVSFSCNVVERAPRKQLTRKPDAVVVTIPSGVDQGLVVVSTDVRTGKSKSSSAPPLPPGSRLWPADAVHGDPPLRILSVDLGHRHSAYAVWELGLQQKSWRAGVLKGSTQTPVYADCTGTGLLCLPGDGEDTPAEEESLRLRSRQIRRRLNLQNSILRVSRLLSLDKFEKTIFEQSDVRDRPNKKGLRIRRRCRTEKTPLSEAEVRKNCDKAAEILIRWADTDAMAKSLAATGNADISFWKYMAVKNPPLSAVVDVAPSTIVPDDGPDRETLKKKRQEEEEKFASSIYENRVKLAGALCSGYDADHRRPATGGLWHDLDRTLIREISYGDRGQKGNPRKLNNEGILRLLRRPPRARPDWREFHRTLNDANRIPKGRTLRGGLSMGRLNFLKEVGDFVKKWSCRPRWPGDRRHIPPGQLFDRQDAEHLEHLRDDRIKRLAHLIVAQALGFEPDIRRGLWKYVDGSTGEILWQHPETRRFFAEGAAGELREVSRPAEIDDDAAARPHTVSAPAHIVVFENLIRYRFQSDRPKTENAGLMQWAHRQIVHFTKQVASLYGLKVAMVYAAFSSKFCSRCGSPGARVSRFDPAWRNQEWFKRRTSNPRSKVDHSLKRASEDPTADETRPWVLIEGGKEFVCANAKCSAHDEPLNADENAAANIGLRFLRGVEDFRTKVNPAGALKGKLRFETGIHSFRPPVSGSPFWSPMAEPAQKKKIGAAAPGADVDEAGDADESGVVVLFRDPSGAFRNKQYWYEGKIFWSNVMMAVEAKIAGASVGAKPVAASWGQAQPQSGPGLAKPGGD

SEQ ID NO: 194 - protein, 1364 aa, Elusimicrobium sp.
MNRIYQGRVTKVEVPDGKDEKGNIKWKKLENWSDILWQHHMLFQDAVNYYTLALAAISGSAVGSDEKSIILREWAVQVQNIWEKAKKKATVFEGPQKRLTSILGLEQNASFDIAAKHILRTSEAKPEQRASALIRLLEEIDKKNHNVVCGERLPFFCPRNIQSKRSPTSKAVSSVQEQKRQEEVRRFHNMQPEEVVKNAVTLDISLFKSSPKIVFLEDPKKARAELLKQFDNACKKHKELVGIKKAFTESIDKHGSSLKVPAPGSKPSGLYPSAIVFKYFPVDITKTVFLKATEKLAMGKDREVTNDPIADARVNDKPHFDYFTNIALIREKEKNRAAWFEFDLAAFIEAIMSPHRFYQDTQKRKEAARKLEEKIKAIEGKGGQFKESDSEDDDVDSLPGFEGDTRIDLLRKLVTDTLGWLGESETPDNNEGKKTEYSISERTLRIFPDIQKQWSELAEKGETTEGKLLEVLKHEQTEHQSDFGSATLYQHLAKPEFHPIWLKSGTEEWHAENPLKAWLNYKELQYELTDKKRPIHFTPAHPVYSPRYFDFPKKSETEEKEVSKNTHSLTTSLASEHIKNSLQFTAGLIRKTNVGKKAIKARFSYSAPRLRRDCLRSENNENLYKAPWLQPMMRALGIDEEKADRQNFANTRITLMAKGLDDIQLGFPVEANSQELQKEVSNGISWKGQFNWGGIASLSALRWPHEKKPKNPPEQPWWGIDSFSCLAVDLGQRYAGAFARLDVSTIEKKGKSRFIGEACDKKWYAKVSRMGLLRLPGEDVKVWRDASKIDKENGFAFRKELFGEKGRSATPLEAEETAELIKLFGANEKDVMPDNWSKELSFPEQNDKLLIVARRAQAAVSRLHRWAWFFDEAKRSDDAIREILESDDTDLKQKVNKNEIEKVKETIISLLKVKQELLPTLLTRLANRVLPLRGRSWEWKKHHQKNDGFILDQTGKAMPNVLIRGQRGLSMDRIEQITELRKRFQALNQSLRRQIGKKAPAKRDDSIPDCCPDLLEKLDHMKEQRVNQTAHMILAEALGLKLAEPPKDKKELNETCDMHGAYAKVDNPVSFIVIEDLSRYRSSQGRSPRENSRLMKWCHRAVRDKLKEMCEVFFPLCERRKAGSAWVSLPPLLETPAAYSSRFCSRSGVAGFRAVEVIPGFELKYPWSWLKDKKDKAGNLAKEALNIRTVSEQLKAFNQDKPEKPRTLLVPIAGGPIFVPISEVGLSSFGLKPQVVQADINAAINLGLRAISDPRIWEIHPRLRTEKRDGRLFAREKRKYGEEKVEVQPSKNEKAKKVKDDRKPNYFADFSGKVDWGFGNIKNESGLTLVSGKALWWTINQLQWERCFDINKRHIEDWSNKQKQ

SEQ ID NO: 195 - protein, 1413 aa, unidentified
  source 1..1413: note = Candidatus Omnitrophota bacterium
MNRIYQGRVTKVEKLKNGKSPDDREELKDWQTALWRHHELFQDAVSYYTLALAAMAEGLPDKHPINVLRKRMEEAWEEFPRKTVTPAKNLRDSVRPWLGLSESASFGDALKKILPPAPENKEVRALAVALLAEKARTLKPQKTSASYWGRFCDDLKKKPNWDYSEEELARKTGSGDWVAGLWSEDALNKIDELAKSLKLSSLVKCVPDGQINPEGARNLVKEALDHLEGVSNGTKKEKNDPGPAKKTNNWLRQHASDVRNFIHKNKNQFSSLPNGRLITERARGGGININKTYAGVLFKAFPCPFTFDYVRAAVPEPKVKKVDQEKKSEQSATWTELEKRILRIGDDPIELARKNNKPIFKAFTALEKWSDQNSKSCWSDFDKCAFEEALKTLNQFNQKTEEREKRRSEAEAELKYMMDENPEWKPKKETEGDDVREVPILKGDPRYEKLVKLFGDLDEEGSEHATGKIYGPSRASLRGFGKLRNEWVDLFTKANDNPREQDLQKAVTGFQREHKLDMGYTAFFLKLCERDYWDIWRDDTEVEVKKIREKRWVKSVVYAAADTRELAEELERLQEPVRYTPAEPQFSRRLFMFSDIKGKQGAKHIREGLVEVSLAVKDQSGKYGTCRVRLHYSAPRLIRDHLSDGSSSMWLQPMMAALGLSSDARGCFTRDSKGNVKEPAVALMSDFVGRKRELRMLLNFPVDLDISKLEENIGKKARWEKQMNTAYEKNKLKQRFHLIWPGMELKETQEPGQFWWDNPTIQKEGMYCLAIDLSQRRAADYALLHAGVNRDSKTFVELGQAGGQSWFTKLCAAGSLRLPGEDTEVIREGKRQIELSGKKGRNATQSEYDQAIALAKQLLHNENSAELESAARDWLGDNAKRFSFPEQNDKLIDLYYGALSRYKTWLRWSWRLTEQHKELWDKTLDEIRKVPYFASWGELAGNGTNEATVQQLQKLIADAAVDLRNFLEKALLHIAYRALPLRENTWRWIENGKDGKGKPLHLLVSDGQSPAEIPWLRGQRGLSIARIEQLENFRRAVLSLNRLLRHEIGTKPEFGSSTCGESLPDPCPDLTDKIVRLKEERVNQTAHLIIAQSLGVRLKGHSLFTEEREKADMHGEHEVIPGRSPVDFVVLEDLSRYTTDKSRSRSENSRLMKWCHRKINEKVKLLAEPFGIPVIEVFASYSSKFDARTGAPGFRAVEVTSEDRPFWRKTIEKQSVAREVFDCLDNLVGKGLNGIHLVLPQNGGPLFIAAVKEDQPLPAIRQADINAAVNIGLRAIAGPSCYHAHPKVRLIKGESGTDKGKWLPRKGKEANKRENAQFGNVDLDLEVKFNRLDIDSDVLKGDNTNLFHDPLNIACYGFATIQNLQHPFLAHASAVFSRQKGAVARLQWEVCRAINSRRLEAWQKKAEKAAVKR

SEQ ID NO: 196 - protein, 747 aa, unidentified
  source 1..747: note = Phycisphaerae bacterium
MATKSYRARILTDSRLAAALDRTHVVFVESLKQMINTYLRMQNGKFGPDHKKLAQIMLSRSNTFAHGVMDQITRDQPTSTLDEEWTDLARRIHKTTGPLFLQAERFATVKNRAIHTKSRGKVIPSPETLAVPAKFWHQVCDSASAYIRSNRELMQQWRKDRAAWLKDKNEWQQKHPEFMQFYNGPYQNFLKLCDDDRITSQLAAEQQPTASKNNRPRKTGKRFARWHLWYKWLSENPEIIEWRNKASASDFKTVTDDVRKQIITKYPQQNKYITRLLDWLEDNNPELKTLENLRRTYVKKFDSFKRPPTLTLPSPYRHPYWFTMELDQFYKKADFENGTIQLLLIDEDDDGNWFFNWMPASLKPDPRLVPSWRAETFETEGRFPPYLGGKIGKKLSRPAPTDAERKAGIAGAKLMIKNNRSELLFTVFEQDCPPRVKWAKTKNRKCPADNAFSSDGKTRKPLRILSIDLGIRHIGAFALTQGTRNDSAWQTESLKKGIINSPSIPPLRQVRRHDYDLKRKRRRHGKPVKGQRSNANLQAHRTNMAQDRFKKGASAIVSLAREHSADLILFENLHSLKFSAFDERWMNRQLRDMNRRHIVELVSEQAPEFGITVKDDINPWMTSRICSNCNLPGFRFSMKKKNPYREKLPREKCTDFGYPVWEPGGHLFRCPHCDHRVNADINAAANLANKFFGLGYWNNGLKYDAETKTFTVHTDKKTPPLIFKPRPQFDLWADSVKTRKQLGPDPF

SEQ ID NO: 197 - protein, 743 aa, unidentified
  source 1..743: note = Planctomycetota bacterium
MSVRSFQARVECDKQTMEHLWRTHKVFNERLPEIIKILFKMKRGECGQNDKQKSLYKSISQSILEANAQNADYLLNSVSIKGWKPGTAKKYRNASFTWADDAAKLSSQGIHVYDKKQVLGDLPGMMSQMVCRQSVEAISGHIELTKKWEKEHNEWLKEKEKWESEDEHKKYLDLREKFEQFEQSIGGKITKRRGRWHLYLKWLSDNPDFAAWRGNKAVINPLSEKAQIRINKAKPNKKNSVERDEFFKANPEMKALDNLHGYYERNFVRRRKTKKNPDGFDHKPTFTLPHPTIHPRWFVFNKPKTNPEGYRKLILPKKAGDLGSLEMRLLTGEKNKGNYPDDWISVKFKADPRLSLIRPVKGRRVVRKGKEQGQTKETDSYEFFDKHLKKWRPAKLSGVKLIFPDKTPKAAYLYFTCDIPDEPLTETAKKIQWLETGDVTKKGKKRKKKVLPHGLVSCAVDLSMRRGTTGFATLCRYENGKIHILRSRNLWVGYKEGKGCHPYRWTEGPDLGHIAKHKREIRILRSKRGKPVKGEESHIDLQKHIDYMGEDRFKKAARTIVNFALNTENAASKNGFYPRADVLLLENLEGLIPDAEKERGINRALAGWNRRHLVERVIEMAKDAGFKRRVFEIPPYGTSQVCSKCGALGRRYSIIRENNRREIRFGYVEKLFACPNCGYCANADHNASVNLNRRFLIEDSFKSYYDWKRLSEKKQKEEIETIESKLMDKLCAMHKISRGSISK

SEQ ID NO: 198 - protein, 1119 aa, unidentified
  source 1..1119: note = Spirochaetota bacterium
MSFTISYPFKLIIKNKDEAKALLDTHQYMNEGVKYYLEKLLMFRQEKIFIGEDETGKRIYIEETEYKKQIEEFYLIKKTELGRNLTLTLDEFKTLMRELYICLVSSSMENKKGFPNAQQASLNIFSPLFDAESKGYILKEENNNISLIHKDYGKILLKRLRDNNLIPIFTKFTDIKKITAKLSPTALDRMIFAQAIEKLLSYESWCKLMIKERFDKEVKIKELENKCENKQERDKIFEILEKYEEERQKTFEQDSGFAKKGKFYITGRMLKGFDEIKEKWLKEKDRSEQNLINILNKYQTDNSKLVGDRNLFEFIIKLENQCLWNGDIDYLKIKRDINKNQIWLDRPEMPRFTMPDFKKHPLWYRYEDPSNSNFRNYKIEVVKDENYITIPLITERNNEYFEENYTFNLAKLKKLSENITFIPKSKNKEFEFIDSNDEEEDKKDQKKSKQYIKYCDTAKNTSYGKSGGIRLYFNRNELENYKDGKKMDSYTVFTLSIRDYKSLFAKEKLQPQIFNTVDNKITSLKIQKKFGNEEQTNFLSYFTQNQITKKDWMDEKTFQNVKELNEGIRVLSVDLGQRFFAAVSCFEIMSEIDNNKLFFNLNDQNHKIIRINDKNYYAKHIYSKTIKLSGEDDDLYKERKINKNYKLSYQERKNKIGIFTRQINKLNQLLKIIRNDEIDKEKFKELIETTKRYVKNTYNDGIIDWNNVDNKILSYENKEDVINLHKELDKKLEIDFKEFIRECRKPIFRSGGLSMQRIDFLEKLNKLKRKWVARTQKSAESIVLTPKFGYKLKEHINELKDNRVKQGVNYILMTALGYIKDNEIKNDSKKKQKEDWVKKNRACQIILMEKLTEYTFAEDRPREENSKLRMWSHRQIFNFLQQKASLWGILVGDVFAPYTSKCLSDNNAPGIRCHQVTKKDLIDNSWFLKIVVKDDAFCDLIEINKENVKNKSIKINDILPLRGGELFASIKDGKLHIVQADINASRNIAKRFLSQINPFRVVLKKDKDETFHLKNEPNYLKNYYSILNFVPTNEELTFFKVEENKDIKPTKRIKMDKHEKESTDEGDDYSKNQIALFRDDSGIFFDKSLWVDGKIFWSVVKNKMTKLLRERNNKKNGSK

SEQ ID NO: 199 - protein, 1447 aa, unidentified
  source 1..1447: note = Verrucomicrobiaceae bacterium
MPLSRIYQGRTNSLIILTPTPQEPWDHKALARFDSPLWRHHALFQDAVNYYQLCLVALASSDGTRPLSKLHEQMKASWDEAKTDTEDSWRVRLARRLGIPAASLFEAALAKVLEGNEAPERARELAGELLLDKIEGDIQQAGRGYWPRFCDPKANPTYDYSATARASASGLTKLAAVIHAENVTEEALKQVAAEMDLSWTVKLQPDKNFVGAEARARLLEAAHHFIKVAESPPTKLAEVLARFPDGLALWQALPEKIAALPEETQVPRNRKASPDLTFATLLFQHFPSLFTAAVLGLSVGKPKSVKAPKVVEKVSARRKANAVTQAVVIEEPEIDFAELGDDPIKLARGERGFVFPAFTSLSFWAVPGPHVPVWKEFDIAAFKEALKTVNQFKLKTSERNALLAEAQRRLDYMDEKTHDWKTGDSDEPGHIPPRLKSDPNFTLIQALTQDEGVSNKATGDQHIPKGVYTGGLRGFYAIKKDWCELWERKADKSQGTPTEEELISIVTDYQRDHVYDVGDVGLFRALCEPRFWPLWQPLTDEQEAERIKAGRAKDMISAYRVWLELQEDVVRLAQPIRFTPAHAENSRRLFMFSDISGSHGAEFGSDGKSLEVSIAYDVDGKLQPVRAKLEFSAPRAARDELEGLSGGSESMRWFQPMMKALDCPEVEMPALEKCAVSLMPDVVKKGGGKWVRLLLNFPATLEPEGLIRHIGKQAMWYKQFNGTYKPRTQQLDTGLHLYWPGLEKAPEAEDAAAWWNREEIRAKGFSVLSVDLGQRDAGAWALLESRSDKAFSRNRQPFIELGEAGGKLWSTALLGLGMLRLPGEDARTGALDDQGKRAVEFHGKAGRNALEAEWQEAREMALLFGGEEAKSRLGPGFDHLSHSKQNEELLRILSRAQSRLARFHRWSCRIHEKPEATGDDVIDYGQVDELLTKTAEAMLENLKALYTNAGGILDSKSKQPLTLVGLRKKLEAQKVEPEKIAAVLKPHAEIIFQRLGTLIPELKQHLRVSLERLANRELPLRHREWVWNEAFEKLEQGNFKKEENPKWIRGQRGLSMARIEQIENLRKRFMSLRRQMSLIPGEQVKQGVEDKGQRQPEPCEDILNKLDRMKQQRVNQTAHLILAQALGLRLRPHLANDAEREEKDIHGEYELIPGRKPVDFIVMEDLSRYLSSQGRAPSENGRLMKWCHRAVLAKLKQMCEPFGIPVLEVPAAYSSRFCALTGVPGFRAVEVHDGNAEDFRWKRLIKKAEKDKSSKDAEAAAMLFDQLHDLNIEAREARKQDKKLPLRTLFAPVAGGPLFIPMVGGGPRQADMNAAINLGLRAIASPTCLRARPKIRAELKDGKHQAMLGNKLEKAAALTLEPPKEPTKELAAQKRTNFFLDEKFVGKFDTAHVTTSGKKLRLSGGMSLWKAIKDGAWQRVKKINDARIAKWKNNPPPEPDPDDEIQF

SEQ ID NO: 200 - RNA, 25 nt, synthetic construct
  source 1..25: mol_type = other RNA
  misc_feature 1..25: note = Synthetic
gggaacaaagctgaagtacttaccc

SEQ ID NO: 201 - DNA, 18 bp, synthetic construct
  source 1..18: mol_type = other DNA
  misc_feature 1..18: note = Synthetic
gggtagggcgggttggga

SEQ ID NO: 202 - DNA, 25 bp, synthetic construct
  misc_feature 25: note = 3ThioMC3-D tag
  misc_feature 25: note = 3ThioMC3-D linker
  source 1..25: mol_type = other DNA
  misc_feature 1..25: note = Synthetic
ttataactattcctaaaaaaaaaaa

SEQ ID NO: 203 - DNA, 26 bp, synthetic construct
  misc_feature 1: note = 5thiomc6-d tag
  misc_feature 1..26: note = Synthetic
  source 1..26: mol_type = other DNA
  misc_feature 1: note = 5thiomc6-d linker
aaaaaaaaaactcccctaataacaat

SEQ ID NO: 204 - RNA, 45 nt, synthetic construct
  source 1..45: mol_type = other RNA
  misc_feature 1..45: note = Synthetic
gggtaggaatagttataatttccctttcccattgttattagggag

SEQ ID NO: 205 - RNA, 12 nt, synthetic construct
  source 1..12: mol_type = other RNA
  misc_feature 12: note = Iowa black quenchere
  misc_feature 1: note = biotin tag
  misc_feature 1..12: note = Synthetic
tctcgtacgttc

SEQ ID NO: 206 - RNA, 24 nt, synthetic construct
  source 1..24: mol_type = other RNA
  misc_feature 24: note = Iowa black quencher
  misc_feature 1: note = biotin tag
  misc_feature 1..24: note = Synthetic
tctcgtacgttctctcgtacgttc

SEQ ID NO: 207 - DNA, 20 bp, synthetic construct
  source 1..20: mol_type = other DNA
  misc_feature 1..20: note = Synthetic
gcgacggcctaaagccagtg

SEQ ID NO: 208 - DNA, 22 bp, synthetic construct
  misc_feature 1..22: note = Synthetic
  source 1..22: mol_type = other DNA
gtctgccagcatttcatgtgag

SEQ ID NO: 209 - DNA, 24 bp, synthetic construct
  misc_feature 1..24: note = Synthetic
  source 1..24: mol_type = other DNA
gaaattaatacgactcactatagg

SEQ ID NO: 210 - DNA, 21 bp, Neisseria gonorrhoeae
  source 1..21: mol_type = other DNA
gaaattaatacgactcactat

SEQ ID NO: 211 - DNA, 31 bp, Neisseria gonorrhoeae; identical to SEQ ID NO: 14
  source 1..31: mol_type = other DNA

SEQ ID NO: 212 - DNA, 21 bp, Neisseria gonorrhoeae
  source 1..21: mol_type = other DNA
gaaattaatacgactcactat

SEQ ID NO: 213 - DNA, 32 bp, Neisseria gonorrhoeae; identical to SEQ ID NO: 16
  source 1..32: mol_type = other DNA

SEQ ID NO: 214 - DNA, 21 bp, Neisseria gonorrhoeae
  source 1..21: mol_type = other DNA
gaaattaatacgactcactat

SEQ ID NO: 215 - DNA, 31 bp, Neisseria gonorrhoeae; identical to SEQ ID NO: 18
  source 1..31: mol_type = other DNA

SEQ ID NO: 216 - DNA, 21 bp, Neisseria gonorrhoeae
  source 1..21: mol_type = other DNA
gaaattaatacgactcactat

SEQ ID NO: 217 - DNA, 32 bp, Neisseria gonorrhoeae; identical to SEQ ID NO: 20
  source 1..32: mol_type = other DNA

SEQ ID NO: 218 - DNA, 21 bp, Neisseria gonorrhoeae
  source 1..21: mol_type = other DNA
gaaattaatacgactcactat

SEQ ID NO: 219 - DNA, 30 bp, Neisseria gonorrhoeae; identical to SEQ ID NO: 22
  source 1..30: mol_type = other DNA

SEQ ID NO: 220 - DNA, 21 bp, Neisseria gonorrhoeae
  source 1..21: mol_type = other DNA
gaaattaatacgactcactat

SEQ ID NO: 221 - DNA, 30 bp, Neisseria gonorrhoeae; identical to SEQ ID NO: 24
  source 1..30: mol_type = other DNA

SEQ ID NO: 222 - RNA, 36 nt, Neisseria gonorrhoeae
  source 1..36: mol_type = other RNA
gatttagactaccccaaaaacgaaggggactaaaac

SEQ ID NO: 223 - RNA, 36 nt, Neisseria gonorrhoeae; identical to SEQ ID NO: 222
  source 1..36: mol_type = other RNA

SEQ ID NO: 224 - RNA, 36 nt, Neisseria gonorrhoeae; identical to SEQ ID NO: 222
  source 1..36: mol_type = other RNA

SEQ ID NO: 225 - DNA, 1332 bp, Neisseria gonorrhoeae
  source 1..1332: mol_type = other DNA
aaggtctgtatttaaatcatgttgcgggaaagcaacattttcaaaaaagttaatttattgttttatattgaaatattatttttcaaaataaaaattccaaaatttacccgaaatttgttccgaaaaatggtttttttttttcggggggtaattggagactgattgggtgtttgcccgatgtttttagcaaatttacaaaaggaagccgatatgcgaaaaaaacttaccgccctcgtattgtccgcactgccgtttgcggcagttgccgatgtcagcctgtacggcgaagtcaaagctggtgtggaaggcaggaacatccggctgcagttgaccgagccaccctcagaaggtcaaacgggcaatacagttactaaggccaaaagccgcatcaggacgaaagtcagtgatttcggctcgtttatcggctttaagggggtggggatttgggcggcgggctgaaggctgtttggcagctcgagcaagacgtatccgttgccggcggcggcgcgacccgttggggtaacagggaatcctttatcggcttggcaggcgaattcggcacggcgctcgccggtcgcgttgcgaatccgtttggcgatgccagcaaagccattgatccttgggacagcaataataatgtggcttcgcaattgggtattttcaaacgccacgacggtatgccggtttccgtgcgttacgattcccccggattttccggtttcagcggcagcattcaatttgttccgagtcaaaacagcaagtccgcctatacgcctgctactttcacgctggaaagtaatcagatgaaaccagttccggctgttgtcggcaagccggggtcggatgtgtattatgccggtctgaattacaaaaatggcggctttttcggaaattatgcccttaaatatgcgaaacacgccaatgaggggcatgatgctttctttttgttcttgctcggcagagcgagtgataccgatccattgaaaaaccatcaggtacaccgcctgacgggcggctatggggaaggcggcttgaatctcgccttggcggctcagttggatttgtctgaaaatgccgacaaaaccaaaaacagtacgaccgaaattgccgccactgcttcctaccgcttcggtaatacagtcccgcgcatcagctatgcccatggtttcgactttgtcgaacgcagtcagaaacgcgaacataccagctatgatcaaatcatcgccggtgtcgattacgatttttccaagcgcacttccgccatcatgtctgccgcttggctgaaacgaaataccggcatcggcaactacactcaaattaatgccgcctccg

(19) United States
(12) Patent Application Publication
(10) Pub. No.: US 2026/0049363 A1
(43) Pub. Date: Feb. 19, 2026

(54) Gonorrhea Diagnostic

(71) Applicants: The Brigham and Women's Hospital, Inc., Boston, MA (US); The General Hospital Corporation, Boston, MA (US); President and Fellows of Harvard College, Cambridge, MA (US)

(72) Inventors: Lao-Tzu Allan-Blitz, Boston, MA (US); Jacob Lemieux, Boston, MA (US); Pardis Sabeti, Cambridge, MA (US)

(21) Appl. No.: 19/304,255

(22) Filed: Aug. 19, 2025

Related U.S. Application Data
(60) Provisional application No. 63/684,799, filed on Aug. 19, 2024.

(51) Int. Cl. C12Q 1/689; C12Q 1/34; C12Q 1/6806; C12Q 1/6825; C12Q 1/686

(52) U.S. Cl. CPC C12Q 1/689; C12Q 1/34; C12Q 1/6806; C12Q 1/6825; C12Q 1/686; C12Q 2600/106; C12Q 2600/156; G01N 2333/922

(57) ABSTRACT

Neisseria gonorrhoeae is one of the most common bacterial sexually transmitted infections (STI). Diagnosis depends on standard nucleic acid amplification testing (NAAT), which is impracticable in most low-resource settings, where the prevalence of this STI is highest. Consequently, such areas utilize syndromic management, which misses a high proportion of cases and leads to antibiotic overuse, contributing to the troubling rise of resistance to the commonly prescribed ciprofloxacin, cefixime and ceftriaxone antibiotics for the treatment of N. gonorrhoeae infections. A specific, highly sensitive and cost-effective lateral flow assay is disclosed that utilizes CRISPR-Cas orthologs, multiplex SHERLOCK technology and isothermal amplification via recombinase polymerase amplification (RPA) for the rapid detection of antibiotic resistance to ciprofloxacin, cefixime and/or ceftriaxone at the point of care. This approach has the potential to increase treatment efficacy in the field and mitigate the spread of antibiotic resistance.

### CROSS REFERENCE TO RELATED APPLICATIONS

This application claims the benefit of U.S. Provisional Application No. 63/684,799 filed Aug. 19, 2024. The entire contents of the above-identified applications are hereby fully incorporated herein by reference.

### STATEMENT REGARDING FEDERALLY SPONSORED RESEARCH

This invention was made with government support under Grant No. (s) U19AI110818 awarded by the National Institutes of Health. The government has certain rights in the invention.

### SEQUENCE LISTING

This application contains a sequence listing filed in electronic form as an xml file entitled BROD-6035US_ST26_revised, created on Oct. 10, 2025, and having a size of 476,882 bytes. The content of the sequence listing is incorporated herein in its entirety.

### TECHNICAL FIELD

The subject matter disclosed herein is generally directed to rapid point-of-care diagnostics related to the use of CRISPR effector systems for the detection of antibiotic resistant Neisseria pathogens.

### BACKGROUND

Antimicrobial resistance in Neisseria gonorrhoeae is a global public health emergency The causative agent of gonorrhea, Neisseria gonorrhoeae is one of the most common bacterial sexually transmitted infections worldwide, with an estimated 87 million cases in 2016. that colonizes urogenital, anal, and nasopharyngeal tissues. The consequences of untreated Neisseria gonorrhoeae infection can be profound, ranging from pelvic inflammatory disease, infertility, and neonatal blindness to an increased risk for HIV infection. Importantly, Neisseria gonorrhea has developed resistance to all antimicrobials used in its treatment. In response, the Centers for Disease Control and Prevention continue to escalate treatment regimens for gonorrhea; yet with standard of care diagnostics based on nucleic acid amplification testing (NAAT) precluding susceptibility determination, all Neisseria gonorrhea infections are treated with third generation cephalosporins, resulting in selective pressure for the emergence of resistance. According to a recent report by the World Health Organization (WHO) over 60 percent of infections are showing resistance to ciprofloxacin, one of the most widely used oral antibacterials. Further complicating the matter, most Neisseria gonorrhoeae infections occur in low-resource settings with high rates of antimicrobial resistance, with insufficient laboratory capacity for NAAT.

Thus, there is an urgent need for the development of rapid molecular assays for predicting antimicrobial susceptibility, or resistance-guided therapy.

Citation or identification of any document in this application is not an admission that such a document is available as prior art to the present disclosure.

### SUMMARY

In a first aspect, the present disclosure provides a nucleic acid detection system for detecting an antibiotic-resistant Neisseria gonorrhoeae pathogen in a patient sample, comprising a first CRISPR-Cas system having collateral cleavage activity and configured to bind one or more N. gonorrhoeae polynucleotide sequences that identify the presence of N. gonorrhoeae; a second CRISPR-Cas system having collateral cleavage activity and configured to bind one or more N. gonorrhoeae polynucleotide sequences that indicate resistance or susceptibility to an antibiotic; a first detection construct comprising a cutting motif configured to generate a first detectable signal when preferentially cut by the one or more CRISPR-Cas systems configured to bind one or more N. gonorrhoeae polynucleotide sequences that identify the presence of N. gonorrhoeae, and a second detection construct comprising cutting motifs configured to generate one or more second detectable signals when preferentially cut by CRISPR-Cas systems configured to bind N. gonorrhoeae polynucleotide sequences that indicate resistance or susceptibility to an antibiotic, wherein the antibiotic is not ciprofloxacin.

In one embodiment, the N. gonorrhoeae polynucleotide sequences that indicate resistance or susceptibility to an antibiotic identify resistance or susceptibility to cefixime and/or ceftriaxone.

In a second aspect, the present disclosure further provides a nucleic acid detection system for detecting an antibiotic-resistant Neisseria gonorrhoeae pathogen in a patient sample, comprising: a first CRISPR-Cas system having collateral cleavage activity and configured to bind one or more N. gonorrhoeae polynucleotide sequences that identify the presence of N. gonorrhoeae; a second CRISPR-Cas system having collateral cleavage activity and configured to bind one or more N. gonorrhoeae polynucleotide sequences that indicate antibiotic resistance; a first detection construct comprising a cutting motif configured to generate a first detectable signal when preferentially cut by the one or more CRISPR-Cas systems configured to bind one or more N. gonorrhoeae polynucleotide sequences that identify the presence of N. gonorrhoeae, and a second detection construct comprising cutting motifs configured to generate one or more second detectable signals when preferentially cut by CRISPR-Cas systems configured to bind N. gonorrhoeae polynucleotide sequences that indicate resistance to ciprofloxacin, and either cefixime or ceftriaxone, or ciprofloxacin, cefixime and ceftriaxone.

In one embodiment, the one or more N. gonorrhoeae polynucleotide sequences that identify the presence of N. gonorrhoeae are located in a porA gene.

In one embodiment, the one or more N. gonorrhoeae polynucleotide sequences that identify the presence of N. gonorrhoeae are located in a nucleotide sequence of SEQ ID NO: 225.

In one embodiment, the one or more N. gonorrhoeae polynucleotide sequences that indicate resistance to cefixime or ceftriaxone are located in a penicillin-binding protein 2 (penA) gene.

In one embodiment, the one or more N. gonorrhoeae polynucleotide sequences that indicate resistance to ceftriaxone comprise single nucleotide polymorphisms (SNPs) in the mosaic allele type 60 of the penA gene.

In one embodiment, the one or more polynucleotide sequences that indicate resistance to ceftriaxone comprise one or more mutations in codons 311, 316, 483, 501, 512, 516, 542 and 545 of the penA gene.

In one embodiment, the one or more mutations comprise A311V, V316T/P and T483S.

In one embodiment, the one or more N. gonorrhoeae polynucleotide sequences that indicate resistance to ceftriaxone further comprise one or more mutations in codons 120 and 121 of the porB gene or a mutation in codon 421 of the ponA gene.

In one embodiment, the one or more N. gonorrhoeae polynucleotide sequences that indicate resistance to cefixime comprise one or more mutations in codons 311, 312, 316, 345, 483, 375, 376, 377, 501, 542 and 551 of the penA gene.

In one embodiment, the one or more polynucleotide sequences that indicate resistance to cefixime comprise one or more mutations chosen from A501V, A501P, A501T, N512Y, A516G, G542S, G545S, P551L and P551S in the penA gene.

In one embodiment, the nucleic acid detection system further comprising amplification reagents. In one embodiment, the amplification reagents comprise one or more primers pairs for amplifying the one or more N. gonorrhoeae polynucleotide sequences that identify the presence of N. gonorrhoeae, the one or more N. gonorrhoeae polynucleotide sequences that indicate antibiotic resistance, or both. The one or more primers pairs for amplifying the one or more N. gonorrhoeae polynucleotide sequences that identify the presence of N. gonorrhoeae may be selected from any one of the nucleotide sequences set forth in SEQ ID NO: 210-221.

In one embodiment, the first and second CRISPR-Cas systems are independently selected from a Type V or a Type VI CRISPR-Cas system.

In a third aspect, a method for detecting antibiotic-resistant N. gonorrhoeae in a patient sample is also disclosed comprising contacting one or more samples with a first CRISPR-Cas system having collateral cleavage activity and configured to bind one or more N. gonorrhoeae polynucleotide sequences that identify the presence of N. gonorrhoeae; a second CRISPR-Cas system having collateral cleavage activity and configured to bind one or more N. gonorrhoeae polynucleotide sequences that indicate antibiotic resistance; a first detection construct comprising a cutting motif configured to generate a first detectable signal when preferentially cut by the one or more CRISPR-Cas systems configured to bind one or more N. gonorrhoeae polynucleotide sequences that identify the presence of N. gonorrhoeae; a second detection construct comprising cutting motifs configured to generate one or more second detectable signals when preferentially cut by the one or more CRISPR-Cas systems configured to bind one or more N. gonorrhoeae polynucleotide sequences that indicate resistance to an antibiotic, wherein the antibiotic is not ciprofloxacin; and detecting the generation of the first detectable signal, the one or more second detectable signals, or both, wherein the detection of the first detectable signal but not the one or more second detectable signals indicates the presence of an antibiotic-sensitive strain of N. gonorrhoeae in the sample, and wherein the detection of the first detectable signal and the one or more second detectable signals indicates the presence of a strain of N. gonorrhoeae in the sample that is resistant to an antibiotic other than ciprofloxacin.

In one embodiment, the one or more N. gonorrhoeae polynucleotide sequences that indicate antibiotic resistance indicate resistance to cefixime and/or ceftriaxone.

In one embodiment, the one or more N. gonorrhoeae polynucleotide sequences that identify the presence of N. gonorrhoeae are located in a porA gene.

In one embodiment, the one or more N. gonorrhoeae polynucleotide sequences that identify the presence of N. gonorrhoeae are located in the nucleotide sequence set forth in SEQ ID NO: 225.

In one embodiment, the one or more N. gonorrhoeae polynucleotide sequences that indicate resistance to cefixime or ceftriaxone are located in a penicillin-binding protein 2 (penA) gene.

In one embodiment, the one or more N. gonorrhoeae polynucleotide sequences that indicate resistance to ceftriaxone comprise single nucleotide polymorphisms (SNPs) in a mosaic allele type 60 of the penA gene.

In one embodiment, the one or more polynucleotide sequences that indicate resistance to ceftriaxone comprise one or more mutations in codons 311, 316, 483, 501, 512, 516, 542 and 545 of the penA gene.

In one embodiment, the one or more mutations comprise A311V, V316T/P and T483S.

In one embodiment, the one or more N. gonorrhoeae polynucleotide sequences that indicate resistance to ceftriaxone comprise one or more mutations in codons 120 and 121 of the porB gene.

In one embodiment, the one or more N. gonorrhoeae polynucleotide sequences that indicate resistance to ceftriaxone comprise a mutation in codon 421 of the ponA gene.

In one embodiment, the one or more N. gonorrhoeae polynucleotide sequences that indicate resistance to cefixime comprise one or more mutations in codons 311, 312, 316, 345, 483, 375, 376, 377, 501, 542 and 551 of the penA gene.

In one embodiment, the one or more polynucleotide sequences that indicate resistance to cefixime comprise one or more mutations chosen from A501V, A501P, A501T, N512Y, A516G, G542S, G545S, P551L and P551S in the penA gene

In one embodiment, the method for detecting antibiotic-resistant N. gonorrhoeae in a patient sample further comprises amplification reagents.

In one embodiment, the amplification reagents comprise one or more primers pairs for amplifying the one or more N. gonorrhoeae polynucleotide sequences that identify the presence of N. gonorrhoeae, the one or more N. gonorrhoeae polynucleotide sequences that indicate antibiotic resistance, or both.

In one embodiment, one or more primers pairs for amplifying the one or more N. gonorrhoeae polynucleotide sequences that identify the presence of N. gonorrhoeae are selected from any one of the nucleotide sequences set forth in SEQ ID NO: 210-221.

In one embodiment, the patient sample is a sample of blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate, a fluid obtained from a joint, or a swab of skin or a mucosal membrane surface.

In one embodiment, nucleic acids in the patient sample are extracted by incubating the sample in 0.05%-0.5% Triton-X at about 25-37° C. for about 5 minutes.

In a fourth aspect, a method for detecting antibiotic-resistant N. gonorrhoeae in a patient sample comprising: contacting one or more samples with a first CRISPR-Cas system having collateral cleavage activity and configured to bind one or more N. gonorrhoeae polynucleotide sequences that identify the presence of N. gonorrhoeae; a second CRISPR-Cas system having collateral cleavage activity and configured to bind one or more N. gonorrhoeae polynucleotide sequences that indicate antibiotic resistance; a first detection construct comprising a cutting motif configured to generate a first detectable signal when preferentially cut by the one or more CRISPR-Cas systems configured to bind one or more N. gonorrhoeae polynucleotide sequences that identify the presence of N. gonorrhoeae, and a second detection construct comprising cutting motifs configured to generate one or more second detectable signals when preferentially cut by the one or more CRISPR-Cas systems configured to bind N. gonorrhoeae polynucleotide sequences that indicate resistance to ciprofloxacin, and either cefixime or ceftriaxone, or ciprofloxacin, cefixime and ceftriaxone, and detecting the generation of the first detectable signal, the one or more second detectable signals, or both, wherein the detection of the first detectable signal but not the one or more second detectable signals indicates the presence of an antibiotic-sensitive strain of N. gonorrhoeae in the sample, and wherein the detection of the first detectable signal and the one or more second detectable signals indicates the presence of a strain of N. gonorrhoeae in the sample that is resistant to ciprofloxacin, and either cefixime or ceftriaxone, or ciprofloxacin, cefixime and ceftriaxone.

In one embodiment, the one or more N. gonorrhoeae polynucleotide sequences that identify the presence of N. gonorrhoeae are located in a porA gene.

In one embodiment, the one or more N. gonorrhoeae polynucleotide sequences that identify the presence of N. gonorrhoeae are located in the nucleotide sequence set forth in SEQ ID NO: 225.

In one embodiment, the one or more N. gonorrhoeae polynucleotide sequences that indicate resistance to ciprofloxacin are located in a gyrAse A (gyrA) gene.

In one embodiment, the one or more N. gonorrhoeae polynucleotide sequences that indicate antibiotic resistance to ciprofloxacin comprise a single nucleotide polymorphism (SNP) in codon 91 of the gyrA gene.

In one embodiment, the SNP at codon 91 of the gyrA gene is amino acid substitution S91F.

In one embodiment, the one or more N. gonorrhoeae polynucleotide sequences that indicate resistance to cefixime or ceftriaxone are located in a penicillin-binding protein 2 (penA) gene.

In one embodiment, the one or more N. gonorrhoeae polynucleotide sequences that indicate resistance to ceftriaxone comprise single nucleotide polymorphisms (SNPs) in the mosaic allele type 60 of the penA gene.

In one embodiment, the one or more polynucleotide sequences that indicate resistance to ceftriaxone comprise one or more mutations in codons 311, 316, 483, 501, 512, 516, 542 and 545 of the penA gene.

In one embodiment, the one or more mutations comprise A311V, V316T/P and T483S.

In one embodiment, the one or more N. gonorrhoeae polynucleotide sequences that indicate resistance to ceftriaxone further comprise one or more mutations in codons 120 and 121 of the porB gene.

In one embodiment, the one or more N. gonorrhoeae polynucleotide sequences that indicate resistance to ceftriaxone further comprise a mutation in codon 421 of the ponA gene.

In one embodiment, the one or more N. gonorrhoeae polynucleotide sequences that indicate susceptibility to cefixime comprise a wild type sequence in one or more codons chosen from codons 311, 312, 316, 345, 483, 375, 376, 377, 501, 542 and 551 of the penA gene.

In one embodiment, the one or more polynucleotide sequences that indicate resistance to cefixime comprise one or more mutations chosen from A501V, A501P, A501T, N512Y, A516G, G542S, G545S, P551L and P551S in the penA gene.

In one embodiment, the method for detecting antibiotic-resistant N. gonorrhoeae in a patient sample further comprising amplification reagents.

In one embodiment, the amplification reagents comprise one or more primers pairs for amplifying the one or more N. gonorrhoeae polynucleotide sequences that identify the presence of N. gonorrhoeae, the one or more N. gonorrhoeae polynucleotide sequences that indicate antibiotic resistance, or both.

In one embodiment, the one or more primers pairs for amplifying the one or more N. gonorrhoeae polynucleotide sequences that identify the presence of N. gonorrhoeae are selected from any one of the nucleotide sequences set forth in SEQ ID NO: 210-221.

In one embodiment, the first and second CRISPR-Cas systems are independently selected from a Type V CRISPR-Cas system or a Type VI CRISPR-Cas system.

In one embodiment, the patient sample is a sample of blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate, a fluid obtained from a joint, or a swab of skin or a mucosal membrane surface.

In one embodiment, nucleic acids in the patient sample are extracted by incubating the sample in 0.05%-0.5% Triton-X at about 25-37° C. for about 5 minutes.

In a fifth aspect, a lateral flow device for detecting an antibiotic-resistant Neisseria gonorrhoeae pathogen in a patient sample is disclosed, comprising at least one lateral flow substrate each having a first and second end, a first lateral flow substrate comprising: a sample loading portion at the first end of the first lateral flow substrate comprising: a first CRISPR-Cas system having collateral cleavage activity and configured to bind one or more N. gonorrhoeae polynucleotide sequences that identify the presence of N. gonorrhoeae; a first detection construct comprising a cutting motif configured to generate a first detectable signal when preferentially cut by the one or more CRISPR-Cas systems configured to bind one or more N. gonorrhoeae polynucleotide sequences that identify the presence of N. gonorrhoeae; and a first capture region comprising a first binding agent that is immobilized to the first capture region between the first and second ends of the first lateral flow substrate.

In one embodiment, the first binding agent binds to and sequesters the first detection construct to the first capture region, wherein detection of the first detectable signal at the first capture region indicates the presence of N. gonorrhoeae in the patient's sample.

In one embodiment, the sample loading portion of the first lateral flow substrate further comprises a second CRISPR-Cas system having collateral cleavage activity and configured to bind one or more N. gonorrhoeae polynucleotide sequences that indicate antibiotic resistance; and a second detection construct comprising a cutting motif configured to generate one or more second detectable signals when preferentially cut by the one or more CRISPR-Cas systems configured to bind one or more N. gonorrhoeae polynucleotide sequences that indicate the presence of an antibiotic-resistant strain of N. gonorrhoeae in the patient's sample.

In one embodiment, the second end of the first lateral flow substrate comprises a second capture region comprising a second binding agent that is immobilized to the second capture region.

In one embodiment, the second binding agent binds to and sequesters the second detection construct to the second capture region, wherein detection of the second detectable signal at the second capture region indicates the presence of an antibiotic-resistant Neisseria gonorrhoeae pathogen in the patient's sample.

In one embodiment, the one or more N. gonorrhoeae polynucleotide sequences that identify the presence of N. gonorrhoeae are located in a porA gene.

In one embodiment, the one or more N. gonorrhoeae polynucleotide sequences that identify the presence of N. gonorrhoeae are located in the nucleotide sequence set forth in SEQ ID NO: 225.

In one embodiment, the one or more N. gonorrhoeae polynucleotide sequences indicate resistance to an antibiotic other than ciprofloxacin.

In one embodiment, the one or more N. gonorrhoeae polynucleotide sequences that indicate resistance to an antibiotic indicate resistance to ciprofloxacin, and either cefixime or ceftriaxone, or ciprofloxacin, cefixime and ceftriaxone.

In one embodiment, the one or more N. gonorrhoeae polynucleotide sequences that indicate resistance to ciprofloxacin are located in a gyrAse A (gyrA) gene.

In one embodiment, the one or more N. gonorrhoeae polynucleotide sequences that indicate resistance to ciprofloxacin comprise a single nucleotide polymorphism (SNP) in codon 91 of the gyrA gene.

In one embodiment, the SNP at codon 91 of the gyrA gene is amino acid substitution S91F.

In one embodiment, the one or more N. gonorrhoeae polynucleotide sequences that indicate resistance to cefixime or ceftriaxone are located in a penicillin-binding protein 2 (penA) gene.

In one embodiment, the one or more N. gonorrhoeae polynucleotide sequences that indicate resistance to ceftriaxone comprise single nucleotide polymorphisms (SNPs) in a mosaic allele type 60 of the penA gene. As used herein, the term “mosaic allele type 60 of the penA gene” refers to a variant form of the Neisseria gonorrhoeae penicillin-binding protein 2 (penA) gene that contains characteristic sequence alterations associated with reduced susceptibility to cephalosporin antibiotics, particularly ceftriaxone. Mosaic allele type 60 may be characterized by the presence of recombinant DNA sequences that have been horizontally transferred between different Neisseria species, resulting in a chimeric penA gene structure. The mosaic allele type 60 comprises specific single nucleotide polymorphisms (SNPs) and insertions that distinguish it from wild-type penA sequences, including but not limited to alterations in the amino acid region spanning positions 375-377, and mutations at key resistance-associated codons such as 311, 316, 483, 501, 512, 516, 542, and 545. The presence of mosaic allele type 60 is predictive of reduced cephalosporin susceptibility and serves as a molecular marker for antibiotic resistance in clinical N. gonorrhoeae isolates. The mosaic structure reflects evolutionary pressure from antibiotic exposure and represents a mechanism by which N. gonorrhoeae has developed resistance to beta-lactam antibiotics through acquisition of foreign DNA sequences from commensal Neisseria species.

In one embodiment, the one or more polynucleotide sequences that indicate resistance to ceftriaxone comprise one or more mutations in codons 311, 316, 483, 501, 512, 516, 542 and 545 of the penA gene.

In one embodiment, the one or more mutations comprise A311V, V316T/P and T483S.

In one embodiment, the one or more N. gonorrhoeae polynucleotide sequences that indicate resistance to ceftriaxone further comprise one or more mutations in codons 120 and 121 of the porB gene.

In one embodiment, the one or more N. gonorrhoeae polynucleotide sequences that indicate resistance to ceftriaxone further comprise a mutation in codon 421 of the ponA gene.

In one embodiment, the one or more N. gonorrhoeae polynucleotide sequences that indicate susceptibility to cefixime comprise a wild type sequence in one or more codons chosen from codons 311, 312, 316, 345, 483, 375, 376, 377, 501, 542 and 551 of the penA gene.

In one embodiment, the one or more polynucleotide sequences that indicate resistance to cefixime comprise one or more mutations chosen from A501V, A501P, A501T, N512Y, A516G, G542S, G545S, P551L and P551S in the penA gene.

In one embodiment, the lateral flow device further comprises amplification reagents.

In one embodiment, the amplification reagents comprise one or more primers pairs for amplifying the one or more N. gonorrhoeae polynucleotide sequences that identify the presence of N. gonorrhoeae, the one or more N. gonorrhoeae polynucleotide sequences that indicate antibiotic resistance, or both.

In one embodiment, the one or more primers pairs for amplifying the one or more N. gonorrhoeae polynucleotide sequences that identify the presence of N. gonorrhoeae are selected from any one of the nucleotide sequences set forth in SEQ ID NO: 210-221.

In one embodiment, the first and second CRISPR-Cas systems are independently selected from a Type V CRISPR-Cas system or a Type VI CRISPR-Cas system.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1A shows estimated new global cases of gonorrhoea in 2016. Estimated numbers (in millions) of incident cases of gonorrhoea in adults (15-49 years of age) by WHO region. These data correspond to 20 new gonococcal infections per 1,000 women and 26 per 1,000 men globally. The highest incidence was in the WHO African region, with 41 cases per 1,000 women and 50 per 1,000 men, followed by the WHO region of the Americas, with 23 cases per 1,000 women and 32 per 1,000 men; the lowest incidence was in the WHO European region, with 7 cases per 1,000 women and 11 per 1,000 men. The World Bank Income Classification is also shown. Reproduced from Unemo et al. Gonorrhoea. Nat Rev Dis Primers 5, 79 (2019). FIG. 1B shows the recommended empirical therapy for gonorrhoea and emergence of antimicrobial resistance (AMR) in Neisseria gonorrhoeae. Each bar represents a gonorrhoea therapy, and the length of the bar represents the time period from when the therapy started to be used until when clinical and/or in vitro resistance threatening the effectiveness of that specific antimicrobial therapy emerged. In vitro verified antimicrobial resistance (AMR) determinants are also shown. PBP2 amino acid alterations that increase the minimum inhibitory concentration (MIC) of extended-spectrum cephalosporins (ESCs; verified, for example, by site-directed mutagenesis or transformation) in nonmosaic and mosaic (in which concomitant epistatic mosaic penA mutations are also needed) penA alleles are noted by an asterisk. Additionally, PBP2 G542S, P551S, and P551L amino acid alterations in nonmosaic penA alleles have been statistically associated with gonococcal strains with decreased susceptibility to ESCs. A grave concern is that during the past decade(s) resistance to azithromycin and decreased susceptibility to the ESC ceftriaxone, the last remaining option for empirical monotherapy, have been reported worldwide. rRNA, ribosomal RNA; SNP, single nucleotide polymorphism. Reproduced from Unemo et al. Gonorrhoea. Nat Rev Dis Primers 5, 79 (2019). FIG. 1C shows an example of targeted proteins that confer antibiotic resistance in Neisseria gonorrhoeae. Transpeptidase penicillin binding protein 2 (PBP2), encoded by penA is a periplasmic transpeptidase and the main lethal target of cephalosporins; most resistant isolates contain mosaic mutations in penA. The efflux pump, MtrCDE, composed of subunits MtrE, MtrC, and MtrD, and its repressor mtrR contribute to N. gonorrhoeae resistance through antimicrobial efflux. The major porin protein, PorB, encoded by penB, is also a main resistance determinant that cannot manifest independently, but requires a concomitant mutation in mtrR. Reproduced from Quillin and Seifert (2018) Neisseria gonorrhoeae host adaptation and pathogenesis. Nat Rev Microbiol. 16(4):226-240.

FIG. 2 shows the mutations in gyrA, penA, porB and mtrR genes within the Neisseria gonorrhoeae genome that confer reduced susceptibility to ciprofloxacin (gyrA), cefixime, and ceftriaxone (reproduced from Allan-Blitz et al., Resistance-guided therapy for Neisseria gonorrhoeae. Clin. Infect. Dis. 75, 1655-1660 (2022)).

FIG. 3A shows an example implementation of three guides targeting different regions of the porA gene (see TABLES XVI and XVII) tested on three N. gonorrhoeae purified isolates as well as a synthetic gyrA template as a control and a negative template control (NTC). FIG. 3B shows the selected PorA Cas13a crRNA 2 guide sequence annealed to its target sequence. *** indicates statistically significant differences in florescence at the P<0.05 level.

FIG. 4A shows the limit of detection of the N. gonorrhoeae Cas13a detection assay using the selected guide-primer set for the porA gene among purified N. gonorrhoeae isolates and a negative template control (NTC). FIG. 4B shows the limit of detection of the Cas13a-based assay using the wild-type guide against synthetic wild-type DNA target. FIG. 4C shows the limit of detection of Cas13a-based assay using the mutant guide against synthetic mutant DNA target. The serial dilutions of synthetic DNA were done in nuclease-free water.

FIG. 5A shows a schematic of a porA lateral flow Sherlock assay for the detection of Neisseria gonorrhoeae in a sample. FIG. 5B shows Neisseria gonorrhoeae detection among 15 urine specimens using the porA lateral flow Sherlock assay. FIG. 5C shows the porA lateral flow Sherlock assay can distinguish Neisseria gonorrhoeae from N. meningitidis (ATCC 13077), N. perflava (ATCC 14799), and N. lactamica (ATCC 23970).

FIG. 6A shows a schematic of the workflow for detergent-based DNA extraction using clinical urine specimen, followed by incubation at 37° for 90 minutes and the Cas13a detection read on lateral flow. FIG. 6B shows the average DNA yield among 5 positive urine specimens using various extraction conditions standardized by subtracting the average DNA found in 3 negative urine specimens. * shows the average DNA yield among 11 positive urine specimens, excluding ID039 with undetectable DNA on Qbit fluorometry.

FIG. 7A shows the effectiveness of the guide RNA to distinguish between mutant and wildtype gyrA target nucleotide sequences with the synthetic mutation at the second position of the spacer region. FIG. 7B shows the effectiveness of the guide RNA to distinguish between mutant and wildtype gyrA target nucleotide sequences with the synthetic mutation at the fourth position of the spacer region.

FIG. 8A-8C shows the efficacy of three different primer sets using the selected gRNA for amplifying the synthetic wildtype and mutant gyrA DNA sequences. The primer set in panel FIG. 8A was selected as it demonstrated the highest amplification while preserving the ability of the gRNAs to distinguish between wild type and mutant target sequences at codon 91 of the gyrA gene.

FIG. 9A shows a Cas13a-based gyrAse A assay for the detection of ciprofloxacin resistance in a pool of 23 purified N. gonorrhoeae isolates. FIG. 9B shows a Cas13a-based assay detecting the gyrA genotype of 23 purified N. gonorrhoeae isolates. FIG. 9C shows an alignment of gyrA DNA sequences encompassing the gyr 91 mutation in 23 samples comprising susceptible and resistant N. gonorrhoeae specimens. The sequences of the wild type and mutant gyrA guide RNAs are also shown.

FIG. 10A shows the efficacy of a Cas13a-based gyrA assay to detect ciprofloxacin resistance using both wild-type and mutant guides for determining gyrA genotype among 3 N. gonorrhoeae isolates. FIG. 10B shows the efficacy of the same Cas13a assay as in FIG. 10A but read on a Qubit 4 fluorometer. NTC, negative template control.

FIG. 11A shows cell lysis, DNA amplification, transcription, binding of Cas13a to amplified RNA, binding of Cas12a to amplified DNA, cleavage of antigen bound reporters and reporter-antibody complex visualized on a lateral flow strip. FIG. 11B shows multiplex amplification of both gyrA and porA with sequential Cas13a detection (blue and orange) and simultaneous Cas12a/Cas13a detection (purple).

FIG. 12A shows CRISPR-Cas13a detection of penA mutations associated with cephalosporin resistance in N. gonorrhoeae. Fluorescence signal over time demonstrates discrimination between wildtype penA sequences (n=11), mutant penA sequences containing resistance-associated mutations (n=18), and negative control specimens (n=5) using wildtype-specific guide RNAs. FIG. 12B shows the analytical sensitivity (limit of detection) of the Cas13a-based mosaic penA detection assay. Serial dilutions from 10⁶ copies/μL to 3.3 copies/μL demonstrate detection capability across clinically relevant target concentrations using wildtype-specific guide RNAs, with NTC (no template control) showing baseline fluorescence throughout the assay period.

The figures herein are for illustrative purposes only and are not necessarily drawn to scale.

### DETAILED DESCRIPTION OF THE EXAMPLE EMBODIMENTS

### I. Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); CRISPR Gene Editing: Methods and Protocols, Editor: Yonglun Luo, Publisher: Springer New York, 2019; and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed disclosure. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, the term “treatment” or “treating” refers to an intervention (e.g. the administration of an agent to a subject) which cures, ameliorates or lessens the symptoms of gonorrhea or removes (or lessens the impact of) its cause(s) (for example, the causative bacterium). In this case, the term is used synonymously with the term “therapy”. Thus, the treatment of infection according to the disclosure may be characterized by the (direct or indirect) bacteriostatic and/or bactericidal action of the compounds of the disclosure. Thus, the compounds of the disclosure find application in methods of killing, or preventing the growth of, bacterial cells.

Additionally, the terms “treatment” or “treating” refers to an intervention (e.g. the administration of an agent to a subject, e.g. an antibiotic or combination of antibiotics) which prevents or delays the onset or progression of a disease or reduces (or eradicates) its incidence within a treated population. In this case, the term treatment is used synonymously with the term “prophylaxis”.

As used herein, an effective amount or a therapeutically effective amount of a compound defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general health of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

As used herein, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The term “antibiotic resistance” refers to a type of drug resistance where a microorganism (e.g., Neisseria gonorrhoeae bacterium) has developed the ability to survive exposure to an antibiotic. Evolutionary stress such as exposure to antibiotics selects for the antibiotic resistant trait. A bacterium may carry several resistance genes. Many antibiotic resistance genes reside on plasmids, whose function is to facilitate the transfer of antibiotic resistance genes from one bacterium to another. “Antibiotic resistance” has an opposite meaning as compared to “antibiotic susceptibility”, that is, a high antibiotic resistance means a low antibiotic susceptibility and vice versa.

As used herein, the phrase “indicate susceptibility” or “indicate antibiotic susceptibility” refers to the molecular characteristic of polynucleotide sequences that serve as predictive biomarkers for antibiotic effectiveness against a pathogen. A polynucleotide sequence “indicates susceptibility” when its presence, absence, or specific nucleotide composition correlates with, predicts, or determines the likelihood that the pathogen harboring such sequence will be effectively inhibited or killed by a specified antibiotic at clinically achievable concentrations. This indication is typically demonstrated through one or more of the following molecular and clinical correlations: (i) association with low minimum inhibitory concentration (MIC) values (e.g., ≤0.015 μg/mL for cephalosporins in N. gonorrhoeae), (ii) correlation with successful clinical treatment outcomes, (iii) presence of wild-type nucleotide sequences at resistance-associated codon positions, (iv) absence of known resistance-conferring mutations, or (v) maintenance of functional antibiotic target sites without resistance-altering modifications. The indication of susceptibility represents a probabilistic relationship based on established genotype-phenotype correlations in clinical isolates, wherein the detection of susceptibility-indicating sequences provides actionable information for therapeutic decision-making. This molecular indication enables resistance-guided therapy by distinguishing susceptible strains from resistant strains at the genetic level prior to phenotypic susceptibility testing.

The term “multiplex polymerase chain reaction” (multiplex PCR) refers to is a modification of polymerase chain reaction. Multiplex-PCR consists of multiple primer sets within a single PCR mixture to produce amplicons of varying sizes that are specific to different DNA sequences. By targeting multiple genes at once, additional information may be gained from a single test run that otherwise would require several times the reagents and more time to perform.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment but may. Furthermore, the features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

Titles or subtitles may be used in the specification for the sole convenience of the reader but are not intended to influence the scope of the present disclosure or to limit any aspect of the disclosure to any subsection, subtitle, or paragraph.

Embodiments disclosed herein utilize Cas proteins possessing non-specific nuclease collateral activity to cleave detectable reporters upon target recognition, providing sensitive and specific diagnostics, including single nucleotide variants, detection based on rRNA sequences, screening for drug resistance, monitoring microbe outbreaks, genetic perturbations, and screening of environmental samples, as described, for example, in PCT/US18/054472 filed Oct. 22, 2018 at [0183]-[0327], incorporated herein by reference. Reference is made to WO 2017/219027, WO2018/107129, US20180298445, US 2018-0274017, US 2018-0305773, WO 2018/170340, U.S. application Ser. No. 15/922,837, filed Mar. 15, 2018 entitled “Devices for CRISPR Effector System Based Diagnostics”, PCT/US18/50091, filed Sep. 7, 2018 “Multi-Effector CRISPR Based Diagnostic Systems”, PCT/US18/66940 filed Dec. 20, 2018 entitled “CRISPR Effector System Based Multiplex Diagnostics”, PCT/US18/054472 filed Oct. 4, 2018 entitled “CRISPR Effector System Based Diagnostic”, U.S. Provisional 62/740,728 filed Oct. 3, 2018 entitled “CRISPR Effector System Based Diagnostics for Hemorrhagic Fever Detection”, U.S. Provisional 62/690,278 filed Jun. 26, 2018 and U.S. Provisional 62/767,059 filed Nov. 14, 2018 both entitled “CRISPR Double Nickase Based Amplification, Compositions, Systems and Methods”, U.S. Provisional 62/690,160 filed Jun. 26, 2018 and U.S. Pat. No. 62,767,077 filed Nov. 14, 2018, both entitled “CRISPR/CAS and Transposase Based Amplification Compositions, Systems, And Methods”, U.S. Provisional 62/690,257 filed Jun. 26, 2018 and 62/767,052 filed Nov. 14, 2018 both entitled “CRISPR Effector System Based Amplification Methods, Systems, And Diagnostics”, U.S. Provisional 62/767,076 filed Nov. 14, 2018 entitled “Multiplexing Highly Evolving Viral Variants With SHERLOCK” and 62/767,070 filed Nov. 14, 2018 entitled “Droplet SHERLOCK.” Reference is further made to WO2017/127807, WO2017/184786, WO 2017/184768, WO 2017/189308, WO 2018/035388, WO 2018/170333, WO 2018/191388, WO 2018/213708, WO 2019/005866, PCT/US18/67328 filed Dec. 21, 2018 entitled “Novel CRISPR Enzymes and Systems”, PCT/US18/67225 filed Dec. 21, 2018 entitled “Novel CRISPR Enzymes and Systems” and PCT/US18/67307 filed Dec. 21, 2018 entitled “Novel CRISPR Enzymes and Systems”, U.S. 62/712,809 filed Jul. 31, 2018 entitled “Novel CRISPR Enzymes and Systems”, U.S. 62/744,080 filed Oct. 10, 2018 entitled “Novel Cas12b Enzymes and Systems” and U.S. 62/751,196 filed Oct. 26, 2018 entitled “Novel Cas12b Enzymes and Systems”, U.S. Pat. No. 715,640 filed August 7, 2-18 entitled “Novel CRISPR Enzymes and Systems”, WO 2016/205711, U.S. Pat. No. 9,790,490, WO 2016/205749, WO 2016/205764, WO 2017/070605, WO 2017/106657, and WO 2016/149661, WO2018/035387, WO2018/194963, Cox D B T, et al., RNA editing with CRISPR-Cas13, Science. 2017 Nov. 24; 358(6366):1019-1027; Gootenberg J S, et al., Multiplexed and portable nucleic acid detection platform with Cas13, Cas12a, and Csm6, Science. 2018 Apr. 27; 360(6387):439-444; Gootenberg J S, et al., Nucleic acid detection with CRISPR-Cas13a/C2c2, Science. 2017 Apr. 28; 356(6336):438-442; Abudayyeh 00, et al., RNA targeting with CRISPR-Cas13, Nature. 2017 Oct. 12; 550(7675):280-284; Smargon A A, et al., Cas13b Is a Type VI-B CRISPR-Associated RNA-Guided RNase Differentially Regulated by Accessory Proteins Csx27 and Csx28. Mol Cell. 2017 Feb. 16; 65(4):618-630.e7; Abudayyeh 00, et al., C2c2 is a single-component programmable RNA-guided RNA-targeting CRISPR effector, Science. 2016 Aug. 5; 353(6299):aaf5573; Yang L, et al., Engineering and optimising deaminase fusions for genome editing. Nat Commun. 2016 Nov. 2; 7:13330, Myhrvold et al., Field deployable viral diagnostics using CRISPR-Cas13, Science 2018 360, 444-448, Shmakov et al. “Diversity and evolution of class 2 CRISPR-Cas systems,” Nat Rev Microbiol. 2017 15(3):169-182, each of which is incorporated herein by reference in its entirety.

All publications, published patent documents, and patent applications cited herein are hereby incorporated by reference to the same extent as though each individual publication, published patent document, or patent application was specifically and individually indicated as being incorporated by reference.

### II. Overview

The World Health Organization has developed an action plan for combating the emergence of resistance, which includes a call for the development of rapid molecular assays for predicting antimicrobial susceptibility, or resistance-guided therapy. Such assays facilitate susceptibility determination without culture, which can be laborious and time-intensive for Neisseria gonorrhoeae, and permit the use of antimicrobials previously thought ineffective, thus alleviating the selective pressure towards antibiotic resistance.

Specific high-sensitivity enzymatic reporter unlocking (SHERLOCK) technology is disclosed that utilizes Cas13a, a CRISPR enzyme, and isothermal amplification via recombinase polymerase amplification (RPA) for the rapid detection of antibiotic resistance at the point-of care. Cas13a-based detection works via complementary binding of programable CRISPR RNA (crRNA) sequences targeting antibiotic resistance mutations, which activates the inherent Cas13a-meated collateral cleavage of an RNA reporter. This cost-effective lateral flow strip assay is amenable to rapid multiplex testing of Neisseria gonorrhoeae isolates for resistance to multiple antibiotics, e.g. cefixime, ceftriaxone and ciproflaxin. The assays have the potential to make point-of-care resistance-guided therapies a reality even in a resource-limited setting.

### III. Neisseria Gonorrhoeae Pathogen

### Microbiology

Neisseria gonorrhoeae is a kidney bean-shaped Gram-negative diplococcus that lacks a polysaccharide capsule and is about 0.6 to 1.0 μm in diameter. Members of the genus Neisseria inhabit the mucous membranes of humans. The Neisseria genus currently consists of at least 23 species, of which about half are human-restricted species, some are animal-restricted and some can be isolated from mucosal surfaces in both humans and animals. N. gonorrhoeae is genomically, morphologically and phenotypically closely related to the other pathogenic Neisseria species, Neisseria meningitidis, which is typically carried as a commensal in the (naso) pharynx of 10-15% of the general population but occasionally causes fatal septicaemia and/or meningitis.

N. gonorrhoeae is oxidase positive, non-spore-forming, non-motile and an obligate aerobe. It has fastidious growth requirements, requiring CO₂ supplementation and enriched media, such as chocolate agar, with various antibiotics to grow in culture. The circular gonococcal genome is approximately 2219 kb in size, with a G+C content of 50%. The bacterium possesses more than one copy of the genome and is therefore polyploid. Most gonococci possess a 24.5 mega-Dalton conjugative plasmid that can facilitate the transfer of other, usually non-transferable, plasmids without mobilization of chromosomal genes. Genetic recombination facilitates antigenic variation of its pili and surface proteins that interact with the immune system. There are at least 70 different strains of N. gonorrhoeae, characterized by the absence or presence of pili, opacity of colonies, auxotyping (nutritional requirements), serotyping and genotyping.

### Antibiotic Resistance

Without an effective vaccine, antibiotics have been the only effective method for controlling gonorrhea, but the efficacy of antibiotics is now in question. The main molecular mechanisms that are used by bacteria to develop antimicrobial resistance are: protective alteration of antibiotic targets, decreased influx of antibiotics into the cell through transport proteins, increased efflux out of the cell via multi-drug efflux pumps, and expression of antibiotic degrading enzymes. Different strains of N. gonorrhoeae have evolved numerous resistance determinants using all these mechanisms to inhibit killing by all major classes of antibiotics. There has been substantial research into β-lactam resistance mechanisms of N. gonorrhoeae. Transpeptidase penicillin binding protein 2 (PBP2), encoded by the penA gene is a periplasmic transpeptidase and the main lethal target of cephalosporins; most resistant isolates contain mosaic mutations in penA. Pump MtrCDE and its repressor mtrR contribute to N. gonorrhoeae resistance through antimicrobial efflux. Variants of the major porin protein, encoded by porB, contribute to resistance to β-lactams, but resistance requires a concomitant mutation in mtrR.

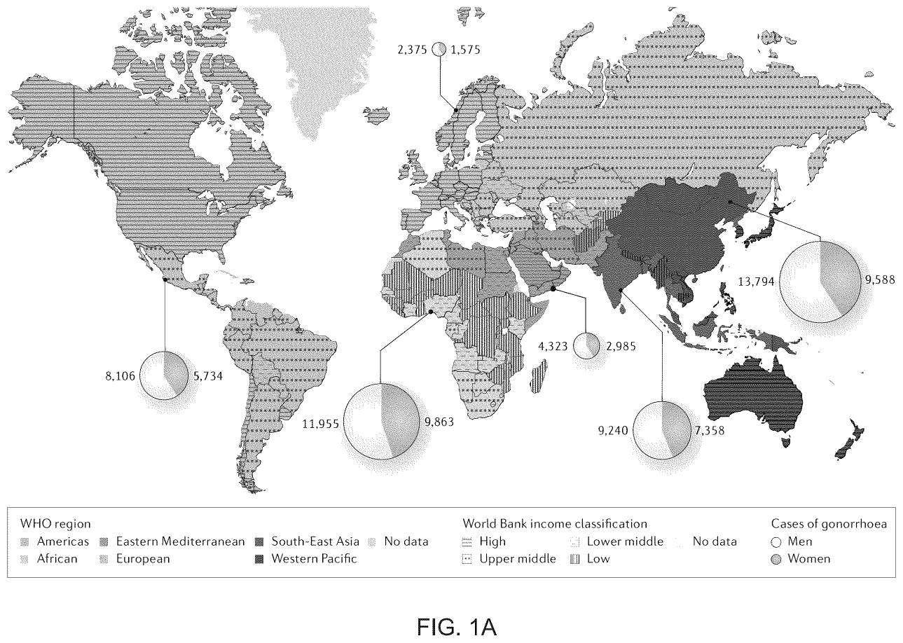

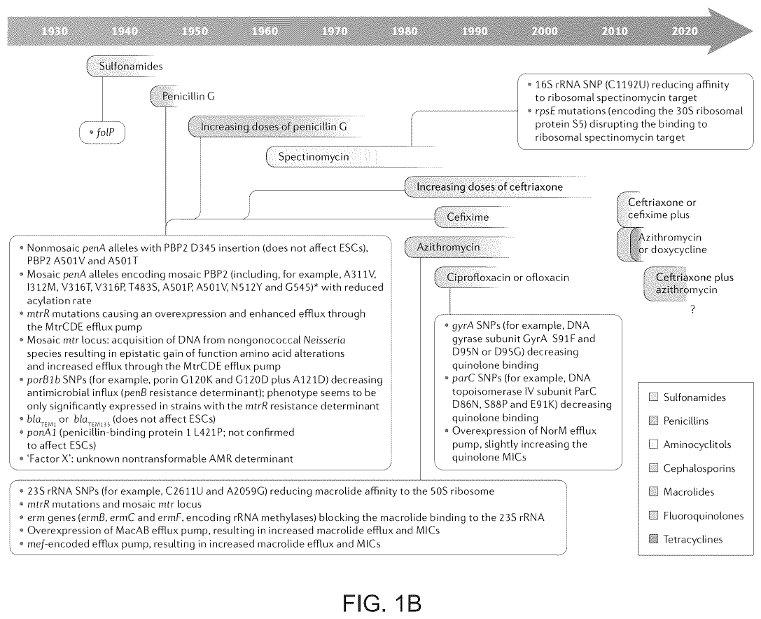

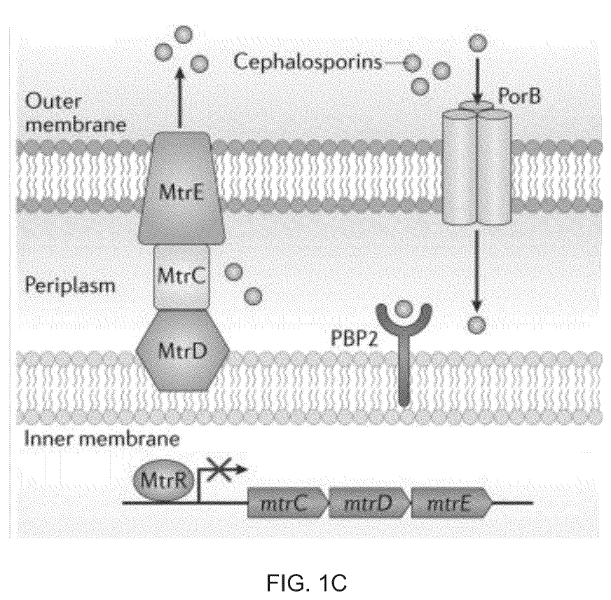

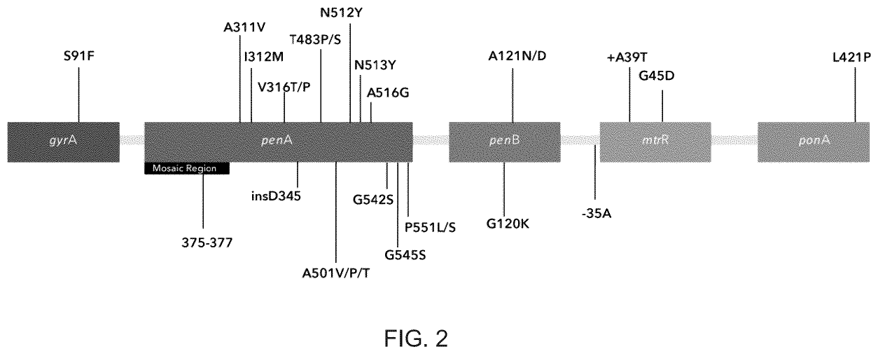

Domestic and international gonococcal surveillance efforts have demonstrated increased resistance worldwide to multiple classes of antibiotics, including β-lactam, polyketide, fluoroquinolone, cephalosporin, aminocyclitol, and macrolides. Penicillin is an exemplary antibiotic for β-lactam (see FIG. 1A). Tetracycline is an exemplary antibiotic for polyketide. Ciprofloxacin is an exemplary antibiotic for fluoroquinolone. Ceftriaxone (like cefixime) is an exemplary antibiotic for cephalosporin. Spectinomycin is an exemplary for aminocyclitol. Azithromycin is an exemplary for macrolide. An especially alarming trend in N. gonorrhoeae drug resistance has been the progressive decrease in susceptibility to the cephalosporins, and the identification of high-level ceftriaxone-resistance.

The first Neisseria gonorrhoeae strain with high-level resistance to ceftriaxone was isolated in 2009 in Japan, which was followed by some isolates with high-level ceftriaxone resistance in 2011 in France and Spain. During subsequent years, ceftriaxone-resistant isolates have been characterized in many countries including Japan, China, Australia, Singapore, Canada, Argentina and several European countries. Furthermore, treatment failures with ceftriaxone were verified in Japan, Australia and in several European countries. In 2014, the first failure of ceftriaxone-azithromycin dual therapy for gonorrhea was verified in the UK. Worryingly, since 2015, an international spread of one ceftriaxone-resistant gonococcal strain, initially described in Japan, has been confirmed, and the first strain with resistance to ceftriaxone plus high-level azithromycin resistance was isolated in 2018 in the UK and Australia.

In 2023, the first isolates of multidrug-resistant (MDR) N. gonorrhoeae with reduced susceptibility to ceftriaxone, cefixime, and azithromycin and resistance to ciprofloxacin, penicillin, and tetracycline were reported in the United States. The MDR N. gonorrhoeae strain was previously identified in the United Kingdom and the Asia-Pacific region. Although ceftriaxone was effective for treatment, the isolates were confirmed to have the presence of a mosaic penA60 allele conferring reduced ceftriaxone susceptibility.

The World Health Organization (WHO) has put forth action plans to combat the emergence of antimicrobial resistance, including calls for the development of molecular assays designed for detection of pathogens and genetic mutations that confer reduced susceptibility to specific antibiotics. Use of genetic markers to guide therapy, known as resistance-guided therapy, is a promising approach to stem the tide of antibiotic resistance.

### a) Fluoroquinolones

The prevalence of quinolone-resistant N. gonorrhoeae (QRNG) ranges from 10 to nearly 100 percent of isolates reported throughout the world, and these drugs are no longer recommended for the empiric treatment of gonorrhea. In several regions, such as the United States and Europe, the prevalence of quinolone resistance has decreased somewhat compared with previous years, likely reflecting decreased use of the drug. However, resistance levels remain high elsewhere despite the avoidance of these drugs for treatment of gonorrhea and the resultant decrease in selection pressure. In 2021, from GISP data, 32.8 percent of isolates in the United States had a ciprofloxacin MIC≥1.0 mcg/mL (indicating resistance), which is increased from 13.3 percent in 2011 but decreased from 35.4 percent in 2019. In vitro and animal studies suggest that certain quinolone resistance mutations also confer a fitness advantage, which could explain the persistence of resistance in the absence of selection pressure (reviewed in Allan-Blitz et al., Resistance-guided therapy for Neisseria gonorrhoeae. Clin. Infect. Dis. 75, 1655-1660 (2022)).

i. Ciprofloxacin

Ciprofloxacin (Compound I; PubChem CID: 2764) is a broad-spectrum antibiotic of the fluoroquinolone class.

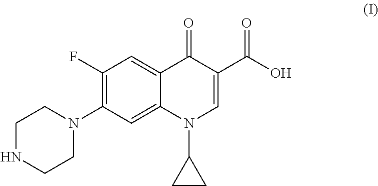 [SMILES: O=C(O)c1cn(C2CC2)c2cc(N3CCNCC3)c(F)cc2c1=O]

It is active against some Gram-positive and many Gram-negative bacteria, including Neisseria gonorrhoeae. Ciprofloxacin inhibits topoisomerase II (or DNA-gyrAse) and topoisomerase IV that are necessary to separate bacterial DNA at cell division. Consequently, inhibition of the topoisomerases results in bacterial DNA fragmentation.

gyrA Gene

Various specific allelic mutations within the 2 genes that encode DNA-gyrAse and topoisomerase IV, gyrAse subunit A (gyrA) and parC, respectively, have been associated with ciprofloxacin resistance among N. gonorrhoeae. Notably, despite the association of various mutations with phenotypic ciprofloxacin resistance, it is the absence of a single mutation at the serine 91 codon of the gyrA gene (see TABLE I below; FIG. 1) that has been shown to be both necessary and sufficient to predict susceptibility (Tanaka et al., Development of fluoroquinolone resistance and mutations involving gyrA and ParC proteins among Neisseria gonorrhoeae isolates in Japan. J Urol. 1998 June; 159 (6): 2215-9). In one embodiment, substitutions in gyrA position 95 are also associated with resistance to ciprofloxacin (mutation from gyrA D95 to G or N) with intermediate ciprofloxacin MICs (0.125-0.5 μg/mL). These mutations can lead to treatment failure, despite reversion of gyrA position 91 from phenylalanine to serine (Rubin et al., Neisseria gonorrhoeae diagnostic escape from a gyrA-based test for ciprofloxacin susceptibility and the effect on zoliflodacin resistance: a bacterial genetics and experimental evolution study. Lancet Microbe 4, e247-e254 (2023)).

TABLE I
DNA GYRASE SUBUNIT A [NEISSERIA GONORRHOEAE]
Accession WP_003691306 (amino acid sequence) (SEQ ID NO: 1)
GenBank: U08817.1 (nucleotide sequence) (SEQ ID NO: 2)
Belland, R. J., Morrison, S. G., Ison, C. and Huang, W. M., Neisseria
gonorrhoeae acquires mutations in analogous regions of gyrA and parC in
fluoroquinolone-resistant isolates. Mol. Microbiol. 14 (2), 371-380 (1994)

| | | |
|---|---|---|
| 1 | M T D A T I R H D H K F A L E T L P V S | 20 |
| 1 | ATGACCGACGCAACCATCCGCCACGACCACAAATTCGCCCTCGAAACCCTGCCCGTCAGC | 60 |
| 21 | L E D E M R K S Y L D Y A M S V I V G R | 40 |
| 61 | CTTGAAGACGAAATGCGCAAAAGCTATCTCGACTACGCCATGAGCGTCATTGTCGGGCGC | 120 |
| 41 | A L P D V R D G L K P V H R R V L Y A M | 60 |
| 121 | GCGCTGCCGGACGTTCGCGACGGCCTAAAGCCGGTGCACCGGCGCGTACTGTACGCGATG | 180 |
| 61 | H E L K N N W N A A Y K K S A R I V G D | 80 |
| 181 | CACGAGCTGAAAAATAACTGGAATGCCGCCTACAAAAAATCGGCGCGCATCGTCGGCGAC | 240 |
| 81 | V I G K Y H P H G D A V Y D T I V R M | 100 |
| 241 | GTCATCGGTAAATACCACCCCCACGGCGAT GCAGTTTACGACACCATCGTCCGTATG | 300 (TTC) |
| 101 | A Q N F A M R Y V L I D G Q G N F G S V | 120 |
| 301 | GCGCAAAATTTCGCTATGCGTTATGTGCTGATAGACGGACAGGGCAACTTCGGATCGGTG | 360 |
| 121 | D G L A A A A M R Y T E I R M A K I S H | 140 |
| 361 | GACGGGCTTGCCGCCGCAGCCATGCGCTATACCGAAATCCGCATGGCGAAAATCTCACAT | 420 |
| 141 | E M L A D I E E E T V N F G P N Y D G S | 160 |
| 421 | GAAATGCTGGCAGACATTGAGGAAGAAACCGTTAATTTCGGCCCGAACTACGACGGTAGC | 480 |
| 161 | E H E P L V L P T R F P T L L V N G S S | 180 |
| 481 | GAACACGAGCCGCTTGTACTGCCGACCCGTTTCCCCACACTGCTCGTCAACGGCTCGTCC | 540 |
| 181 | G I A V G M A T N I P P H N L T D T I N | 200 |
| 541 | GGTATCGCCGTCGGTATGGCGACCAACATCCCGCCGCACAACCTCACCGACACCATCAAC | 600 |
| 201 | A C L R L L D E P K T E I D E L I D I I | 220 |
| 601 | GCCTGTCTGCGTCTTTTGGACGAACCCAAAACCGAAATCGACGAACTGATCGACATTATC | 660 |
| 221 | Q A P D F P T G A T I Y G L G G V R E G | 240 |
| 661 | CAAGCCCCCGACTTCCCGACCGGGGCAACCATCTACGGCTTGGGCGGCGTGCGCGAAGGC | 720 |
| 241 | Y K T G R G R V V M R G K T H I E P I G | 260 |
| 721 | TATAAAACAGGCCGCGGCCGCGTTGTTATGCGCGGTAAGACCCATATCGAACCCATAGGC | 780 |
| 261 | K N G E R E R I V I D E I P Y Q V N K A | 280 |
| 781 | AAAAACGGCGAACGCGAACGCATCGTTATCGACGAAATCCCCTATCAGGTCAACAAAGCC | 840 |
| 281 | K L V E K I G D L V R E K T L E G I S E | 300 |
| 841 | AAGTTGGTCGAGAAAATCGGCGATTTGGTTCGGGAAAAAACACTGGAAGGCATTTCCGAG | 900 |
| 301 | L R D E S D K S G M R V V I E L K R N E | 320 |
| 901 | CTCCGCGACGAATCCGACAAATCCGGTATGCGCGTCGTTATCGAGCTGAAACGCAACGAA | 960 |
| 321 | N A E V V L N Q L Y K L T P L Q D S F G | 340 |
| 961 | AATGCCGAAGTCGTCTTAAACCAACTCTACAAACTGACTCCGCTGCAAGACAGTTTCGGC | 1020 |
| 341 | I N M V V L V D G Q P R L L N L K Q I L | 360 |
| 1021 | ATCAATATGGTGGTTTTGGTCGACGGACAACCGCGCCTGTTAAACCTGAAACAGATTCTC | 1080 |
| 361 | S E F L R H R R E V V T R R T L F R L K | 380 |
| 1081 | TCCGAATTCCTGCGCCACCGCCGCGAAGTCGTTACCCGACGTACGCTTTTCCGGCTGAAG | 1140 |
| 381 | K A R H E G H I A E R K A V A L S N I D | 400 |
| 1141 | AAGGCACGCCATGAAGGGCATATCGCCGAACGGAAAGCCGTCGCACTGTCCAATATCGAT | 1200 |
| 401 | E I I K L I K E S P N A A E A K E K L L | 420 |
| 1201 | GAAATCATCAAGCTCATCAAAGAATCGCCCAACGCGGCCGAGGCCAAAGAAAAACTGCTT | 1260 |
| 421 | A R P W A S S L V E E M L T R S G L D L | 440 |
| 1261 | GCGCGCCCTTGGGCCAGCAGCCTCGTTGAAGAAATGCTGACGCGTTCCGGTCTGGATTTG | 1320 |
| 441 | E M M R P E G L V A N I G L K K Q G Y Y | 460 |
| 1321 | GAAATGATGCGTCCGGAAGGATTGGTCGCAAACATTGGTCTGAAAAAACAAGGTTATTAC | 1380 |
| 461 | L S E I Q A D A I L R M S L R N L T G L | 480 |
| 1381 | CTGAGCGAGATTCAGGCAGATGCTATTTTACGCATGAGCCTGCGAAACCTGACCGGCCTC | 1440 |
| 481 | D Q K E I I E S Y K N L M G K I I D F V | 500 |
| 1441 | GATCAGAAAGAAATTATCGAAAGCTACAAAAACCTGATGGGTAAAATCATCGACTTTGTG | 1500 |
| 501 | D I L S K P E R I T Q I I R D E L E E I | 520 |
| 1501 | GATATCCTCTCCAAACCCGAACGCATTACCCAAATCATCCGTGACGAACTGGAAGAAATC | 1560 |
| 521 | K T N Y G D E R R S E I N P F G G D I A | 540 |
| 1561 | AAAACCAACTATGGCGACGAACGCCGCAGCGAAATCAACCCGTTCGGCGGCGACATTGCC | 1621 |
| 541 | D E D L I P Q R E M V V T L T H G G Y I | 560 |
| 1621 | GATGAAGACCTGATTCCGCAACGCGAAATGGTCGTGACCCTGACCCACGGCGGCTATATA | 1680 |
| 561 | K T Q P T T D Y Q A Q R R G G R G K Q A | 580 |
| 1681 | AAAACCCAGCCGACCACCGACTATCAGGCTCAGCGTCGCGGCGGGCGCGGCAAACAGGCG | 1740 |
| 581 | A A T K D E D F I E T L F V A N T H D Y | 600 |
| 1741 | GCTGCCACCAAAGACGAAGACTTTATCGAAACCCTGTTTGTTGCCAACACGCATGACTAT | 1800 |
| 601 | L M C F T N L G K C H W I K V Y K L P E | 620 |
| 1801 | TTGATGTGTTTTACCAACCTCGGCAAGTGCCACTGGATTAAGGTTTACAAACTGCCCGAA | 1860 |
| 621 | G G R N S R G R P I N N V I Q L E E G E | 640 |
| 1861 | GGCGGACGCAACAGCCGCGGCCGTCCGATTAACAACGTCATCCAGCTGGAAGAAGGCGAA | 1920 |
| 641 | K V S A I L A V R E F P E D Q Y V F F A | 660 |
| 1921 | AAAGTCAGCGCGATTCTGGCAGTACGCGAGTTTCCCGAAGACCAATACGTCTTCTTCGCC | 1980 |
| 661 | T A Q G M V K K V Q L S A F K N V R A Q | 680 |
| 1981 | ACCGCGCAGGGAATGGTGAAAAAAGTCCAACTTTCCGCCTTTAAAAACGTCCGCGCCCAA | 2040 |
| 681 | G I K A I A L K E G D Y L V G A A Q T G | 700 |
| 2041 | GGCATTAAAGCCATCGCACTCAAAGAAGGCGACTACCTCGTCGGCGCTGCGCAAACAGGC | 2100 |
| 701 | G A D D I M L F S N L G K A I R E N E Y | 720 |
| 2101 | GGTGCGGACGACATTATGTTGTTCTCCAACTTGGGCAAAGCCATCCGCTTCAACGAATAC | 2160 |
| 721 | W E K S G N D E A E D A D I E T E I S D | 740 |
| 2161 | TGGGAAAAATCCGGCAACGACGAAGCGGAAGATGCCGACATCGAAACCGAGATTTCAGAC | 2220 |
| 741 | D L E D E T A D N E N T L P S G K N G V | 760 |
| 2221 | GACCTCGAAGACGAAACCGCCGACAACGAAAACACCCTGCCAAGCGGCAAAAACGGCGTG | 2280 |
| 761 | R P S G R G S G G L R G M R L P A D G K | 780 |
| 2281 | CGTCCGTCCGGTCGCGGCAGCGGCGGTTTGCGCGGTATGCGCCTGCCTGCCGACGGCAAA | 2340 |
| 781 | I V S L I T F A P E T E E S G L Q V L T | 800 |
| 2341 | ATCGTCAGCCTGATTACCTTCGCCCCTGAAACCGAAGAAAGCGGTTTGCAAGTTTTAACC | 2400 |
| 801 | A T A N G Y G K R T P I A D Y S R K N K | 820 |
| 2401 | GCCACCGCCAACGGATACGGAAAACGCACCCCGATTGCCGATTACAGCCGCAAAAACAAA | 2460 |
| 821 | G G Q G S I A I N T G E R N G D L V A A | 840 |
| 2461 | GGCGGGCAAGGCAGTATTGCCATTAACACCGGCGAGCGCAACGGCGATTTGGTCGCCGCA | 2520 |
| 841 | T L V G E T D D L M L I T S G G V L I R | 860 |
| 2521 | ACCTTGGTCGGCGAAACCGACGATTTGATGCTGATTACCAGCGGCGGCGTGCTTATCCGT | 2580 |
| 861 | T K V E Q I R E T G R A A A G V K L I N | 880 |
| 2581 | ACCAAAGTCGAACAAATCCGCGAAACCGGCCGCGCCGCAGCAGGCGTGAAACTGATTAAC | 2640 |
| 881 | L D E G E T L V S L E R V A E D E S E L | 900 |
| 2641 | TTGGACGAAGGCGAAACCTTGGTATCGCTGGAACGTGTTGCCGAAGACGAATCCGAACTC | 2700 |
| 901 | S G A S V I S N V T E P E A E N * | 917 |
| 2701 | TCCGGCGCTTCTGTAATTTCCAATGTAACCGAACCGGAAGCCGAGAACTGA | 2751 |

### b) Cephalosporins

The 4 principal genes involved in cephalosporin resistance are penA, penB, mtrR, and ponA (see FIG. 1) penA and ponA encode penicillin-binding protein 2 and penicillin-binding protein respectively that are required for bacterial cell wall synthesis. penB encodes PorB, an outer membrane porin. mtrR encodes a transcriptional repressor that binds to and represses an adjacent, but divergent, promoter used for transcription of an efflux pump operon (mtrCDE) encoding a tripartite export system that expels antimicrobials from the bacterial periplasmic space (reviewed in Allan-Blitz et al., Resistance-guided therapy for Neisseria gonorrhoeae. Clin. Infect. Dis. 75, 1655-1660 (2022)).

i. Cefixime

Cefixime (Compound II; PubChem CID: 5362065) is a third generation cephalosporin. By binding to the penicillin-binding protein 2, cefixime inhibits the final transpeptidation step of peptidoglycan synthesis thereby arresting cell wall assembly which results in bacterial cell death.

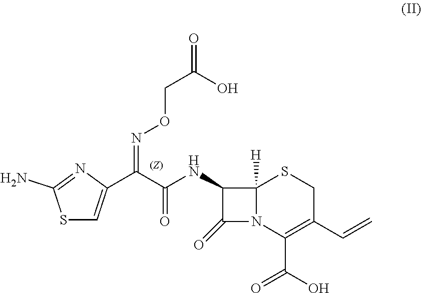 [SMILES: C=CC1=C(C(=O)O)N2C(=O)[C@@H](NC(=O)/C(=N\OCC(=O)O)c3csc(N)n3)[C@H]2SC1]

penA Gene

penA encodes penicillin-binding protein 2 (PBP2), a member of a family of proteins that are required for bacterial peptidoglycan synthesis. PBPs catalyze the polymerization of the glycan strand (transglycosylation) and the cross-linking between glycan chains (transpeptidation). Notably, there are 2 regions within the penA gene: a mosaic region that is composed of inserted DNA sequences from other commensal Neisseria subspecies, and a nonmosaic region specific to N. gonorrhoeae. Mutations within each region have been shown to be associated with a decreased susceptibility to cephalosporins. Further, there are different mosaic strains of N. gonorrhoeae, with penA34 being the most common form in North America. The penA34 mosaic insertion within the penA gene has been repeatedly associated with cefixime resistance in N. gonorrhoeae; that sequence was present in 98% of 270 isolates with reduced cefixime susceptibility in a US study.

Non-penA34 alleles are more common in Europe and Asia. Further, nonmosaic penA mutations are also associated with cephalosporin resistance. Non-penA mutations, such as those in penB (G120K and A121N/D), mtrR (35A deletion in the promoter region, +A39T, and G45D), and ponA (L412P), likely contribute to the degree of cefixime resistance but are neither necessary nor sufficient to reduce susceptibility independent of penA. Specific loci of mutations within the penA region that appear to be frequently associated with mosaic penA patterns include 1312M, V316T, N512Y, and G545S. Importantly, studies using gene transformation techniques demonstrated that those mutations are not sufficient alone to confer reduced susceptibility to cefixime. However, reversion back to the wild type in a strain with cefixime resistance resulted in reduction of the minimum inhibitory concentration (MIC) to levels comparable with those of wild-type penA strains. Thus, other mutations are likely important in addition to those found in the mosaic penA region.

Wild-type penA can be distinguished from other mosaic forms of penA (with the exception of penA49) by determining the amino acid sequence of region 375-377. Nonmosaic penA mutations that appear to be critical to conferring reduced susceptibility to cefixime include point mutations within A501V/P/T, G542S, and P551L/S (see TABLE II below). Deng et al proposed that susceptibility to cefixime could be reliably predicted by detecting the absence of mosaic substitutions within the penA gene amino acid positions 375-377 and the absence of 3 critical mutations in the nonmosaic region of the penA gene: 501, 542, and 551. The authors conclude that an assay that detects any of the above resistance mutations would have a 99.5% (95% CI, 98.3%-99.9%) sensitivity for predicting reduced susceptibility to cefixime (Deng et al., Using the genetic characteristics of Neisseria gonorrhoeae strains with decreased susceptibility to cefixime to develop a molecular assay to predict cefixime susceptibility. Sex Health 2019; 16:488-99). Subsequent work applied an analysis of the same mutations to an external dataset of N. gonorrhoeae strains from the United States and found a 95.9% (95% CI, 97.1%-99.4%) sensitivity for the determination of decreased cefixime susceptibility (Deng et al., Six penA codons accurately and reliably predict cefixime decreased susceptibility in Neisseria gonorrhoeae. J Infect Dis 2020; 221:851-2). Importantly, the reported 95.9% sensitivity equated to a failure of capturing reduced susceptibility to cefixime among 8 strains. Those 8 strains did not contain the expected mosaic penA mutations (reviewed in Allan-Blitz et al., Resistance-guided therapy for Neisseria gonorrhoeae. Clin. Infect. Dis. 75, 1655-1660 (2022)).

TABLE II
PENICILLIN BINDING PROTEIN 2 (penA) [NEISSERIA GONORRHOEAE]
Accession No: P08149 (amino acid sequence) (SEQ ID NO: 3)
GenBank: X07468.1 (nucleotide sequence) (SEQ ID NO: 4)
Spratt, B.G. Hybrid penicillin-binding proteins in penicillin-resistant strains of
Neisseria gonorrhoeae Nature 332 (6160), 173-176 (1988)

| | | |
|---|---|---|
| 1 | M L I K S E Y K P R M L P K E E Q V K K | 20 |
| 103 | ATGTTGATTAAAAGCGAATATAAGCCCCGGATGCTGCCCAAAGAAGAGCAGGTCAAAAAG | 162 |
| 21 | P M T S N G R I S F V L M M A V L F A | 40 |
| 163 | CCGATGACCAGTAACGGACGGATTAGCTTCGTCCTGATG ATGGCGGTCTTGTTTGCC | 222 |
| 41 | C L I A R G L Y L Q T V T Y N F L K E Q | 60 |
| 223 | TGTCTGATTGCCCGCGGGCTGTATCTGCAGACGGTAACGTATAACTTTTTGAAAGAACAG | 282 |
| 61 | G D N R I V R T Q A L P A T R G T V S D | 80 |
| 283 | GGCGACAACCGGATTGTGCGGACTCAAGCATTGCCGGCTACACGCGGTACGGTTTCGGAC | 342 |
| 81 | R N G A V L A L S A P T E S L F A V P K | 100 |
| 343 | CGGAACGGTGCGGTTTTGGCGTTGAGCGCGCCGACGGAGTCCCTGTTTGCCGTGCCTAAA | 402 |
| 101 | D M K E M P S A A Q L E R L S E L V D V | 120 |
| 403 | GATATGAAGGAAATGCCGTCTGCCGCCCAATTGGAACGCCTGTCCGAGCTTGTCGATGTG | 462 |
| 121 | P V D V L R N K L E Q K G K S F I W I K | 140 |
| 463 | CCGGTCGATGTTTTGAGGAACAAACTCGAACAGAAAGGCAAGTCGTTTATTTGGATCAAG | 522 |
| 141 | R Q L D P K V A E E V K A L G L E N F V | 160 |
| 523 | CGGCAGCTCGATCCCAAGGTTGCCGAAGAGGTCAAAGCCTTGGGTTTGGAAAACTTTGTA | 582 |
| 161 | F E K E L K R H Y P M G N L F A H V I G | 180 |
| 583 | TTTGAAAAAGAATTAAAACGCCATTACCCGATGGGCAACCTGTTTGCACACGTCATCGGA | 642 |
| 181 | F T D I D G K G Q E G L E L S L E D S L | 200 |
| 643 | TTTACCGATATTGACGGCAAAGGTCAGGAAGGTTTGGAACTTTCGCTTGAAGACAGCCTG | 702 |
| 201 | Y G E D G A E V V L R D R Q G N I V D S | 220 |
| 703 | TATGGCGAAGACGGCGCGGAAGTTGTTTTGCGGGACCGGCAGGGCAATATTGTGGACAGC | 762 |
| 221 | L D S P R N K A P Q N G K D I I L S L D | 240 |
| 763 | TTGGACTCCCCGCGCAATAAAGCACCGCAAAACGGCAAAGACATCATCCTTTCCCTCGAT | 822 |
| 241 | Q R I Q T L A Y E E L N K A V E Y H Q A | 260 |
| 823 | CAGAGGATTCAGACCTTGGCCTATGAAGAGTTGAACAAGGCGGTCGAATACCATCAGGCA | 882 |
| 261 | K A G T V V V L D A R T G E I L A L A N | 280 |
| 883 | AAAGCCGGAACGGTGGTGGTTTTGGATGCCCGCACGGGGGAAATCCTCGCCTTGGCCAAT | 942 |
| 281 | T P A Y D P N R P G R A D S E Q R R N R | 300 |
| 943 | ACGCCCGCCTACGATCCCAACAGACCCGGCCGGGCAGACAGCGAACAGCGGCGCAACCGT | 1002 |
| 301 | A V T D M I E P G S K P F I A K A | 320 |
| 1003 | GCCGTAACCGATATGATCGAACCTGGTTCG AAACCGTTC ATTGCGAAGGCA | 1062 |
| 321 | L D A G K T D L N E R L N T Q P Y K I G | 340 |
| 1063 | TTGGATGCGGGCAAAACCGATTTGAACGAACGGCTGAATACGCAGCCTTATAAAATCGGA | 1122 |
| 341 | P S P V R D T H V Y P S L D V R G I M Q | 360 |
| 1123 | CCGTCTCCCGTGCGCGATACCCATGTTTACCCCTCTTTGGATGTGCGCGGCATTATGCAG | 1182 |
| 361 | K S S N V G T S K L S A R F E M Y | 380 |
| 1183 | AAATCGTCCAACGTCGGCACAAGCAAACTGTCTGCGCGTTTC GAAATGTAT | 1242 |
| 381 | D F Y H E L G I G V R M H S G F P G E T | 400 |
| 1243 | GACTTCTATCATGAATTGGGCATCGGTGTGCGTATGCACTCGGGCTTTCCGGGGGAAACT | 1302 |
| 401 | A G L L R N W R R W R P I E Q A T M S F | 420 |
| 1303 | GCAGGTTTGTTGAGAAATTGGCGCAGGTGGCGGCCCATCGAACAGGCGACGATGTCTTTC | 1362 |
| 421 | G Y G L Q L S L L Q L A R A Y T A L T H | 440 |
| 1363 | GGTTACGGTCTGCAATTGAGCCTGCTGCAATTGGCGCGCGCCTATACCGCACTGACGCAC | 1422 |
| 441 | D G V L L P L S F E K Q A V A P Q G K R | 460 |
| 1423 | GACGGCGTTTTGCTGCCGCTCAGCTTTGAGAAGCAGGCGGTTGCGCCGCAAGGCAAACGC | 1482 |
| 461 | I F K E S T A R E V R N L M V S V T E P | 480 |
| 1483 | ATATTCAAAGAATCGACCGCGCGCGAGGTACGCAATCTGATGGTTTCCGTAACCGAGCCG | 1542 |
| 481 | G G T G T A G A V D G F D V G A K T G T | 500 |
| 1543 | GGCGGCACCGGTACGGCGGGTGCGGTGGACGGTTTCGATGTCGGCGCTAAAACCGGCACG | 1602 |
| 501 | R K F V N G R Y A D K H V T F I G | 520 |
| 1603 | CGCAAGTTCGTCAACGGGCGTTATGCCGAC AAACACGTC ACCTTTATCGGT | 1662 |
| 521 | F A P A K N P R V I V A V T I D E P T A | 540 |
| 1663 | TTTGCCCCCGCCAAAAACCCCCGTGTGATTGTGGCGGTAACCATCGACGAACCGACTGCC | 1722 |
| 541 | H Y Y G V V A G P F K K I M G G S | 560 |
| 1723 | CAC TATTAC GGCGTAGTGGCAGGG CCCTTCAAAAAAATTATGGGCGGCAGC | 1782 |
| 561 | L N I L G I S P T K P L T A A A V K T P | 580 |
| 1783 | CTGAACATCTTGGGCATTTCCCCGACCAAGCCACTGACCGCCGCAGCCGTCAAAACACCG | 1842 |
| 581 | S * | 582 |
| 1843 | TCTTAA | 1848 |

### PorB Gene

penB encodes PorB, an outer membrane porin. Mutations at residues G120 and A121 decrease the permeability of antibiotics, including cephalosporins.

TABLE III
NEISSERIA GONORRHOEAE PORIN PROTEIN GENE (porB)
Accession AAA25500.1 (amino acid sequence) (SEQ ID NO: 5)
GenBank: J03017 (nucleotide sequence) (SEQ ID NO: 6)
Gotschlich, E. C., Seiff, M. E., Blake, M. S. and Koomey, M., Porin protein of
Neisseria gonorrhoeae: cloning and gene structure Proc. Natl. Acad. Sci. U.S.A.
84 (22), 8135-8139 (1987)

| | | |
|---|---|---|
| 1 | M K K S L I A L T L A A L P V A A M A D | 20 |
| 1 | ATGAAAAAATCCCTGATTGCCCTGACTTTGGCAGCCCTTCCTGTTGCGGCAATGGCCGAT | 60 |
| 21 | V T L Y G A I K A G V Q T Y R S V E H T | 40 |
| 61 | GTCACCCTGTACGGTGCCATCAAAGCCGGCGTACAAACTTACCGTTCTGTAGAACATACA | 120 |
| 41 | D G K V S K V E T G S E I A D F G S K I | 60 |
| 121 | GACGGCAAGGTAAGTAAAGTGGAAACCGGCAGCGAAATCGCCGACTTCGGTTCAAAAATC | 180 |
| 61 | G F K G Q E D L G N G L K A V W Q L E Q | 80 |
| 181 | GGCTTCAAAGGCCAAGAAGACCTCGGCAACGGCCTGAAGGCCGTTTGGCAGTTGGAACAA | 240 |
| 81 | G A S V A G T N T G W G N K Q S F V G H | 100 |
| 241 | GGTGCCTCCGTCGCCGGCACTAACACCGGCTGGGGCAACAAACAATCCTTCGTCGGCTTG | 300 |
| 101 | K G G F G T I R A G S L N S P L K N T | 120 |
| 301 | AAGGGCGGCTTCGGTACCATCCGCGCCGGTAGCCTGAACAGCCCCCTGAAAAACACC | 360 |
| 121 | N V N A W E S G K F T G N V L E I S G | 140 |
| 361 | AACGTCAATGCTTGGGAATCCGGCAAATTTACCGGCAATGTGCTGGAAATCAGCGGA | 420 |
| 141 | M A Q R E H R Y L S V R Y D S P E F A G | 160 |
| 421 | ATGGCCCAACGGGAACACCGCTACCTGTCCGTACGCTACGATTCTCCCGAATTTGCCGGC | 480 |
| 161 | F S G S V Q Y A P K D N S G S N G E S Y | 180 |
| 481 | TTCAGCGGCAGCGTACAATACGCACCTAAAGACAATTCAGGCTCAAACGGCGAATCTTAC | 540 |
| 181 | H V G L N Y Q N S G F F A Q Y A G L F Q | 200 |
| 541 | CACGTTGGCTTGAACTACCAAAACAGCGGCTTCTTCGCACAATACGCCGGCTTGTTCCAA | 600 |
| 201 | R Y G E G T K K I E Y D D Q T Y S I P S | 220 |
| 601 | AGATACGGCGAAGGCACTAAAAAAATCGAATACGATGATCAAACTTATAGTATCCCCAGT | 660 |
| 221 | L F V E K L Q V H R L V G G Y D N N A L | 240 |
| 661 | CTGTTTGTTGAAAAACTGCAAGTTCACCGTTTGGTAGGCGGTTACGACAATAATGCCCTG | 720 |
| 241 | Y V S V A A Q Q Q D A K L Y G A M S G N | 260 |
| 721 | TACGTTTCCGTAGCCGCACAACAACAAGATGCCAAATTGTATGGAGCAATGAGCGGTAAT | 780 |
| 261 | S H N S Q T E V A A T A A Y R E G N V T | 280 |
| 781 | TCGCACAACTCTCAAACCGAAGTTGCCGCTACCGCGGCATACCGTTTCGGCAATGTAACG | 840 |
| 281 | P R V S Y A H G F K G T V D S A N H D N | 300 |
| 841 | CCCCGCGTTTCTTACGCCCACGGCTTCAAAGGCACTGTTGATAGTGCAAACCACGACAAT | 900 |
| 301 | T Y D Q V V V G A E Y D F S K R T S A L | 320 |
| 901 | ACTTATGACCAAGTGGTTGTCGGTGCGGAATACGACTTCTCCAAACGCACTTCTGCCTTG | 960 |
| 321 | V S A G W L Q G G K G A D K I V S T A S | 340 |
| 961 | GTTTCTGCCGGCTGGTTGCAAGGAGGCAAAGGCGCAGACAAAATCGTATCGACTGCCAGC | 1020 |
| 341 | A V V L R H F K F * | 349 |
| 1021 | GCCGTCGTTCTGCGCCACAAATTCTAA | 1047 |

### PonA Gene

The ponA gene, which encodes penicillin-binding protein 1, has been associated to a lesser extent with resistance to cephalosporins.

TABLE IV
NEISSERIA GONORRHOEAE PENICILLIN BINDING PROTEIN 1 (ponA)
Accession No.: AAB52536.1 (amino acid sequence) (SEQ ID NO: 7)
GenBank: U72876 (nucleotide sequence) (SEQ ID NO: 8)
Ropp, P. A. and Nicholas, R. A., Cloning and characterization of the ponA gene
encoding penicillin-binding protein 1 from Neisseria gonorrhoeae and Neisseria
meningitidis. J. Bacteriol. 179 (8), 2783-2787 (1997)

| | | |
|---|---|---|
| 1 | M I K K I L T T C F G L F F G F C V F G | 20 |
| 21 | ATGATTAAAAAGATTTTAACTACTTGTTTTGGTTTGTTTTTTGGTTTTTGTGTATTTGGA | 60 |
| 61 | V G L V A I A I L V T Y P K L P S L D S | 40 |
| 41 | GTGGGTCTGGTTGCCATTGCTATTTTGGTAACGTATCCGAAACTGCCGTCTTTGGATTCT | 120 |
| 121 | L Q H Y Q P K M P L T I Y S A D G E V I | 60 |
| 61 | TTGCAGCATTACCAGCCTAAAATGCCGTTGACTATTTATTCGGCGGATGGAGAAGTCATC | 180 |
| 181 | G M Y G E Q R R E F T K I G D F P E V L | 80 |
| 81 | GGTATGTATGGGGAGCAGCGGCGCGAATTTACAAAAATCGGCGATTTCCCCGAGGTGTTG | 240 |
| 241 | R N A V I A A E D K R F Y R H W G V D V | 100 |
| 101 | CGGAATGCGGTTATTGCCGCCGAGGATAAACGCTTTTACCGGCATTGGGGGGTGGATGTT | 300 |
| 301 | W G V A R A A V G N V V S G S V Q S G A | 120 |
| 121 | TGGGGTGTTGCCCGCGCTGCCGTCGGCAATGTCGTGTCCGGCAGCGTGCAGTCGGGTGCG | 360 |
| 361 | S T I T Q Q V A K N F Y L S S E K T F T | 140 |
| 141 | AGTACGATTACACAGCAGGTGGCGAAAAATTTTTATTTGAGCAGTGAAAAAACGTTCACA | 420 |
| 421 | R K F N E V L L A Y K I E Q S L S K D K | 160 |
| 161 | CGCAAATTCAATGAGGTGTTGCTTGCCTATAAAATCGAGCAGTCTTTAAGCAAAGACAAA | 480 |
| 481 | I L E L Y F N Q I Y L G Q R A Y G F A S | 180 |
| 181 | ATCCTTGAGTTGTATTTCAATCAGATTTACCTCGGTCAGCGCGCCTATGGTTTTGCATCT | 540 |
| 541 | A A Q I Y F N K N V R D L T L A E A A M | 200 |
| 201 | GCCGCGCAAATCTATTTCAATAAGAATGTCCGAGATTTGACTTTGGCGGAAGCCGCCATG | 600 |
| 601 | L A G L P K A P S A Y N P I V N P E R A | 220 |
| 221 | CTTGCGGGACTGCCCAAGGCTCCGTCTGCCTATAATCCGATTGTTAATCCGGAGCGTGCC | 660 |
| 661 | K L R Q K Y I L N N M L E E K M I T V Q | 240 |
| 241 | AAGTTGCGCCAGAAGTATATTTTGAACAATATGCTCGAGGAGAAGATGATTACCGTGCAA | 720 |
| 721 | Q R D Q A L N E E L H Y E R F V R K I D | 260 |
| 261 | CAGCGCGATCAGGCATTGAATGAGGAACTGCATTATGAGCGGTTTGTTCGGAAAATCGAT | 780 |
| 781 | Q S A L Y V A E M V R R E L Y E K Y G E | 280 |
| 281 | CAGAGTGCTTTATATGTGGCGGAAATGGTGCGTCGGGAACTGTATGAGAAATATGGTGAA | 840 |
| 841 | D A Y T Q G F K V Y T T V R T D H Q K A | 300 |
| 301 | GATGCCTATACGCAGGGTTTTAAGGTTTATACCACGGTCCGCACCGATCATCAGAAGGCG | 900 |
| 901 | A T E A L R K A L R N F D R G S S Y R G | 320 |
| 321 | GCAACCGAGGCATTGCGCAAGGCTCTACGGAATTTCGATCGCGGCAGCAGCTACCGCGGT | 960 |
| 961 | A E N Y I D L S K S E D V E E T V S Q Y | 340 |
| 341 | GCGGAAAACTATATCGATTTGAGTAAGAGTGAAGATGTCGAGGAGACTGTCAGCCAGTAT | 1020 |
| 1021 | L S G L Y T V D K M V P A V V L D V T K | 360 |
| 361 | CTGTCGGGACTCTATACCGTCGATAAAATGGTTCCCGCCGTTGTGTTGGATGTTACTAAA | 1080 |
| 1081 | K K N V V I Q L P G G R R V A L D R R A | 380 |
| 381 | AAGAAAAATGTCGTCATACAGCTGCCCGGCGGCAGGCGGGTTGCGCTTGACAGGCGCGCC | 1140 |
| 1141 | L G F A A R A V D N E K M G E D R I R R | 400 |
| 401 | TTGGGTTTTGCGGCCCGAGCGGTCGATAATGAGAAAATGGGGGAGGACCGTATCCGCAGG | 1200 |
| 1201 | G A V I R V K N N G G R W A V V Q E P L | 420 |
| 421 | GGCGCGGTCATCCGTGTCAAAAACAACGGGGGCGTTGGGCGGTGGTTCAAGAGCCGTTG | 1260 |
| 1261 | Q G A L V S L D A K T G A V R A L V G | 440 |
| 441 | CAGGGGGCTTTGGTTTCGCTGGATGCAAAAACCGGAGCTGTGCGCGCGCTGGTCGGC | 1320 |
| 1321 | G Y D F H S K T F N R A V Q A M R Q A G | 460 |
| 461 | GGTTATGATTTTCACAGCAAAACATTCAATCGTGCCGTTCAGGCAATGCGGCAGCCGGGT | 1380 |
| 1381 | S T F K P F V Y S A A L S K G M T A S T | 480 |
| 481 | TCGACCTTTAAGCCGTTTGTCTATTCGGCGGCATTATCTAAGGGGATGACCGCGTCCACA | 1440 |
| 1441 | V V N D A P I S L P G K G P N G S V W T | 500 |
| 501 | GTGGTTAACGATGCGCCGATTTCCCTGCCGGGGAAAGGGCCGAACGGTTCGGTTTGGACA | 1500 |
| 1501 | P K N S D G R Y S G Y I T L R Q A L T A | 520 |
| 521 | CCTAAAAATTCAGACGGCAGATATTCCGGCTACATTACTTTGAGACAGGCTCTGACGGCT | 1560 |
| 1561 | S K N M V S I R I L M S I G V G Y A Q Q | 540 |
| 541 | TCCAAGAATATGGTTTCCATCCGTATTTTGATGTCTATCGGTGTCGGTTACGCGCAACAG | 1620 |
| 1621 | Y I R R F G F R P S E L P A S L S M A L | 560 |
| 561 | TATATCCGGCGTTTCGGCTTCAGGCCGTCCGAGCTGCCGGCAAGCCTGTCTATGGCTTTA | 1680 |
| 1681 | G T G E T T P L K V A E A Y S V F A N G | 580 |
| 581 | GGTACGGGCGAGACGACGCCGTTGAAAGTGGCGGAGGCATATAGTGTATTTGCGAACGGC | 1740 |
| 1741 | G Y R V S S H V I D K I Y D R D G R L R | 600 |
| 601 | GGATATAGGGTTTCTTCGCACGTGATCGATAAGATTTATGACAGAGACGGCAGGTTGCGC | 1800 |
| 1801 | A Q M Q P L V A G Q N A P Q A I D P R N | 620 |
| 621 | GCCCAAATGCAACCTTTGGTGGCAGGGCAAAATGCGCCTCAGGCAATCGATCCGCGCAAT | 1860 |
| 1861 | A Y I M Y K I M Q D V V R V G T A R G A | 640 |
| 641 | GCCTATATTATGTATAAGATTATGCAGGATGTGGTCCGTGTCGGTACGGCAAGGGGGGCA | 1920 |
| 1921 | A A L G R T D I A G K T G T T N D N K D | 660 |
| 661 | GCTGCGTTGGGAAGAACGGATATTGCCGGTAAAACGGGTACGACCAACGACAATAAAGAT | 1980 |
| 1981 | A W F V G F N P D V V T A V Y I G F D K | 680 |
| 681 | GCGTGGTTTGTCGGTTTTAACCCTGATGTGGTTACTGCCGTATATATCGGCTTCGACAAA | 2040 |
| 2041 | P K S M G R A G Y G G T I A V P V W V D | 700 |
| 701 | CCTAAGAGTATGGGGCGTGCCGGCTACGGCGGTACGATTGCGGTGCCGGTTTGGGTGGAC | 2100 |
| 2101 | Y M R F A L K G K Q G K G M K M P E G V | 720 |
| 721 | TATATGCGTTTTGCGTTGAAAGGAAAGCAGGGCAAAGGGATGAAAATGCCTGAAGGTGTG | 2160 |
| 2161 | V S S N G E Y Y M K E R M V T D P G L M | 740 |
| 741 | GTCAGCAGCAATGGCGAATACTATATGAAGGAACGTATGGTAACCGATCCGGGCTTGATG | 2220 |
| 2221 | L D N S G I A P Q P S R R A K E D D E A | 760 |
| 761 | CTGGACAACAGCGGTATTGCGCCGCAACCTTCCCGACGGGCAAAAGAAGATGATGAAGCG | 2280 |
| 2281 | A V E N E Q Q G R S D E T R Q D V Q E T | 780 |
| 781 | GCAGTAGAAAACGAACAGCAGGGAAGGTCTGACGAAACGCGTCAGGACGTACAGGAAACG | 2340 |
| 2341 | P V L P S N T D S K Q Q Q L D S L F * | 799 |
| | CCGGTGCTTCCGAGCAATACGGATTCCAAACAGCAGCAGTTGGATTCCCTGTTTTAA | 2397 |

mtrR Gene

mtrR encodes a transcriptional repressor of the gene locus known as mtr, which encodes an efflux pump. Deletion of a single adenine residue from the promoter region of the mtrR gene results in upregulation of the efflux pump and thereby reduced susceptibility to cephalosporins (see—35A in TABLE V below).

TABLE V
NEISSERIA GONORRHOEAE mtrR AND mtrC GENES
Accession No.: CAA81045.1 (amino acid sequence) (SEQ ID NO: 9)
GenBank: Z25796 (nucleotide sequence) (SEQ ID NO: 10)
Pan, W. and Spratt, B. G. Regulation of the permeability of the gonococcal
cell envelope by the mtr system. 11 (4), 769-775 (1994)

| | | |
|---|---|---|
| 1 | GATCAGCAAA CAGCAGGCGG CCTTTTTCAG GATAAACCGT ACCGTCGTCA | 50 |
| 51 | AATTTGATGC CGACCGCAAT CGCACCATCC GCCGCCAGCA GCTTGCCTTC | 100 |
| 101 | GGCTATCTGC CGGCGCAGTT TCATCACTTC GGATGCAGAC TGGGTAACGT | 150 |
| 151 | TCACATACAT CGGATTGGTT TGGCGGATGG TGGCTAAAAC AGTTGTATCG | 200 |
| 201 | CCCGCATTCA ACAGCGTACC TTCGGAAACT TTGGACTGAC CGATAAAGCC | 250 |
| 251 | CGAAATCGGC GCGGTAATGC GCGAACGGTT CAGATTGATG CCGGCGGATT | 300 |
| 301 | TGATCGCCGC CTGCGCCGCT TTAACGCCCG CCTCGGCAGA ACGTTTCGCC | 350 |
| 351 | GTTACCGCAG CATCGTACTC TTGTTTACTG ATGGCATCGG CGGAAACCAG | 400 |
| 401 | CGGTTTGTAA CGCGCCAAAT CCGCATCCGC TTTGGCAAGC GTTGCCTGTG | 450 |
| 451 | CCGTTGCCAG TTGCGCGCGC GCGCTTTCCA GACCTGCTTC ATAAGTGGAA | 500 |
| 501 | CTGTCGATCT GATACAGCGG CTGCCCGGCG CGGACATAAC TGCCTTCTTG | 550 |
| 551 | GAACAGGCGT TTTTGGATGA TGCCGCCGAC TTGGGCGCGG ACATCGGCGG | 600 |
| 601 | TACGCAGCGA TTCCAAACGC CCCGGCAACT CGACGGTCAA TGCGACGGTT | 650 |
| 651 | TGCGGATGGA CGGTTACGAC GCCGACGACG GGCGCGGGGG CTTCCCGACC | 700 |
| 701 | CGCAGGCTGC CCGCCCTGCG CCGCGTCTCT GCCTTTACCG CAAGACGACA | 750 |
| 751 | GTGCCAATGC AACGGCGGCA GCCAACGCGG CCGCACGCAT CGCCTTAGAA | 800 |
| 801 | GCATAAAAAG CCATTATTTA TCCTATCTGT CTGGTTTGAT GTAAAGGGTT | 850 |
| 851 | TTGCCAATCA ACAGGCATTC TTATTTCAGG ATATAAAAAC CGCCTGCTTT | 900 |
| 901 | GATACCCGAA TGTTCGAACG GGTTGCAAAG CAGGTTATAC CTGTTTTCAA | 950 |
| 951 | AGTTGAGATG CAGTCTCAAT TTTATGGGTT TCATTATACA TACACGATTG | 1000 |
| 1001 | CACGGAT AA AGTCTTTTTT ATAATCCGCC CTCGTCAAAC CGACCCGAAA | 1050 |
| 1051 | CGAAAACGCC ATTATGAGAA AAACCAAAAC CGAAGCCTTG AAAACCAAAG | 1100 |
| 1101 | AACACCTGAT GCTTGCCGCC TTGGAAACCT TTTACCGCAA AGGGATTGCC | 1150 |
| 1151 | CGCACCTCGC TCAACGAAAT CGCCCAAGCC GCCGGCGTAA CGCGCGGCGC | 1200 |
| 1201 | GCTCTATTGG CATTTCAAAA ATAAGGAAGA CTTGTTTGAC GCGTTGTTCC | 1250 |
| 1251 | AACGTATCTG CGACGACATC GAAAACTGCA TCGCGCAAGA TGCCGCAGAT | 1300 |
| 1301 | GCCGAAGGAG GTTCTTGGAC GGTATTCCGC CACACGCTGC TGCACTTTTT | 1350 mtrR |
| 1351 | CGAGCGGCTG CAAAGCAACG ACATCTACTA CAAATTCCAC AACATCCTGT | 1400 |
| 1401 | TTTTAAAATG CGAACACACG GAGCAAAACG CCGCCGTTAT CGCCATTGCC | 1450 |
| 1451 | CGCAAGCATC AGGCAATCTG GCGCGAGAAA ATTACCGCCG TTTTGACCGA | 1500 |
| 1501 | AGCGGTGGAA AATCAGGATT TGGCTGACGA TTTGGACAAG GAAACGGCGG | 1550 |
| 1551 | TCATCTTCAT CAAATCGACG TTGGACGGGC TGATTTGGCG TTGGTTCTCT | 1600 |
| 1601 | TCCGGCGAAA GTTTCGATTT GGGCAAAACC GCCCCGCGCA TCATCGGGAT | 1650 |
| 1651 | AATGATGGAC AACTTGGAAA ACCATCCCTG CCTGCGCCGG AAATAATCAA | 1700 |
| 1701 | GCCTTGGTAA CAATGCCGTC TGAAACAAAC AAACCCTTTC AAACGGCATC | 1750 |
| 1751 | AAAATGACAC AAAGCATTCT TCTAAAAATA CATATTCACT AAATTGCATT | 1800 |
| 1801 | TTTAATTTCC CCTATCATCG CATGAACATT GTCTTGGTCA AAATGTCCGT | 1850 |
| 1851 | TTTCTTCTGA ATAAACTTCT AACAAATAAT GTTCAATGAA CGTTTTATCT | 1900 |
| 1901 | GTCATCAACG ATACATCTTT GGCAATGTCT TCATACGACT CAAAATCATC | 1950 |
| 1951 | TTCATGCCAT GGATCATATT TATCCATGAT TTTTTGAATT TCATTTTTCA | 2000 |
| 2001 | TATCATTTAC CTTCCAATAT TTATTTACAA TTAATAACAA TACCATTCGA | 2050 |
| 2051 | ATGTAAACTG CATTTTTCTC CGGCATTCTT GCAAACAAAA ACCGAAAATC | 2100 |
| 2101 | CCGTCATTCC CGCGCAGGCG GGAATTC | 2127 |

TABLE VI
NEISSERIA GONORRHOEAE MULTIPLE TRANSFERABLE
RESISTANCE REPRESSOR (MtrR) GENE
Accession No.: ADO32806.1 (amino acid sequence) (SEQ ID NO: 11)
GenBank: Z25796 (nucleotide sequence) (SEQ ID NO: 12)
Liao,M., Gu, W.M., Yang, Y. and Dillon,J.A.Analysis of mutations in multiple loci
of Neisseria gonorrhoeae isolates reveals effects of PIB, PBP2 and mtrR on reduced
susceptibility to ceftriaxone. J. Antimicrob. Chemother. 66 (5), 1016-1023 (2011)

| | | |
|---|---|---|
| 1 | M R K T K T E A L K T K E H L M L A A L | 20 |
| 1064 | ATGAGAAAAACCAAAACCGAAGCCTTGAAAACCAAAGAACACCTGATGCTTGCCGCCTTG | 1123 |
| 21 | E T F Y R K G I A R T S L N E I A Q A | 40 |
| 1124 | GAAACCTTTTACCGCAAAGGGATTGCCCGCACCTCGCTCAACGAAATCGCCCAA GCC | 1183 |
| 41 | G V T R A L Y W H F K N K E D L F D A | 60 |
| 1184 | GGCGTAACGCGC GCGCTCTATTGGCATTTCAAAAATAAGGAAGACTTGTTTGACGCG | 1243 |
| 61 | L F Q R I C D D I E N C I A Q D A A D A | 80 |
| 1244 | TTGTTCCAACGTATCTGCGACGACATCGAAAACTGCATCGCGCAAGATGCCGCAGATGCC | 1303 |
| 81 | E G G S W T V F R H T L L H F F E R L Q | 100 |
| 1304 | GAAGGAGGTTCTTGGACGGTATTCCGCCACACGCTGCTGCACTTTTTCGAGCGGCTGCAA | 1363 |
| 101 | S N D I Y Y K F H N I L F L K C E H T E | 120 |
| 1364 | AGCAACGACATCTACTACAAATTCCACAACATCCTGTTTTTAAAATGCGAACACACGGAG | 1423 |
| 121 | Q N A A V I A I A R K H Q A I W R E K I | 140 |
| 1424 | CAAAACGCCGCCGTTATCGCCATTGCCCGCAAGCATCAGGCAATCTGGCGCGAGAAAATT | 1483 |
| 141 | T A V L T E A V E N Q D L A D D L D K E | 160 |
| 1484 | ACCGCCGTTTTGACCGAAGCGGTGGAAAATCAGGATTTGGCTGACGATTTGGACAAGGAA | 1543 |
| 161 | T A V I F I K S T L D G L I W R W F S S | 180 |
| 1544 | ACGGCGGTCATCTTCATCAAATCGACGTTGGACGGGCTGATTTGGCGTTGGTTCTCTTCC | 1603 |
| 181 | G E S F D L G K T A P R I I G I M M D N | 200 |
| 1604 | GGCGAAAGTTTCGATTTGGGCAAAACCGCCCCGCGCATCATCGGGATAATGATGGACAAC | 1663 |
| 201 | L E N H P C L R R K * | 211 |
| 1664 | TTGGAAAACCATCCCTGCCTGCGCCGGAAATAA | 1696 |

Surveillance of N. gonorrhoeae samples in the United States demonstrates that the percentage of isolates with elevated cefixime MICs (≥0.25 mcg/mL) increased from 0.1 percent in 2006 to 1.4 percent in 2010, started to decline in 2015, and was 0.2 percent in 2021 [26]. Among 23 European countries reporting on cefixime susceptibility in the World Health Organization Gonococcal Surveillance Programme (GASP), four countries (Belgium, Denmark, Greece, and Norway) reported resistance rates ≥5 percent. Although no confirmed treatment failures with cefixime have been reported in the United States as of 2020, such failures have been reported in Canada, Europe, and Japan. Furthermore, in a retrospective study of patients treated for culture-confirmed N. gonorrhoeae infection with a single oral dose of cefixime 400 mg at a Canadian clinic, 13 of 133 patients who returned for a test of cure were again culture positive, and nine (6.8 percent) were considered treatment failures [32]. With regards to location of infection, clinical failure occurred in 4 of 76 urethral, 2 of 7 pharyngeal, and 3 of 39 rectal infections (5.3, 28.6, and 7.7 percent, respectively). Although the possibility that some of these cases represented reinfection instead of treatment failure cannot be definitively excluded, these data highlight the increasing concern of the inadequacy of cefixime for gonococcal infections.

ii. Ceftriaxone

Ceftriaxone (Compound III, PubChem CID: 5479530) is a third generation cephalosporin that selectively and irreversibly inhibits bacterial cell wall synthesis by binding to penicillin-binding proteins (PBPs).

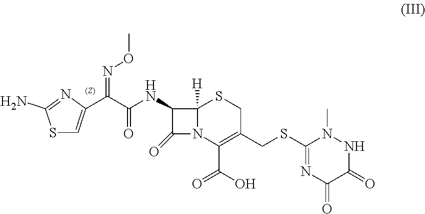 [SMILES: CO/N=C(\C(=O)N[C@@H]1C(=O)N2C(C(=O)O)=C(CSc3nc(=O)c(=O)[nH]n3C)CS[C@H]12)c1csc(N)n1]

The peptidoglycan cell wall is made up of pentapeptide units attached to a polysaccharide backbone with alternating units of N-acetylglucosamine and N-acetylmuramic acid. PBPs act on a terminal D-alanyl-D-alanine moiety on a pentapeptide unit and catalyze the formation of a peptide bond between the penultimate D-alanine and a glycine unit on an adjacent peptidoglycan strand, releasing the terminal D-alanine unit in the process. The structure of ceftriaxone mimics the D-alanyl-D-alanine moiety, and the PBP attacks the beta-lactam ring in ceftriaxone as if it were its normal D-alanyl-D-alanine substrate. The peptidoglycan cross-linking activity of PBPs is a construction and repair mechanism that normally helps to maintain bacterial cell wall integrity, so the inhibition of PBPs by ceftriaxone leads to damage and destruction of the cell wall and eventually to cell lysis.

As with cefixime, previous reports have strongly associated the presence of penA mosaicism with decreased susceptibility to ceftriaxone. However, unlike cefixime, a substantial number of strains have been identified in which penA mosaicism is neither necessary nor sufficient to predict reduced susceptibility to ceftriaxone. A series of alterations in both penA and non-penA alleles have been identified as potentially important targets: penA (A311V, A501V/P/T, A516G, N512Y, N513Y, G542S, G545S, I312M, P551L/S, V316T/P, insD345), ponA (L421P), penB (G120/A121), and mtrR (−35delA).

One molecular assay for the determination of intermediate susceptibility to ceftriaxone among N. gonorrhoeae isolates developed by Peterson et al reported a 99.8% (95% CI, 99.0%-100.0%) sensitivity and an 89.0% (95% CI, 87.5%-90.4%) specificity for the prediction of ceftriaxone susceptibility in the absence of 3 or more of the genetic alterations in penA (A311V, A501V/P/T, N513Y, G545S), ponA (L421P), penB (G120/A121), and mtrR (−35delA) (Peterson et al., Multiplex real-time PCR assays for the prediction of cephalosporin, ciprofloxacin and azithromycin antimicrobial susceptibility of positive Neisseria gonorrhoeae nucleic acid amplification test samples. J Antimicrob Chemother 2020; 75:3485-90).

Regarding non-penA mutations, a consensus appears to be that the non-penA mutations [ponA (L421P), penB (G120/A121), and mtrR (−35delA)] are all important in contributing to reduced ceftriaxone susceptibility specifically among nonmosaic strains.

### IV. Crispr-Cas Detection Systems

In an embodiment, the disclosure provides a point-of-care CRISPR-Cas system for the detection of antibiotic resistance mutations in gyrA, penA, porB, ponA and mtrR genes in samples taken from patients infected with Neisseria gonorrhoeae. In an embodiment, the CRISPR Cas system comprises i) two or more CRISPR systems, each CRISPR system comprising a Cas protein and a guide molecule comprising a guide sequence capable of binding a corresponding target molecule and designed to form a complex with the Cas protein; and ii) a set of detection constructs, each detection construct comprising a cutting motif sequence that is preferentially cut by one of the activated CRISPR effector proteins. As used herein, the term “preferentially cut” or “preferential cutting” refers to the selective cleavage activity exhibited by activated CRISPR effector proteins toward specific nucleotide sequences or cutting motifs within detection constructs. Preferential cutting occurs when a CRISPR effector protein demonstrates enhanced cleavage efficiency, increased binding affinity, or selective recognition for particular nucleotide sequences compared to other sequences present in the reaction mixture. This selectivity is based on the sequence-specific collateral nuclease activity of different CRISPR protein orthologs, wherein each ortholog exhibits distinct nucleotide preferences. For example, certain Cas13 orthologs may preferentially cut adenine (A) residues, while others show preference for uracil (U), cytosine (C), or guanosine (G) residues. The preferential cutting activity enables multiplex detection by allowing different CRISPR systems to selectively cleave their corresponding detection constructs without significant cross-reactivity. Preferential cutting may be quantified by comparing the rate of cleavage, the extent of substrate turnover, or the kinetic parameters (such as kcat/KM) between the preferred cutting motif and non-preferred sequences under standardized reaction conditions. The degree of preference may range from modest (2-5 fold) to highly selective (>10-fold) differences in cleavage efficiency. This selectivity is exploited in the present detection systems to enable specific signal generation only when the appropriate target-CRISPR system pairing is activated, thereby providing specificity in multiplex diagnostic assays.

### a) CRISPR-Cas Effector Proteins

In general, a CRISPR-Cas or CRISPR system as used herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). When the CRISPR protein is a C2c2 protein, a tracrRNA is not required. C2c2 has been described in Abudayyeh et al. (2016) “C2c2 is a single-component programmable RNA-guided RNA-targeting CRISPR effector”; Science; DOI: 10.1126/science.aaf5573; and Shmakov et al. (2015) “Discovery and Functional Characterization of Diverse Class 2 CRISPR-Cas Systems”, Molecular Cell, DOI: dx.doi.org/10.1016/j.molcel.2015.10.008; which are incorporated herein in their entirety by reference. Cas13b has been described in Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molcel.2016.12.023, which is incorporated herein in its entirety by reference.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of 5′ end of the protospacer). In other embodiments, the PAM may be a 3′ PAM (i.e., located downstream of 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein His A, C or U. In certain embodiments, the effector protein may be Leptotrichia shahii C2c2p, more preferably Leptotrichia shahii DSM 19757 C2c2, and 3′ PAM is a 5′ H.

In the context of formation of a CRISPR complex, “target molecule or “target sequence” refers to a molecule harboring a sequence, or a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell. A target sequence may comprise DNA polynucleotides.

As such, a CRISPR system may comprise RNA-targeting effector proteins. A CRISPR system may comprise DNA-targeting effector proteins. In some embodiments, a CRISPR system may comprise a combination of RNA- and DNA-targeting effector proteins, or effector proteins that target both RNA and DNA.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains.

In one example embodiment, the effector protein comprises one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional patent application entitled “Novel Type VI CRISPR Orthologs and Systems,” filed on Apr. 12, 2017.

In an embodiment of the disclosure, a HEPN domain comprises at least one RxxxxH motif comprising the sequence of R {N/H/K} X2X3X4H (SEQ ID NO: 13). In an embodiment of the disclosure, a HEPN domain comprises a RxxxxH motif comprising the sequence of R {N/H} X2X3X4H (SEQ ID NO:14). In an embodiment of the disclosure, a HEPN domain comprises the sequence of R {N/K} X2X3X4H (SEQ ID NO: 15). In certain embodiments, X2 is R, S, D, E, Q, N, G, Y, or H. In certain embodiments, X3 is I, S, T, V, or L. In certain embodiments, X4 is L, F, N, Y, V, I, S, D, E, or A.

Additional effectors for use according to the disclosure can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related or are only partially structurally related. An “orthologue” of a protein as used herein is a protein of a different species which performs the same or a similar function as the protein it is an orthologue of. Orthologous proteins may but need not be structurally related or are only partially structurally related.

### a) Cas13 Proteins

In an embodiment, the Type VI RNA-targeting Cas enzyme is C2c2. In another example embodiment, the Type VI RNA-targeting Cas enzyme is Cas13b. In another embodiment, the Cas13b protein is from an organism of a genus selected from the group consisting of: Bergeyella, Prevotella, Porphyromonas, Bacterioides, Alistipes, Riemerella, Myroides, Capnocytophaga, Porphyromonas, Flavobacterium, Porphyromonas, Chryseobacterium, Paludibacter, Psychroflexus, Riemerella, Phaeodactylibacter, Sinomicrobium, and Reichenbachiella.

In particular embodiments, the homologue or orthologue of a Type VI protein such as C2c2 as referred to herein has a sequence homology or identity of at least 30%, or at least 40%, or at least 50%, or at least 60%, or at least 70%, or at least 80%, more preferably at least 85%, even more preferably at least 90%, such as, for instance, at least 95% with a Type VI protein such as C2c2 (e.g., based on the wild-type sequence of any of Leptotrichia shahii C2c2, Lachnospiraceae bacterium MA2020 C2c2, Lachnospiraceae bacterium NK4A179 C2c2, Clostridium aminophilum (DSM 10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2). In further embodiments, the homologue or orthologue of a Type VI protein such as C2c2 as referred to herein has a sequence identity of at least 30%, or at least 40%, or at least 50%, or at least 60%, or at least 70%, or at least 80%, more preferably at least 85%, even more preferably at least 90%, such as for instance at least 95% with the wild type C2c2 (e.g., based on the wild-type sequence of any of Leptotrichia shahii C2c2, Lachnospiraceae bacterium MA2020 C2c2, Lachnospiraceae bacterium NK4A179 C2c2, Clostridium aminophilum (DSM 10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2).

In certain other example embodiments, the CRISPR system effector protein is a C2c2 nuclease. The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. Regarding C2c2 CRISPR systems, reference is made to U.S. Provisional 62/351,662 filed on Jun. 17, 2016 and U.S. Provisional 62/376,377 filed on Aug. 17, 2016. Reference is also made to U.S. Provisional 62/351,803 filed on Jun. 17, 2016. Reference is also made to U.S. Provisional entitled “Novel Crispr Enzymes and Systems” filed Dec. 8, 2016 bearing Broad Institute No. 10035.PA4. Reference is further made to East-Seletsky et al. “Two distinct RNase activities of CRISPR-C2c2 enable guide-RNA processing and RNA detection” Nature doi: 10/1038/nature19802 and Abudayyeh et al. “C2c2 is a single-component programmable RNA-guided RNA targeting CRISPR effector” bioRxiv doi: 10.1101/054742.

RNase function in CRISPR systems is known, for example, mRNA targeting has been reported for certain type III CRISPR-Cas systems (Hale et al., 2014, Genes Dev, vol. 28, 2432-2443; Hale et al., 2009, Cell, vol. 139, 945-956; Peng et al., 2015, Nucleic acids research, vol. 43, 406-417) and provides significant advantages. In the Staphylococcus epidermis type III-A system, transcription across targets results in cleavage of the target DNA and its transcripts, mediated by independent active sites within the Cas10-Csm ribonucleoprotein effector protein complex (see, Samai et al., 2015, Cell, vol. 151, 1164-1174). A CRISPR-Cas system, composition or method targeting RNA via the present effector proteins is thus provided.

In an embodiment, the Cas protein may be a C2c2 ortholog of an organism of a genus which includes but is not limited to Leptotrichia, Listeria, Corynebacter, Sutterella, Legionella, Treponema, Filifactor, Eubacterium, Streptococcus, LactoBacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter. Species of organism of such a genus can be as otherwise herein discussed.

Some methods of identifying orthologues of CRISPR-Cas system enzymes may involve identifying tracr sequences in genomes of interest. Identification of tracr sequences may relate to the following steps: Search for the direct repeats or tracr mate sequences in a database to identify a CRISPR region comprising a CRISPR enzyme. Search for homologous sequences in the CRISPR region flanking the CRISPR enzyme in both the sense and antisense directions. Look for transcriptional terminators and secondary structures. Identify any sequence that is not a direct repeat or a tracr mate sequence but has more than 50% identity to the direct repeat or tracr mate sequence as a potential tracr sequence. Take the potential tracr sequence and analyze for transcriptional terminator sequences associated therewith.

It will be appreciated that any of the functionalities described herein may be engineered into CRISPR enzymes from other orthologs, including chimeric enzymes comprising fragments from multiple orthologs. Examples of such orthologs are described elsewhere herein. Thus, chimeric enzymes may comprise fragments of CRISPR enzyme orthologs of an organism which includes, but is not limited to, Leptotrichia, Listeria, Corynebacter, Sutterella, Legionella, Treponema, Filifactor, Eubacterium, Streptococcus, LactoBacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter. A chimeric enzyme can comprise a first fragment and a second fragment, and the fragments can be of CRISPR enzyme orthologs of organisms of genera herein mentioned or of species herein mentioned; advantageously the fragments are from CRISPR enzyme orthologs of different species.

In embodiments, the C2c2 protein as referred to herein also encompasses a functional variant of C2c2 or a homologue or an orthologue thereof. A “functional variant” of a protein as used herein refers to a variant of such protein which retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide or peptide. Functional variants may be naturally occurring or may be man-made. Advantageous embodiments can involve engineered or non-naturally occurring Type VI RNA-targeting effector protein.

In an embodiment, the C2c2 or an ortholog or homolog thereof, may comprise one or more mutations. The mutations may be artificially introduced mutations and may include, but are not limited to, one or more mutations in a catalytic domain. Examples of catalytic domains with reference to a Cas9 enzyme may include, but are not limited to, RuvC I, RuvC II, RuvC III and HNH domains.

In certain example embodiments, the C2c2 effector protein may be from an organism selected from the group consisting of; Leptotrichia, Listeria, Corynebacter, Sutterella, Legionella, Treponema, Filifactor, Eubacterium, Streptococcus, LactoBacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, and Campylobacter.

In certain embodiments, the effector protein may be a Listeria sp. C2c2p, preferably Listeria seeligeri C2c2p, more preferably Listeria seeligeri serovar ½b str. SLCC3954 C2c2p and the crRNA sequence may be 44 to 47 nucleotides in length, with a 5′ 29-nt direct repeat (DR) and a 15-nt to 18-nt spacer.

In certain embodiments, the effector protein may be a Leptotrichia sp. C2c2p, preferably Leptotrichia shahii C2c2p, more preferably Leptotrichia shahii DSM 19757 C2c2p and the crRNA sequence may be 42 to 58 nucleotides in length, with a 5′ direct repeat of at least 24 nt, such as a 5′ 24-28-nt direct repeat (DR) and a spacer of at least 14 nt, such as a 14-nt to 28-nt spacer, or a spacer of at least 18 nt, such as 19, 20, 21, 22, or more nt, such as 18-28, 19-28, 20-28, 21-28, or 22-28 nt.

In certain example embodiments, the effector protein may be a Leptotrichia sp., Leptotrichia wadei F0279, or a Listeria sp., preferably Listeria newyorkensis FSL M6-0635.

In certain example embodiments, the C2c2 effector proteins of the disclosure include, without limitation, the following 21 ortholog species (including multiple CRISPR loci: Leptotrichia shahii; Leptotrichia wadei (Lw2); Listeria seeligeri; Lachnospiraceae bacterium MA2020; Lachnospiraceae bacterium NK4A179; Clostridium aminophilum DSM 10710; Carnobacterium gallinarum DSM 4847; Carnobacterium gallinarum DSM 4847 (second CRISPR Loci); Paludibacter propionicigenes WB4; Listeria weihenstephanensis FSL R9-0317; Listeriaceae bacterium FSL M6-0635; Leptotrichia wadei F0279; Rhodobacter capsulatus SB 1003; Rhodobacter capsulatus R121; Rhodobacter capsulatus DE442; Leptotrichia buccalis C-1013-b; Herbinix hemicellulosilytica; Eubacterium rectale; Eubacteriaceae bacterium CHKCI004; Blautia sp. Marseille-P2398; and Leptotrichia sp. oral taxon 879 str. F0557. Twelve (12) further non-limiting examples are: Lachnospiraceae bacterium NK4A144; Chloroflexus aggregans; Demequina aurantiaca; Thalassospira sp. TSL5-1; Pseudobutyrivibrio sp. OR37; Butyrivibrio sp. YAB3001; Blautia sp. Marseille-P2398; Leptotrichia sp. Marseille-P3007; Bacteroides ihuae; Porphyromonadaceae bacterium KH3CP3RA; Listeria riparia; and Insolitispirillum peregrinum.

In certain embodiments, the C2c2 protein according to the disclosure is, or is derived from, one of the orthologues as described in the table below, or is a chimeric protein of two or more of the orthologues as described in the table below, or is a mutant or variant of one of the orthologues as described in the table below (or a chimeric mutant or variant), including dead C2c2, split C2c2, destabilized C2c2, etc. as defined herein elsewhere, with or without fusion with a heterologous/functional domain.

In certain example embodiments, the C2c2 effector protein is from an organism of a genus selected from the group consisting of: Leptotrichia, Listeria, Corynebacter, Sutterella, Legionella, Treponema, Filifactor, Eubacterium, Streptococcus, LactoBacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma,

### Campylobacter, and Lachnospira.

In certain example embodiments, the C2c2 effector protein is selected from TABLE VII below.

TABLE VII

| C2c2 orthologue | Code | Multi Letter |
|---|---|---|
| Leptotrichia shahii | C2-2 | Lsh |
| L. wadei F0279 (Lw2) | C2-3 | Lw2 |
| Listeria seeligeri | C2-4 | Lse |
| Lachnospiraceae bacterium MA2020 | C2-5 | LbM |
| Lachnospiraceae bacterium NK4A179 | C2-6 | LbNK179 |
| Clostridium aminophilum DSM 10710 | C2-7 | Ca |
| Carnobacterium gallinarum DSM 4847 | C2-8 | Cg |
| Carnobacterium gallinarum DSM 4847 | C2-9 | Cg2 |
| Paludibacter propionicigenes WB4 | C2-10 | Pp |
| Listeria weihenstephanensis FSL R9-0317 | C2-11 | Lwei |
| Listeriaceae bacterium FSL M6-0635 | C2-12 | LbFSL |
| Leptotrichia wadei F0279 | C2-13 | Lw |
| Rhodobacter capsulatus SB 1003 | C2-14 | Rc |
| Rhodobacter capsulatus R121 | C2-15 | Rc |
| Rhodobacter capsulatus DE442 | C2-16 | Rc |
| Leptotrichia buccalis C-1013-b | C2-17 | LbuC2c2 |
| Herbinix hemicellulosilytics | C2-18 | HheC2c2 |
| Eubacterium rectale | C2-19 | EreC2c2 |
| Eubacteriaceae bacterium CHKC1004 | C2-20 | EbaC2c2 |
| Blautia sp. Marseille-P2398 | C2-21 | BsmC2c2 |
| Leptotrichia sp. oral taxon 879 str. F0557 | C2-22 | LspC2c2 |
| Lachnospiraceae bacterium NK4a144 | | |
| Chloroflexus aggregans | | |
| Demequina aurantiaca | | |
| Thalassospira sp. TSL5-1 | | |
| PseudoButyrivibrio sp. OR37 | | |
| Butyrivibrio sp. YAB3001 | | |
| Blautia sp. Marseille-P2398 | | |
| Leptotrichia sp. Marseille-P300 | | |
| Bacteroides ihuae | | |
| Porphyromonadaceae bacterium KH3CP3RA | | |
| Listeria riparia | | |
| Insolitispirillum peregrinum | | |

The wild type protein sequences of the above species are listed in TABLE VIII below. In certain embodiments, a nucleic acid sequence encoding the C2c2 protein is provided.

TABLE VIII

| | | |
|---|---|---|
| C2c2-2 | | L. shahii (Lsh) (SEQ ID NO: 16) |
| C2c2-2 | | L. shahii (Lsh) WP_018451595.1 (SEQ ID NO: 17) |
| c2c2-3 | | L. wadei (Lw2) (SEQ ID NO: 18) |
| c2c2-4 | | Listeria seeligeri (SEQ ID NO: 19) |
| c2c2-5 | 1 | Lachnospiraceae bacterium MA2020 (SEQ ID NO: 20) |
| c2c2-6 | 2 | Lachnospiraceae bacterium NK4A179 (SEQ ID NO: 21) |
| c2c2-7 | 3 | Clostridium aminophilum DSM 10710 (SEQ ID NO: 22) |
| c2c2-8 | 5 | Carnobacterium gallinarum DSM 4847 (SEQ ID NO: 23) |
| c2c2-9 | 6 | Carnobacterium gallinarum DSM 4847 (SEQ ID NO: 24) |
| c2c2-10 | 7 | Paludibacter propionicigenes WB4 (SEQ ID NO: 25) |
| c2c2-11 | 9 | Listeria weihenstephanensis FSL R9-0317 (SEQ ID NO: 26) |
| c2c2-12 | 10 | Listeriaceae bacterium FSL M6-0635 = Listeria newyorkensis |
| | | FSL M6-0635 (SEQ ID NO: 27) |
| c2c2-13 | 12 | Leptotrichia wadei F0279 (SEQ ID NO: 28) |
| c2c2-14 | 15 | Rhodobacter capsulatus SB 1003 (SEQ ID NO: 29) |
| c2c2-15 | 16 | Rhodobacter capsulatus R121 (SEQ ID NO: 30) |
| c2c2-16 | 17 | Rhodobacter capsulatus DE442 (SEQ ID NO: 31) |
| LbuC2c2 (C2-17) | | Leptotrichia buccalis C-1013-b (SEQ ID NO: 32) |
| HheC2c2 (C2-18) | | Herbinix hemicellulosilytica (SEQ ID NO: 33) |
| EreC2c2 (C2-19) | | Eubacterium rectale (SEQ ID NO: 34) |
| EbaC2C2 (C2-20) | | Eubacteriaceae bacterium CHKCI004 (SEQ ID NO: 35) |
| C2c2 (C2-21) | | Blautia sp. Marseille-P2398 (SEQ ID NO: 36) |
| C2c2 (C2-22) | | Leptotrichia sp. Oral taxon 879 str. F0557 (SEQ ID NO: 37) |
| C2c2 NK4A144 (C2-23) | | Lachnospiraceae bacterium NK4A144 |
| C2c2 Chloro_agg (C2-24) | | RNA-binding protein S1 Chloroflexus aggregans (SEQ ID NO: 38) |
| C2c2 Dem_Aur (C2-25) | | Demequina aurantiaca (SEQ ID NO: 39) |
| C2c2 Thal_Sp_TSL5 (C2-26) | | Thalassospira sp. TSL5-1 (SEQ ID NO: 40) |
| C2c2 Pseudo_sp (C2-27) | | PseudoButyrivibrio sp. OR37 (SEQ ID NO: 41) |
| C2c2_Buty_sp (C2-28) | | Butyrivibrio sp. YAB3001 |
| C2c2_Blautia_sp (C2-29) | | Blautia sp. Marseille-P2398 (SEQ ID NO: 42) |
| C2c2_Lepto_sp_Marseille (C2-30) | | Leptotrichia sp. Marseille-P3007 (SEQ ID NO: 43) |
| C2c2_Bacteroides_ihuae (C2-31) | | Bacteroides ihuae (SEQ ID NO: 44) |
| C2c2_Porph_bacterium (C2-32) | | Porphyromonadaceae bacterium KH3CP3RA |
| C2c2_Listeria_riparia (C2-33) | | Listeria riparia (SEQ ID NO: 45) |
| C2c2_insolitis_peregrinum (C2-34) | | Insolitispirillum peregrinum (SEQ ID NO: 46) |

In an embodiment of the disclosure, there is provided effector protein which comprises an amino acid sequence having at least 80% sequence homology to the wild-type sequence of any of Leptotrichia shahii C2c2, Lachnospiraceae bacterium MA2020 C2c2, Lachnospiraceae bacterium NK4A179 C2c2, Clostridium aminophilum (DSM 10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2.

In an embodiment of the disclosure, the effector protein comprises an amino acid sequence having at least 80% sequence homology to a Type VI effector protein consensus sequence including, but not limited to, a consensus sequence described herein.

According to the disclosure, a consensus sequence can be generated from multiple C2c2 orthologs, which can assist in locating conserved amino acid residues, and motifs, including but not limited to catalytic residues and HEPN motifs in C2c2 orthologs that mediate C2c2 function. One such consensus sequence, generated from the 33 orthologs mentioned above using Geneious alignment is SEQ ID NO: 47.

In another non-limiting example, a sequence alignment tool to assist generation of a consensus sequence and identification of conserved residues is the MUSCLE alignment tool (www.ebi.ac.uk/Tools/msa/muscle/). For example, using MUSCLE, the following amino acid locations conserved among C2c2 orthologs can be identified in Leptotrichia wadei C2c2:K2; K5; V6; E301; L331; 1335; N341; G351; K352; E375; L392; L396; D403; F446; 1466; 1470; R474 (HEPN); H475; H479 (HEPN), E508; P556; L561; 1595; TABLE Y596; F600; Y669; I673; F681; L685; Y761; L676; L779; Y782; L836; D847; Y863; L869; 1872; K879; 1933; L954; 1958; R961; Y965; E970; R971; D972; R1046 (HEPN), H1051 (HEPN), Y1075; D1076; K1078; K1080; I1083; I1090.

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this disclosure, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017. In particular embodiments, the Cas13b enzyme is derived from Bergeyella zoohelcum. In certain other example embodiments, the effector protein comprises an amino acid sequence having at least 80% sequence homology to any of the sequences listed in TABLE XXIII.

TABLE XXIII

| | |
|---|---|
| B-01 | Bergeyella zoohelcum |
| B-02 | Prevotella intermedia |
| B-03 | Prevotella buccae |
| B-04 | Alistipes sp. ZOR0009 |
| B-05 | Prevotella sp. MA2016 |
| B-06 | Riemerella anatipestifer |
| B-07 | Prevotella aurantiaca |
| B-08 | Prevotella saccharolytica |
| B-09 | Prevotella intermedia |
| B-10 | Capnocytophaga canimorsus |
| B-11 | Porphyromonas gulae |
| B-12 | Prevotella sp. P5-125 |
| B-13 | Flavobacterium branchiophilum |
| B-14 | Porphyromonas gingivalis |
| B-15 | Prevotella intermedia |

In certain example embodiments, the wild type sequence of the Cas13b orthologue is found in TABLE XXIV below.

TABLE XXIV

| SPECIES / SEQ ID NO (US 2021/0108267) | ACCESSION # |
|---|---|
| Bergeyella zoohelcum (SEQ ID NO: 48) | WP_002664492 |
| Prevotella intermedia (SEQ ID NO: 49) | WP_036860899 |
| Prevotella buccae (SEQ ID NO: 50) | |
| Porphyromonas gingivalis (SEQ ID NO: 51) | |
| Bacteroides pyogenes (SEQ ID NO: 52) | |
| Alistipes sp. ZOR00096 (SEQ ID NO: 53) | |
| Prevotella sp. MA2016 (SEQ ID NO: 54) | |
| Prevotella sp. MA2016 (SEQ ID NO: 55) | |
| Riemerella anatipestifer (SEQ ID NO: 56) | |
| Prevotella aurantiaca (SEQ ID NO: 57) | |
| Prevotella saccharolytica (SEQ ID NO: 58) | |
| HMPREF9712_03108 [Myroides odoratimimus CCUG 10230] (SEQ ID NO: 59) | |
| Prevotella intermedia (SEQ ID NO: 60) | |
| Capnocytophaga canimorsus (SEQ ID NO: 61) | |
| Porphyromonas gulae (SEQ ID NO: 62) | |
| Prevotella sp. P5-125 (SEQ ID NO: 63) | |
| Flavobacterium branchiophilum (SEQ ID NO: 64) | |
| Myroides odoratimimus (SEQ ID NO: 65) | |
| Flavobacterium columnare (SEQ ID NO: 66) | |
| Porphyromonas gingivalis (SEQ ID NO: 67) | |
| Porphyromonas sp. COT-052 OH4946 (SEQ ID NO: 68) | |
| Prevotella intermedia (SEQ ID NO: 69) | |
| PIN17_0200 [Prevotella intermedia 17] (SEQ ID NO: 70) | AFJ07523 |
| Prevotella intermedia (SEQ ID NO: 71) | BAU18623 |
| HMPREF6485_0083 [Prevotella buccae ATCC 33574] (SEQ ID NO: 72) | EFU31981 |
| HMPREF9144_1146 [Prevotella pallens ATCC 700821] (SEQ ID NO: 73) | EGQ18444 |
| HMPREF9714_02132 [Myroides odoratimimus CCUG 12901] (SEQ ID NO: 74) | EHO08761 |
| HMPREF9711_00870 [Myroides odoratimimus CCUG 3837] (SEQ ID NO: 75) | EKB06014 |
| HMPREF9699_02005 [Bergeyella zoohelcum ATCC 43767] (SEQ ID NO: 76) | EKB54193 |
| HMPREF9151_01387 [Prevotella saccharolytica F0055] (SEQ ID NO: 77) | EKY00089 |
| A343_1752 [Porphyromonas gingivalis JCVI SC001] (SEQ ID NO: 78) | EOA10535 |
| HMPREF1981_03090 [Bacteroides pyogenes F0041] (SEQ ID NO: 79) | ERI81700 |
| HMPREF1553_02065 [Porphyromonas gingivalis F0568] (SEQ ID NO: 80) | ERJ65637 |
| HMPREF1988_01768 [Porphyromonas gingivalis F0185] (SEQ ID NO: 81) | ERJ81987 |
| HMPREF1990_01800 [Porphyromonas gingivalis W4087] (SEQ ID NO: 82) | ERJ87335 |
| M573_117042 [Prevotella intermedia ZT] (SEQ ID NO: 83) | KJJ86756 |
| A2033_10205 [Bacteroidetes bacterium GWA2_31_9] (SEQ ID NO: 84) | OFX18020.1 |
| SAMN05421542_0666 [Chryseobacterium jejuense] (SEQ ID NO: 85) | SDI27289.1 |
| SAMN05444360_11366 [Chryseobacterium carnipullorum] (SEQ ID NO: 86) | SHM52812.1 |
| SAMN05421786_1011119 [Chryseobacterium ureilyticum] (SEQ ID NO: 87) | SIS70481.1 |
| Segatella buccae (SEQ ID NO: 88) | WP_004343581 |
| Porphyromonas gingivalis (SEQ ID NO: 89) | WP_005873511 |
| Porphyromonas gingivalis (SEQ ID NO: 90) | WP_005874195 |
| Prevotella pallens (SEQ ID NO: 91) | WP_006044833 |
| Myroides odoratimimus (SEQ ID NO: 92) | WP_006261414 |
| Myroides odoratimimus (SEQ ID NO: 93) | WP_006265509 |
| Prevotella sp. MSX73 (SEQ ID NO: 94) | WP_007412163 |
| Porphyromonas gingivalis (SEQ ID NO: 95) | WP_012458414 |
| Paludibacter propionicigenes (SEQ ID NO: 96) | WP_013446107 |
| Porphyromonas gingivalis (SEQ ID NO: 97) | WP_013816155 |
| Flavobacterium columnare (SEQ ID NO: 98) | WP_014165541 |
| Psychroflexus torquis (SEQ ID NO: 99) | WP_015024765 |
| Riemerella anatipestifer (SEQ ID NO: 100) | WP_015345620 |
| Hoylesella pleuritidis (SEQ ID NO: 101) | WP_021584635 |
| Porphyromonas gingivalis (SEQ ID NO: 102) | WP_021663197 |
| Porphyromonas gingivalis (SEQ ID NO: 103) | WP_021665475 |
| Porphyromonas gingivalis (SEQ ID NO: 104) | WP_021677657 |
| Porphyromonas gingivalis (SEQ ID NO: 105) | WP_021680012 |
| Porphyromonas gingivalis (SEQ ID NO: 106) | WP_023846767 |
| Porphyromonas gingivalis (SEQ ID NO:107) | WP_036884929 |
| Prevotella pleuritidis (SEQ ID NO: 108) | WP_036931485 |
| Porphyromonas gingivalis (SEQ ID NO: 109) | WP_039417390 |
| Porphyromonas gulae (SEQ ID NO: 110) | WP_039418912 |
| Porphyromonas gulae (SEQ ID NO: 111) | WP_039419792 |
| Porphyromonas gulae (SEQ ID NO: 112) | WP_039426176 |
| Porphyromonas gulae (SEQ ID NO: 113) | WP_039431778 |
| Porphyromonas gulae (SEQ ID NO: 114) | WP_039437199 |
| Porphyromonas gulae (SEQ ID NO: 115) | WP_039442171 |
| Porphyromonas gulae (SEQ ID NO: 116) | WP_039445055 |
| Capnocytophaga cynodegmi (SEQ ID NO: 117) | WP_041989581 |
| Prevotella sp. P5-119 (SEQ ID NO: 118) | WP_042518169 |
| Prevotella sp. P4-76 (SEQ ID NO: 119) | WP_044072147 |
| Prevotella sp. P5-60 (SEQ ID NO: 120) | WP_044074780 |
| Phaeodactylibacter xiamenensis (SEQ ID NO: 121) | WP_044218239 |
| Flavobacterium sp. 316 (SEQ ID NO: 122) | WP_045968377 |
| Porphyromonas gulae (SEQ ID NO: 123) | WP_046201018 |
| Chryseobacterium sp. YR477 (SEQ ID NO: 124) | WP_047431796 |
| Riemerella anatipestifer (SEQ ID NO: 125) | WP_049354263 |
| Porphyromonas gingivalis (SEQ ID NO: 126) | WP_052912312 |
| Porphyromonas gingivalis (SEQ ID NO: 127) | WP_058019250 |
| Flavobacterium columnare (SEQ ID NO: 128) | WP_060381855 |
| Porphyromonas gingivalis (SEQ ID NO: 129) | WP_061156470 |
| Porphyromonas gingivalis (SEQ ID NO: 130) | WP_061156637 |
| Riemerella anatipestifer (SEQ ID NO: 131) | WP_061710138 |
| Flavobacterium covae (SEQ ID NO: 132) | WP_063744070 |
| Riemerella anatipestifer (SEQ ID NO: 133) | WP_064970887 |
| Sinomicrobium oceani (SEQ ID NO: 134) | WP_072319476.1 |
| Reichenbachiella agariperforans (SEQ ID NO: 135) | WP_073124441.1 |

In certain example embodiments, the RNA-targeting effector protein is a Cas13c effector protein as disclosed in U.S. Provisional Patent Application No. 62/525,165 filed Jun. 26, 2017, and PCT Application No. US 2017/047193 filed Aug. 16, 2017. In certain example embodiments, the Cas13c protein may be from an organism of a genus such as Fusobacterium or Anaerosalibacter. Example wildtype orthologue sequences of Cas13c are provided in TABLE XXV below.

TABLE XXV

| | SEQ ID NO: | | SEQ ID NO: | | SEQ ID NO: |
|---|---|---|---|---|---|
| EHO19081 | 136 | WP_035935671 | 137 | WP_005959231 | 138 |
| WP_094899336 | 139 | WP_035906563 | 140 | WP_027128616 | 141 |
| WP_040490876 | 142 | WP_042678931 | 143 | WP_062624740 | 144 |
| WP_047396607 | 145 | WP_062627846 | 146 | WP_096402050 | 147 |

In certain example embodiments, the Cas13 protein may be selected from any of the following.

TABLE XXVI

| | | SEQ |
|---|---|---|
| | | ID |
| ID | Species | NO: |
| Cas13a1 | Leptotrichia shahii | 148 |
| Cas13a2 | Leptotrichia wadei (Lw2) | 149 |
| Cas13a3 | Listeria seeligeri | 150 |
| Cas13a4 | Lachnospiraceae bacterium MA2020 | 151 |
| Cas13a5 | Lachnospiraceae bacterium NK4A179 | 152 |
| Cas13a6 | [Clostridium] aminophilum DSM 10710 | 153 |
| Cas13a7 | Carnobacterium gallinarum DSM 4847 | 154 |
| Cas13a8 | Carnobacterium gallinarum DSM 4847 | 155 |
| Cas13a9 | Paludibacter propionicigenes WB4 | 156 |
| Cas13a10 | Listeria weihenstephanensis FSL R9-0317 | 157 |
| Cas13a11 | Listeriaceae bacterium FSL M6-0635 | 158 |
| Cas13a12 | Leptotrichia wadei F0279 | 159 |
| Cas13a13 | Rhodobacter capsulatus SB 1003 | 160 |
| Cas13a14 | Rhodobacter capsulatus R121 | 161 |
| Cas13a15 | Rhodobacter capsulatus DE442 | 162 |
| Cas13a16 | Leptotrichia buccalis C-1013-b | 163 |
| Cas13a17 | Herbinix hemicellulosilytica | 164 |
| Cas13a18 | Eubacterium rectale | 165 |
| Cas13a19 | Eubacteriaceae bacterium CHKCI004 | 166 |
| Cas13a20 | Blautia sp. Marseille-P2398 | 167 |
| Cas13a21 | Leptotrichia sp. oral taxon 879 str. F0557 | 168 |
| Cas13b1 | Bergeyella zoohelcum | 169 |
| Cas13b2 | Prevotella intermedia | 170 |
| Cas13b3 | Prevotella buccae | 171 |
| Cas13b4 | Alistipes sp. ZOR0009 | 172 |
| Cas13b5 | Prevotella sp. MA2016 | 173 |
| Cas13b6 | Riemerella anatipestifer | 174 |
| Cas13b7 | Prevotella aurantiaca | 175 |
| Cas13b8 | Prevotella saccharolytica | 176 |
| Cas13b9 | Prevotella intermedia | 177 |
| Cas13b10 | Capnocytophaga canimorsus | 178 |
| Cas13b11 | Porphyromonas gulae | 179 |
| Cas13b12 | Prevotella sp. P5-125 | 180 |
| Cas13b13 | Flavobacterium branchiophilum | 181 |
| Cas13b14 | Porphyromonas gingivalis | 182 |
| Cas13b15 | Prevotella intermedia | 183 |
| Cas13c1 | Fusobacterium necrophorum subsp. funduliforme | 184 |
| | ATCC 51357 contig00003 | |
| Cas13c2 | Fusobacterium necrophorum DJ-2 contig0065, whole | 185 |
| | genome shotgun sequence | |
| Cas13c3 | Fusobacterium necrophorum BFTR-1 contig0068 | 186 |
| Ca13c4 | Fusobacterium necrophorum subsp. funduliforme | 187 |
| | 1_1_36S cont1.14 | |
| Cas13c5 | Fusobacterium perfoetens ATCC 29250 | 188 |
| | T364DRAFT_scaffold00009.9_C | |
| Cas13c6 | Fusobacterium ulcerans ATCC 49185 cont2.38 | 189 |
| Cas13c7 | Anaerosalibacter sp. ND1 genome assembly | 190 |
| | Anaerosalibacter massiliensis ND1 | |

### b) Cas12 Proteins

In certain example embodiments, the assays may comprise multiple Cas12 orthologs or one or more orthologs in combination with one or more Cas13 orthologs. In certain example embodiments, the Cas12 orthologs are Cpf1 orthologs. In certain other example embodiments, the Cas12 orthologs are C2c1 orthologs.

### Cpf1 Orthologs

The present disclosure encompasses the use of a Cpf1 effector protein, derived from a Cpf1 locus denoted as subtype V-A. Herein such effector proteins are also referred to as “Cpf1p”, e.g., a Cpf1 protein (and such effector protein or Cpf1 protein or protein derived from a Cpf1 locus is also called “CRISPR enzyme”). Presently, the subtype V-A loci encompasses cas1, cas2, a distinct gene denoted cpf1 and a CRISPR array. Cpf1 (CRISPR-associated protein Cpf1, subtype PREFRAN) is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in all Cas9 proteins, and the RuvC-like domain is contiguous in the Cpf1 sequence, in contrast to Cas9 where it contains long inserts including the HNH domain. Accordingly, in particular embodiments, the CRISPR-Cas enzyme comprises only a RuvC-like nuclease domain.

The programmability, specificity, and collateral activity of the RNA-guided Cpf1 also make it an ideal switchable nuclease for non-specific cleavage of nucleic acids. In one embodiment, a Cpf1 system is engineered to provide and take advantage of collateral non-specific cleavage of RNA. In another embodiment, a Cpf1 system is engineered to provide and take advantage of collateral non-specific cleavage of ssDNA. Accordingly, engineered Cpf1 systems provide platforms for nucleic acid detection and transcriptome manipulation. Cpf1 is developed for use as a mammalian transcript knockdown and binding tool. Cpf1 is capable of robust collateral cleavage of RNA and ssDNA when activated by sequence-specific targeted DNA binding.

The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related or are only partially structurally related. An “orthologue” of a protein as used herein is a protein of a different species which performs the same or a similar function as the protein it is an orthologue of. Orthologous proteins may but need not be structurally related or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22(4):359-66. doi: 10.1002/pro.2225). See also Shmakov et al. (2015) for application in the field of CRISPR-Cas loci. Homologous proteins may but need not be structurally related, or are only partially structurally related.

The Cpf1 gene is found in several diverse bacterial genomes, typically in the same locus with cas1, cas2, and cas4 genes and a CRISPR cassette (for example, FNFX1_1431-FNFX1 1428 of Francisella cf. novicida Fx1). Thus, the layout of this putative novel CRISPR-Cas system appears to be like that of type II-B. Furthermore, like Cas9, the Cpf1 protein contains a readily identifiable C-terminal region that is homologous to the transposon ORF-B and includes an active RuvC-like nuclease, an arginine-rich region, and a Zn finger (absent in Cas9). However, unlike Cas9, Cpf1 is also present in several genomes without a CRISPR-Cas context and its relatively high similarity with ORF-B suggests that it might be a transposon component. It was suggested that if this was a genuine CRISPR-Cas system and Cpf1 is a functional analog of Cas9 it would be a novel CRISPR-Cas type, namely type V (See Annotation and Classification of CRISPR-Cas Systems. Makarova K S, Koonin E V. Methods Mol Biol. 2015; 1311:47-75). However, as described herein, Cpf1 is denoted to be in subtype V-A to distinguish it from C2c1p which does not have an identical domain structure and is hence denoted to be in subtype V-B.

In particular embodiments, the effector protein is a Cpf1 effector protein from an organism from a genus comprising Streptococcus, Campylobacter, Nitratifractor, Staphylococcus, Parvibaculum, Roseburia, Neisseria, Gluconacetobacter, Azospirillum, Sphaerochaeta, LactoBacillus, Eubacterium, Corynebacter, Carnobacterium, Rhodobacter, Listeria, Paludibacter, Clostridium, Lachnospiraceae, Clostridiaridium, Leptotrichia, Francisella, Legionella, AlicycloBacillus, Methanomethyophilus, Porphyromonas, Prevotella, Bacteroidetes, Helcococcus, Leptospira, Desulfovibrio, Desulfonatronum, Opitutaceae, Tuberibacillus, Bacillus, Brevibacilus, Methylobacterium or Acidaminococcus.

In further particular embodiments, the Cpf1 effector protein is from an organism selected from S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, and C. sordellii.

The effector protein may comprise a chimeric effector protein comprising a first fragment from a first effector protein (e.g., a Cpf1) ortholog and a second fragment from a second effector (e.g., a Cpf1) protein ortholog, and, wherein the first and second effector protein orthologs are different. At least one of the first and second effector protein (e.g., a Cpf1) orthologs may comprise an effector protein (e.g., a Cpf1) from an organism comprising Streptococcus, Campylobacter, Nitratifractor, Staphylococcus, Parvibaculum, Roseburia, Neisseria, Gluconacetobacter, Azospirillum, Sphaerochaeta, LactoBacillus, Eubacterium, Corynebacter, Carnobacterium, Rhodobacter, Listeria, Paludibacter, Clostridium, Lachnospiraceae, Clostridiaridium, Leptotrichia, Francisella, Legionella, AlicycloBacillus, Methanomethyophilus, Porphyromonas, Prevotella, Bacteroidetes, Helcococcus, Leptospira, Desulfovibrio, Desulfonatronum, Opitutaceae, Tuberibacillus, Bacillus, Brevibacilus, Methylobacterium or Acidaminococcus; e.g., a chimeric effector protein comprising a first fragment and a second fragment wherein each of the first and second fragments is selected from a Cpf1 of an organism comprising Streptococcus, Campylobacter, Nitratifractor, Staphylococcus, Parvibaculum, Roseburia, Neisseria, Gluconacetobacter, Azospirillum, Sphaerochaeta, LactoBacillus, Eubacterium, Corynebacter, Carnobacterium, Rhodobacter, Listeria, Paludibacter, Clostridium, Lachnospiraceae, Clostridiaridium, Leptotrichia, Francisella, Legionella, AlicycloBacillus, Methanomethyophilus, Porphyromonas, Prevotella, Bacteroidetes, Helcococcus, Leptospira, Desulfovibrio, Desulfonatronum, Opitutaceae, Tuberibacillus, Bacillus, Brevibacilus, Methylobacterium or Acidaminococcus wherein the first and second fragments are not from the same bacteria; for instance a chimeric effector protein comprising a first fragment and a second fragment wherein each of the first and second fragments is selected from a Cpf1 of S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis 1, Prevotella albensis, Lachnospiraceae bacterium MC2017 1, Butyrivibrio proteoclasticus, Peregrinibacteria bacterium GW2011_GWA2 33 10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae, wherein the first and second fragments are not from the same bacteria.

In a more preferred embodiment, the Cpf1p is derived from a bacterial species selected from Francisella tularensis 1, Prevotella albensis, Lachnospiraceae bacterium MC2017 1, Butyrivibrio proteoclasticus, Peregrinibacteria bacterium GW2011_GWA2 33 10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. In certain embodiments, the Cpf1p is derived from a bacterial species selected from Acidaminococcus sp. BV3L6, Lachnospiraceae bacterium MA2020. In certain embodiments, the effector protein is derived from a subspecies of Francisella tularensis 1, including but not limited to Francisella tularensis subsp. Novicida.

In some embodiments, the Cpf1p is derived from an organism from the genus of Eubacterium. In some embodiments, the CRISPR effector protein is a Cpf1 protein derived from an organism from the bacterial species of Eubacterium rectale. In some embodiments, the amino acid sequence of the Cpf1 effector protein corresponds to NCBI Reference Sequence WP_055225123.1, NCBI Reference Sequence WP_055237260.1, NCBI Reference Sequence WP_055272206.1, or GenBank ID OLA16049.1. In some embodiments, the Cpf1 effector protein has a sequence homology or sequence identity of at least 60%, more particularly at least 70, such as at least 80%, more preferably at least 85%, even more preferably at least 90%, such as for instance at least 95%, with NCBI Reference Sequence WP_055225123.1, NCBI Reference Sequence WP_055237260.1, NCBI Reference Sequence WP_055272206.1, or GenBank ID OLA16049.1. The skilled person will understand that this includes truncated forms of the Cpf1 protein whereby the sequence identity is determined over the length of the truncated form. In some embodiments, the Cpf1 effector recognizes the PAM sequence of TTTN or CTTN.

In particular embodiments, the homologue or orthologue of Cpf1 as referred to herein has a sequence homology or identity of at least 80%, more preferably at least 85%, even more preferably at least 90%, such as for instance at least 95% with Cpf1. In further embodiments, the homologue or orthologue of Cpf1 as referred to herein has a sequence identity of at least 80%, more preferably at least 85%, even more preferably at least 90%, such as for instance at least 95% with the wild type Cpf1. Where the Cpf1 has one or more mutations (mutated), the homologue or orthologue of said Cpf1 as referred to herein has a sequence identity of at least 80%, more preferably at least 85%, even more preferably at least 90%, such as for instance at least 95% with the mutated Cpf1.

In an embodiment, the Cpf1 protein may be an ortholog of an organism of a genus which includes, but is not limited to Acidaminococcus sp, Lachnospiraceae bacterium or Moraxella bovoculi; in particular embodiments, the type V Cas protein may be an ortholog of an organism of a species which includes, but is not limited to Acidaminococcus sp. BV3L6; Lachnospiraceae bacterium ND2006 (LbCpf1) or Moraxella bovoculi 237. In particular embodiments, the homologue or orthologue of Cpf1 as referred to herein has a sequence homology or identity of at least 80%, more preferably at least 85%, even more preferably at least 90%, such as for instance at least 95% with one or more of the Cpf1 sequences disclosed herein. In further embodiments, the homologue or orthologue of Cpf1 as referred to herein has a sequence identity of at least 80%, more preferably at least 85%, even more preferably at least 90%, such as for instance, at least 95% with the wild type FnCpf1, AsCpf1 or LbCpf1.

In particular embodiments, the Cpf1 protein of the disclosure has a sequence homology or identity of at least 60%, more particularly at least 70, such as at least 80%, more preferably at least 85%, even more preferably at least 90%, such as for instance at least 95% with FnCpf1, AsCpf1 or LbCpf1. In further embodiments, the Cpf1 protein as referred to herein has a sequence identity of at least 60%, such as at least 70%, more particularly at least 80%, more preferably at least 85%, even more preferably at least 90%, such as for instance at least 95% with the wild type AsCpf1 or LbCpf1. In particular embodiments, the Cpf1 protein of the present disclosure has less than 60% sequence identity with FnCpf1. The skilled person will understand that this includes truncated forms of the Cpf1 protein whereby the sequence identity is determined over the length of the truncated form.

Further Cpf1 orthologs include NCBI WP_055225123.1, NCBI WP_055237260.1, NCBI WP_055272206.1, and GenBank OLA16049.1.

### C2c1 Orthologs

The present disclosure encompasses the use of a C2c1 effector proteins, derived from a C2c1 locus denoted as subtype V-B. Herein such effector proteins are also referred to as “C2c1p”, e.g., a C2c1 protein (and such effector protein or C2c1 protein or protein derived from a C2c1 locus is also called “CRISPR enzyme”). Presently, the subtype V-B loci encompasses cas1-Cas4 fusion, cas2, a distinct gene denoted C2c1 and a CRISPR array. C2c1 (CRISPR-associated protein C2c1) is a large protein (about 1100-1300 amino acids) that contains a RuvC-like nuclease domain homologous to the corresponding domain of Cas9 along with a counterpart to the characteristic arginine-rich cluster of Cas9. However, C2c1 lacks the HNH nuclease domain that is present in all Cas9 proteins, and the RuvC-like domain is contiguous in the C2c1 sequence, in contrast to Cas9 where it contains long inserts including the HNH domain. Accordingly, in particular embodiments, the CRISPR-Cas enzyme comprises only a RuvC-like nuclease domain.

C2c1 (also known as Cas12b) proteins are RNA guided nucleases. Its cleavage relies on a tracr RNA to recruit a guide RNA comprising a guide sequence and a direct repeat, where the guide sequence hybridizes with the target nucleotide sequence to form a DNA/RNA heteroduplex. Based on current studies, C2c1 nuclease activity also requires relies on recognition of PAM sequence. C2c1 PAM sequences are T-rich sequences. In some embodiments, the PAM sequence is 5′ TTN 3′ or 5′ ATTN 3′, wherein N is any nucleotide. In a particular embodiment, the PAM sequence is 5′ TTC 3′. In a particular embodiment, the PAM is in the sequence of Plasmodium falciparum.

C2c1 creates a staggered cut at the target locus, with a 5′ overhang, or a “sticky end” at the PAM distal side of the target sequence. In some embodiments, 5′ overhang is 7 nt. See Lewis and Ke, Mol Cell. 2017 Feb. 2; 65(3):377-379.

The disclosure provides C2c1 (Type V-B; Cas12b) effector proteins and orthologues. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related or are only partially structurally related. An “orthologue” of a protein as used herein is a protein of a different species which performs the same or a similar function as the protein it is an orthologue of. Orthologous proteins may but need not be structurally related or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22(4):359-66. doi: 10.1002/pro.2225). See also Shmakov et al. (2015) for application in the field of CRISPR-Cas loci. Homologous proteins may but need not be structurally related or are only partially structurally related.

The C2c1 gene is found in several diverse bacterial genomes, typically in the same locus with cas1, cas2, and cas4 genes and a CRISPR cassette. Thus, the layout of this putative novel CRISPR-Cas system appears to be like that of type II-B. Furthermore, like Cas9, the C2c1 protein contains an active RuvC-like nuclease, an arginine-rich region, and a Zn finger (absent in Cas9).

In particular embodiments, the effector protein is a C2c1 effector protein from an organism from a genus comprising AlicycloBacillus, Desulfovibrio, Desulfonatronum, Opitutaceae, Tuberibacillus, Bacillus, BreviBacillus, Candidatus, Desulfatirhabdium, Citrobacter, Elusimicrobia, Methylobacterium, Omnitrophica, Phycisphaerae, Planctomycetes, Spirochaetes, and Verrucomicrobiaceae.

In further particular embodiments, the C2c1 effector protein is from a species selected from AlicycloBacillus acidoterrestris (e.g., ATCC 49025), AlicycloBacillus contaminans (e.g., DSM 17975), AlicycloBacillus macrosporangiidus (e.g. DSM 17980), Bacillus hisashii strain C4, Candidatus Lindo bacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13 46 10, Spirochaetes bacterium GWB1_27_13, Verrucomicrobiaceae bacterium UBA2429, TuberiBacillus calidus (e.g., DSM 17572), Bacillus thermoamylovorans (e.g., strain B4166), BreviBacillus sp. CF112, Bacillus sp. NSP2.1, Desulfatirhabdium butyrativorans (e.g., DSM 18734), AlicycloBacillus herbarius (e.g., DSM 13609), Citrobacter freundii (e.g., ATCC 8090), BreviBacillus agri (e.g., BAB-2500), Methylobacterium nodulans (e.g., ORS 2060).

The effector protein may comprise a chimeric effector protein comprising a first fragment from a first effector protein (e.g., a C2c1) ortholog and a second fragment from a second effector (e.g., a C2c1) protein ortholog, and wherein the first and second effector protein orthologs are different. At least one of the first and second effector protein (e.g., a C2c1) orthologs may comprise an effector protein (e.g., a C2c1) from an organism comprising AlicycloBacillus, Desulfovibrio, Desulfonatronum, Opitutaceae, TuberiBacillus, Bacillus, BreviBacillus, Candidatus, Desulfatirhabdium, Elusimicrobia, Citrobacter, Methylobacterium, Omnitrophicai, Phycisphaerae, Planctomycetes, Spirochaetes, and Verrucomicrobiaceae; e.g., a chimeric effector protein comprising a first fragment and a second fragment wherein each of the first and second fragments is selected from a C2c1 of an organism comprising AlicycloBacillus, Desulfovibrio, Desulfonatronum, Opitutaceae, TuberiBacillus, Bacillus, BreviBacillus, Candidatus, Desulfatirhabdium, Elusimicrobia, Citrobacter, Methylobacterium, Omnitrophicai, Phycisphaerae, Planctomycetes, Spirochaetes, and Verrucomicrobiaceae wherein the first and second fragments are not from the same bacteria; for instance a chimeric effector protein comprising a first fragment and a second fragment wherein each of the first and second fragments is selected from a C2c1 of AlicycloBacillus acidoterrestris (e.g., ATCC 49025), AlicycloBacillus contaminans (e.g., DSM 17975), AlicycloBacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG 13_46_10, Spirochaetes bacterium GWB1_27_13, Verrucomicrobiaceae bacterium UBA2429, TuberiBacillus calidus (e.g., DSM 17572), Bacillus thermoamylovorans (e.g., strain B4166), BreviBacillus sp. CF112, Bacillus sp. NSP2.1, Desulfatirhabdium butyrativorans (e.g., DSM 18734), AlicycloBacillus herbarius (e.g., DSM 13609), Citrobacter freundii (e.g., ATCC 8090), BreviBacillus agri (e.g., BAB-2500), Methylobacterium nodulans (e.g., ORS 2060), wherein the first and second fragments are not from the same bacteria.

In a more preferred embodiment, the C2c1p is derived from a bacterial species selected from AlicycloBacillus acidoterrestris (e.g., ATCC 49025), AlicycloBacillus contaminans (e.g., DSM 17975), AlicycloBacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG 13 46_10, Spirochaetes bacterium GWB1_27_13, Verrucomicrobiaceae bacterium UBA2429, TuberiBacillus calidus (e.g., DSM 17572), Bacillus thermoamylovorans (e.g., strain B4166), BreviBacillus sp. CF112, Bacillus sp. NSP2.1, Desulfatirhabdium butyrativorans (e.g., DSM 18734), AlicycloBacillus herbarius (e.g., DSM 13609), Citrobacter freundii (e.g., ATCC 8090), BreviBacillus agri (e.g., BAB-2500), Methylobacterium nodulans (e.g., ORS 2060). In certain embodiments, the C2c1p is derived from a bacterial species selected from AlicycloBacillus acidoterrestris (e.g., ATCC 49025), AlicycloBacillus contaminans (e.g., DSM 17975).

In particular embodiments, the homologue or orthologue of C2c1 as referred to herein has a sequence homology or identity of at least 80%, more preferably at least 85%, even more preferably at least 90%, such as for instance at least 95% with C2c1. In further embodiments, the homologue or orthologue of C2c1 as referred to herein has a sequence identity of at least 80%, more preferably at least 85%, even more preferably at least 90%, such as for instance at least 95% with the wild type C2c1. Where the C2c1 has one or more mutations (mutated), the homologue or orthologue of said C2c1 as referred to herein has a sequence identity of at least 80%, more preferably at least 85%, even more preferably at least 90%, such as for instance at least 95% with the mutated C2c1.

In an embodiment, the C2c1 protein may be an ortholog of an organism of a genus which includes, but is not limited to AlicycloBacillus, Desulfovibrio, Desulfonatronum, Opitutaceae, TuberiBacillus, Bacillus, BreviBacillus, Candidatus, Desulfatirhabdium, Elusimicrobia, Citrobacter, Methylobacterium, Omnitrophicai, Phycisphaerae, Planctomycetes, Spirochaetes, and Verrucomicrobiaceae; in particular embodiments, the type V Cas protein may be an ortholog of an organism of a species which includes, but is not limited to AlicycloBacillus acidoterrestris (e.g., ATCC 49025), AlicycloBacillus contaminans (e.g., DSM 17975), AlicycloBacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13_46_10, Spirochaetes bacterium GWB1_27_13, Verrucomicrobiaceae bacterium UBA2429, TuberiBacillus calidus (e.g., DSM 17572), Bacillus thermoamylovorans (e.g., strain B4166), BreviBacillus sp. CF112, Bacillus sp. NSP2.1, Desulfatirhabdium butyrativorans (e.g., DSM 18734), AlicycloBacillus herbarius (e.g., DSM 13609), Citrobacter freundii (e.g., ATCC 8090), BreviBacillus agri (e.g., BAB-2500), Methylobacterium nodulans (e.g., ORS 2060). In particular embodiments, the homologue or orthologue of C2c1 as referred to herein has a sequence homology or identity of at least 80%, more preferably at least 85%, even more preferably at least 90%, such as for instance at least 95% with one or more of the C2c1 sequences disclosed herein. In further embodiments, the homologue or orthologue of C2c1 as referred to herein has a sequence identity of at least 80%, more preferably at least 85%, even more preferably at least 90%, such as for instance at least 95% with the wild type AacC2c1 or BthC2c1.

In particular embodiments, the C2c1 protein of the disclosure has a sequence homology or identity of at least 60%, more particularly at least 70, such as at least 80%, more preferably at least 85%, even more preferably at least 90%, such as for instance at least 95% with AacC2c1 or BthC2c1. In further embodiments, the C2c1 protein as referred to herein has a sequence identity of at least 60%, such as at least 70%, more particularly at least 80%, more preferably at least 85%, even more preferably at least 90%, such as for instance at least 95% with the wild type AacC2c1. In particular embodiments, the C2c1 protein of the present disclosure has less than 60% sequence identity with AacC2c1. The skilled person will understand that this includes truncated forms of the C2c1 protein whereby the sequence identity is determined over the length of the truncated form.

In certain methods according to the present disclosure, the CRISPR-Cas protein is preferably mutated with respect to a corresponding wild-type enzyme such that the mutated CRISPR-Cas protein lacks the ability to cleave one or both DNA strands of a target locus containing a target sequence. In particular embodiments, one or more catalytic domains of the C2c1 protein are mutated to produce a mutated Cas protein which cleaves only one DNA strand of a target sequence.

In particular embodiments, the CRISPR-Cas protein may be mutated with respect to a corresponding wild-type enzyme such that the mutated CRISPR-Cas protein lacks substantially all DNA cleavage activity. In some embodiments, a CRISPR-Cas protein may be considered to substantially lack all DNA and/or RNA cleavage activity when the cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form.

In certain embodiments of the methods provided herein the CRISPR-Cas protein is a mutated CRISPR-Cas protein which cleaves only one DNA strand, i.e. a nickase. More particularly, in the context of the present disclosure, the nickase ensures cleavage within the non-target sequence, i.e. the sequence which is on the opposite DNA strand of the target sequence and which is 3′ of the PAM sequence. By means of further guidance, and without limitation, an arginine-to-alanine substitution (R911A) in the Nuc domain of C2c1 from AlicycloBacillus acidoterrestris converts C2c1 from a nuclease that cleaves both strands to a nickase (cleaves a single strand). It will be understood by the skilled person that where the enzyme is not AacC2c1, a mutation may be made at a residue in a corresponding position.

In certain embodiments, the C2c1 protein is a catalytically inactive C2c1 which comprises a mutation in the RuvC domain. In some embodiments, the catalytically inactive C2c1 protein comprises a mutation corresponding to amino acid positions D570, E848, or D977 in AlicycloBacillus acidoterrestris C2c1. In some embodiments, the catalytically inactive C2c1 protein comprises a mutation corresponding to D570A, E848A, or D977A in AlicycloBacillus acidoterrestris C2c1.

The programmability, specificity, and collateral activity of the RNA-guided C2c1 also make it an ideal switchable nuclease for non-specific cleavage of nucleic acids. In one embodiment, a C2c1 system is engineered to provide and take advantage of collateral non-specific cleavage of RNA. In another embodiment, a C2c1 system is engineered to provide and take advantage of collateral non-specific cleavage of ssDNA. Accordingly, engineered C2c1 systems provide platforms for nucleic acid detection and transcriptome manipulation, and inducing cell death. C2c1 is developed for use as a mammalian transcript knockdown and binding tool. C2c1 is capable of robust collateral cleavage of RNA and ssDNA when activated by sequence-specific targeted DNA binding.

Collateral activity was recently leveraged for a highly sensitive and specific nucleic acid detection platform termed SHERLOCK that is useful for many clinical diagnoses (Gootenberg, J. S. et al. Nucleic acid detection with CRISPR-Cas13a/C2c2. Science 356, 438-442 (2017)).

According to the disclosure, engineered C2c1 systems are optimized for DNA or RNA endonuclease activity.

### Guide Sequences

As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

As used herein, the term “guide sequence,” “crRNA,” “guide RNA,” or “single guide RNA,” or “gRNA” refers to a polynucleotide comprising any polynucleotide sequence having sufficient complementarity with a target nucleic acid sequence to hybridize with the target nucleic acid sequence and to direct sequence-specific binding of an RNA-targeting complex comprising the guide sequence and a CRISPR effector protein to the target nucleic acid sequence. In some example embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence, and hence a nucleic acid-targeting guide may be selected to target any target nucleic acid sequence. The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoide cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the disclosure, a guide nucleic acid comprises ribonucleotides and non-ribonucleotides. In one such embodiment, a guide comprises one or more ribonucleotides and one or more deoxyribonucleotides. In an embodiment of the disclosure, the guide comprises one or more non-naturally occurring nucleotide or nucleotide analog such as a nucleotide with phosphorothioate linkage, a locked nucleic acid (LNA) nucleotide comprising a methylene bridge between the 2′ and 4′ carbons of the ribose ring or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′phosphorothioate (MS), S-constrained ethyl (cEt), or 2′-O-methyl 3′thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33 (9): 985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33 (9): 985-989; Li et al., Nature Biomedical Engineering, 2017, 1, 0066 DOI: 10.1038/s41551-017-0066). In some embodiments, 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucleotides and/or nucleotide analogs in a region that binds to Cas13. In an embodiment of the disclosure, deoxyribonucleotides and/or nucleotide analogs are incorporated in engineered guide structures, such as, without limitation, stem-loop regions, and the seed region. For Cas13 guide, in certain embodiments, the modification is not in 5′-handle of the stem-loop regions. Chemical modification in 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either 3′ or 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at 5′ and/or 3′ end of the guide are chemically modified with 2′-O-methyl (M), 2′-O-methyl 3′ phosphorothioate (MS), S-constrained ethyl (cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at 5′ and/or 3′ end of the guide are chemically modified with 2′-O-Me, 2′-F or S-constrained ethyl (cEt). Such chemically modified guide can mediate enhanced levels of gene disruption (see Ragdarm et al., 0215, PNAS, E7110-E7111). In an embodiment of the disclosure, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6: e25312, DOI: 10.7554).

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer.

In general, the CRISPR-Cas, CRISPR-Cas9 or CRISPR system may be as used in the foregoing documents, such as WO 2014/093622 (PCT/US2013/074667) and refers collectively to transcripts and other elements involved in the expression of or directing the activity of CRISPR-associated (“Cas”) genes, including sequences encoding a Cas gene, in particular a Cas9 gene in the case of CRISPR-Cas9, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. The section of the guide sequence through which complementarity to the target sequence is important for cleavage activity is referred to herein as the seed sequence. A target sequence may comprise any polynucleotide, such as DNA or RNA polynucleotides. In some embodiments, direct repeats may be identified in silico by searching for repetitive motifs that fulfill any or all of the following criteria: 1. found in a 2 Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used.

In embodiments of the disclosure the terms guide sequence and guide RNA, i.e. RNA capable of guiding Cas to a target genomic locus, are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments of CRISPR-Cas systems, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and advantageously tracr RNA is 30 or 50 nucleotides in length. However, an aspect of the disclosure is to reduce off-target interactions, e.g., reduce the guide interacting with a target sequence having low complementarity. Indeed, in the examples, it is shown that the disclosure involves mutations that result in the CRISPR-Cas system being able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in the context of the present disclosure the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

### a) Guide Modifications

In certain embodiments, guides of the disclosure comprise non-naturally occurring nucleic acids and/or non-naturally occurring nucleotides and/or nucleotide analogs, and/or chemical modifications. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the disclosure, a guide nucleic acid comprises ribonucleotides and non-ribonucleotides. In one such embodiment, a guide comprises one or more ribonucleotides and one or more deoxyribonucleotides. In an embodiment of the disclosure, the guide comprises one or more non-naturally occurring nucleotide or nucleotide analog such as a nucleotide with phosphorothioate linkage, organophosphate linkage, a locked nucleic acid (LNA) nucleotide comprising a methylene bridge between the 2′ and 4′ carbons of the ribose ring or bridged nucleic acids (BNA). Other examples of modified nucleotides include 2′-O-methyl analogs, 2′-deoxy analogs, 2-thiouridine analogs, N6-methyladenosine analogs, or 2′-fluoro analogs. Further examples of modified bases include, but are not limited to, 2-aminopurine, 5-bromo-uridine, pseudouridine (Ψ), N1-methylpseudouridine (me1Ψ), 5-methoxyuridine (5moU), inosine, 7-methylguanosine. Examples of guide RNA chemical modifications include, without limitation, incorporation of 2′-O-methyl (M), 2′-O-methyl-3′-phosphorothioate (MS), phosphorothioate (PS), S-constrained ethyl (cEt), or 2′-O-methyl-3′-thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015; Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI: 10.1038/s41551-017-0066). In some embodiments, 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target DNA and one or more deoxyribonucleotides and/or nucleotide analogs in a region that binds to Cas9, Cpf1, or C2c1. In an embodiment of the disclosure, deoxyribonucleotides and/or nucleotide analogs are incorporated in engineered guide structures, such as, without limitation, 5′ and/or 3′ end, stem-loop regions, and the seed region. In certain embodiments, the modification is not in 5′-handle of the stem-loop regions. Chemical modification in 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either 3′ or 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at 5′ and/or 3′ end of the guide are chemically modified with 2′-O-methyl (M), 2′-O-methyl-3′-phosphorothioate (MS), S-constrained ethyl (cEt), or 2′-O-methyl-3′-thioPACE (MSP). Such modification can enhance genome editing efficiency (see Hendel et al., Nat. Biotechnol. (2015) 33 (9): 985-989). In certain embodiments, all of the phosphodiester bonds of a guide are substituted with phosphorothioates (PS) for enhancing levels of gene disruption. In certain embodiments, more than five nucleotides at 5′ and/or 3′ end of the guide are chemically modified with 2′-O-Me, 2′-F or S-constrained ethyl (cEt). Such chemically modified guide can mediate enhanced levels of gene disruption (see Ragdarm et al., 0215, PNAS, E7110-E7111). In an embodiment of the disclosure, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles.

In certain embodiments, the CRISPR system as provided herein can make use of a crRNA or analogous polynucleotide comprising a guide sequence, wherein the polynucleotide is an RNA, a DNA or a mixture of RNA and DNA, and/or wherein the polynucleotide comprises one or more nucleotide analogs. The sequence can comprise any structure, including but not limited to a structure of a native crRNA, such as a bulge, a hairpin or a stem loop structure. In certain embodiments, the polynucleotide comprising the guide sequence forms a duplex with a second polynucleotide sequence which can be an RNA or a DNA sequence.

In certain embodiments, use is made of chemically modified guide RNAs. Examples of guide RNA chemical modifications include, without limitation, incorporation of 2′-O-methyl (M), 2′-O-methyl 3′phosphorothioate (MS), or 2′-O-methyl 3′thioPACE (MSP) at one or more terminal nucleotides. Such chemically modified guide RNAs can comprise increased stability and increased activity as compared to unmodified guide RNAs, though on-target vs. off-target specificity is not predictable. (See, Hendel, 2015, Nat Biotechnol. 33 (9): 985-9, doi: 10.1038/nbt.3290, published online 29 Jun. 2015). Chemically modified guide RNAs further include, without limitation, RNAs with phosphorothioate linkages and locked nucleic acid (LNA) nucleotides comprising a methylene bridge between the 2′ and 4′ carbons of the ribose ring.

In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10 to 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay. Similarly, cleavage of a target RNA may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions.

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (me1Ψ), 5-methoxyuridine (5moU), inosine, 7-methylguanosine, 2′-O-methyl-3′-phosphorothioate (MS), S-constrained ethyl (cEt), phosphorothioate (PS), or 2′-O-methyl-3′-thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in 3′-terminus are chemically modified. In certain embodiments, none of the nucleotides in 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 or 10 nucleotides in 3′-terminus are chemically modified. Such chemical modifications at 3′-terminus of the Cpf1 CrRNA improve gene cutting efficiency (see Li, et al., Nature Biomedical Engineering, 2017, 1:0066). In a specific embodiment, 5 nucleotides in 3′-terminus are replaced with 2′-fluoro analogues. In a specific embodiment, 10 nucleotides in 3′-terminus are replaced with 2′-fluoro analogues. In a specific embodiment, 5 nucleotides in 3′-terminus are replaced with 2′-O-methyl (M) analogs.

In some embodiments, the loop of 5′-handle of the guide is modified. In some embodiments, the loop of 5′-handle of the guide is modified to have a deletion, an insertion, a split, or chemical modifications. In certain embodiments, the loop comprises 3, 4, or 5 nucleotides. In certain embodiments, the loop comprises the sequence of UCUU, UUUU, UAUU, or UGUU.

A guide sequence, and hence a nucleic acid-targeting guide RNA may be selected to target any target nucleic acid sequence. In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nuclear RNA (snoRNA), double stranded RNA (dsRNA), non-coding RNA (ncRNA), long non-coding RNA (lncRNA), and small cytoplasmic RNA (scRNA). In some preferred embodiments, the target sequence may be a sequence within an RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In certain embodiments, the spacer length of the guide RNA is less than 28 nucleotides. In certain embodiments, the spacer length of the guide RNA is at least 18 nucleotides and less than 28 nucleotides. In certain embodiments, the spacer length of the guide RNA is between 19 and 28 nucleotides. In certain embodiments, the spacer length of the guide RNA is between 19 and 25 nucleotides. In certain embodiments, the spacer length of the guide RNA is 20 nucleotides. In certain embodiments, the spacer length of the guide RNA is 23 nucleotides. In certain embodiments, the spacer length of the guide RNA is 25 nucleotides.

In certain embodiments, modulations of cleavage efficiency can be exploited by introduction of mismatches, e.g. 1 or more mismatches, such as 1 or 2 mismatches between spacer sequence and target sequence, including the position of the mismatch along the spacer/target. The more central (i.e. not 3′ or 5′) for instance a double mismatch is, the more cleavage efficiency is affected. Accordingly, by choosing mismatch position along the spacer, cleavage efficiency can be modulated. By means of example, if less than 100% cleavage of targets is desired (e.g. in a cell population), 1 or more, such as preferably 2 mismatches between spacer and target sequence may be introduced in the spacer sequences. The more central along the spacer of the mismatch position, the lower the cleavage percentage.

### b) Single Nucleotide Polymorphisms (SNPs)

The term “single nucleotide polymorphism” (SNP) refers to a DNA sequence variation occurring when a single nucleotide—A, T, C, or G—in the genome that differs between members of a paired chromosomes in a microorganism. For example, two sequenced DNA fragments from different microorganisms, AAGCCTA to AAGCTTA, contain a difference in a single nucleotide. In this case we say that there are two alleles: C and T. Almost all common SNPs have only two alleles.

In certain example embodiments, the cleavage efficiency may be exploited to design single guides that can distinguish two or more targets that vary by a single nucleotide, such as a single nucleotide polymorphism (SNP), variation, or (point) mutation. The CRISPR effector may have reduced sensitivity to SNPs (or other single nucleotide variations) and continue to cleave SNP targets with a certain level of efficiency. Thus, for two targets, or a set of targets, a guide RNA may be designed with a nucleotide sequence that is complementary to one of the targets i.e. the on-target SNP. The guide RNA is further designed to have a synthetic mismatch. As used herein a “synthetic mismatch” refers to a non-naturally occurring mismatch that is introduced upstream or downstream of the naturally occurring SNP, such as at most 5 nucleotides upstream or downstream, for instance 4, 3, 2, or 1 nucleotide upstream or downstream, preferably at most 3 nucleotides upstream or downstream, more preferably at most 2 nucleotides upstream or downstream, most preferably 1 nucleotide upstream or downstream (i.e. adjacent the SNP). When the CRISPR effector binds to the on-target SNP, only a single mismatch will be formed with the synthetic mismatch and the CRISPR effector will continue to be activated and a detectable signal produced. When the guide RNA hybridizes to an off-target SNP, two mismatches will be formed, the mismatch from the SNP and the synthetic mismatch, and no detectable signal generated. Thus, the systems disclosed herein may be designed to distinguish SNPs within a population. For, example the systems may be used to distinguish pathogenic strains that differ by a single SNP or detect certain disease specific SNPs, such as but not limited to, disease associated SNPs, such as without limitation cancer associated SNPs.

In certain embodiments, the guide RNA is designed such that the SNP is located on position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the spacer sequence (starting at the 5′ end). In certain embodiments, the guide RNA is designed such that the SNP is located on position 1, 2, 3, 4, 5, 6, 7, 8, or 9 of the spacer sequence (starting at the 5′ end). In certain embodiments, the guide RNA is designed such that the SNP is located on position 2, 3, 4, 5, 6, or 7 of the spacer sequence (starting at the 5′ end). In certain embodiments, the guide RNA is designed such that the SNP is located on position 3, 4, 5, or 6 of the spacer sequence (starting at the 5′ end). In certain embodiments, the guide RNA is designed such that the SNP is located on position 3 of the spacer sequence (starting at the 5′ end).

In certain embodiments, the guide RNA is designed such that the mismatch (e.g. the synthetic mismatch, i.e. an additional mutation besides a SNP) is located on position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the spacer sequence (starting at the 5′ end). In certain embodiments, the guide RNA is designed such that the mismatch is located on position 1, 2, 3, 4, 5, 6, 7, 8, or 9 of the spacer sequence (starting at the 5′ end). In certain embodiments, the guide RNA is designed such that the mismatch is located on position 4, 5, 6, or 7 of the spacer sequence (starting at the 5′ end. In certain embodiments, the guide RNA is designed such that the mismatch is located at position 3, 4, 5, or 6 of the spacer, preferably position 3. In certain embodiments, the guide RNA is designed such that the mismatch is located on position 5 of the spacer sequence (starting at the 5′ end).

In certain embodiments, said mismatch is 1, 2, 3, 4, or 5 nucleotides upstream or downstream, preferably 2 nucleotides, preferably downstream of said SNP or other single nucleotide variation in said guide RNA.

In certain embodiments, the guide RNA is designed such that the mismatch is located 2 nucleotides upstream of the SNP (i.e. one intervening nucleotide).

In certain embodiments, the guide RNA is designed such that the mismatch is located 2 nucleotides downstream of the SNP (i.e. one intervening nucleotide).

In certain embodiments, the guide RNA is designed such that the mismatch is located on position 5 of the spacer sequence (starting at the 5′ end) and the SNP is located on position 3 of the spacer sequence (starting at the 5′ end).

In certain embodiments, the guide RNA comprises a spacer which is truncated relative to a wild type spacer. In certain embodiments, the guide RNA comprises a spacer which comprises less than 28 nucleotides, preferably between and including 20 to 27 nucleotides.

In certain embodiments, the guide RNA comprises a spacer which consists of 20-25 nucleotides or 20-23 nucleotides, such as preferably 20 or 23 nucleotides.

In certain embodiments, the one or more guide RNAs are designed to detect a single nucleotide polymorphism in a target RNA or DNA, or a splice variant of an RNA transcript.

Example orthologs are provided in TABLE XXVII below.

TABLE XXVII
Host

AlicycloBacillus macrosporangiidus strain DSM 17980 (SEQ ID
NO: 191)
Bacillus hisashii strain C4 (SEQ ID NO: 192)
Candidatus Lindowbacteria bacterium RIFCSPLOWO2 (SEQ ID
NO: 193)
Elusimicrobia bacterium RIFOXYA12 (SEQ ID NO: 194)
Omnitrophica WOR_2 bacterium RIFCSPHIGHO2 (SEQ ID NO: 195)
Phycisphaerae bacterium ST-NAGAB-D1 (SEQ ID NO: 196)
Planctomycetes bacterium RBG_13_46_10 (SEQ ID NO: 197)
Spirochaetes bacterium GWB1_27_13 (SEQ ID NO: 198)
Verrucomicrobiaceae bacterium UBA2429(SEQ ID NO: 199)

### Detection Constructs

As used herein, a “detection construct” refers to a molecule that can be cleaved or otherwise deactivated by an activated CRISPR system effector protein described herein. The term “detection construct” may also be referred to in the alternative as a “masking construct.” Depending on the nuclease activity of the CRISPR effector protein, the detection construct may be an RNA-based detection construct or a DNA-based detection construct. The Nucleic Acid-based detection constructs comprises a nucleic acid element that is cleavable by a CRISPR effector protein. Cleavage of the nucleic acid element releases agents or produces conformational changes that allow a detectable signal to be produced. Example constructs demonstrating how the nucleic acid element may be used to prevent or mask generation of detectable signal are described below and embodiments of the disclosure comprise variants of the same. Prior to cleavage, or when the detection construct is in an ‘active’ state, the detection construct blocks the generation or detection of a positive detectable signal. It will be understood that in certain example embodiments a minimal background signal may be produced in the presence of an active detection construct. A positive detectable signal may be any signal that can be detected using optical, fluorescent, chemiluminescent, electrochemical or other detection methods known in the art. The term “positive detectable signal” is used to differentiate from other detectable signals that may be detectable in the presence of the detection construct. For example, in certain embodiments a first signal may be detected when the masking agent is present (i.e. a negative detectable signal), which then converts to a second signal (e.g. the positive detectable signal) upon detection of the target molecules and cleavage or deactivation of the masking agent by the activated CRISPR effector protein.

In certain example embodiments, the detection construct may comprise an HCR initiator sequence and a cutting motif, or a cleavable structural element, such as a loop or hairpin, that prevents the initiator from initiating the HCR reaction. The cutting motif may be preferentially cut by one of the activated CRISPR effector proteins. Upon cleavage of the cutting motif or structure element by an activated CRISPR effector protein, the initiator is then released to trigger the HCR reaction, detection thereof indicating the presence of one or more targets in the sample. In certain example embodiments, the detection construct comprises a hairpin with an RNA loop. When an activated CRISPR effector protein cuts the RNA loop, the initiator can be released to trigger the HCR reaction.

In certain example embodiments, the detection construct may suppress generation of a gene product. The gene product may be encoded by a reporter construct that is added to the sample. The detection construct may be an interfering RNA involved in an RNA interference pathway, such as a short hairpin RNA (shRNA) or small interfering RNA (siRNA). The detection construct may also comprise microRNA (miRNA). While present, the detection construct suppresses expression of the gene product. The gene product may be a fluorescent protein or other RNA transcript or proteins that would otherwise be detectable by a labeled probe, aptamer, or antibody but for the presence of the detection construct. Upon activation of the effector protein the detection construct is cleaved or otherwise silenced allowing for expression and detection of the gene product as the positive detectable signal.

In specific embodiments, the detection construct comprises a silencing RNA that suppresses generation of a gene product encoded by a reporting construct, wherein the gene product generates the detectable positive signal when expressed.

In certain example embodiments, the detection construct may sequester one or more reagents needed to generate a detectable positive signal such that release of the one or more reagents from the detection construct results in generation of the detectable positive signal. The one or more reagents may combine to produce a colorimetric signal, a chemiluminescent signal, a fluorescent signal, or any other detectable signal and may comprise any reagents known to be suitable for such purposes. In certain example embodiments, the one or more reagents are sequestered by RNA aptamers that bind the one or more reagents. The one or more reagents are released when the effector protein is activated upon detection of a target molecule and the RNA or DNA aptamers are degraded.

In certain example embodiments, the detection construct may be immobilized on a solid substrate in an individual discrete volume (defined further below) and sequesters a single reagent. For example, the reagent may be a bead comprising a dye. When sequestered by the immobilized reagent, the individual beads are too diffuse to generate a detectable signal, but upon release from the detection construct are able to generate a detectable signal, for example by aggregation or simple increase in solution concentration. In certain example embodiments, the immobilized masking agent is a RNA- or DNA-based aptamer that can be cleaved by the activated effector protein upon detection of a target molecule.

In certain other example embodiments, the detection construct binds to an immobilized reagent in solution thereby blocking the ability of the reagent to bind to a separate labeled binding partner that is free in solution. Thus, upon application of a washing step to a sample, the labeled binding partner can be washed out of the sample in the absence of a target molecule. However, if the effector protein is activated, the detection construct is cleaved to a degree sufficient to interfere with the ability of the detection construct to bind the reagent thereby allowing the labeled binding partner to bind to the immobilized reagent. Thus, the labeled binding partner remains after the wash step indicating the presence of the target molecule in the sample. In certain aspects, the detection construct that binds the immobilized reagent is a DNA or RNA aptamer. The immobilized reagent may be a protein and the labeled binding partner may be a labeled antibody. Alternatively, the immobilized reagent may be streptavidin and the labeled binding partner may be labeled biotin. The label on the binding partner used in the above embodiments may be any detectable label known in the art. In addition, other known binding partners may be used in accordance with the overall design described herein.

In certain example embodiments, the detection construct may comprise a ribozyme. Ribozymes are RNA molecules having catalytic properties. Ribozymes, both naturally and engineered, comprise or consist of RNA that may be targeted by the effector proteins disclosed herein. The ribozyme may be selected or engineered to catalyze a reaction that either generates a negative detectable signal or prevents generation of a positive control signal. Upon deactivation of the ribozyme by the activated effector protein the reaction generating a negative control signal, or preventing generation of a positive detectable signal, is removed thereby allowing a positive detectable signal to be generated. In one example embodiment, the ribozyme may catalyze a colorimetric reaction causing a solution to appear as a first color. When the ribozyme is deactivated, the solution then turns to a second color, the second color being the detectable positive signal. An example of how ribozymes can be used to catalyze a colorimetric reaction are described in Zhao et al. “Signal amplification of glucosamine-6-phosphate based on ribozyme glmS,” Biosens Bioelectron. 2014; 16:337-42 and provide an example of how such a system could be modified to work in the context of the embodiments disclosed herein. Alternatively, ribozymes, when present can generate cleavage products of, for example, RNA transcripts. Thus, detection of a positive detectable signal may comprise detection of non-cleaved RNA transcripts that are only generated in the absence of the ribozyme.

In some embodiments, the detection construct may be a ribozyme that generates a negative detectable signal, and wherein a positive detectable signal is generated when the ribozyme is deactivated.

In certain example embodiments, the one or more reagents is a protein, such as an enzyme, capable of facilitating generation of a detectable signal, such as a colorimetric, chemiluminescent, or fluorescent signal, that is inhibited or sequestered such that the protein cannot generate the detectable signal by the binding of one or more DNA or RNA aptamers to the protein. Upon activation of the effector proteins disclosed herein, the DNA or RNA aptamers are cleaved or degraded to an extent that they no longer inhibit the protein's ability to generate the detectable signal. In certain example embodiments, the aptamer is a thrombin inhibitor aptamer. In certain example embodiments the thrombin inhibitor aptamer has a sequence of GGGAACAAAGCUGAAGUACUUACCC (SEQ ID NO:200). When this aptamer is cleaved, thrombin will become active and will cleave a peptide colorimetric or fluorescent substrate. In certain example embodiments, the colorimetric substrate is para-nitroanilide (pNA) covalently linked to the peptide substrate for thrombin. Upon cleavage by thrombin, pNA is released and becomes yellow in color and easily visible to the eye. In certain example embodiments, the fluorescent substrate is 7-amino-4-methylcoumarin a blue fluorophore that can be detected using a fluorescence detector. Inhibitory aptamers may also be used for horseradish peroxidase (HRP), beta-galactosidase, or calf alkaline phosphatase (CAP) and within the general principals laid out above.

In certain embodiments, RNAse or DNAse activity is detected colorimetrically via cleavage of enzyme-inhibiting aptamers. One potential mode of converting DNAse or RNAse activity into a colorimetric signal is to couple the cleavage of a DNA or RNA aptamer with the re-activation of an enzyme that is capable of producing a colorimetric output. In the absence of RNA or DNA cleavage, the intact aptamer will bind to the enzyme target and inhibit its activity. The advantage of this readout system is that the enzyme provides an additional amplification step: once liberated from an aptamer via collateral activity (e.g. Cpf1 collateral activity), the colorimetric enzyme will continue to produce colorimetric product, leading to a multiplication of signal.

In certain embodiments, an existing aptamer that inhibits an enzyme with a colorimetric readout is used. Several aptamer/enzyme pairs with colorimetric readouts exist, such as thrombin, protein C, neutrophil elastase, and subtilisin. These proteases have colorimetric substrates based upon pNA and are commercially available. In certain embodiments, a novel aptamer targeting a common colorimetric enzyme is used. Common and robust enzymes, such as beta-galactosidase, horseradish peroxidase, or calf intestinal alkaline phosphatase, could be targeted by engineered aptamers designed by selection strategies such as SELEX. Such strategies allow for quick selection of aptamers with nanomolar binding efficiencies and could be used for the development of additional enzyme/aptamer pairs for colorimetric readout.

In certain embodiments, the detection construct may be a DNA or RNA aptamer and/or may comprise a DNA or RNA-tethered inhibitor.

In certain embodiments, the detection construct may comprise a DNA or RNA oligonucleotide to which a detectable ligand and a masking component are attached.

In certain embodiments, RNAse or DNase activity is detected colorimetrically via cleavage of RNA-tethered inhibitors. Many common colorimetric enzymes have competitive, reversible inhibitors: for example, beta-galactosidase can be inhibited by galactose. Many of these inhibitors are weak, but their effect can be increased by increases in local concentration. By linking local concentration of inhibitors to DNase RNAse activity, colorimetric enzyme and inhibitor pairs can be engineered into DNase and RNAse sensors. The colorimetric DNase or RNAse sensor based upon small-molecule inhibitors involves three components: the colorimetric enzyme, the inhibitor, and a bridging RNA or DNA that is covalently linked to both the inhibitor and enzyme, tethering the inhibitor to the enzyme. In the uncleaved configuration, the enzyme is inhibited by the increased local concentration of the small molecule; when the DNA or RNA is cleaved (e.g. by Cas13 or Cas12 collateral cleavage), the inhibitor will be released and the colorimetric enzyme will be activated.

In certain embodiments, the aptamer or DNA- or RNA-tethered inhibitor may sequester an enzyme, wherein the enzyme generates a detectable signal upon release from the aptamer or DNA or RNA tethered inhibitor by acting upon a substrate. In some embodiments, the aptamer may be an inhibitor aptamer that inhibits an enzyme and prevents the enzyme from catalyzing generation of a detectable signal from a substance. In some embodiments, the DNA- or RNA-tethered inhibitor may inhibit an enzyme and may prevent the enzyme from catalyzing generation of a detectable signal from a substrate.

In certain embodiments, RNAse activity is detected colorimetrically via formation and/or activation of G-quadruplexes. G quadruplexes in DNA can complex with heme (iron (III)-protoporphyrin IX) to form a DNAzyme with peroxidase activity. When supplied with a peroxidase substrate (e.g. ABTS: (2,2′-Azinobis [3-ethylbenzothiazoline-6-sulfonic acid]-diammonium salt)), the G-quadruplex-heme complex in the presence of hydrogen peroxide causes oxidation of the substrate, which then forms a green color in solution. An example G-quadruplex forming DNA sequence is: GGGTAGGGCGGGTTGGGA (SEQ ID NO:201). By hybridizing an additional DNA or RNA sequence, referred to herein as a “staple,” to this DNA aptamer, formation of the G-quadraplex structure will be limited. Upon collateral activation, the staple will be cleaved allowing the G quadraplex to form and heme to bind. This strategy is particularly appealing because color formation is enzymatic, meaning there is additional amplification beyond collateral activation.

In certain embodiments, the detection construct may comprise an RNA oligonucleotide designed to bind a G-quadruplex forming sequence, wherein a G-quadruplex structure is formed by the G-quadruplex forming sequence upon cleavage of the detection construct, and wherein the G-quadruplex structure generates a detectable positive signal.

In certain example embodiments, the detection construct may be immobilized on a solid substrate in an individual discrete volume (defined further below) and sequesters a single reagent. For example, the reagent may be a bead comprising a dye. When sequestered by the immobilized reagent, the individual beads are too diffuse to generate a detectable signal, but upon release from the detection construct are able to generate a detectable signal, for example by aggregation or simple increase in solution concentration. In certain example embodiments, the immobilized masking agent is a DNA- or RNA-based aptamer that can be cleaved by the activated effector protein upon detection of a target molecule.

In one example embodiment, the detection construct comprises a detection agent that changes color depending on whether the detection agent is aggregated or dispersed in solution. For example, certain nanoparticles, such as colloidal gold, undergo a visible purple to red color shift as they move from aggregates to dispersed particles. Accordingly, in certain example embodiments, such detection agents may be held in aggregate by one or more bridge molecules. At least a portion of the bridge molecule comprises RNA or DNA. Upon activation of the effector proteins disclosed herein, the RNA or DNA portion of the bridge molecule is cleaved allowing the detection agent to disperse and resulting in the corresponding change in color. In certain example embodiments, the detection agent is a colloidal metal. The colloidal metal material may include water-insoluble metal particles or metallic compounds dispersed in a liquid, a hydrosol, or a metal sol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the A13+, Ru3+, Zn2+, Fe3+, Ni2+ and Ca2+ ions.

When the RNA or DNA bridge is cut by the activated CRISPR effector, the aforementioned color shift is observed. In certain example embodiments the particles are colloidal metals. In certain other example embodiments, the colloidal metal is a colloidal gold. In certain example embodiments, the colloidal nanoparticles are 15 nm gold nanoparticles (AuNPs). Due to the unique surface properties of colloidal gold nanoparticles, maximal absorbance is observed at 520 nm when fully dispersed in solution and appear red in color to the naked eye. Upon aggregation of AuNPs, they exhibit a red-shift in maximal absorbance and appear darker in color, eventually precipitating from solution as a dark purple aggregate. In certain example embodiments the nanoparticles are modified to include DNA linkers extending from the surface of the nanoparticle. Individual particles are linked together by single-stranded RNA (ssRNA) or single-stranded DNA bridges that hybridize on each end to at least a portion of the DNA linkers. Thus, the nanoparticles will form a web of linked particles and aggregate, appearing as a dark precipitate. Upon activation of the CRISPR effectors disclosed herein, the ssRNA or ssDNA bridge will be cleaved, releasing the AU NPS from the linked mesh and producing a visible red color. Example DNA linkers and bridge sequences are listed below. Thiol linkers on the end of the DNA linkers may be used for surface conjugation to the AuNPS. Other forms of conjugation may be used. In certain example embodiments, two populations of AuNPs may be generated, one for each DNA linker. This will help facilitate proper binding of the ssRNA bridge with proper orientation. In certain example embodiments, a first DNA linker is conjugated by 3′ end while a second DNA linker is conjugated by 5′ end.

TABLE XXVI

| | |
|---|---|
| C2c2 colorimetric DNA1 (SEQ ID NO: 202) | TTATAACTATTCCTAAAAAAAAAAA/3ThioMC3-D/ |
| C2c2 colorimetric DNA2 (SEQ ID NO: 203) | /5ThioMC6- |
| | D/AAAAAAAAAACTCCCCTAATAACAAT |
| C2c2 colorimetric bridge (SEQ ID NO: 204) | GGGUAGGAAUAGUUAUAAUUUCCCUUUCCCAUU |
| | GUUAUUAGGGAG |

In certain other example embodiments, the detection construct may comprise an RNA or DNA oligonucleotide to which are attached a detectable label and a masking agent of that detectable label. An example of such a detectable label/masking agent pair is a fluorophore and a quencher of the fluorophore. Quenching of the fluorophore can occur as a result of the formation of a non-fluorescent complex between the fluorophore and another fluorophore or non-fluorescent molecule. This mechanism is known as ground-state complex formation, static quenching, or contact quenching. Accordingly, the RNA or DNA oligonucleotide may be designed so that the fluorophore and quencher are in sufficient proximity for contact quenching to occur. Fluorophores and their cognate quenchers are known in the art and can be selected for this purpose by one having ordinary skill in the art. The particular fluorophore/quencher pair is not critical in the context of this disclosure, only that selection of the fluorophore/quencher pairs ensures masking of the fluorophore. Upon activation of the effector proteins disclosed herein, the RNA or DNA oligonucleotide is cleaved thereby severing the proximity between the fluorophore and quencher needed to maintain the contact quenching effect. Accordingly, detection of the fluorophore may be used to determine the presence of a target molecule in a sample.

In certain other example embodiments, the detection construct may comprise one or more RNA oligonucleotides to which are attached one or more metal nanoparticles, such as gold nanoparticles. In some embodiments, the detection construct comprises a plurality of metal nanoparticles crosslinked by a plurality of RNA or DNA oligonucleotides forming a closed loop. In one embodiment, the detection construct comprises three gold nanoparticles crosslinked by three RNA or DNA oligonucleotides forming a closed loop. In some embodiments, the cleavage of the RNA or DNA oligonucleotides by the CRISPR effector protein leads to a detectable signal produced by the metal nanoparticles.

In certain other example embodiments, the detection construct may comprise one or more RNA or DNA oligonucleotides to which are attached one or more quantum dots. In some embodiments, the cleavage of the RNA or DNA oligonucleotides by the CRISPR effector protein leads to a detectable signal produced by the quantum dots.

In one example embodiment, the detection construct may comprise a quantum dot. The quantum dot may have multiple linker molecules attached to the surface. At least a portion of the linker molecule comprises RNA or DNA. The linker molecule is attached to the quantum dot at one end and to one or more quenchers along the length or at terminal ends of the linker such that the quenchers are maintained in sufficient proximity for quenching of the quantum dot to occur. The linker may be branched. As above, the quantum dot/quencher pair is not critical, only that selection of the quantum dot/quencher pair ensures masking of the fluorophore. Quantum dots and their cognate quenchers are known in the art and can be selected for this purpose by one having ordinary skill in the art. Upon activation of the effector proteins disclosed herein, the RNA or DNA portion of the linker molecule is cleaved thereby eliminating the proximity between the quantum dot and one or more quenchers needed to maintain the quenching effect. In certain example embodiments the quantum dot is streptavidin conjugated. RNA or DNA are attached via biotin linkers and recruit quenching molecules with the sequences

(SEQ ID NO: 205)
/5Biosg/UCUCGUACGUUC/3IAbRQSp/,
or
(SEQ ID NO: 206)
/5Biosg/UCUCGUACGUUCUCUCGUACGUUC/3IAbRQSp/

where /5Biosg/is a biotin tag and /3lAbRQSp/is an Iowa black quencher. Upon cleavage, by the activated effectors disclosed herein the quantum dot will fluoresce visibly.

In specific embodiments, the detectable ligand may be a fluorophore and the masking component may be a quencher molecule.

In a similar fashion, fluorescence energy transfer (FRET) may be used to generate a detectable positive signal. FRET is a non-radiative process by which a photon from an energetically excited fluorophore (i.e. “donor fluorophore”) raises the energy state of an electron in another molecule (i.e. “the acceptor”) to higher vibrational levels of the excited singlet state. The donor fluorophore returns to the ground state without emitting a fluoresce characteristic of that fluorophore. The acceptor can be another fluorophore or non-fluorescent molecule. If the acceptor is a fluorophore, the transferred energy is emitted as fluorescence characteristic of that fluorophore. If the acceptor is a non-fluorescent molecule the absorbed energy is loss as heat. Thus, in the context of the embodiments disclosed herein, the fluorophore/quencher pair is replaced with a donor fluorophore/acceptor pair attached to the oligonucleotide molecule. When intact, the detection construct generates a first signal (negative detectable signal) as detected by the fluorescence or heat emitted from the acceptor. Upon activation of the effector proteins disclosed herein the RNA oligonucleotide is cleaved and FRET is disrupted such that fluorescence of the donor fluorophore is now detected (positive detectable signal).

In certain example embodiments, the detection construct comprises the use of intercalating dyes which change their absorbance in response to cleavage of long RNAs or DNAs to short nucleotides. Several such dyes exist. For example, pyronine-Y will complex with RNA and form a complex that has an absorbance at 572 nm. Cleavage of the RNA results in loss of absorbance and a color change. Methylene blue may be used in a similar fashion, with changes in absorbance at 688 nm upon RNA cleavage. Accordingly, in certain example embodiments the detection construct comprises a RNA and intercalating dye complex that changes absorbance upon the cleavage of RNA by the effector proteins disclosed herein.

In certain example embodiments, the detection construct may comprise an initiator for an HCR reaction. See e.g. Dirks and Pierce. PNAS 101, 15275-15728 (2004). HCR reactions utilize the potential energy in two hairpin species. When a single-stranded initiator having a portion of complementary to a corresponding region on one of the hairpins is released into the previously stable mixture, it opens a hairpin of one species. This process, in turn, exposes a single-stranded region that opens a hairpin of the other species. This process, in turn, exposes a single stranded region identical to the original initiator. The resulting chain reaction may lead to the formation of a nicked double helix that grows until the hairpin supply is exhausted. Detection of the resulting products may be done on a gel or colorimetrically. Example colorimetric detection methods include, for example, those disclosed in Lu et al. “Ultra-sensitive colorimetric assay system based on the hybridization chain reaction-triggered enzyme cascade amplification ACS Appl Mater Interfaces, 2017, 9(1):167-175, Wang et al. “An enzyme-free colorimetric assay using hybridization chain reaction amplification and split aptamers” Analyst 2015, 150, 7657-7662, and Song et al. “Noncovalent fluorescent labeling of hairpin DNA probe coupled with hybridization chain reaction for sensitive DNA detection.” Applied Spectroscopy, 70(4):686-694 (2016).

In certain example embodiments, the detection construct suppresses generation of a detectable positive signal until cleaved by an activated CRISPR effector protein. In some embodiments, the detection construct may suppress generation of a detectable positive signal by masking the detectable positive signal or generating a detectable negative signal instead.

### Amplification of Target

In certain example embodiments, target RNAs and/or DNAs may be amplified prior to activating the CRISPR effector protein. Any suitable RNA or DNA amplification technique may be used. In certain example embodiments, the RNA or DNA amplification is an isothermal amplification. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM).

In certain example embodiments, the RNA or DNA amplification is NASBA, which is initiated with reverse transcription of target RNA by a sequence-specific reverse primer to create a RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. Importantly, each of the new target RNAs can be detected by the guide RNAs thus further enhancing the sensitivity of the assay. Binding of the target RNAs by the guide RNAs then leads to activation of the CRISPR effector protein and the methods proceed as outlined above. The NASBA reaction has the additional advantage of being able to proceed under moderate isothermal conditions, for example at approximately 41° C., making it suitable for systems and devices deployed for early and direct detection in the field and far from clinical laboratories.

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, an RNA polymerase promoter, such as a T7 promoter, is added to one of the primers. This results in an amplified double-stranded DNA product comprising the target sequence and an RNA polymerase promoter. After, or during, the RPA reaction, an RNA polymerase is added that will produce RNA from the double-stranded DNA templates. The amplified target RNA can then in turn be detected by the CRISPR effector system. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

In an embodiment of the disclosure may comprise nickase-based amplification. The nicking enzyme may be a CRISPR protein. Accordingly, the introduction of nicks into dsDNA can be programmable and sequence-specific. FIG. 115 depicts an embodiment of the disclosure, which starts with two guides designed to target opposite strands of a dsDNA target. According to the disclosure, the nickase can be Cpf1, C2c1, Cas9 or any ortholog or CRISPR protein that cleaves or is engineered to cleave a single strand of a DNA duplex. The nicked strands may then be extended by a polymerase. In an embodiment, the locations of the nicks are selected such that extension of the strands by a polymerase is towards the central portion of the target duplex DNA between the nick sites. In certain embodiments, primers are included in the reaction capable of hybridizing to the extended strands followed by further polymerase extension of the primers to regenerate two dsDNA pieces: a first dsDNA that includes the first strand Cpf1 guide site or both the first and second strand Cpf1 guide sites, and a second dsDNA that includes the second strand Cpf1 guide site or both the first and second strand Cpf1 guide sites. These pieces continue to be nicked and extended in a cyclic reaction that exponentially amplifies the region of the target between nicking sites.

The amplification can be isothermal and selected for temperature. In one embodiment, the amplification proceeds rapidly at 37° C. degrees. In other embodiments, the temperature of the isothermal amplification may be chosen by selecting a polymerase (e.g. Bsu, Bst, Phi29, Klenow fragment etc.).operable at a different temperature.

Thus, whereas nicking isothermal amplification techniques use nicking enzymes with fixed sequence preference (e.g. in nicking enzyme amplification reaction or NEAR), which requires denaturing of the original dsDNA target to allow annealing and extension of primers that add the nicking substrate to the ends of the target, use of a CRISPR nickase wherein the nicking sites can be programed via guide RNAs means that no denaturing step is necessary, enabling the entire reaction to be truly isothermal. This also simplifies the reaction because these primers that add the nicking substrate are different than the primers that are used later in the reaction, meaning that NEAR requires two primer sets (i.e. 4 primers) while Cpf1 nicking amplification only requires one primer set (i.e. two primers). This makes nicking Cpf1 amplification much simpler and easier to operate without complicated instrumentation to perform the denaturation and then cooling to the isothermal temperature.

Accordingly, in certain example embodiments the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present disclosure.

A salt, such as magnesium chloride (MgCl2), potassium chloride (KCl), or sodium chloride (NaCl), may be included in an amplification reaction, such as PCR, to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present disclosure and as described herein.

Other components of a biological or chemical reaction may include a cell lysis component to break open or lyse a cell for analysis of the materials therein. A cell lysis component may include, but is not limited to, a detergent, a salt as described above, such as NaCl, KCl, ammonium sulfate [(NH4)2SO4], or others. Detergents that may be appropriate for the disclosure may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application and may be specific to the reaction in some cases. Amplification reactions may include dNTPs and nucleic acid primers used at any concentration appropriate for the disclosure, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the disclosure may be any specific or general polymerase known in the art and useful or the disclosure, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or aptamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification of nucleic acids may be performed using specific thermal cycle machinery or equipment and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification.

In certain embodiments, detection of DNA with the methods or systems of the disclosure requires transcription of the (amplified) DNA into RNA prior to detection.

It will be evident that detection methods of the disclosure can involve nucleic acid amplification and detection procedures in various combinations. The nucleic acid to be detected can be any naturally occurring or synthetic nucleic acid, including but not limited to DNA and RNA, which may be amplified by any suitable method to provide an intermediate product that can be detected. Detection of the intermediate product can be by any suitable method including but not limited to binding and activation of a CRISPR protein which produces a detectable signal moiety by direct or collateral activity.

### Enrichment CRISPR Systems

In certain example embodiments, target RNA or DNA may first be enriched prior to detection or amplification of the target RNA or DNA. In certain example embodiments, this enrichment may be achieved by binding of the target nucleic acids by a CRISPR effector system.

Current target-specific enrichment protocols require single-stranded nucleic acid prior to hybridization with probes. Among various advantages, the present embodiments can skip this step and enable direct targeting to double-stranded DNA (either partly or completely double-stranded). In addition, the embodiments disclosed herein are enzyme-driven targeting methods that offer faster kinetics and easier workflow allowing for isothermal enrichment. In certain example embodiments enrichment may take place at temperatures as low as 20-37° C. In certain example embodiments, a set of guide RNAs to different target nucleic acids are used in a single assay, allowing for detection of multiple targets and/or multiple variants of a single target.

In certain example embodiments, a dead CRISPR effector protein may bind the target nucleic acid in solution and then subsequently be isolated from said solution. For example, the dead CRISPR effector protein bound to the target nucleic acid, may be isolated from the solution using an antibody or other molecule, such as an aptamer, that specifically binds the dead CRISPR effector protein.

In other example embodiments, the dead CRISPR effector protein may bound to a solid substrate. A fixed substrate may refer to any material that is appropriate for or can be modified to be appropriate for the attachment of a polypeptide or a polynucleotide. Possible substrates include, but are not limited to, glass and modified functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. In some embodiments, the solid support comprises a patterned surface suitable for immobilization of molecules in an ordered pattern. In certain embodiments a patterned surface refers to an arrangement of different regions in or on an exposed layer of a solid support. In some embodiments, the solid support comprises an array of wells or depressions in a surface. The composition and geometry of the solid support can vary with its use. In some embodiments, the solids support is a planar structure such as a slide, chip, microchip and/or array. As such, the surface of the substrate can be in the form of a planar layer. In some embodiments, the solid support comprises one or more surfaces of a flowcell. The term “flowcell” as used herein refers to a chamber comprising a solid surface across which one or more fluid reagents can be flowed. Example flowcells and related fluidic systems and detection platforms that can be readily used in the methods of the present disclosure are described, for example, in Bentley et al. Nature 456:53-59 (2008), WO 04/0918497, U.S. Pat. No. 7,057,026; WO 91/06678; WO 07/123744; U.S. Pat. Nos. 7,329,492; 7,211,414; 7,315,019; 7,405,281, and US 2008/0108082. In some embodiments, the solid support or its surface is non-planar, such as the inner or outer surface of a tube or vessel. In some embodiments, the solid support comprise microspheres or beads. “Microspheres,” “bead,” “particles,” are intended to mean within the context of a solid substrate to mean small discrete particles made of various material including, but not limited to, plastics, ceramics, glass, and polystyrene. In certain embodiments, the microspheres are magnetic microspheres or beads. Alternatively or additionally, the beads may be porous. The bead sizes range from nanometers, e.g. 100 nm, to millimeters, e.g. 1 mm.

A sample containing, or suspected of containing, the target nucleic acids may then be exposed to the substrate to allow binding of the target nucleic acids to the bound dead CRISPR effector protein. Non-target molecules may then be washed away. In certain example embodiments, the target nucleic acids may then be released from the CRISPR effector protein/guide RNA complex for further detection using the methods disclosed herein. In certain example embodiments, the target nucleic acids may first be amplified as described herein.

In certain example embodiments, the CRISPR effector may be labeled with a binding tag. In certain example embodiments the CRISPR effector may be chemically tagged. For example, the CRISPR effector may be chemically biotinylated. In another example embodiment, a fusion may be created by adding additional sequence encoding a fusion to the CRISPR effector. One example of such a fusion is an AviTag™, which employs a highly targeted enzymatic conjugation of a single biotin on a unique 15 amino acid peptide tag. In certain embodiments, the CRISPR effector may be labeled with a capture tag such as, but not limited to, GST, Myc, hemagglutinin (HA), green fluorescent protein (GFP), flag, His tag, TAP tag, and Fc tag. The binding tag, whether a fusion, chemical tag, or capture tag, may be used to either pull down the CRISPR effector system once it has bound a target nucleic acid or to fix the CRISPR effector system on the solid substrate.

In certain example embodiments, the guide RNA may be labeled with a binding tag. In certain example embodiments, the entire guide RNA may be labeled using in vitro transcription (IVT) incorporating one or more biotinylated nucleotides, such as, biotinylated uracil. In some embodiments, biotin can be chemically or enzymatically added to the guide RNA, such as, the addition of one or more biotin groups to the 3′ end of the guide RNA. The binding tag may be used to pull down the guide RNA/target nucleic acid complex after binding has occurred, for example, by exposing the guide RNA/target nucleic acid to a streptavidin coated solid substrate.

In specific embodiments, the solid substrate may be a flow cell. In certain embodiments, a flow cell may be a device for detecting the presence or amount of an analyte in a test sample. The flow cell device may have immobilized reagent means which produce an electrically or optically detectable response to an analyte which may be contained in a test sample.

Accordingly, in certain example embodiments, an engineered or non-naturally-occurring CRISPR effector may be used for enrichment purposes. In an embodiment, the modification may comprise mutation of one or more amino acid residues of the effector protein. The one or more mutations may be in one or more catalytically active domains of the effector protein. The effector protein may have reduced or abolished nuclease activity compared with an effector protein lacking said one or more mutations. The effector protein may not direct cleavage of the RNA strand at the target locus of interest. In a preferred embodiment, the one or more mutations may comprise two mutations. In a preferred embodiment the one or more amino acid residues are modified in a C2c2 effector protein, e.g., an engineered or non-naturally-occurring effector protein or C2c2. In particular embodiments, the one or more modified of mutated amino acid residues are one or more of those in C2c2 corresponding to R597, H602, R1278 and H1283 (referenced to Lsh C2c2 amino acids), such as mutations R597A, H602A, R1278A and H1283A, or the corresponding amino acid residues in Lsh C2c2 orthologues.

As such, the enrichment CRISPR system may comprise a catalytically inactive CRISPR effector protein. In specific embodiments, the catalytically inactive CRISPR effector protein is a catalytically inactive C2c2.

In particular embodiments, the one or more modified of mutated amino acid residues are one or more of those in C2c2 corresponding to K2, K39, V40, E479, L514, V518, N524, G534, K535, E580, L597, V602, D630, F676, L709, 1713, R717 (HEPN), N718, H722 (HEPN), E773, P823, V828, 1879, Y880, F884, Y997, L1001, F1009, L1013, Y1093, L1099, L1111, Y1114, L1203, D1222, Y1244, L1250, L1253, K1261, 11334, L1355, L1359, R1362, Y1366, E1371, R1372, D1373, R1509 (HEPN), H1514 (HEPN), Y1543, D1544, K1546, K1548, V1551, 11558, according to C2c2 consensus numbering. In certain embodiments, the one or more modified of mutated amino acid residues are one or more of those in C2c2 corresponding to R717 and R1509. In certain embodiments, the one or more modified of mutated amino acid residues are one or more of those in C2c2 corresponding to K2, K39, K535, K1261, R1362, R1372, K1546 and K1548. In certain embodiments, said mutations result in a protein having an altered or modified activity. In certain embodiments, said mutations result in a protein having a reduced activity, such as reduced specificity. In certain embodiments, said mutations result in a protein having no catalytic activity (i.e. “dead” C2c2). In an embodiment, said amino acid residues correspond to Lsh C2c2 amino acid residues, or the corresponding amino acid residues of a C2c2 protein from a different species. Devices that can facilitate these steps. In some embodiments, to reduce the size of a fusion protein of the Cas13b effector and the one or more functional domains, the C-terminus of the Cas13b effector can be truncated while still maintaining its RNA binding function. For example, at least 20 amino acids, at least 50 amino acids, at least 80 amino acids, or at least 100 amino acids, or at least 150 amino acids, or at least 200 amino acids, or at least 250 amino acids, or at least 300 amino acids, or at least 350 amino acids, or up to 120 amino acids, or up to 140 amino acids, or up to 160 amino acids, or up to 180 amino acids, or up to 200 amino acids, or up to 250 amino acids, or up to 300 amino acids, or up to 350 amino acids, or up to 400 amino acids, may be truncated at the C-terminus of the Cas13b effector. Specific examples of Cas13b truncations include C-terminal 4984-1090, C-terminal A1026-1090, and C-terminal 41053-1090, C-terminal 4934-1090, C-terminal 4884-1090, C-terminal A834-1090, C-terminal 4784-1090, and C-terminal 4734-1090, wherein amino acid positions correspond to amino acid positions of Prevotella sp. P5-125 Cas13b protein.

The above enrichment systems may also be used to deplete a sample of certain nucleic acids. For example, guide RNAs may be designed to bind non-target RNAs to remove the non-target RNAs from the sample. In one example embodiment, the guide RNAs may be designed to bind nucleic acids that do carry a particular nucleic acid variation. For example, in a given sample a higher copy number of non-variant nucleic acids may be expected. Accordingly, the embodiments disclosed herein may be used to remove the non-variant nucleic acids from a sample, to increase the efficiency with which the detection CRISPR effector system can detect the target variant sequences in a given sample.

Amplification and/or Enhancement of Detectable Positive Signal

In certain example embodiments, further modification may be introduced that further amplify the detectable positive signal. For example, activated CRISPR effector protein collateral activation may be used to generate a secondary target or additional guide sequence, or both. In one example embodiment, the reaction solution would contain a secondary target that is spiked in at high concentration. The secondary target may be distinct from the primary target (i.e. the target for which the assay is designed to detect) and in certain instances may be common across all reaction volumes. A secondary guide sequence for the secondary target may be protected, e.g. by a secondary structural feature such as a hairpin with a RNA loop, and unable to bind the second target or the CRISPR effector protein. Cleavage of the protecting group by an activated CRISPR effector protein (i.e. after activation by formation of complex with the primary target(s) in solution) and formation of a complex with free CRISPR effector protein in solution and activation from the spiked in secondary target. In certain other example embodiments, a similar concept is used with a second guide sequence to a secondary target sequence. The secondary target sequence may be protected a structural feature or protecting group on the secondary target. Cleavage of a protecting group off the secondary target then allows additional CRISPR effector protein/second guide sequence/secondary target complex to form. In yet another example embodiment, activation of CRISPR effector protein by the primary target(s) may be used to cleave a protected or circularized primer, which is then released to perform an isothermal amplification reaction, such as those disclosed herein, on a template that encodes a secondary guide sequence, secondary target sequence, or both. Subsequent transcription of this amplified template would produce more secondary guide sequence and/or secondary target sequence, followed by additional CRISPR effector protein collateral activation.

### V. Diagnostic Devices

The systems described herein can be embodied on diagnostic devices. A number of substrates and configurations may be used. The devices may be capable of defining multiple individual discrete volumes within the device. As used herein an “individual discrete volume” refers to a discrete space, such as a container, receptacle, or other defined volume or space that can be defined by properties that prevent and/or inhibit migration of target molecules, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof that can contain a sample within a defined space. Individual discrete volumes may be identified by molecular tags, such as nucleic acid barcodes. By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the use of non-walled, or semipermeable discrete volumes is that some reagents, such as buffers, chemical activators, or other agents may be passed through the discrete volume, while other materials, such as target molecules, may be maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example polyethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain embodiments, the compartment is an aqueous droplet in a water-in-oil emulsion. In specific embodiments, any of the applications, methods, or systems described herein requiring exact or uniform volumes may employ the use of an acoustic liquid dispenser.

In some embodiments, the individual discrete volumes may be droplets.

In certain example embodiments, the device comprises a flexible material substrate on which a number of spots may be defined. Flexible substrate materials suitable for use in diagnostics and biosensing are known within the art. The flexible substrate materials may be made of plant derived fibers, such as cellulosic fibers, or may be made from flexible polymers such as flexible polyester films and other polymer types. Within each defined spot, reagents of the system described herein are applied to the individual spots. Each spot may contain the same reagents except for a different guide RNA or set of guide RNAs, or where applicable, a different detection aptamer to screen for multiple targets at once. Thus, the systems and devices herein may be able to screen samples from multiple sources (e.g. multiple clinical samples from different individuals) for the presence of the same target, or a limited number of targets, or aliquots of a single sample (or multiple samples from the same source) for the presence of multiple different targets in the sample. In certain example embodiments, the elements of the systems described herein are freeze dried onto the paper or cloth substrate. Example flexible material based substrates that may be used in certain example devices are disclosed in Pardee et al. Cell. 2016, 165(5):1255-66 and Pardee et al. Cell. 2014, 159(4):950-54. Suitable flexible material-based substrates for use with biological fluids, including blood are disclosed in International Patent Application Publication No. WO/2013/071301 entitled “Paper based diagnostic test” to Shevkoplyas et al. U.S. Patent Application Publication No. 2011/0111517 entitled “Paper-based microfluidic systems” to Siegel et al. and Shafiee et al. “Paper and Flexible Substrates as Materials for Biosensing Platforms to Detect Multiple Biotargets” Scientific Reports 5:8719 (2015). Further flexible based materials, including those suitable for use in wearable diagnostic devices are disclosed in Wang et al. “Flexible Substrate-Based Devices for Point-of-Care Diagnostics” Cell 34(11):909-21 (2016). Further flexible based materials may include nitrocellulose, polycarbonate, methylethyl cellulose, polyvinylidene fluoride (PVDF), polystyrene, or glass (see e.g., US20120238008). In certain embodiments, discrete volumes are separated by a hydrophobic surface, such as but not limited to wax, photoresist, or solid ink.

In specific embodiments, each individual discrete volume further comprises one or more detection aptamers comprising a masked RNA polymerase promoter binding site or a masked primer binding site. As such, each individual discrete volume may further comprise nucleic acid amplification reagents.

In specific embodiments, the target molecule may be a target DNA and the individual discrete volumes further comprise a primer that binds the target DNA and comprises an RNA polymerase promoter.

Samples sources that may be analyzed using the systems and devices described herein include biological samples of a subject or environmental samples. Environmental samples may include surfaces or fluids. The biological samples may include, but are not limited to, saliva, blood, plasma, sera, stool, urine, sputum, mucous, lymph, synovial fluid, spinal fluid, cerebrospinal fluid, a swab from skin or a mucosal membrane, or combination thereof. In an example embodiment, the environmental sample is taken from a solid surface, such as a surface used in the preparation of food or other sensitive compositions and materials.

In other example embodiments, the elements of the systems described herein may be place on a single use substrate, such as swab or cloth that is used to swab a surface or sample fluid. For example, the system could be used to test for the presence of a pathogen on a food by swabbing the surface of a food product, such as a fruit or vegetable. Similarly, the single use substrate may be used to swab other surfaces for detection of certain microbes or agents, such as for use in security screening. Single use substrates may also have applications in forensics, where the CRISPR systems are designed to detect, for example identifying DNA SNPs that may be used to identify a suspect, or certain tissue or cell markers to determine the type of biological matter present in a sample. Likewise, the single use substrate could be used to collect a sample from a patient-such as a saliva sample from the mouth—or a swab of the skin. In other embodiments, a sample or swab may be taken of a meat product on order to detect the presence of absence of contaminants on or within the meat product.

### Lateral Flow Strip

In certain embodiments, the diagnostic device is or comprises a flow strip. For instance, a lateral flow strip allows for RNAse (e.g. C2c2) detection by color. The RNA reporter is modified to have a first molecule (such as for instance FITC) attached to the 5′ end and a second molecule (such as for instance biotin) attached to the 3′ end (or vice versa). The lateral flow strip is designed to have two capture lines with anti-first molecule (e.g. anti-FITC) antibodies hybridized at the first line and anti-second molecule (e.g. anti-biotin) antibodies at the second downstream line. As the reaction flows down the strip, uncleaved reporter will bind to anti-first molecule antibodies at the first capture line, while cleaved reporters will liberate the second molecule and allow second molecule binding at the second capture line. Second molecule sandwich antibodies, for instance conjugated to nanoparticles, such as gold nanoparticles, will bind any second molecule at the first or second line and result in a strong readout/signal (e.g. color). As more reporter is cleaved, more signal will accumulate at the second capture line and less signal will appear at the first line. In certain aspects, the disclosure relates to the use of a follow strip as described herein for detecting nucleic acids or polypeptides. In certain aspects, the disclosure relates to a method for detecting nucleic acids or polypeptides with a flow strip as defined herein, e.g. (lateral) flow tests or (lateral) flow immunochromatographic assays.

The embodiments disclosed herein are directed to lateral flow detection devices that comprise SHERLOCK systems. Reference is made to Gootenberg, et al., “Multiplexed and portable nucleic acid detection platform with Cas13, Cas12a, and Csm6,” Science. 2018 Apr. 27; 360(6387):439-444. doi: 10.1126/science.aaq0179, and International Patent Publication No, WO 2019/071051, each specifically incorporated herein by reference. The device may comprise a lateral flow substrate for detecting a SHERLOCK reaction. Substrates suitable for use in lateral flow assays are known in the art. These may include but are not necessarily limited to membranes or pads made of cellulose and/or glass fiber, polyesters, nitrocellulose, or absorbent pads (J Saudi Chem Soc 19(6):689-705; 2015).

The SHERLOCK system, i.e. one or more CRISPR systems and corresponding reporter constructs are added to the lateral flow substrate at a defined reagent portion of the lateral flow substrate, typically on one end of the lateral flow substrate. In one embodiment, reporting constructs used within the context of the present disclosure comprise a first molecule and a second molecule linked by an RNA or DNA linker. In one embodiment, the lateral flow substrate further comprises a sample portion equivalent to, continuous with, or adjacent to the reagent portion. In one embodiment, the lateral flow strip further comprises a first capture line, typically a horizontal line running across the device, but other configurations are possible. In one embodiment, the first capture region is proximate to and on the same end of the lateral flow substrate as the sample loading portion. In one embodiment, a first binding agent that specifically binds the first molecule of the reporter construct is fixed or otherwise immobilized to the first capture region. In one embodiment, the second capture region is located towards the opposite end of the lateral flow substrate from the first binding region. In one embodiment, a second binding agent is fixed or otherwise immobilized at the second capture region that can specifically bind to the second molecule of the reporter construct, or the second binding agent may bind a detectable ligand. For example, the detectable ligand may be a particle, such as a colloidal particle, that when it aggregates can be detected visually. The particle may be modified with an antibody that specifically binds the second molecule on the reporter construct. If the reporter construct is not cleaved it will facilitate accumulation of the detectable ligand at the first binding region. If the reporter construct is cleaved the detectable ligand is released to flow to the second binding region. In such an embodiment, the second binding agent is an agent capable of specifically or non-specifically binding the detectable ligand on the antibody on the detectable ligand. Examples of suitable binding agents for such an embodiment include, but are not limited to, protein A and protein G.

Lateral support substrates may be located within a housing (see for example, “Rapid Lateral Flow Test Strips” Merck Millipore 2013). The housing may comprise at least one opening for loading samples and a second single opening or separate openings that allow for reading of detectable signal generated at the first and second capture regions.

The SHERLOCK system may be freeze-dried to the lateral flow substrate and packaged as a ready to use device, or the SHERLOCK system may be added to the reagent portion of the lateral flow substrate at the time of using the device. Samples to be screened are loaded at the sample loading portion of the lateral flow substrate. The samples must be liquid samples or samples dissolved in an appropriate solvent, usually aqueous. The liquid sample reconstitutes the SHERLOCK reagents such that a SHERLOCK reaction can occur. The liquid sample begins to flow from the sample portion of the substrate towards the first and second capture regions. Intact reporter construct is bound at the first capture region by binding between the first binding agent and the first molecule. Likewise, the detection agent will begin to collect at the first binding region by binding to the second molecule on the intact reporter construct. If target molecule(s) are present in the sample, the CRISPR effector protein collateral effect is activated. As activated CRISPR effector protein comes into contact with the bound reporter construct, the reporter constructs are cleaved, releasing the second molecule to flow further down the lateral flow substrate towards the second binding region. The released second molecule is then captured at the second capture region by binding to the second binding agent, where additional detection agent may also accumulate by binding to the second molecule. Accordingly, if the target molecule(s) is not present in the sample, a detectable signal will appear at the first capture region, and if the target molecule(s) is present in the sample, a detectable signal will appear at the location of the second capture region.

Specific binding-integrating molecules comprise any members of binding pairs that can be used in the present disclosure. Such binding pairs are known to those skilled in the art and include, but are not limited to, antibody-antigen pairs, enzyme-substrate pairs, receptor-ligand pairs, and streptavidin-biotin. In addition to such known binding pairs, novel binding pairs may be specifically designed. A characteristic of binding pairs is the binding between the two members of the binding pair.

Oligonucleotide Linkers having molecules on either end may comprise DNA if the CRISPR effector protein has DNA collateral activity (Cpf1 and C2c1) or RNA if the CRISPR effector protein has RNA collateral activity. Oligonucleotide linkers may be single stranded or double stranded, and in certain embodiments, they could contain both RNA and DNA regions. Oligonucleotide linkers may be of varying lengths, such as 5-10 nucleotides, 10-20 nucleotides, 20-50 nucleotides, or more.

In some embodiments, the polypeptide identifier elements include affinity tags, such as hemagglutinin (HA) tags, Myc tags, FLAG tags, V5 tags, chitin binding protein (CBP) tags, maltose-binding protein (MBP) tags, GST tags, poly-His tags, and fluorescent proteins (for example, green fluorescent protein (GFP), yellow fluorescent protein (YFP), cyan fluorescent protein (CFP), dsRed, mCherry, Kaede, Kindling, and derivatives thereof, FLAG tags, Myc tags, AU1 tags, T7 tags, OLLAS tags, Glu-Glu tags, VSV tags, or a combination thereof. Other Affinity tags are well known in the art. Such labels can be detected and/or isolated using methods known in the art (for example, by using specific binding agents, such as antibodies, that recognize a particular affinity tag). Such specific binding agents (for example, antibodies) can further contain, for example, detectable labels, such as isotope labels and/or nucleic acid barcodes such as those described herein.

For instance, a lateral flow strip allows for RNAse (e.g. Cas13a) detection by color. The RNA reporter is modified to have a first molecule (such as for instance FITC) attached to the 5′ end and a second molecule (such as for instance biotin) attached to the 3′ end (or vice versa). The lateral flow strip is designed to have two capture lines with anti-first molecule (e.g. anti-FITC) antibodies hybridized at the first line and anti-second molecule (e.g. anti-biotin) antibodies at the second downstream line. As the SHERLOCK reaction flows down the strip, uncleaved reporter will bind to anti-first molecule antibodies at the first capture line, while cleaved reporters will liberate the second molecule and allow second molecule binding at the second capture line. Second molecule sandwich antibodies, for instance conjugated to nanoparticles, such as gold nanoparticles, will bind any second molecule at the first or second line and result in a strong readout/signal (e.g. color). As more reporter is cleaved, more signal will accumulate at the second capture line and less signal will appear at the first line. In certain aspects, the disclosure relates to the use of a follow strip as described herein for detecting nucleic acids or polypeptides. In certain aspects, the disclosure relates to a method for detecting nucleic acids or polypeptides with a flow strip as defined herein, e.g. (lateral) flow tests or (lateral) flow immunochromatographic assays.

In certain example embodiments, a lateral flow device comprises a lateral flow substrate comprising a first end for application of a sample. The first region is loaded with a detectable ligand, such as those disclosed herein, for example a gold nanoparticle. The gold nanoparticle may be modified with a first antibody, such as an anti-FITC antibody. The first region also comprises a detection construct. In one example embodiment, a RNA detection construct and a CRISPR effector system (a CRISPR effector protein and one or more guide sequences configured to bind to one or more target sequences) as disclosed herein. In one example embodiment, and for purposes of further illustration, the RNA construct may comprise a FAM molecule on a first end of the detection construction and a biotin on a second end of the detection construct. Upstream of the flow of solution from the first end of the lateral flow substrate is a first test band. The test band may comprise a biotin ligand. Accordingly, when the RNA detection construct is present it its initial state, i.e. in the absence of target, the FAM molecule on the first end will bind the anti-FITC antibody on the gold nanoparticle, and the biotin on the second end of the RNA construct will bind the biotin ligand allowing for the detectable ligand to accumulate at the first test, generating a detectable signal. Generation of a detectable signal at the first band indicate the absence of the target ligand. In the presence of target, the CRISPR effector complex forms and the CRISPR effector protein is activated resulting in cleavage of the RND detection construct. In the absence of intact RNA detection construct the colloidal gold will flow past the second strip. The lateral flow device may comprise a second band, upstream of the first band. The second band may comprise a molecule capable of binding the antibody-labeled colloidal gold molecule, for example an anti-rabbit antibody capable of binding a rabbit anti-FTIC antibody on the colloidal gold. Therefore, in the presence of one or more targets, the detectable ligand will accumulate at the second band, indicating the presence of the one or more targets in the sample.

### Microfluidic Device

In certain example embodiments, the device is a microfluidic device that generates and/or merges different droplets (i.e. individual discrete volumes). For example, a first set of droplets may be formed containing samples to be screened and a second set of droplets formed containing the elements of the systems described herein. The first and second set of droplets are then merged and then diagnostic methods as described herein are carried out on the merged droplet set. Microfluidic devices disclosed herein may be silicone-based chips and may be fabricated using a variety of techniques, including, but not limited to, hot embossing, molding of elastomers, injection molding, LIGA, soft lithography, silicon fabrication and related thin film processing techniques. Suitable materials for fabricating the microfluidic devices include, but are not limited to, cyclic olefin copolymer (COC), polycarbonate, poly(dimethylsiloxane) (PDMS), and poly(methylacrylate) (PMMA). In one embodiment, soft lithography in PDMS may be used to prepare the microfluidic devices. For example, a mold may be made using photolithography which defines the location of flow channels, valves, and filters within a substrate. The substrate material is poured into a mold and allowed to set to create a stamp. The stamp is then sealed to a solid support, such as but not limited to, glass. Due to the hydrophobic nature of some polymers, such as PDMS, which absorbs some proteins and may inhibit certain biological processes, a passivating agent may be necessary (Schoffner et al. Nucleic Acids Research, 1996, 24:375-379). Suitable passivating agents are known in the art and include, but are not limited to, silanes, parylene, n-Dodecyl-b-D-matoside (DDM), pluronic, Tween-20, other similar surfactants, polyethylene glycol (PEG), albumin, collagen, and other similar proteins and peptides.

In certain example embodiments, the system and/or device may be adapted for conversion to a flow-cytometry readout in or allow to all of sensitive and quantitative measurements of millions of cells in a single experiment and improve upon existing flow-based methods, such as the PrimeFlow assay. In certain example embodiments, cells may be cast in droplets containing unpolymerized gel monomer, which can then be cast into single-cell droplets suitable for analysis by flow cytometry. A detection construct comprising a fluorescent detectable label may be cast into the droplet comprising unpolymerized gel monomer. Upon polymerization of the gel monomer to form a bead within a droplet. Because gel polymerization is through free-radical formation, the fluorescent reporter becomes covalently bound to the gel. The detection construct may be further modified to comprise a linker, such as an amine. A quencher may be added post-gel formation and will bind via the linker to the reporter construct. Thus, the quencher is not bound to the gel and is free to diffuse away when the reporter is cleaved by the CRISPR effector protein. Amplification of signal in droplet may be achieved by coupling the detection construct to a hybridization chain reaction (HCR initiator) amplification. DNA/RNA hybrid hairpins may be incorporated into the gel which may comprise a hairpin loop that has a RNase sensitive domain. By protecting a strand displacement toehold within a hairpin loop that has a RNase sensitive domain, HCR initiators may be selectively deprotected following cleavage of the hairpin loop by the CRISPR effector protein. Following deprotection of HCR initiators via toehold mediated strand displacement, fluorescent HCR monomers may be washed into the gel to enable signal amplification where the initiators are deprotected.

An example of microfluidic device that may be used in the context of the disclosure is described in Hour et al. “Direct Detection and drug-resistance profiling of bacteremia using inertial microfluidics” Lap Chip. 15(10):2297-2307 (2016).

In systems described herein, may further be incorporated into wearable medical devices that assess biological samples, such as biological fluids, of a subject outside the clinic setting and report the outcome of the assay remotely to a central server accessible by a medical care professional. The device may include the ability to self-sample blood, such as the devices disclosed in U.S. Patent Application Publication No. 2015/0342509 entitled “Needle-free Blood Draw to Peeters et al., U.S. Patent Application Publication No. 2015/0065821 entitled “Nanoparticle Phoresis” to Andrew Conrad.

In some embodiments, the individual discrete volumes are microwells.

In certain example embodiments, the device may comprise individual wells, such as microplate wells. The size of the microplate wells may be the size of standard 6, 24, 96, 384, 1536, 3456, or 9600 sized wells. In certain example embodiments, the elements of the systems described herein may be freeze dried and applied to the surface of the well prior to distribution and use.

The devices disclosed herein may further comprise inlet and outlet ports, or openings, which in turn may be connected to valves, tubes, channels, chambers, and syringes and/or pumps for the introduction and extraction of fluids into and from the device. The devices may be connected to fluid flow actuators that allow directional movement of fluids within the microfluidic device. Example actuators include, but are not limited to, syringe pumps, mechanically actuated recirculating pumps, electroosmotic pumps, bulbs, bellows, diaphragms, or bubbles intended to force movement of fluids. In certain example embodiments, the devices are connected to controllers with programmable valves that work together to move fluids through the device. In certain example embodiments, the devices are connected to the controllers discussed in further detail below. The devices may be connected to flow actuators, controllers, and sample loading devices by tubing that terminates in metal pins for insertion into inlet ports on the device.

As shown herein the elements of the system are stable when freeze dried, therefore embodiments that do not require a supporting device are also contemplated, i.e. the system may be applied to any surface or fluid that will support the reactions disclosed herein and allow for detection of a positive detectable signal from that surface or solution. In addition to freeze-drying, the systems may also be stably stored and utilized in a pelletized form. Polymers useful in forming suitable pelletized forms are known in the art.

In some embodiments, the individual discrete volumes are defined on a solid substrate. In some embodiments, the individual discrete volumes are spots defined on a substrate. In some embodiments, the substrate may be a flexible materials substrate, for example, including, but not limited to, a paper substrate, a fabric substrate, or a flexible polymer-based substrate. In specific embodiments, the flexible materials substrate is a paper substrate or a flexible polymer based substrate.

In certain embodiments, the CRISPR effector protein is bound to each discrete volume in the device. Each discrete volume may comprise a different guide RNA specific for a different target molecule. In certain embodiments, a sample is exposed to a solid substrate comprising more than one discrete volume each comprising a guide RNA specific for a target molecule. Not being bound by a theory, each guide RNA will capture its target molecule from the sample and the sample does not need to be divided into separate assays. Thus, a valuable sample may be preserved. The effector protein may be a fusion protein comprising an affinity tag. Affinity tags are well known in the art (e.g., HA tag, Myc tag, Flag tag, His tag, biotin). The effector protein may be linked to a biotin molecule and the discrete volumes may comprise streptavidin. In other embodiments, the CRISPR effector protein is bound by an antibody specific for the effector protein. Methods of binding a CRISPR enzyme has been described previously (see, e.g., US20140356867A1).

The devices disclosed herein may also include elements of point of care (POC) devices known in the art for analyzing samples by other methods. See, for example St John and Price, “Existing and Emerging Technologies for Point-of-Care Testing” (Clin Biochem Rev. 2014 August; 35(3): 155-167).

The present disclosure may be used with a wireless lab-on-chip (LOC) diagnostic sensor system (see e.g., U.S. Pat. No. 9,470,699 “Diagnostic radio frequency identification sensors and applications thereof”). In certain embodiments, the present disclosure is performed in a LOC controlled by a wireless device (e.g., a cell phone, a personal digital assistant (PDA), a tablet) and results are reported to said device.

Radio frequency identification (RFID) tag systems include an RFID tag that transmits data for reception by an RFID reader (also referred to as an interrogator). In a typical RFID system, individual objects (e.g., store merchandise) are equipped with a relatively small tag that contains a transponder. The transponder has a memory chip that is given a unique electronic product code. The RFID reader emits a signal activating the transponder within the tag through the use of a communication protocol. Accordingly, the RFID reader is capable of reading and writing data to the tag. Additionally, the RFID tag reader processes the data according to the RFID tag system application. Currently, there are passive and active type RFID tags. The passive type RFID tag does not contain an internal power source, but is powered by radio frequency signals received from the RFID reader. Alternatively, the active type RFID tag contains an internal power source that enables the active type RFID tag to possess greater transmission ranges and memory capacity. The use of a passive versus an active tag is dependent upon the particular application.

### Lab-On-the Chip

Lab-on-the chip technology is well described in the scientific literature and consists of multiple microfluidic channels, input or chemical wells. Reactions in wells can be measured using radio frequency identification (RFID) tag technology since conductive leads from RFID electronic chip can be linked directly to each of the test wells. An antenna can be printed or mounted in another layer of the electronic chip or directly on the back of the device. Furthermore, the leads, the antenna and the electronic chip can be embedded into the LOC chip, thereby preventing shorting of the electrodes or electronics. Since LOC allows complex sample separation and analyses, this technology allows LOC tests to be done independently of a complex or expensive reader. Rather a simple wireless device such as a cell phone or a PDA can be used. In one embodiment, the wireless device also controls the separation and control of the microfluidics channels for more complex LOC analyses. In one embodiment, a LED and other electronic measuring or sensing devices are included in the LOC-RFID chip. Not being bound by a theory, this technology is disposable and allows complex tests that require separation and mixing to be performed outside of a laboratory.

In preferred embodiments, the LOC may be a microfluidic device. The LOC may be a passive chip, wherein the chip is powered and controlled through a wireless device. In certain embodiments, the LOC includes a microfluidic channel for holding reagents and a channel for introducing a sample. In certain embodiments, a signal from the wireless device delivers power to the LOC and activates mixing of the sample and assay reagents. Specifically, in the case of the present disclosure, the system may include a masking agent, CRISPR effector protein, and guide RNAs specific for a target molecule. Upon activation of the LOC, the microfluidic device may mix the sample and assay reagents. Upon mixing, a sensor detects a signal and transmits the results to the wireless device. In certain embodiments, the unmasking agent is a conductive RNA molecule. The conductive RNA molecule may be attached to the conductive material. Conductive molecules can be conductive nanoparticles, conductive proteins, metal particles that are attached to the protein or latex or other beads that are conductive. In certain embodiments, if DNA or RNA is used then the conductive molecules can be attached directly to the matching DNA or RNA strands. The release of the conductive molecules may be detected across a sensor. The assay may be a one step process.

Since the electrical conductivity of the surface area can be measured precisely quantitative results are possible on the disposable wireless RFID electro-assays. Furthermore, the test area can be very small allowing for more tests to be done in a given area and therefore resulting in cost savings. In certain embodiments, separate sensors each associated with a different CRISPR effector protein and guide RNA immobilized to a sensor are used to detect multiple target molecules. Not being bound by a theory, activation of different sensors may be distinguished by the wireless device.

In addition to the conductive methods described herein, other methods may be used that rely on RFID or Bluetooth as the basic low cost communication and power platform for a disposable RFID assay. For example, optical means may be used to assess the presence and level of a given target molecule. In certain embodiments, an optical sensor detects unmasking of a fluorescent masking agent.

In certain embodiments, the device of the present disclosure may include handheld portable devices for diagnostic reading of an assay (see e.g., Vashist et al., Commercial Smartphone-Based Devices and Smart Applications for Personalized Healthcare Monitoring and Management, Diagnostics 2014, 4(3), 104-128; mReader from Mobile Assay; and Holomic Rapid Diagnostic Test Reader).

As noted herein, certain embodiments allow detection via colorimetric change which has certain attendant benefits when embodiments are utilized in POC situations and or in resource poor environments where access to more complex detection equipment to readout the signal may be limited. However, portable embodiments disclosed herein may also be coupled with hand-held spectrophotometers that enable detection of signals outside the visible range. An example of a hand-held spectrophotometer device that may be used in combination with the present disclosure is described in Das et al. “Ultra-portable, wireless smartphone spectrophotometer for rapid, non-destructive testing of fruit ripeness.” Nature Scientific Reports. 2016, 6:32504, DOI: 10.1038/srep32504. Finally, in certain embodiments utilizing quantum dot-based detection constructs, use of a handheld UV light, or other suitable device, may be successfully used to detect a signal owing to the near complete quantum yield provided by quantum dots.

### VI. Multiplex Detection

Multiplex CRISPR detection is an advanced technique that allows for the simultaneous detection of multiple genetic targets in a single assay. This method leverages the CRISPR/Cas system, which is known for its precision in targeting specific DNA sequences.

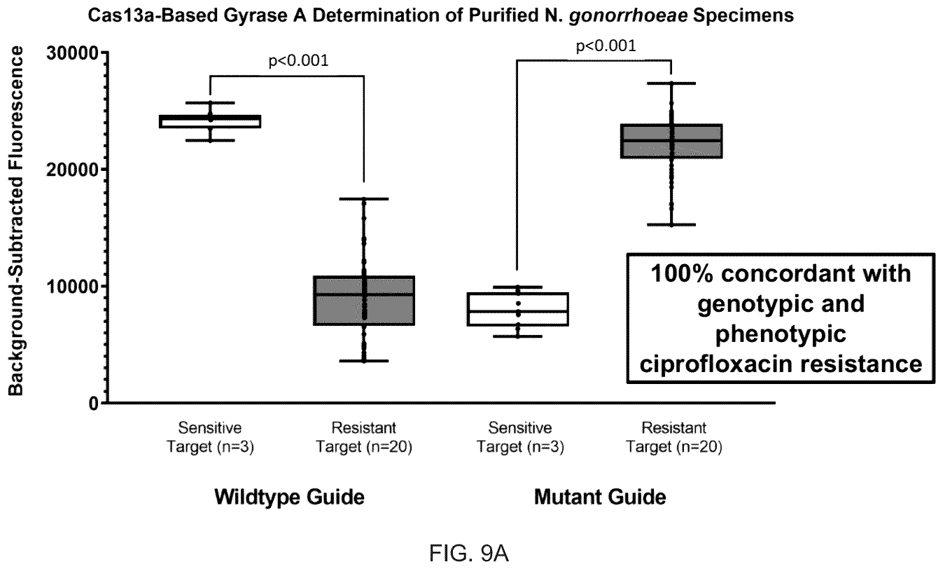

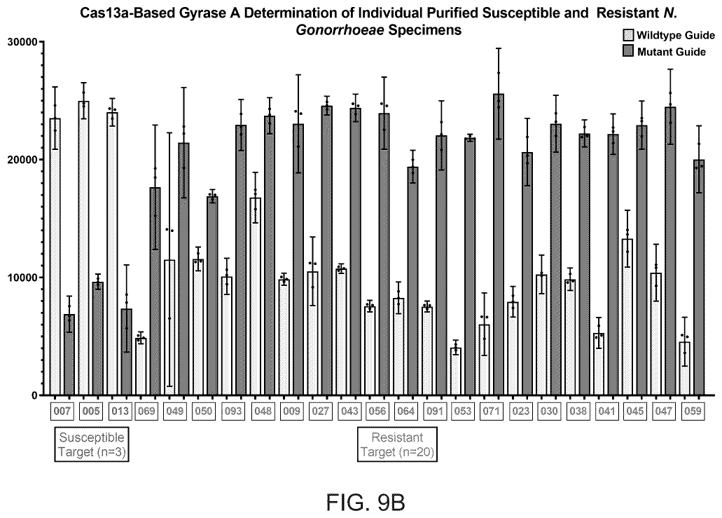

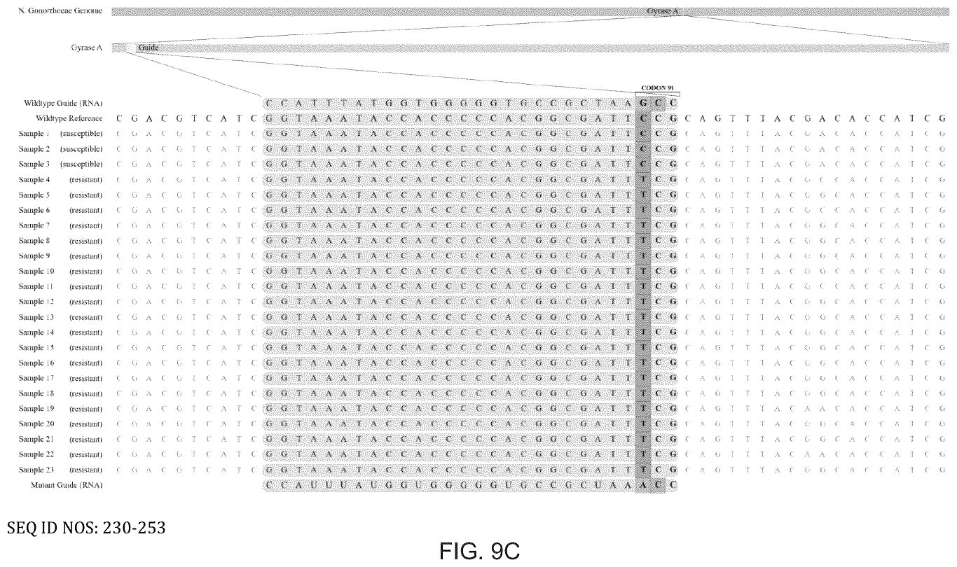

In one embodiment, different orthologs with different sequence specificities may be used. Cutting motifs may be used to take advantage of the sequence specificities of different orthologs. The detection construct can comprise a cutting motif preferentially cut by a Cas protein. A cutting motif sequence can be a particular nucleotide base, a repeat nucleotide base in a homopolymer, or a heteropolymer of bases. The cutting motif can be a dinucleotide sequence, a trinucleotide sequence or more complex motifs comprising 4, 5, 6, 7, 8, 9, or 10 nucleotide motifs. For example, one orthologue may preferentially cut A, while others preferentially cut C, G, U/T. Accordingly, detection constructs completely comprising, or comprised of a substantial portion, of a single nucleotide may be generated, each with a different fluorophore that can be detected at differing wavelengths. In this way up to four different targets may be screened in a single individual discrete volume. In certain example embodiments, different orthologues from a same class of CRISPR effector protein may be used, such as two Cas13a orthologues, two Cas13b orthologues, or two Cas13c orthologues. The nucleotide preferences of various Cas13 proteins is shown in FIGS. 9A-9B. In certain other example embodiments, different orthologues with different nucleotide editing preferences may be used such as a Cas13a and Cas13b orthologs, or a Cas13a and a Cas13c orthologs, or a Cas13b orthologs and a Cas13c orthologs etc. In certain example embodiments, a Cas13 protein with a polyU preference and a Cas13 protein with a polyA preference are used. In certain example embodiments, the Cas13 protein with a polyU preference is a Prevotella intermedia Cas13b, and the Cas13 protein with a polyA preference is a Prevotella sp. MA2106 Cas13b protein (PsmCas13b). In certain example embodiments, the Cas13 protein with a polyU preference is a Leptotrichia wadei Cas13a (LwaCas13a) protein and the Cas13 protein with a poly A preference is a Prevotella sp. MA2106 Cas13b protein. In certain example embodiments, the Cas13 protein with a polyU preference is Capnocytophaga canimorsus Cas 13b protein (CcaCas13b).

Neisseria gonorrhoeae Target Sequences

### Identification of Mutational Drivers of Antibiotic Resistance

Unique Cas orthologs (Cas12a, Cas12b, Cas13a, Cas13b) with different collateral cleavage activities permit multiplexing within one reaction. Multiplex pathogen and AMR detection has a high sensitivity and specificity using three approaches (fluorescence, lateral flow, microfluidics). In one embodiment, one or more guide nucleic acids are designed to detect one or more mutations that confer antibiotic resistance or susceptibility in Neisseria gonorrhoeae. As used herein, “wild type sequence” refers to the naturally occurring, unmutated nucleotide or amino acid sequence of a gene or protein that is characteristic of the predominant form found in antibiotic-susceptible clinical isolates of N. gonorrhoeae. In the context of the penA gene, wild type sequences represent the original, non-mosaic nucleotide sequences at specific codon positions that have not undergone mutation or horizontal gene transfer from other Neisseria species.

### a) Cefixime

Detection of Cefixime Resistant Neisseria gonorrhoeae

In one embodiment, the following one or more guide nucleic acids are designed to detect one or more mutations that confer resistance to cefixime in Neisseria gonorrhoeae.

In one embodiment the one or more mutations are located in at least one codon chosen from positions 375, 376, 377, 501, 542 and 551 of the penA gene (see TABLE II).

In one embodiment the one or more mutations are located in at least two codons chosen from positions 375, 376, 377, 501, 542 and 551 of the penA gene (see TABLE II).

In one embodiment, the one or more guide nucleic acids are designed to detect one or more mutations in the penA gene located in at least three codons chosen from positions 375, 376, 377, 501, 542 and 551 of the penA gene (see TABLE II).

In one embodiment, the one or more guide nucleic acids are designed to detect one or more mutations in the penA gene located in at least four codons chosen from positions 375, 376, 377, 501, 542 and 551 of the penA gene (see TABLE II).

In one embodiment, the one or more guide nucleic acids are designed to detect mutations in the penA gene chosen from codons 501, 542 and 551 of the penA gene (see TABLE II).

In one embodiment, the one or more guide nucleic acids are designed to detect one or more mutations in the penA gene chosen from A501V, A501P, A501T, N512Y, A516G, G542S, G545S, P551L and P551S (see TABLE II).

In one embodiment, one or more guide nucleic acids are designed to detect mutations in the penA gene including, but not limited to, a penA34 mosaic insertion in one or more of the codons chosen from positions 375-377 of the penA gene (see TABLE II).

In one embodiment, a guide nucleic acid is designed to detect nonmosaic penA amino acid substitution A501V/P/T (see TABLE II).

In one embodiment, a guide nucleic acid is designed to detect nonmosaic penA amino acid substitution G542S (see TABLE II).

In one embodiment, a guide nucleic acid is designed to detect nonmosaic penA amino acid substitution P551L/S (see TABLE II).

In one embodiment, one or more guide nucleic acids are designed to detect nonmosaic penA amino acid substitutions A501V/P/T and G542S (see TABLE II).

In one embodiment, one or more guide nucleic acids are designed to detect nonmosaic penA amino acid substitutions A501V/P/T and P551L/S (see TABLE II).

In one embodiment, one or more guide nucleic acids are designed to detect nonmosaic penA amino acid substitutions A501V/P/T, P551L/S and G542S (see TABLE II).

In one embodiment, one or more guide nucleic acids are designed to detect one or more nonmosaic penA mutations comprising A501V/P/T (see TABLE II).

In one embodiment, one or more guide nucleic acids are designed to detect one or more nonmosaic penA mutations comprising G542S.

In one embodiment, one or more guide nucleic acids are designed to detect one or more nonmosaic penA mutations comprising P551L/S.

In one embodiment, one or more guide nucleic acids are designed to detect one or more nonmosaic penA mutations comprising A501V/P/T and G542S.

In one embodiment, one or more guide nucleic acids are designed to detect one or more nonmosaic penA mutations comprising A501V/P/T and P551L/S.

In one embodiment, one or more guide nucleic acids are designed to detect one or more nonmosaic penA mutations comprising A501V/P/T, P551L/S and G542S.

In one embodiment, specific loci of mutations within the penA region that appear to be frequently associated with mosaic penA patterns include 1312M, V316T, N512Y, and G545S.

In one embodiment, one or more guide nucleic acids are designed to detect one or more mutations located in at least one codon chosen from positions 311, 312, 316, 345, 483, 375, 376, 377, 501, 542 and 551 of the penA gene (see TABLES II and XXIX).

TABLE XXIX

| | | Amino acid alteration at number position | | | | | | | | | |
|---|---|---|---|---|---|---|---|---|---|---|---|
| penA type | Mosaicism | A311 | I312 | V316 | D345 insertion | T483 | A501 | N512 | G542 | G545 | P551 |
| 0 (Wild-type) | Non-mosaic | | | | | | | | | | |
| 1 | Non-mosaic | | | | x | | | | | | |
| 2 | Non-mosaic | | | | x | | | | | | |
| 3 | Non-mosaic | | | | x | | | | | | |
| 4 | Non-mosaic | | | | x | | | | S | | |
| 5 | Non-mosaic | | | | x | | | | S | | |
| 7 | Non-mosaic | | | | x | | S | | S | | |
| 9 | Non-mosaic | | | | x | | | | | | L |
| 10 | Mosaic | | M | T | | | | Y | | S | |
| 11 | Non-mosaic | | | | x | | V | | | | L |
| 12 | Non-mosaic | | | | x | | | | | | S |
| 13 | Non-mosaic | | | | x | | V | | | | S |
| 14 | Non-mosaic | | | | x | | | | | | |
| 15 | Non-mosaic | | | | | | | | | | |
| 17 | Non-mosaic | | | | x | | V | | S | | |
| 18 | Non-mosaic | | | | x | | T | | S | | |
| 19 | Non-mosaic | | | | x | | | | | | |
| 21 | Non-mosaic | | | | x | | V | | | | |
| 22 | Non-mosaic | | | | x | | | | | | |
| 26 | Mosaic | | M | T | | | | Y | | S | |
| 27 | Mosaic | | M | T | | | | Y | | S | |
| 30 | Mosaic | | M | T | | V | | Y | | S | |
| 31 | Mosaic | | M | T | | | | Y | | S | |
| 32 | Mosaic | | M | T | | | | Y | | S | |
| 34 | Mosaic | | M | T | | | | Y | | S | |
| 35 | Mosaic | | M | T | | | | | | | |
| 37 | Mosaic | V | M | P | | S | | Y | | S | |
| 38 | Mosaic | | | | | | | | | | |
| 39 | Semi-mosaic | | | | x | | | | | | |
| 40 | Non-mosaic | | | | x | | | | | | |
| 41 | Non-mosaic | | | | x | | | | S | | |
| 42 | Mosaic | | M | T | | | P | Y | | S | |
| 43 | Non-mosaic | | | | x | | V | | | | |
| 44 | Non-mosaic | | | | x | | T | | | | L |
| 45 | Non-mosaic | | | | | | | | | | |
| 46 | Non-mosaic | | | | x | | | | | | |
| 47 | Semi-mosaic | | M | T | | | | | | | |
| 48 | Non-mosaic | | | | x | | | | | | |
| 49 | Non-mosaic | | | | x | | T | | | | |
| 50 | Non-mosaic | | | | x | | | | | | A |
| 51 | Mosaic | | M | T | | | | Y | | S | |
| 52 | Mosaic | | M | T | | | | Y | | S | |
| 53 | Mosaic | | | T | | | | Y | | S | A |
| 54 | Non-mosaic | | | | x | | V | | | | A |
| 55 | Mosaic | | M | T | | | | Y | | S | |
| 56 | Non-mosaic | | | | x | | V | | | | G |
| 57 | Non-mosaic | | | | x | | V | | | | A |
| 58 | Mosaic | | | | | | | Y | | S | A |
| 59 | Mosaic | | | | | S | | Y | | S | |
| 60 | Mosaic | V | M | T | | S | | Y | | S | |
| 61 | Non-mosaic | | | | x | | | | | | L |
| 62 | Mosaic | | | | | | | Y | | | |
| 63 | Mosaic | | M | T | | | | | | | |
| 64 | Mosaic | V | M | T | | S | | Y | | S | |

penA types: mosaicism and amino acid alterations associated with cefixime-decreased susceptible Neisseria gonorrhoeae infections (minimum inhibitory concentration of ≥0.12 μg mL⁻¹)
Reproduced from Table 3 of Deng X, Allan-Blitz L T, Klausner J D. Using the genetic characteristics of Neisseria gonorrhoeae strains with decreased susceptibility to cefixime to develop a molecular assay to predict cefixime susceptibility. Sex Health. 2019 September; 16(5): 488-499.
The one-letter abbreviation of amino acid indicates the substitution of the amino acid described in the header row. The lack of a one-letter abbreviation indicates that the position contains the wild-type amino acid.
The x indicates the insertion of an aspartate at amino acid 345 position.

In one embodiment, any one of the cefixime resistant Neisseria gonorrhoeae identified above further comprise a mutation at codon 91 or 95 in the gyrA gene (see TABLE I).

In one embodiment, any one of the cefixime resistant Neisseria gonorrhoeae identified above further comprise a S91F mutation in the gyrA gene.

In one embodiment, any one of the cefixime resistant Neisseria gonorrhoeae identified above further comprise a D95G or D95N mutation in the gyrA gene.

In one embodiment, any one of the cefixime resistant Neisseria gonorrhoeae identified above further comprise D95G and D95N mutations in the gyrA gene.

In one embodiment, any one of the cefixime resistant Neisseria gonorrhoeae identified above further comprise S91F, D95G and D95N mutations in the gyrA gene.

In one embodiment, any one of the cefixime resistant Neisseria gonorrhoeae identified above further comprise a wild type sequence at codon S91 in the gyrA gene.

In one embodiment, any one of the cefixime resistant Neisseria gonorrhoeae identified above further comprise a wild type sequence at codon D95 in the gyrA gene.

In one embodiment, any one of the cefixime resistant Neisseria gonorrhoeae identified above further comprise a wild type sequence at codons S91 and D95 in the gyrA gene.

Detection of Cefixime Susceptible Neisseria gonorrhoeae

In one embodiment, the following one or more guide nucleic acids are designed to detect wild type sequences that confer susceptibility to cefixime in Neisseria gonorrhoeae.

In one embodiment one or more guide nucleic acids are designed to detect wild type sequences at one or more codons chosen from A311, 1312, V316, D345, T483, A501, N512, G542, G545, and P551 of the penA gene (see TABLE II).

In one embodiment one or more guide nucleic acids are designed to detect wild type sequences at two or more codons chosen from A311, 1312, V316, D345, T483, A501, N512, G542, G545, and P551 of the penA gene (see TABLE II).

In one embodiment one or more guide nucleic acids are designed to detect wild type sequences at three or more codons chosen from A311, 1312, V316, D345, T483, A501, N512, G542, G545, and P551 of the penA gene (see TABLE II).

In one embodiment one or more guide nucleic acids are designed to detect wild type sequences at four or more codons chosen from A311, 1312, V316, D345, T483, A501, N512, G542, G545, and P551 of the penA gene (see TABLE II).

In one embodiment, any one of the cefixime susceptible Neisseria gonorrhoeae identified above further comprise a mutation at codon 91 or 95 in the gyrA gene (see TABLE I).

In one embodiment, any one of the cefixime susceptible Neisseria gonorrhoeae identified above further comprise a S91F mutation in the gyrA gene.

In one embodiment, any one of the cefixime susceptible Neisseria gonorrhoeae identified above further comprise a D95G or D95N mutation in the gyrA gene.

In one embodiment, any one of the cefixime susceptible Neisseria gonorrhoeae identified above further comprise D95G and D95N mutations in the gyrA gene.

In one embodiment, any one of the cefixime susceptible Neisseria gonorrhoeae identified above further comprise S91F, D95G and D95N mutations in the gyrA gene.

In one embodiment, any one of the cefixime susceptible Neisseria gonorrhoeae identified above do not comprise a mutation at codon 91 or 95 in the gyrA gene (see TABLE I).

In one embodiment, any one of the cefixime susceptible Neisseria gonorrhoeae identified above do not comprise a S91F mutation in the gyrA gene.

In one embodiment, any one of the cefixime susceptible Neisseria gonorrhoeae identified above do not comprise a D95G or D95N mutation in the gyrA gene.

In one embodiment, any one of the cefixime susceptible Neisseria gonorrhoeae identified above do not comprise D95G and D95N mutations in the gyrA gene.

In one embodiment, any one of the cefixime susceptible Neisseria gonorrhoeae identified above do not comprise S91F, D95G and D95N mutations in the gyrA gene.

In one embodiment, any one of the cefixime susceptible Neisseria gonorrhoeae identified above further comprise a wild type sequence at codon S91 and/or D95 in the gyrA gene.

### b) Ceftriaxone

Detection of Ceftriaxone Resistant Neisseria gonorrhoeae

In one embodiment, the following one or more guide nucleic acids are designed to detect one or more mutations that confer resistance to ceftriaxone in Neisseria gonorrhoeae.

In one embodiment, one or more guide nucleic acids are designed to detect one or more mutations in the penA gene comprising mutations in the mosaic allele penA60.001 of the penA gene (see TABLE II).

In one embodiment, one or more guide nucleic acids are designed to detect the one or more mutations in the penA gene comprising mutations in the mosaic allele penA60.001 and in at least one of the codons at positions 311, 316, and 483 of the penA gene (see TABLE II).

In one embodiment, one or more guide nucleic acids are designed to detect the one or more mutations in the penA gene comprising mutations in the mosaic allele penA60.001 and in at least two of the codons at positions 311, 316, and 483 of the penA gene (see TABLE II).

In one embodiment, one or more guide nucleic acids are designed to detect one or more mutations in the penA gene comprising mutations in the mosaic allele penA60.001 and in codons 311, 316, and 483 of the penA gene (see TABLE II).

In one embodiment, a guide nucleic acid is designed to detect penA amino acid substitution A311V (see TABLE II).

In one embodiment, a guide nucleic acid is designed to detect penA amino acid substitution V316T/P (see TABLE II).

In one embodiment, a guide nucleic acid is designed to detect penA amino acid substitution T483S (see TABLE II).

In one embodiment, a guide nucleic acid is designed to detect penA amino acid substitutions A311V and V316T/P (see TABLE II).

In one embodiment, a guide nucleic acid is designed to detect penA amino acid substitutions A311V and T483S (see TABLE II).

In one embodiment, a guide nucleic acid is designed to detect penA amino acid substitutions A311V, V316T/P and T483S (see TABLE II).

In one embodiment, a guide nucleic acid is designed to detect amino acid substitutions G120X and/or A121X in the porB gene (see TABLE III).

In one embodiment, a guide nucleic acid is designed to detect amino acid substitution L421P in the ponA gene (see TABLE IV).

In one embodiment, a guide nucleic acid is designed to detect amino acid substitution A501P/V/T in the penA gene.

In one embodiment, a guide nucleic acid is designed to detect amino acid substitution G542S in the penA gene.

In one embodiment, a guide nucleic acid is designed to detect amino acid substitution N512Y in the penA gene.

In one embodiment, a guide nucleic acid is designed to detect amino acid substitution A516G in the penA gene.

In one embodiment, a guide nucleic acid is designed to detect amino acid substitution G545S in the penA gene.

In one embodiment, one or more guide nucleic acids are designed to detect one or more mutations located in at least one codon chosen from codons 311, 316, 483, 501, 512, 516, 542 and 545 of the penA gene (see TABLES II and XX).

In one embodiment, one or more guide nucleic acids are designed to detect one or more mutations located at codons 120 and 121 in the porB gene.

In one embodiment, one or more guide nucleic acids are designed to detect a mutation located at codon 421 in the ponA gene.

In one embodiment, a guide nucleic acid is designed to detect amino acid substitution L421P in the ponA gene.

In one embodiment, a guide nucleic acid is designed to detect amino acid substitution G120X in the penA gene.

In one embodiment, a guide nucleic acid is designed to detect amino acid substitution G121X in the penA gene.

TABLE XXX
Sensitivity and specificity values of applying various genetic alterations
contributing to ceftriaxone resistance in N. gonorrhoeae to the
PathogenWatch database and comparison with previous studies

| Genetic mutations | Parameter | Pathogen Watch | Prior studies |
|---|---|---|---|
| MtrR promoter del | Sensitivity | 88.9% | 95.3%^{a}, 98.3%^{b} |
| | Specificity | 62.9% | 52.8%^{a}, 65.2%^{b} |
| porB at least 1 of G120X, A121X | Sensitivity | 98.4% | 96.6%^{a}, 94.8%^{b} |
| | Specificity | 44.6% | 42.1%^{a}, 50.8%^{b} |
| porB both G120X, A121X | Sensitivity | 96.2% | 94.3%^{a} |
| | Specificity | 61.2% | 52.3%^{a} |
| ponA L421P | Sensitivity | 99.2% | 99.6%^{a}, 100%^{b} |
| | Specificity | 55.7% | 45.4%^{a}, 57.4%^{b} |
| penA A311V | Sensitivity | 2.4% | 3.9%^{a} |
| | Specificity | 100.0% | 99.9%^{a} |
| penA A501P/V/T | Sensitivity | 27.4% | 36.9%^{a} |
| | Specificity | 93.0% | 93.6%^{a} |
| penA N512Y | Sensitivity | 61.4% | 42.0%^{a} |
| | Specificity | 89.1% | 75.8%^{a} |
| penA A516G | Sensitivity | 38.6% | 55.0%^{a} |
| | Specificity | 17.3% | 30.5%^{a} |
| penA G542S | Sensitivity | 12.8% | 19.3%^{a} |
| | Specificity | 93.2% | 86.3%^{a} |
| penA G545S | Sensitivity | 61.4% | 41.8%^{a} |
| | Specificity | 90.1% | 85.9%^{a} |
| Peterson et al. Multiplex real-time PCR assays | Sensitivity | 96.5% | 99.8% |
| for the prediction of cephalosporin, | Specificity | 70.3% | 89.0% |
| ciprofloxacin and azithromycin antimicrobial | | | |
| susceptibility of positive Neisseria | | | |
| gonorrhoeae nucleic acid amplification test | | | |
| samples. J Antimicrob Chemother. | | | |
| 2020; 75(12): 3485-3490. | | | |
| Peterson assay. | | | |
| Any three of: ponA L421P, at least 1 of porB | | | |
| G120X and A121X, mtrR -35delA, penA | | | |
| A311V, penA A501, penA N512Y, penA | | | |
| G545S | | | |

^{a}Lin E Y, Adamson P C, Ha S M, Klausner J D. Reliability of Genetic Alterations in Predicting Ceftriaxone Resistance in Neisseria gonorrhoeae Globally. Microbiol Spectr. 2022 10(2): e0206521. doi: 10.1128/spectrum.02065-21.
^{b}Peterson et al. (2015) Molecular assay for detection of genetic markers associated with decreased susceptibility to cephalosporins in Neisseria gonorrhoeae. J Clin Microbiol 53: 2042-2048 with the definition of decreased ceftriaxone susceptibility as ≥0.06 mg/liter as opposed to >0.064 mg/liter.
X: refers to any amino acid substitution

In one embodiment, any one of the ceftriaxone resistant Neisseria gonorrhoeae identified above further comprise a mutation at codon 91 or 95 in the gyrA gene (see TABLE I).

In one embodiment, any one of the ceftriaxone resistant Neisseria gonorrhoeae identified above further comprise a S91F mutation in the gyrA gene.

In one embodiment, any one of the ceftriaxone resistant Neisseria gonorrhoeae identified above further comprise a D95G or D95N mutation in the gyrA gene.

In one embodiment, any one of the ceftriaxone resistant Neisseria gonorrhoeae identified above further comprise D95G and D95N mutations in the gyrA gene.

In one embodiment, any one of the ceftriaxone resistant Neisseria gonorrhoeae identified above further comprise S91F, D95G and D95N mutations in the gyrA gene.

In one embodiment, any one of the ceftriaxone resistant Neisseria gonorrhoeae identified above further comprise a wild type sequence at codon S91 in the gyrA gene.

In one embodiment, any one of the ceftriaxone resistant Neisseria gonorrhoeae identified above further comprise a wild type sequence at codon D95 in the gyrA gene.

In one embodiment, any one of the ceftriaxone resistant Neisseria gonorrhoeae identified above further comprise a wild type sequence at codons S91 and D95 in the gyrA gene.

Detection of Ceftriaxone Susceptible Neisseria gonorrhoeae

In one embodiment, the following one or more guide nucleic acids are designed to detect wild type sequences that confer susceptibility to ceftriaxone in Neisseria gonorrhoeae.

In one embodiment one or more guide nucleic acids are designed to detect wild type sequences at one or more codons chosen from codons A311, V316, T483, T483, A501, N512, A516, G542, and G545 of the penA gene (see TABLE II).

In one embodiment one or more guide nucleic acids are designed to detect wild type sequences at two or more codons chosen from codons A311, V316, T483, T483, A501, N512, A516, G542, and G545 of the penA gene (see TABLE II).

In one embodiment one or more guide nucleic acids are designed to detect wild type sequences at three or more codons chosen from codons A311, V316, T483, T483, A501, N512, A516, G542, and G545 of the penA gene (see TABLE II).

In one embodiment one or more guide nucleic acids are designed to detect wild type sequences at four or more codons chosen from codons A311, V316, T483, T483, A501, N512, A516, G542, and G545 of the penA gene (see TABLE II).

In one embodiment one or more guide nucleic acids are designed to detect wild type sequences at codon L421 in the ponA gene (see TABLE IV).

In one embodiment one or more guide nucleic acids are designed to detect wild type sequences at codon G120 and/or A121 in the porB gene (see TABLE III).

In one embodiment, any one of the ceftriaxone susceptible Neisseria gonorrhoeae identified above further comprise a mutation at codon 91 or 95 in the gyrA gene (see TABLE I).

In one embodiment, any one of the ceftriaxone susceptible Neisseria gonorrhoeae identified above further comprise a S91F mutation in the gyrA gene.

In one embodiment, any one of the ceftriaxone susceptible Neisseria gonorrhoeae identified above further comprise a D95G or D95N mutation in the gyrA gene.

In one embodiment, any one of the ceftriaxone susceptible Neisseria gonorrhoeae identified above further comprise D95G and D95N mutations in the gyrA gene.

In one embodiment, any one of the ceftriaxone susceptible Neisseria gonorrhoeae identified above further comprise S91F, D95G and D95N mutations in the gyrA gene.

In one embodiment, any one of the ceftriaxone susceptible Neisseria gonorrhoeae identified above do not comprise a mutation at codon 91 or 95 in the gyrA gene (see TABLE I).

In one embodiment, any one of the ceftriaxone susceptible Neisseria gonorrhoeae identified above do not comprise a S91F mutation in the gyrA gene.

In one embodiment, any one of the ceftriaxone susceptible Neisseria gonorrhoeae identified above do not comprise a D95G or D95N mutation in the gyrA gene.

In one embodiment, any one of the ceftriaxone susceptible Neisseria gonorrhoeae identified above do not comprise D95G and D95N mutations in the gyrA gene.

In one embodiment, any one of the ceftriaxone susceptible Neisseria gonorrhoeae identified above do not comprise S91F, D95G and D95N mutations in the gyrA gene.

In one embodiment, any one of the ceftriaxone susceptible Neisseria gonorrhoeae identified above further comprise a wild type sequence at codon S91 and/or D95 in the gyrA gene.

### c) Cefixime and Ceftriaxone

Detection of Cefixime and Ceftriaxone Resistant Neisseria gonorrhoeae

In one embodiment, the following one or more guide nucleic acids are designed to detect one or more mutations that confer resistance to cefixime and ceftriaxone in Neisseria gonorrhoeae.

In one embodiment, one or more guide nucleic acids are designed to detect at least one mutation located in any one of the codons chosen from positions 311, 312, 316, 345, 483, 375, 376, 377, 501, 542 and 551 of the penA gene and (2) at least one mutation located in the mosaic allele penA60.001 and in any one of the codons chosen from positions 311, 316, 483, 501, 512, 516, 542 and 545 of the penA gene (see TABLE II) and/or in codon 120 or 121 in the porB gene and/or 421 in the ponA gene.

In one embodiment, one or more guide nucleic acids are designed to detect (1) at least one mutation chosen from A501V, A501P, A501T, N512Y, A516G, G542S, G545S, P551L and P551S in the penA gene, (2) at least one mutation chosen from A311V, V316T/P and T483S in the penA gene, and optionally (3) G120X and/or A121X in the porB gene and/or (4) L421P in the ponA gene.

In one embodiment, any one of the cefixime and ceftriaxone resistant Neisseria gonorrhoeae identified above further comprise a mutation at codon 91 or 95 in the gyrA gene (see TABLE I).

In one embodiment, any one of the cefixime and ceftriaxone resistant Neisseria gonorrhoeae identified above further comprise a S91F mutation in the gyrA gene.

In one embodiment, any one of the cefixime and ceftriaxone resistant Neisseria gonorrhoeae identified above further comprise a D95G or D95N mutation in the gyrA gene.

In one embodiment, any one of the cefixime and ceftriaxone resistant Neisseria gonorrhoeae identified above further comprise D95G and D95N mutations in the gyrA gene.

In one embodiment, any one of the cefixime and ceftriaxone resistant Neisseria gonorrhoeae identified above further comprise S91F, D95G and D95N mutations in the gyrA gene.

In one embodiment, any one of the cefixime and ceftriaxone resistant Neisseria gonorrhoeae identified above further comprise a wild type sequence at codon S91 in the gyrA gene.

In one embodiment, any one of the cefixime and ceftriaxone resistant Neisseria gonorrhoeae identified above further comprise a wild type sequence at codon D95 in the gyrA gene.

In one embodiment, any one of the cefixime and ceftriaxone resistant Neisseria gonorrhoeae identified above further comprise a wild type sequence at codons S91 and D95 in the gyrA gene.

Detection of Cefixime and Ceftriaxone Susceptible Neisseria gonorrhoeae

In one embodiment, the following one or more guide nucleic acids are designed to detect wild type sequences that confer susceptibility to cefixime and ceftriaxone in Neisseria gonorrhoeae.

In one embodiment one or more guide nucleic acids are designed to detect (1) wild type sequences at one or more codons chosen from A311, 1312, V316, D345, T483, A501, N512, G542, G545, and P551 of the penA gene, and (2) wild type sequences at one or more codons chosen from A311, V316, T483, T483, A501, N512, A516, G542, and G545 of the penA gene and optionally (3) wild type sequences at one or more codons chosen from G120 and/or A121 in the porB gene and/or L421 in the ponA gene.

In one embodiment, any one of the cefixime and ceftriaxone susceptible Neisseria gonorrhoeae identified above further comprise a mutation at codon 91 or 95 in the gyrA gene (see TABLE I).

In one embodiment, any one of the cefixime and ceftriaxone susceptible Neisseria gonorrhoeae identified above further comprise a S91F mutation in the gyrA gene.

In one embodiment, any one of the cefixime and ceftriaxone susceptible Neisseria gonorrhoeae identified above further comprise a D95G or D95N mutation in the gyrA gene.

In one embodiment, any one of the cefixime and ceftriaxone susceptible Neisseria gonorrhoeae identified above further comprise D95G and D95N mutations in the gyrA gene.

In one embodiment, any one of the cefixime and ceftriaxone susceptible Neisseria gonorrhoeae identified above further comprise S91F, D95G and D95N mutations in the gyrA gene.

In one embodiment, any one of the cefixime and ceftriaxone susceptible Neisseria gonorrhoeae identified above further comprise a wild type sequence at codon S91 in the gyrA gene.

In one embodiment, any one of the cefixime and ceftriaxone susceptible Neisseria gonorrhoeae identified above further comprise a wild type sequence at codon D95 in the gyrA gene.

In one embodiment, any one of the cefixime and ceftriaxone susceptible Neisseria gonorrhoeae identified above further comprise a wild type sequence at codons S91 and D95 in the gyrA gene.

### VII. Applications

### a) Resistance-Guided Therapy

The embodiments disclosed herein utilize RNA targeting effectors to provide a robust CRISPR-based diagnostic for multiplexed applications by performing detection on lateral flow strips. Embodiments disclosed herein can detect both DNA and RNA with comparable levels of sensitivity and can differentiate targets from non-targets based on single base pair differences.

A low-cost, point-of-care Cas13a-based Neisseria gonorrhoeae assay has a variety of novel applications. First and foremost, the assay will permit detection of the Neisseria pathogen in settings previously limited to syndromic management. Syndromic management has been shown to have poor correlation with etiologic-based therapy; further, syndromic management misses a high proportion of cases (Otieno et al. Evaluation of syndromic management of sexually transmitted infections within the Kisumu Incidence Cohort Study. Int J STD AIDS. 2014; 25(12):851-9; Verwijs et al. Targeted point-of-care testing compared with syndromic management of urogenital infections in women (WISH): a cross-sectional screening and diagnostic accuracy study. Lancet Infect Dis. 2019; 19(6):658-69). Thus, areas with already high prevalence of Neisseria gonorrhoeae infection and high rates of antimicrobial resistance, are compelled to utilize a treatment strategy that results in both overuse of antibiotics, likely contributing to antimicrobial resistance, and which permits a high proportion of infections to go untreated, compounding the associated morbidity. The assay will introduce low-cost, point-of-care pathogen detection, permitting more selective therapeutic approaches, reducing the overuse of antibiotics, and increasing case finding capabilities. In addition, the assay will permit resistance-guided therapy, by providing real-time information on susceptibility to several antibiotics currently in use for the treatment of gonorrhea, including ciprofloxacin, cefixime and ceftriaxone.

Beyond resource-limited settings, the assay should facilitate home-testing for Neisseria gonorrhoeae. Self-testing for other sexually transmitted pathogens including HIV and human papilloma virus, have showed the potential for dramatic increases in case finding. Self-testing for Neisseria gonorrhoeae has the potential to result in reduced sexual risk behaviors among those who self-identify as positive, and thus decrease transmission, particularly among core groups of high-risk individuals and at venues where transmission of sexually transmitted infection remains high.

### b) Detection of Single Nucleotide Variants

In some embodiments, one or more identified target sequences may be detected using guide RNAs that are specific for and bind to the target sequence as described herein. The systems and methods of the present disclosure can distinguish even between single nucleotide polymorphisms (SNPs) present among different microbial species and therefore, use of multiple guide RNAs in accordance with the disclosure may further expand on or improve the number of target sequences that may be used to distinguish between species or detect antibiotic resistance.

### c) Monitoring Microbe Outbreaks

In some embodiments, a CRISPR system or methods of use thereof as described herein may be used to determine the evolution of an antibiotic resistant pathogen outbreak. The method may comprise detecting one or more target sequences from a plurality of samples from one or more subjects, wherein the target sequence is a sequence from a microbe causing the outbreaks. Such a method may further comprise determining a pattern of antibiotic resistant pathogen transmission, or a mechanism involved in a disease outbreak caused by an antibiotic resistant pathogen.

The pattern of pathogen transmission may comprise continued new transmissions from the natural reservoir of the pathogen or subject-to-subject transmissions (e.g. human-to-human transmission) following a single transmission from the natural reservoir or a mixture of both. In one embodiment, the pattern of the pathogen transmission is the early pattern of the pathogen transmission, i.e. at the beginning of the pathogen outbreak. Determining the pattern of the pathogen transmission at the beginning of the outbreak increases likelihood of stopping the outbreak at the earliest possible time thereby reducing the possibility of local and international dissemination.

In any method described above, sequencing the target sequence or fragment thereof may be a near-real-time sequencing. Sequencing the target sequence or fragment thereof may be carried out according to previously described methods. Sequencing the target sequence or fragment thereof may comprise parallel sequencing of a plurality of target sequences. Sequencing the target sequence or fragment thereof may comprise Illumina sequencing.

Analyzing the target sequence or fragment thereof that hybridizes to one or more of the selected probes may be an identifying analysis, wherein hybridization of a selected probe to the target sequence or a fragment thereof indicates the presence of the target sequence within the sample.

The present disclosure may also be used in concert with other methods of diagnosing disease, identifying pathogens and optimizing treatment based upon detection of nucleic acids, such as mRNA in crude, non-purified samples.

In some cases, the patient is presented to the medical staff for diagnostics of particular symptoms. The method of the disclosure makes it possible not only to identify which disease causes these symptoms but at the same time determine whether the patient suffers from another disease he was not aware of.

### d) Screening Environmental Samples

The methods disclosed herein may also be used to screen environmental samples for N. gonorrhoeae. For example, in some embodiments, the disclosure provides a method of detecting microbes, comprising: exposing a CRISPR system as described herein to a sample; activating an RNA effector protein via binding of one or more guide RNAs to one or more microbe-specific target RNAs or one or more trigger RNAs such that a detectable positive signal is produced. The positive signal can be detected and is indicative of the presence of one or more microbes in the sample. In some embodiments, the CRISPR system may be on a substrate as described herein, and the substrate may be exposed to the sample. In other embodiments, the same CRISPR system, and/or a different CRISPR system may be applied to multiple discrete locations on the substrate. In further embodiments, the different CRISPR system may detect a different microbe at each location. As described in further detail above, a substrate may be a flexible materials substrate, for example, including, but not limited to, a paper substrate, a fabric substrate, or a flexible polymer-based substrate.

In accordance with the disclosure, the substrate may be exposed to the sample passively, by temporarily immersing the substrate in a fluid to be sampled, by applying a fluid to be tested to the substrate, or by contacting a surface to be tested with the substrate. Any means of introducing the sample to the substrate may be used as appropriate.

As described herein, a sample for use with the disclosure may be a biological or environmental sample, such as a freshwater sample, a wastewater sample, a saline water sample, or a combination thereof. In further embodiments, a biological sample may be obtained from a source including, but not limited to, a tissue sample, saliva, blood, plasma, sera, stool, urine, sputum, mucous, lymph, synovial fluid, cerebrospinal fluid, ascites, pleural effusion, seroma, pus, or swab of skin or a mucosal membrane surface. In some particular embodiments, an environmental sample or biological samples may be crude samples and/or the one or more target molecules may not be purified or amplified from the sample prior to application of the method. Identification of microbes may be useful and/or needed for any number of applications, and thus any type of sample from any source deemed appropriate by one of skill in the art may be used in accordance with the disclosure.

In other embodiments, a sample may be an environmental sample, such as water, or a surface such as industrial or medical surface. In some embodiments, methods such as disclosed in US patent publication No. 2013/0190196 may be applied for detection of nucleic acid signatures, specifically RNA levels, directly from crude cellular samples with a high degree of sensitivity and specificity. Sequences specific to Neisseria pathogens of interest may be identified or selected by comparing the coding sequences from the pathogen of interest to all coding sequences in other organisms by BLAST software.

Several embodiments of the present disclosure involve the use of procedures and approaches known in the art to successfully fractionate clinical blood samples. See, e.g. the procedure described in Han Wei Hour et al., Microfluidic Devices for Blood Fractionation, Micromachines 2011, 2, 319-343; Ali Asgar S. Bhagat et al., Dean Flow Fractionation (DFF) Isolation of Circulating Tumor Cells (CTCs) from Blood, 15th International Conference on Miniaturized Systems for Chemistry and Life Sciences, Oct. 2-6, 2011, Seattle, WA; and International Patent Publication No. WO2011109762, the disclosures of which are herein incorporated by reference in their entirety. Blood samples are commonly expanded in culture to increase sample size for testing purposes. In some embodiments of the present disclosure, blood or other biological samples may be used in methods as described herein without the need for expansion in culture.

Further, several embodiments of the present disclosure involve the use of procedures and approaches known in the art to successfully isolate pathogens from whole blood using spiral microchannel, as described in Han Wei Hour et al., Pathogen Isolation from Whole Blood Using Spiral Microchannel, Case No. 15995JR, Massachusetts Institute of Technology, manuscript in preparation, the disclosure of which is herein incorporated by reference in its entirety.

Owing to the increased sensitivity of the embodiments disclosed herein, in certain example embodiments, the assays and methods may be run on crude samples or samples where the target molecules to be detected are not further fractionated or purified from the sample.

### VIII. Kits

In one aspect, the disclosure provides kits containing any one or more of the elements disclosed in the above methods and compositions. In one aspect, the disclosure provides a kit comprising one or more of the components described herein. In one embodiment, the kit comprises the compositions herein and instructions for using the kit. In one embodiment, the kit comprises a lateral flow strip, reagent and instructions for using the kit. Elements may be provided individually or in combinations, and may be provided in any suitable container, such as a vial, a bottle, or a tube. The kits may include synthetic RNA or DNA comprising the target nucleotide sequence as controls. In one embodiment, the kit includes instructions in one or more languages, for example in more than one language. The instructions may be specific to the applications and methods described herein.

In one embodiment, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In one embodiment, the buffer is alkaline. In one embodiment, the buffer has a pH from about 7 to about 10. In one embodiment, the kit comprises one or more oligonucleotides for RPA amplification.

The invention is further described in the following examples, which do not limit the scope of the invention described in the claims.

Further embodiments are illustrated in the following Examples which are given for illustrative purposes only and are not intended to limit the scope of the invention

### EXAMPLES

### Example 1: A Point of Care Multiplex Lateral Flow Assay for the Detection of Ciprofloxacin-Resistant N. Gonorrhoeae

### 1) Materials And Methods

Synthetic DNA Preparation and DNA Extraction from Purified Isolates

Assays using both synthetic N. gonorrhoeae DNA and purified N. gonorrhoeae isolates were tested. Synthetic DNA samples were prepared by serial dilution from commercially purchased (Integrated DNA Technologies, USA), double-stranded DNA (dsDNA) of the gyrA target region into nuclease-free water. Purified isolates were stored in glycerol at −80° C. prior to extraction. Whole-genomic DNA from N. gonorrhoeae purified isolates was extracted using the DNeasy Blood and Tissue Kit (Qiagen, Germany). The starting volume for extraction was 400 μL, and extracted DNA was eluted into 100 μL of nuclease-free water.

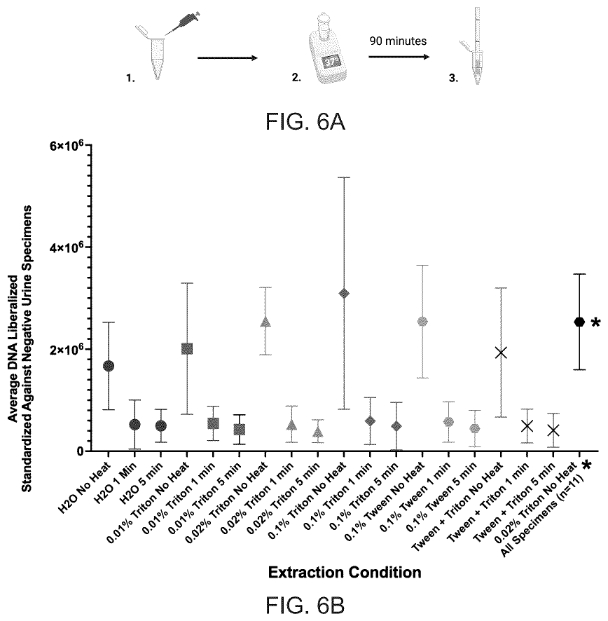

In one embodiment, the genomic DNA of a N. gonorrhoeae isolate can be extracted in in 0.05%-0.5% Triton-X at a temperature of 37° C. for 1-5 minutes (FIGS. 6A and 6B).

With each isolate, minimum inhibitory concentrations (MICs) in micrograms per milliliter were provided for ciprofloxacin, obtained using standard methods, as well as the anatomic site of collection. Additionally, non-gonococcal Neisseria isolates were purchased from American Type Culture Collection (ATCC), and the Massachusetts General Hospital Clinical Microbiology Laboratory cultured those isolates: N. meningitidis (ATCC 13077), N. perflava (ATCC 14799), and N. lactamica (ATCC 23970). The performance of the porA assay was also assessed on those isolates.

The concentration of extracted N. gonorrhoeae DNA was quantified using quantitative polymerase chain reaction (qPCR). The forward and reverse primer sequences for the N. gonorrhoeae gyrA gene were 5′ GCGACGGCCTAAAGCCAGTG 3′ (SEQ ID NO:207) and 5′ GTCTGCCAGCATTTCATGTGAG 3′ (SEQ ID NO:208), respectively. The qPCR mixtures contained 1× FastStart SYBR Green Master Mix (Sigma Aldrich, USA), 0.5 μM of each primer, and DNA template in a 1:9 template to master mix ratio. The final qPCR volume was adjusted to 10 μL with nuclease-free water and loaded in triplicate on a 384-well plate, which was run on a QuantStudio 6 (Applied Biosystems, USA) with the following cycle conditions: heat activation at 95° C. for 3 minutes, 40 cycles of a denaturing step at 95° C. for 15 seconds, an annealing step at 60° C. for 1 minute, and an extension step at 72° C., followed by a final extension step at 68° C. for 2 minutes. Amplification data was collected during the second extension stage and analyzed those data using the standard curve module of the Applied Biosystems Analysis Software. Isolates were quantified against a standard curve, which showed an average concentration of 1,000 copies per milliliter across isolates. Subsequently, thermal DNA extraction was assessed by resuspending three purified isolates in 100 μL of nuclease-free water and heating the isolates to 95° C. for 10 minutes in accordance with prior protocols.

Guide RNA and Primer Design for N. gonorrhoeae Detection

Cas13a gRNAs have two components: the fixed “handle” region to which the Cas13a protein binds and a 28-nucleotide “spacer” region complementary to the target. The nucleotide sequence of the spacer can be chosen by the user to confer the specificity of the assay. The porA gene of N. gonorrhoeae was selected for pathogen detection as it has been used previously (Whiley et al., Evidence that the gonococcal porA pseudogene is present in a broad range of Neisseria gonorrhoeae strains; suitability as a diagnostic target (2006) Pathology 38:445-448). An online software package ADAPT (Activity-Informed Design with All-Inclusive Patrolling of Targets; https://adapt.run) was used for the design of the guide RNA (Metsky et al., Designing sensitive viral diagnostics with machine learning (2022) Nat Biotechnol 40:1123-1131), which applies an algorithm for optimal Cas13a gRNAs design, and selected three gRNAs from the output of that software.

Forward and reverse RPA primers were designed using National Center for Biotech-nology Information Primer-Basic Local Alignment Search Tool (BLAST) and synthesized by Integrated DNA Technologies (USA). Two primer sets per guide location were selected (total of six primer sets), each primer being 27-35 nucleotides in length. The primer sets had melting temperatures between 58° C. and 68° C. and produced amplicons of 140-200 base pairs in length. A T7 RNA polymerase promoter sequence (5′ GAAATTAATACGACTCACTATAGG 3′ (SEQ ID NO: 209) was added to the 5′ end of the forward primers of each set to allow for T7 transcription.

### One-Pot SHERLOCK Assay

SHERLOCK reactions were performed using 45 nM C2c2 LwaCas13a (GenScript Biotech Corp, USA) resuspended in 1x storage buffer (SB: 50 mM Tris [pH 7.5], 600 mM KCl, 5% glycerol, and 2 mM dithiothreitol [DTT]) such that the resuspended protein was at 2.25 μM, 1 U/μL murine RNase inhibitor (NEB), 10 U/μL T7 RNA polymerase (Lucigen Corporation, USA), 136 nM RNaseAlert substrate v2 (ThermoFisher Scientific, USA), 1× SHINE Buffer {SHINE: 20 mM HEPES [4-(2-hydroxyethyl)-1-piperazineethanesulfonic acid] (pH 8.0), 60 mM KCl, and 5% polyethylene glycol (PEG)}, and 2 mM of each rNTP (NEB).

The TwistAmp Basic Kit lyophilized pellets was rehydrated (one pellet per 73.42-μL master mix volume) using the prepared master mix. 14 mM MgAOc (TwistDx, United Kingdom) was added after resuspension to activate the RPA pellets. The master mix was then subdivided into aliquots for each guide-primer set pair being analyzed. 22.5 nM gRNA (Integrated DNA Technologies, USA) and 320 nM each of the RPA primers (Integrated DNA Technologies, USA) was then added to each aliquot. SHERLOCK reactions were prepared to 70 μL and loaded as 20-μL triplicates into a 384-well plate, with a ratio of 1:5 master mix to sample. Fluorescence was measured using a BioTek Cytation 5 plate reader (BioTek, USA) over 3 hours at 37° C., with readings every 5 minutes (excitation, 485; emission, 528) for quantitative detection.

### Lateral Flow Detection

To convert to lateral flow readout, the SHERLOCK master mix was modified to exchange substrate v2 for a biotinylated fluorescein (FAM) reporter at a final concentration of 1 μM. Samples were incubated at 37° C. for 90 minutes per existing protocols to allow for optimal RPA amplification. Following incubation, 80-μL HybriDetect assay buffer (Milennia Biotec, Germany) was added to each sample in a 1:5 dilution along with a HybriDetect lateral flow strip (Milennia Biotec, Germany). Strips were inspected and images were documented using a smartphone camera 3-5 minutes after the strips were added.

### Confirmatory DNA Sequencing

Whole-genome sequencing was performed on extracted DNA samples following the Illumina DNA Prep manufacturer protocol (Illumina, USA). Pooled libraries were constructed using the Illumina DNA Prep Kit. Library concentrations were measured on a Qubit4 Fluorometer using the Qubit High Sensitivity 1×dsDNA kit (ThermoFisher Scientific, USA), while the average library size was measured on an Agilent TapeStation 4150 using the Agilent High Sensitivity D1000 ScreenTape kit (Agilent Technologies, USA). Genomic sequencing was conducted on an Illumina MiniSeq instrument (Illumina, USA).

### Data Analysis

Baseline fluorescence (at 0 minutes) was subtracted from fluorescence values through reaction progression. The final 10 fluorescence values of each replicate was then averaged to provide the reported fluorescence values. Mean differences in fluorescence were compared using Student's t-test, with significance defined as P<0.05. Lateral flow readouts were interpreted by visual inspection. All figures were generated using PRISM Software version 9.5.1 (GraphPad, USA).

### General Protocols

Provided are two ways to perform a C2c2 diagnostic test for DNA and RNA. The first is a two-step reaction where amplification and C2c2 detection are done separately. The second is where everything is combined in one reaction and this is called a two-step reaction.

TABLE XXXI
CRISPR Effector Only - No amplification

| Component | Volume (μL) |
|---|---|
| Protein (44 nM final) | 2 |
| crRNA (12 nM final) | 1 |
| background target (100 ng total) | 1 |
| Target RNA (variable) | 1 |
| RNA sensor probe (125 nM) | 4 |
| MgCl₂ (6 mM final) | 2 |
| Reaction Buffer 10x | 2 |
| RNAse Inhibitors (murine from NEB) | 2 |
| H₂O | 5 |
| Total | 20 |

Reaction buffer is: 40 mM Tris-HCl, 60 mM NaCl, pH 7.3

Perform this reaction for 20 min-3 hrs at 37° C. Read out with excitation: 485 nm/20 nm, emission: 528 nm/20 nm. A signal for single molecule sensitivity may be detected beginning at 20 min but of course sensitivity is higher for longer reaction times.

TABLE XXXII
Two step reaction: RPA amplification mix

| Component | Volume (μL) |
|---|---|
| Primer A (100 μM) | 0.48 |
| Primer B (100 μM) | 0.48 |
| RPA Buffer | 59 |
| MgAc | 5 |
| Target (variable concentration) | 5 |
| ATP (100 μM from NEB kit) | 2 |
| GTP (100 μM from NEB kit) | 2 |
| UTP (100 μM from NEB kit) | 2 |
| CTP (100 μM from NEB kit) | 2 |
| T7 Polymerase (from NEB kit) | 2 |
| H₂O | 25 |
| Total | 104.96 |

Mix this reaction together and then re-suspend two to three tubes of freeze-dried enzyme mix). Add 5 μL of 280 mM MgAc to the mix to begin the reaction. Preform reaction for 10-20 min.

Applicants evaluated the sensitivity of detection after 1:10 to 1:1000 dilution prior to and after Recombinase Polymerase Amplification (RPA) amplification. Diluting out the post-amplification by 1:100 dramatically improved sensitivity of the assay by X fold.

TABLE XXXIII
C2c2 detection mix

| | Volume |
|---|---|
| Component | (μL) |
| Protein (44 nM final) | 2 |
| crRNA (12 nM final) | 1 |
| background target (100 ng total) | 1 |
| RPA reaction | 1 |
| RNA sensor probe (125 nM) | 4 |
| MgCl₂ (6 mM final) | 2 |
| Reaction Buffer 10x | 2 |
| RNAse Inhibitors (murine from NEB) | 2 |
| H₂O | 5 |
| Total | 20 |

Reaction buffer is: 40 mM Tris-HCl, 60 mM NaCl, pH 7.3

Perform this for 20 min-3 hours. Minimum detection time is about 20 min to see single molecule sensitivity. Performing the reaction for longer only boosts the sensitivity.

TABLE XXXIV
One pot reaction:

| Component | Volume (μL) |
|---|---|
| Primer A (100 μM) | 0.48 |
| Primer B (100 μM) | 0.48 |
| RPA Buffer | 59 |
| MgAc | 5 |
| Lw2C2c2 (44 nM final) | 2 |
| crRNA (12 nM final) | 2 |
| Background RNA (from 250 ng/μL) | 2 |
| RNAse alert substr (after resuspending in 20 μL) | 5 |
| murine RNAse inhib from NEB | 10 |
| Target (variable concentration) | 5 |
| ATP (100 μM from NEB kit) | 2 |
| GTP (100 μM from NEB kit) | 2 |
| UTP (100 μM from NEB kit) | 2 |
| CTP (100 μM from NEB kit) | 2 |
| T7 Polymerase (from NEB kit) | 2 |
| H₂O | 4 |
| Total | 104.96 |

The NEB kit referenced is the HighScribe T7 High Yield Kit. To resuspend buffer, use a 1.5× concentration: resuspend three tubes of freeze dried substrate in 59 μL of buffer and use in the mix above. Each reaction is 20 μL so this is enough for 5 reactions worth. Single molecule sensitivity with this reaction has been observed in as early as 30-40 min.

TABLE XXXV
REAGENTS AND STOCK CONCENTRATIONS USED IN THE DEVELOPMENT
OF THE CAS13A-BASED NEISSERIA GONORRHOEAE AND gyrA
ASSAYS, QPCR, DNA SEQUENCING, AND LATERAL FLOW.

| | | | Stock | |
|---|---|---|---|---|
| Reagent | Reaction | Source | Concentration | Notes |
| C2c2 LwaCas13a | SHERLOCK | GenScript | 5 mg/mL | |
| Rnase Inhibitor | SHERLOCK | NEB | 40 U/μL | Murine |
| T7 RNA | SHERLOCK | Lucigen | 50 U/μL | NextGen |
| Polymerase | | | | |
| Reaction Buffer | SHERLOCK | N/A | 5X | 0.1M HEPES pH |
| | | | | 8.0; 300 mM KCl; |
| | | | | 25% PEG-8000 |
| rNTPs | SHERLOCK | NEB | 25 mM of each | |
| | | | nucleotide | |
| RNase Alert | SHERLOCK | Thermo Fisher | 2 μM | |
| Substrate v2 | | Scientific | | |
| MgAc | SHERLOCK | TwistDx | 280 mM | TwistAmp Basic |
| | | | | Kit |
| Storage Buffer | SHERLOCK | N/A | 1X | 50 mM Tris pH 7.5; |
| | | | | 600 mM KCl; 5% |
| | | | | glycerol; 2 mM |
| | | | | DTT |
| RPA Primers | SHERLOCK | Integrated DNA | 50 μM of each | |
| (forward, reverse) | | Technologies | primer | |
| Cas13a crRNA | SHERLOCK | Integrated DNA | 2.5 μM of each | |
| (wildtype, mutant, | | Technologies | | |
| por A) | | | | |
| Nuclease-Free H2O | | Thermo Fisher | N/A | Invitrogen |
| | | Scientific | | |
| RPA Pellets | SHERLOCK | TwistDx | N/A | TwistAmp Basic |
| (lyophilized) | | | | Kit |
| Synthetic DNA | SHERLOCK | Integrated DNA | 10¹⁰ copies/μL | |
| Target (wildtype, | | Technologies | | |
| mutant) | | | | |
| PCR Primers | qPCR | Integrated DNA | 100 μM of each | |
| (forward, reverse) | | Technologies | primer | |
| FastStart SYBR | qPCR | Roche | 2X | |
| Green Master | | | | |
| Lateral Flow | SHERLOCK | Milenia Biotec | N/A | HybriDetect - |
| Dipsticks | | | | Universal Lateral |
| | | | | Flow Assay Kit |
| HybriDetect Assay | SHERLOCK | Milenia Biotec | N/A | HybriDetect - |
| Buffer | | | | Universal Lateral |
| | | | | Flow Assay Kit |

2) a Cas13a-Based porA Assay for N. Gonorrhoeae Detection

### Selection of Primer-Guide Pairs

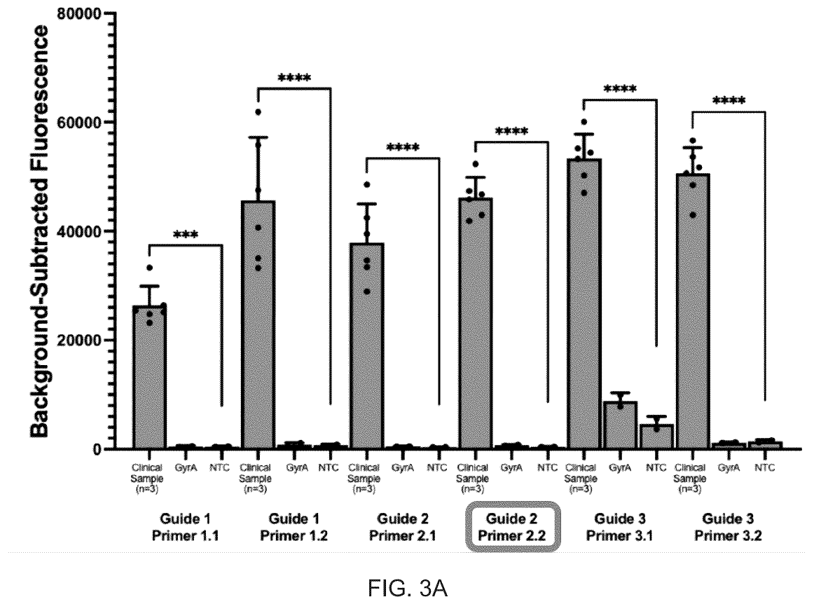

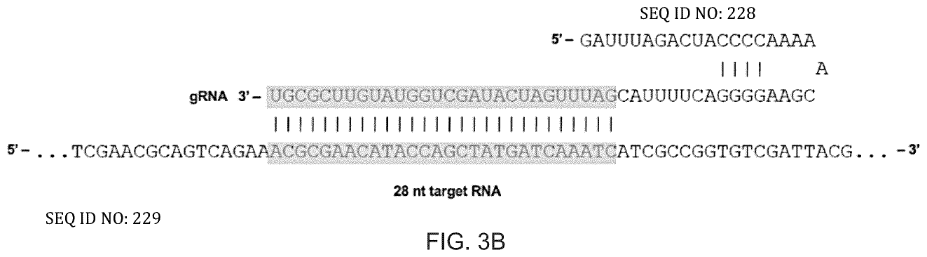

To create an assay for N. gonorrhoeae detection, six porA primer-guide pairs were designed and their performance was evaluated both in terms of sensitivity and cross-reactivity, using a fluorescence-based readout (FIG. 3). Three purified N. gonorrhoeae isolates were tested using both negative template controls as well as synthetic gyrA as a positive control (FIG. 3A).

Guide 2 and primer set 2.2 were selected because they produced both a high fluorescent signal and excellent discrimination between synthetic N. gonorrhoeae purified isolates and the negative controls (FIG. 3A). Guide 3 primer set 1 was excluded due to cross-reactivity with the gyrA control.

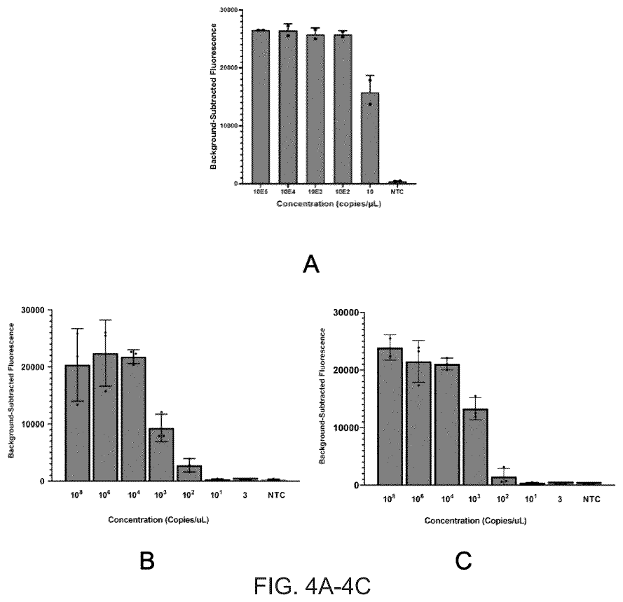

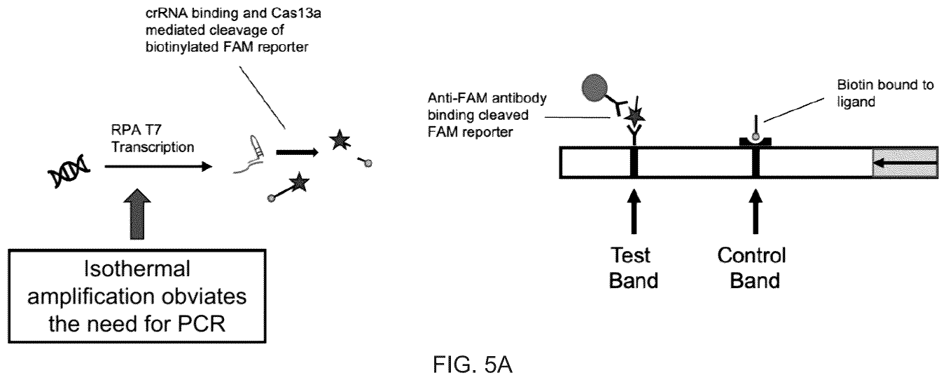

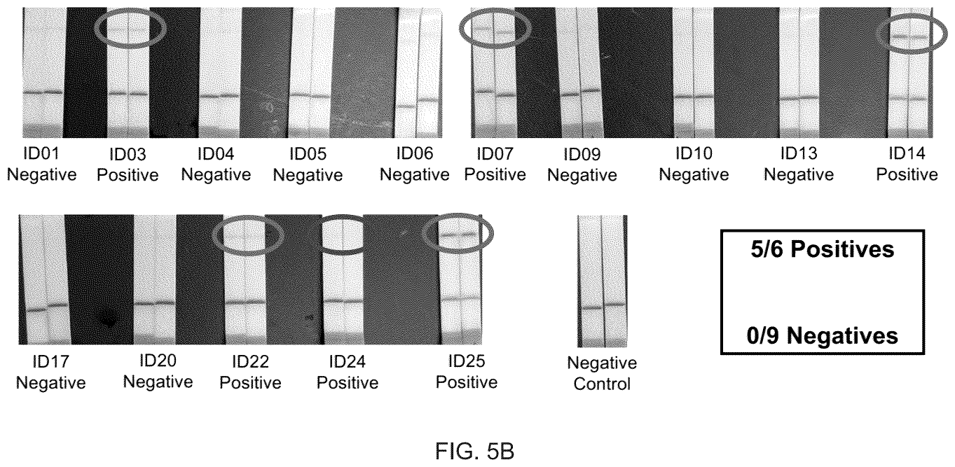

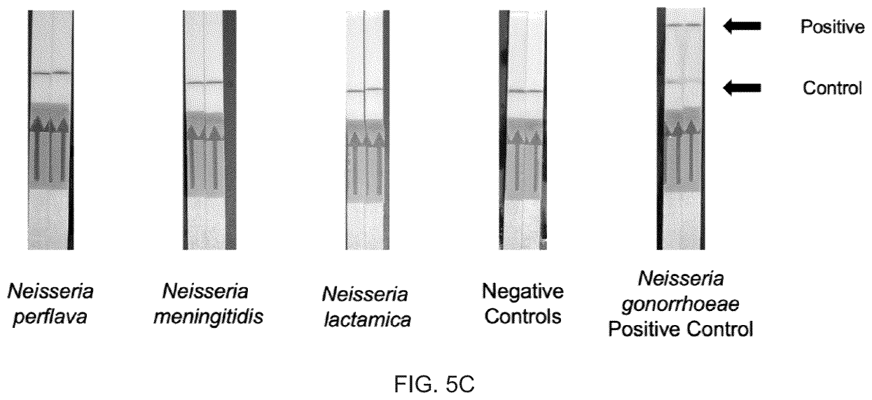

Having selected the optimal gRNA and primer set for porA detection, the limit of detection (LoD) was determined using serial dilutions in nuclease-free water as well as the detection of purified N. gonorrhoeae isolates using a fluorescence-based readout. The porA assay had an LoD of 10,000 copies per milliliter (FIG. 4). The assay was then tested on 14 purified isolates and 3 non-gonococcal Neisseria isolates: N. meningitidis, N. perflava, and N. lactamica. The assay detected all 14 N. gonorrhoeae isolates, with peak fluorescence occurring after 20 minutes and did not detect any of the non-gonococcal Neisseria isolates (FIG. 5).

The N. gonorrhoeae porA detection assay was then assessed using a lateral flow readout, substituting the standard fluorescence reporter with a biotinylated FAM reporter compatible with the test strips (FIG. 5A). Based on prior protocols, 90 minutes were allocated for the assay. Visual inspection of the test strips 3-5 minutes after specimen introduction revealed detection of all 14 purified isolates tested in triplicate (FIG. 5B) and excellent discrimination between N. gonorrhoeae and three non-gonococcal Neisseria isolates (FIG. 5C).

Having shown that a lateral flow-based N. gonorrhoeae detection assay can be developed, Applicants explored the possibility of simplifying upstream DNA extraction to facilitate deployment in low-resource settings. To do so, fluorescence from the N. gonorrhoeae detection assay was measured on three purified isolates that underwent thermal DNA extraction. The extracted DNA was quantified using PCR and DNA concentrations above 1,000,000 copies per milliliter were detected using the selected guide-primer set combination.

TABLE XXXVI
NEISSERIA GONORRHOEAE porA PRIMER SETS AND GUIDE
RNAS

| PRIMER | SEQUENCE | SEQ ID NO |
|---|---|---|
| Por A RPA Primer 1 | GAAATTAATACGACTCACTAT | 210 |
| (forward) | | |
| Por A RPA Primer 1.1 | GTACCTGATGGTTTTTCAATGGATCGGTATC | 211 |
| (reverse) | | |
| Por A RPA Primer 1 | GAAATTAATACGACTCACTAT | 212 |
| (forward) | | |
| Por A RPA Primer 1.2 | TACCTGATGGTTTTTCAATGGATCGGTATCAC | 213 |
| (reverse) | | |
| Por A RPA Primer 2.1 | GAAATTAATACGACTCACTAT | 214 |
| (forward) | | |
| Por A RPA Primer 2.1 | GAAGTGCGCTTGGAAAAATCGTAATCGACAC | 215 |
| (reverse) | | |
| Por A RPA Primer 2.2 | GAAATTAATACGACTCACTAT | 216 |
| (forward) | | |
| Por A RPA Primer 2.2 | GAAGTGCGCTTGGAAAAATCGTAATCGACACC | 217 |
| (reverse) | | |
| Por A RPA Primer 3.1 | GAAATTAATACGACTCACTAT | 218 |
| (forward) | | |
| Por A RPA Primer 3.1 | CATATTTAAGGGCATAATTTCCGAAAAAGC | 219 |
| (reverse) | | |
| Por A RPA Primer 3.2 | GAAATTAATACGACTCACTAT | 220 |
| (forward) | | |
| Por A RPA Primer 3.2 | GCATATTTAAGGGCATAATTTCCGAAAAAG | 221 |
| (reverse) | | |
| Por A Cas13a crRNA 1 | GAUUUAGACUACCCCAAAAACGAAGGGGACU | 222 |
| | AAAAC | |
| Por A Cas13a crRNA 2 | GAUUUAGACUACCCCAAAAACGAAGGGGACU | 223 |
| | AAAAC | |
| Por A Cas13a crRNA 3 | GAUUUAGACUACCCCAAAAACGAAGGGGACU | 224 |
| | AAAAC | |

Selected porA Primer Set 2.1 and Cas13a Guide RNA 2 are Outlined

TABLE XXXVII
NEISSERIA GONORRHOEAE porA PSEUDOGENE, STRAIN FA1090
GenBank: AJ223447.1 (nucleotide sequence)(SEQ ID NO: 225)
Feavers, I. M. and Maiden, M. C., A gonococcal porA pseudogene: implications for understanding the
evolution and pathogenicity of Neisseria gonorrhoeae Mol. Microbiol. 30 (3), 647-656 (1998)

| | |
|---|---|
| Start (0) | |
| \| | |
| AAGGTCTGTATTTAAATCATGTTGCGGGAAAGCAACATTTTCAAAAAAGTTAATTTATTGTTTTATATTGAAATATTATTTTTCAAAATAAAAATTCCAA | |
| \|+++\|++++\|++++\|++++\|++++\|++++\|++++\|++++\|++++\|++++\|++++\|++++\|++++\|++++\|++++\|++++\|++++\|++++\|++++\|++++\| | 100 |
| TTCCAGACATAAATTTAGTACAACGCCCTTTCGTTGTAAAAGTTTTTTCAATTAAATAACAAAATATAACTTTATAATAAAAAGTTTTATTTTTAAGGTT | |
| AATTTACCCGAAATTTGTTCCGAAAAATGGTTTTTTTTTTTCGGGGGGTAATTGGAGACTGATTGGGTGTTTGCCCGATGTTTTTAGCAAATTTACAAAA | |
| ++++\|++++\|++++\|++++\|++++\|++++\|++++\|++++\|++++\|++++\|++++\|++++\|++++\|++++\|++++\|++++\|++++\|++++\|++++\|++++\| | 200 |
| TTAAATGGGCTTTAAACAAGGCTTTTTACCAAAAAAAAAAAGCCCCCCATTAACCTCTGACTAACCCACAAACGGGCTACAAAAATCGTTTAAATGTTTT | |
| GGAAGCCGATATGCGAAAAAAACTTACCGCCCTCGTATTGTCCGCACTGCCGTTTGCGGCAGTTGCCGATGTCAGCCTGTACGGCGAAGTCAAAGCTGGT | |
| ++++\|++++\|++++\|++++\|++++\|++++\|++++\|++++\|++++\|++++\|++++\|++++\|++++\|++++\|++++\|++++\|++++\|++++\|++++\|++++\| | 300 |
| CCTTCGGCTATACGCTTTTTTTGAATGGGGGGAGCATAACAGGCGTGACGGCAAACGCCGTCAACGGCTACAGTCGGACATGCCGCTTCAGTTTCGACCA | |
| GTGGAAGGCAGGAACATCCGGCTGCAGTTGACCGAGCCACCCTCAGAAGGTCAAACGGGCAATACAGTTACTAAGGCCAAAAGCCGCATCAGGACGAAAG | |
| ++++\|++++\|++++\|++++\|++++\|++++\|++++\|++++\|++++\|++++\|++++\|++++\|++++\|++++\|++++\|++++\|++++\|++++\|++++\|++++\| | 400 |
| CACCTTCCGTCCTTGTAGGCCGACGTCAACTGGCTCGGTGGGAGTCTTCCAGTTTGCCCGTTATGTCAATGATTCCGGTTTTCGGCGTAGTCCTGCTTTC | |
| TCAGTGATTTCGGCTCGTTTATCGGCTTTAAGGGGGTGGGGATTTGGGCGGCGGGCTGAAGGCTGTTTGGCAGCTCGAGCAAGACGTATCCGTTGCCGGC | |
| ++++\|++++\|++++\|++++\|++++\|++++\|++++\|++++\|++++\|++++\|++++\|++++\|++++\|++++\|++++\|++++\|++++\|++++\|++++\|++++\| | 500 |
| AGTCACTAAAGCCGAGCAAATAGCCGAAATTCCCCCACCCCTAAACCCGCCGCCCGACTTCCGACAAACCGTCGAGCTCGTTCTGCATAGGCAACGGCCG | |
| GGCGGCGCGACCCGTTGGGGTAACAGGGAATCCTTTATCGGCTTGGCAGGCGAATTCGGCACGGCGCTCGCCGGTCGCGTTGCGAATCCGTTTGGCGATG | |
| ++++\|++++\|++++\|++++\|++++\|++++\|++++\|++++\|++++\|++++\|++++\|++++\|++++\|++++\|++++\|++++\|++++\|++++\|++++\|++++\| | 600 |
| CCGCCGCGCTGGGCAACCCCATTGTCCCTTAGGAAATAGCCGAACCGTCCGCTTAAGCCGTGCCGCGAGCGGCCAGCGCAACGOTTAGGCAAACCGCTAC | |
| 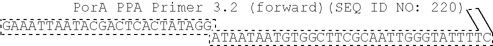 | |
| 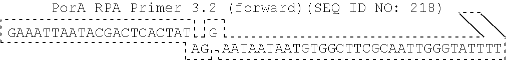 | |
| CCAGCAAAGCCATTGATCCTTGGGACAGCAATAATAATGTGGCTTCGCAATTGGGTATTTTCAAACGCCACGACGGTATGCCGGTTTCCGTGCGTTACGA | |
| ++++\|++++\|++++\|++++\|++++\|++++\|++++\|++++\|++++\|++++\|++++\|++++\|++++\|++++\|++++\|++++\|++++\|++++\|++++\|++++\| | 700 |
| GGTCGTTTCGGTAACTAGGAACCCTGTCGTTATTATTACACCGAAGCGTTAACCCATAAAAGTTTGCGGTGCTGCCATACGGCCAAAGGCACGCAATGCT | |
| TTCCCCCGGATTTTCCGGTTTCAGCGGCAGCATTCAATTTGTTCCGAGTCAAAACAGCAAGTCCGCCTATACGCCTGCTACTTTCACGCTGGAAAGTAAT | |
| ++++\|++++\|++++\|++++\|++++\|++++\|++++\|++++\|++++\|++++\|++++\|++++\|++++\|++++\|++++\|++++\|++++\|++++\|++++\|++++\| | 800 |
| AAGGGGGCCTAAAAGGCCAAAGTCGCCGTCGTAAGTTAAACAAGGCTCAGTTTTGTCGTTCAGGCGGATATGCGGACGATGAAAGTGCGACCTTTCATTA | |
| 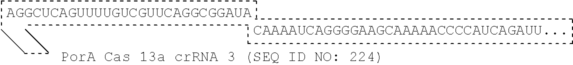 | |
| 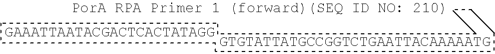 | |
| CAGATGAAACCAGTTCCGGCTGTTGTCGGCAAGCCGGGGTCGGATGTGTATTATGCCGGTCTGAATTACAAAAATGGCGGCTTTTTCGGAAATTATGCCC | 900 |
| ++++\|++++\|++++\|++++\|++++\|++++\|++++\|++++\|++++\|++++\|++++\|++++\|++++\|++++\|++++\|++++\|++++\|++++\|++++\|++++\| | |
| GTCTACTTTGGTCAAGGCCGACAACAGCCGTTCGGCCCCAGCCTACACATAATACGGCCAGACTTAATGTTTTTACCGCCGAAAAAGCCTTTAATACGGG | |
| 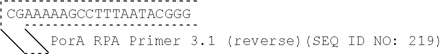 | |
| 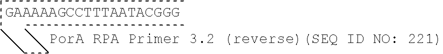 | |
| 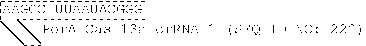 | |
| TTAAATATGCGAAACACGCCAATGAGGGGCATGATGCTTTCTTTTTGTTCTTGCTCGGCAGAGCGAGTGATACCGATCCATTGAAAAACCATCAGGTACA | |
| ++++\|++++\|++++\|++++\|++++\|++++\|++++\|++++\|++++\|++++\|++++\|++++\|++++\|++++\|++++\|++++\|++++\|++++\|++++\|++++\| | 1000 |
| AATTTATACGCTTTGTGCGGTTACTCCCCGTACTACGAAAGAAAAACAAGAACGAGCCGTCTCGCTCACTATGGCTAGGTAACTTTTTGGTAGTCCATGT | |
| 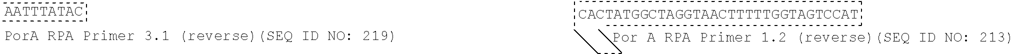 | |
| 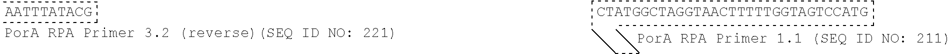 | |
| 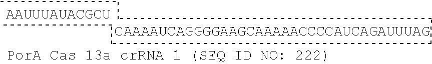 | |
| CCGCCTGACGGGCGGCTATGGGGAAGGCGGCTTGAATCTCGCCTTGGCGGCTCAGTTGGATTTGTCTGAAAATGCCGACAAAACCAAAAACAGTACGACC | |
| ++++\|++++\|++++\|++++\|++++\|++++\|++++\|++++\|++++\|++++\|++++\|++++\|++++\|++++\|++++\|++++\|++++\|++++\|++++\|++++\| | 1100 |
| GGCGGACTGCCCGCCGATACCCCTTCCGCCGAACTTAGAGCGGAACCGCCGAGTCAACCTAAACAGACTTTTACGGCTGTTTTGGTTTTTGTCATGCTGG | |
| 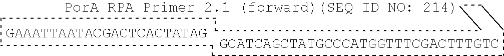 | |
| 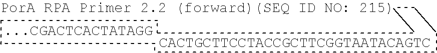 | |
| GAAATTGCCGCCACTGCTTCCTACCGCTTCGGTAATACAGTCCCGCGCATCAGCTATGCCCATGGTTTCGACTTTGTCGAACGCAGTCAGAAACGCGAAC | |
| ++++\|++++\|++++\|++++\|++++\|++++\|++++\|++++\|++++\|++++\|++++\|++++\|++++\|++++\|++++\|++++\|++++\|++++\|++++\|++++\| | 1200 |
| CTTTAACGGCGGTGACGAAGGATGGCGAAGCCATTATGTCAGGGCGCGTAGTCGATACGGGTACCAAAGCTGAAACAGCTTGCGTCAGTCTTTGCGCTTG | |
| 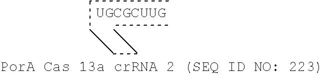 | |
| ATACCAGCTATGATCAAATCATCGCCGGTGTOGATTACGATTTTTCCAAGCGCACTTCCGCCATCATGTCTGCCGCTTGGCTGAAACGAAATACCGGCAT | |
| ++++\|++++\|++++\|++++\|++++\|++++\|++++\|++++\|++++\|++++\|++++\|++++\|++++\|++++\|++++\|++++\|++++\|++++\|++++\|++++\| | 1300 |
| TATGGTCGATACTAGTTTAGTAGCGGCCACAGCTAATGCTAAAAAGGTTCGCGTGAAGGCGGTAGTACAGACGGCGAACCGACTTTGCTTTATGGCCGTA | |
| 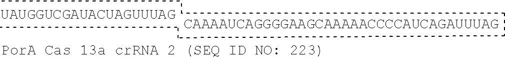 | |
| 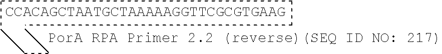 | |
| 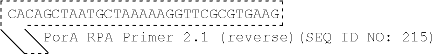 | |

| | |
|---|---|
| End | (1332) |
| \| | |
| CGGCAACTACACTCAAATTAATGCCGCCTCCG | 3′ |
| ++++\|++++\|++++\|++++\|++++\|++++\|+\| | 1332 |
| GCCGTTGATGTGAGTTTAATTACGGCGGAGGC | 5′ |

3) A Cas13a-Based gyrA Assay for the Detection of Ciprofloxacin-Resistant N. Gonorrhoeae

To create an assay for detecting N. gonorrhoeae resistance to ciprofloxacin, two guide pairs (wild type and mutant) were designed to target the point mutation in codon 91 of the gyrA gene and three flanking primer sets. The mutation of interest was placed three nucleotides distal to the Cas hairpin, previously shown to be the optimal position.

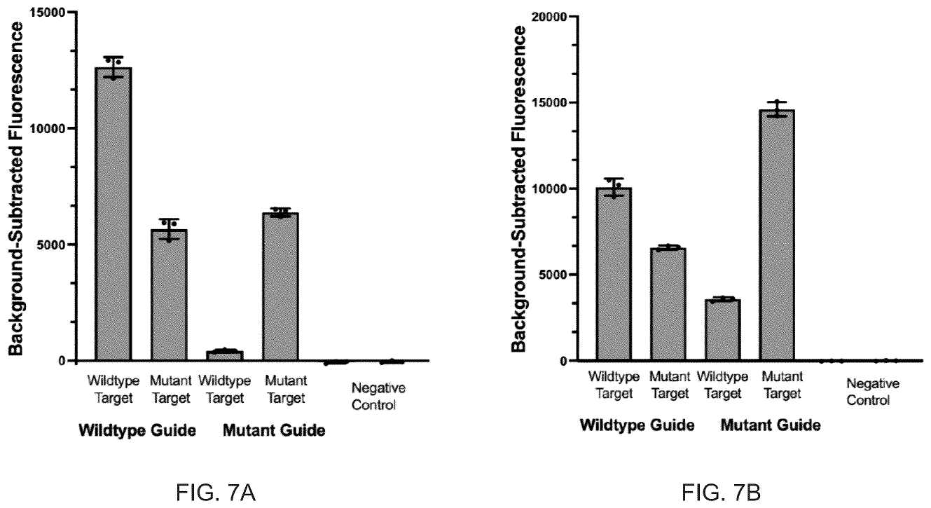

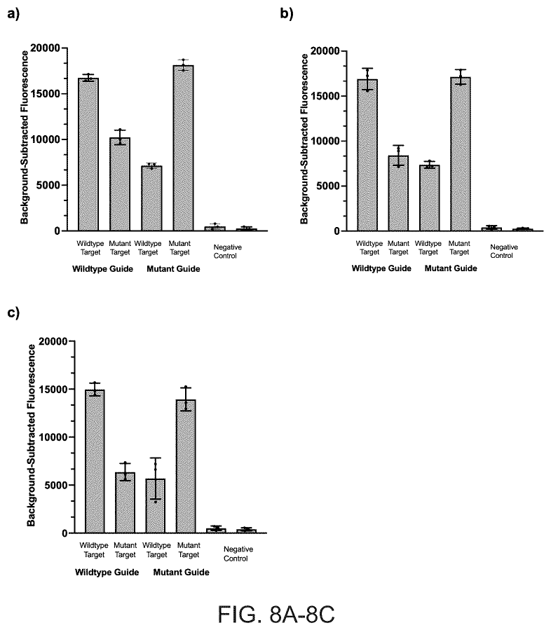

An additional synthetic mismatch was placed in either the second position or the fourth position of the spacer region. Placing the synthetic mutation at the second position produced the highest fluorescence and greatest discrimination between the wild-type and mutant synthetic DNA targets (FIGS. 7A-7B). Three forward and reverse primer sets were tested for use with that guide and selected the set that produced the highest fluorescence signal and greatest discrimination between the wild-type and mutant synthetic DNA targets (FIG. 8).

The in vitro LoD of the fluorescence-based gyrA assay was evaluated via serial dilutions in nuclease-free water of synthetic wild-type and mutant DNA targets. The gyrA assay had an LoD of 1,000,000 copies per milliliter for both wild-type (FIG. 4B) and mutant targets (FIG. 4C).

To further assess the performance of the gyrA assay, 23 purified N. gonorrhoeae isolates were analyzed for susceptibility to ciprofloxacin determined phenotypically by culture and genotypically by sequencing to detect mutation codon 91 of the gyrA gene. A standard MIC breakpoint of ≥1 μg/mL was used to define ciprofloxacin resistance (TABLE XXII).

TABLE XXXVIII
CHARACTERISTICS OF PURIFIED
N. GONORRHOEAE ISOLATES

| Year | | Ciprofloxacin | Resistance | GyrA | GyrA |
|---|---|---|---|---|---|
| collect- | Anatomic | MIC | interpre- | genotype | concor- |
| ed | site | (pg/mL)^{a} | tation | (PCR) | dance |
| 2014 | Pharynx | 50.015 | Susceptible | Wild type | Yes |
| 2014 | Pharynx | 50.015 | Susceptible | Wild type | Yes |
| 2014 | Pharynx | 50.015 | Susceptible | Wild type | Yes |
| 2014 | Urethra | 8.000 | Resistant | Mutant | Yes |
| 2013 | Urethra | 8.000 | Resistant | Mutant | Yes |
| 2013 | Urethra | 8.000 | Resistant | Mutant | Yes |
| 2013 | Urethra | >16.000 | Resistant | Mutant | Yes |
| 2014 | Urethra | >16.000 | Resistant | Mutant | Yes |
| 2014 | Urethra | 8.000 | Resistant | Mutant | Yes |
| 2014 | Urethra | >16.000 | Resistant | Mutant | Yes |
| 2014 | Urethra | 8.000 | Resistant | Mutant | Yes |
| 2014 | Urethra | 8.000 | Resistant | Mutant | Yes |
| 2011 | Urethra | 16.000 | Resistant | Mutant | Yes |
| 2011 | Urethra | 16.000 | Resistant | Mutant | Yes |
| 2012 | Urethra | 16.000 | Resistant | Mutant | Yes |
| 2011 | Urethra | 16.000 | Resistant | Mutant | Yes |
| 2011 | Urethra | 16.000 | Resistant | Mutant | Yes |
| 2014 | Urethra | >16.000 | Resistant | Mutant | Yes |
| 2014 | Urethra | 1.000 | Resistant | Mutant | Yes |
| 2014 | Urethra | 1.000 | Resistant | Mutant | Yes |
| 2013 | Urethra | 1.000 | Resistant | Mutant | Yes |
| 2014 | Urethra | 16.000 | Resistant | Mutant | Yes |
| 2013 | Urethra | 16.000 | Resistant | Mutant | Yes |

Of the 23 isolates, 20 with MICs between 1 and >16 μg/mL were deemed resistant, and three with MICs<0.015 μg/mL were deemed susceptible. Of the 20 N. gonorrhoeae isolates with MICs≥1 μg/mL, 100% had mutant gyrA genotypes by DNA sequencing. Of the 3 N. gonorrhoeae isolates with MICs<0.015 μg/mL, 100% had no mutation at codon 91 of the gyrA gene by DNA sequencing.

The discrimination of the selected wild-type and mutant Cas13a guides for codon 91 of the gyrA gene was assessed among all 23 isolates. All of the 20 ciprofloxacin-resistant gyrA mutant specimens were detected by the mutant Cas13a assay, while none of the three wild-type isolates were detected by the mutant Cas13a assay, showing a 100% agreement. FIG. 9A showed the pooled performance among all specimens, while FIG. 9B shows the performance on each individual specimen. FIG. 9C shows the DNA sequence alignment for all 23 isolates with the wild-type and mutant gRNAs.

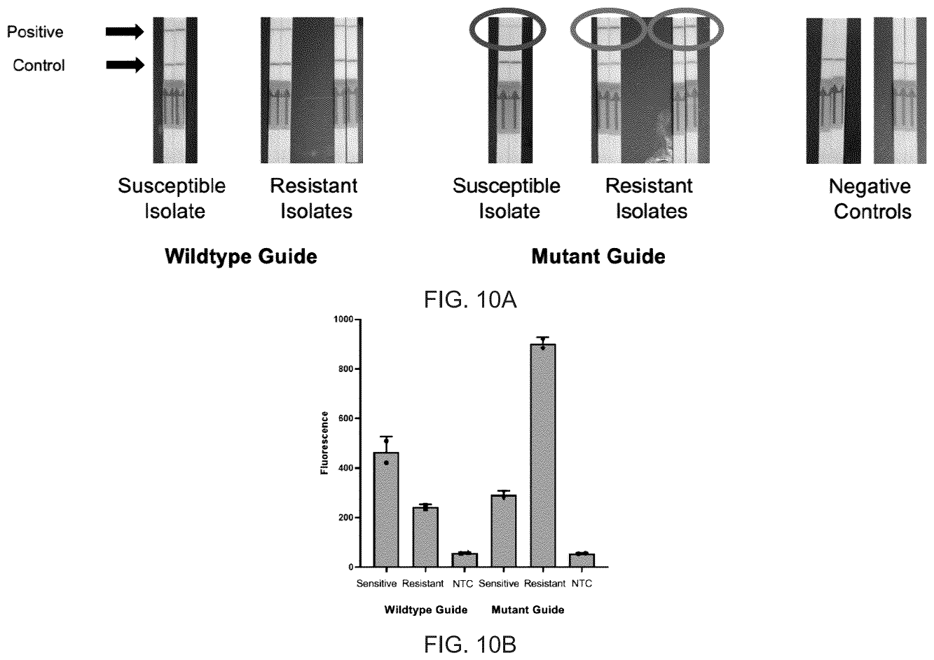

Applicants next aimed to convert the gyrA resistance assay into a portable format suitable for use in resource-limited settings. A lateral flow format was tested, again substituting the standard fluorescence reporter with a biotinylated FAM reporter compatible with the test strips. FIG. 10A shows the performance of the gyrA lateral flow on three purified isolates (one with known phenotypic and genotypic susceptibility to ciprofloxacin and two with known resistance). Each isolate was tested in duplicate. The wild-type guide failed to discriminate visually between resistant and susceptible isolates. The mutant guide demonstrated promising discrimination; however, a faint positive line in the susceptible isolate was detected.

Given the technical limitations of the gyrA assay using a lateral flow readout, the performance of the assay was tested using a portable quantitative fluorescence detector. Such a detector, the Qubit 4 Fluorometer (ThermoFisher Scientific, USA), permits low-cost detection in the absence of a plate reader (Cytation 5, BioTek, USA). The one-pot SHERLOCK reaction was incubated for 90 minutes at 37° C. The reaction was then transferred to Qbit Assay tubes, diluted with nuclease-free water to 200 μL. Green fluorescence was detected on the blue excitation setting (430-495 excitation filter; 510-580 emission filter). FIG. 10B shows successful discrimination for both the wild-type and mutant isolates using that method.

4) A Multiplex Cas12a/CAS13a-Based Lateral Flow Assay for the Detection of Ciprofloxacin-Resistant Neisseria gonorrhoeae at the Point of Care

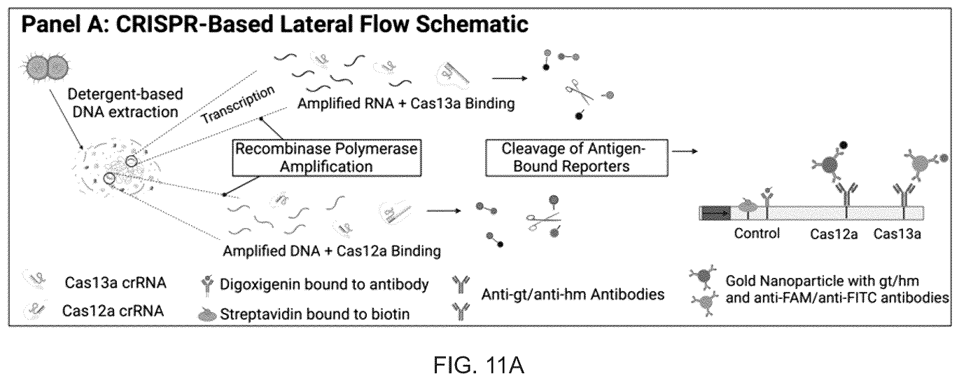

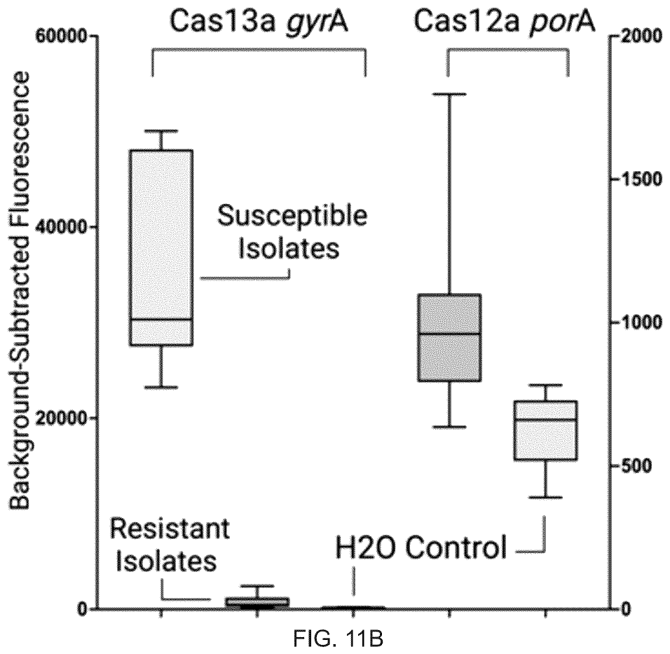

Two unique targets (porA and gyrA) were amplified within one reaction using isothermal recombinase polymerase amplification (RPA). Two distinct amplified products were detected using gel electrophoresis. A Cas12a-porA and Cas13a-gyrA system for simultaneous detection against 10 purified isolates (two with ciprofloxacin resistance) was then tested on a lateral flow strip (FIG. 11A). Robust Cas13a-mediated gyrA discrimination was detected (FIG. 11B). Cas12a-mediated fluorescence was less intense, but still distinguishable from water controls.

### Example 2: Crispr-Cas13A Detection of Pena Mutations for Cephalosporin Resistance Determination

### Materials And Methods

Assays using both synthetic N. gonorrhoeae DNA and purified clinical isolates were tested. Synthetic DNA templates representing wildtype penA sequences and mutant penA sequences containing key resistance-associated mutations (A501V/P/T, G542S, P551L/S, and mosaic allele patterns at positions 375-377) were obtained from Integrated DNA Technologies (USA). Serial dilutions were prepared from stock concentrations down to single copy levels in nuclease-free water.

Clinical N. gonorrhoeae isolates with characterized penA genotypes and known cephalosporin susceptibility profiles were obtained from laboratory collections. Isolates were stored in glycerol at −80° C. prior to extraction. Whole-genomic DNA extraction was performed using the DNeasy Blood and Tissue Kit (Qiagen, Germany) following the protocol described in Example 1.

Cas13a guide RNAs were designed using ADAPT software to specifically target wildtype penA sequences for the detection of mosaic penA mutations and other resistance-associated alterations. The guide design strategy focused on detecting the absence of key mutations including A501V/P/T, G542S, P551L/S, and mosaic insertion patterns at codons 375-377 that are associated with cephalosporin resistance as described in TABLE II.

The guide sequences were designed such that the presence of resistance mutations would disrupt guide RNA binding and prevent Cas13a activation. Forward and reverse RPA primers flanking the penA target regions were designed with lengths of 27-35 nucleotides, melting temperatures between 58-68° C., and amplicon sizes of 140-200 base pairs. T7 RNA polymerase promoter sequences (5′ GAAATTAATACGACTCACTATAGG 3′; SEQ ID NO: 209) were incorporated at the 5′ terminus of forward primers.

SHERLOCK reactions utilized the same conditions as described in Example 1. Reactions contained 45 nM C2c2 LwaCas13a, 1 U/μL murine RNase inhibitor, 10 U/μL T7 RNA polymerase, 136 nM RNaseAlert substrate v2, 1× SHINE Buffer, and 2 mM of each rNTP. Each reaction received 22.5 nM penA wildtype-specific gRNA designed for mosaic mutation detection and 320 nM each of the corresponding RPA primers.

### Results

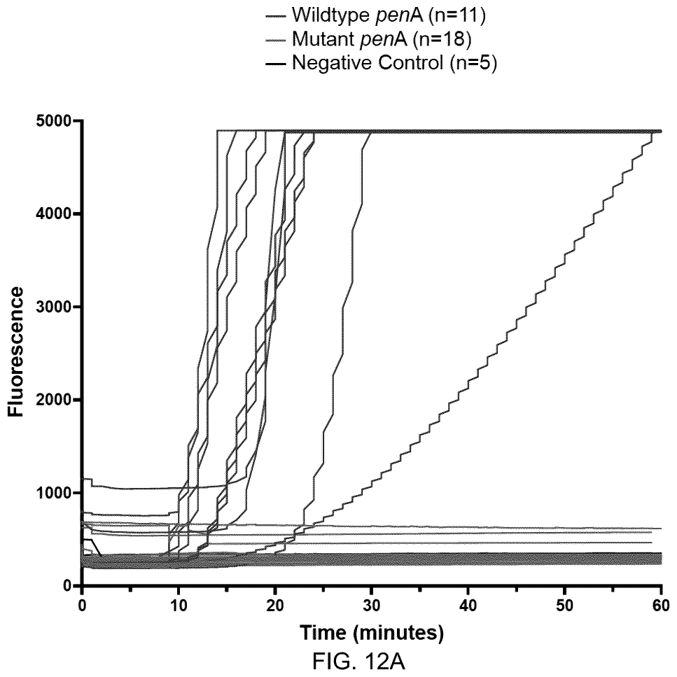

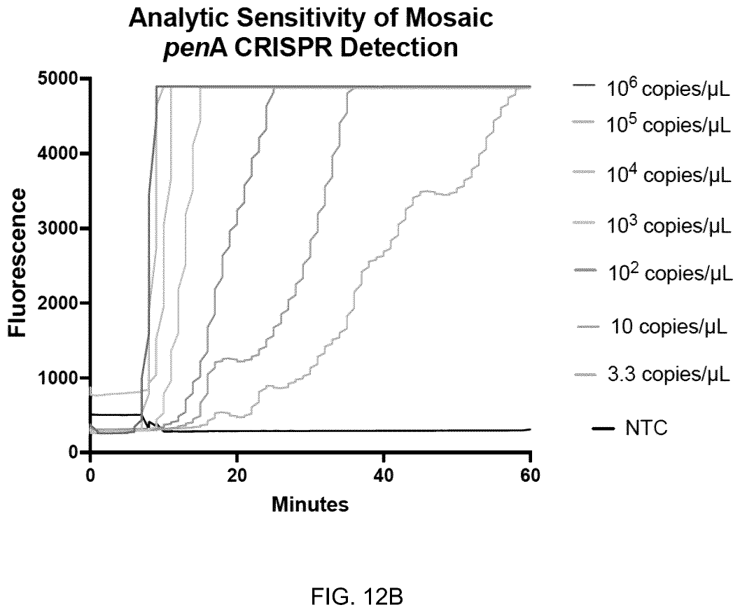

The penA mosaic mutation detection assay was evaluated using 29 clinical isolates representing diverse penA genotypes. Isolates with wildtype penA sequences (n=11) demonstrated robust fluorescence activation, indicating successful guide RNA binding and Cas13a activation. Isolates containing resistance-associated penA mutations including mosaic patterns (n=18) showed minimal fluorescence activation, as mutations disrupted guide RNA binding and prevented Cas13a activation. Negative control reactions (n=5) remained at baseline fluorescence (FIG. 12A).

Serial dilution analysis using synthetic wildtype penA templates established the limit of detection for the mosaic penA detection assay at 3.3 copies/μL, with consistent signal generation observed from 10⁶ to 3.3 copies/μL. No template control (NTC) reactions showed no detectable signal (FIG. 12B).

Among the 11 isolates generating positive signals (wildtype penA detected), all remained fully susceptible to both cefixime (MICs≤0.015 μg/mL) and ceftriaxone (MICs≤0.015 μg/mL), demonstrating 100% positive predictive value for cephalosporin susceptibility.

Among the 18 isolates with mutant penA sequences, none of them showed fluorescence response due to disrupted guide binding. The discordant isolates contained single penA mutations without additional resistance determinants, consistent with literature reports that multiple genetic alterations are typically required for high-level cephalosporin resistance.

Positive fluorescence signals indicate wildtype penA sequences and predict cephalosporin susceptibility, supporting the use of cefixime or ceftriaxone. Negative signals indicate resistance-associated mutations and suggest potential treatment failure with cephalosporins, warranting alternative antimicrobial selection. The assay enables rapid resistance-guided therapy decisions for point-of-care applications.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

1. A nucleic acid detection system for detecting an antibiotic-resistant Neisseria gonorrhoeae pathogen in a patient sample, comprising:
a first CRISPR-Cas system having collateral cleavage activity and configured to bind one or more N. gonorrhoeae polynucleotide sequences that identify the presence of N. gonorrhoeae;
a second CRISPR-Cas system having collateral cleavage activity and configured to bind one or more N. gonorrhoeae polynucleotide sequences that indicate resistance or susceptibility to an antibiotic;
a first detection construct comprising a cutting motif configured to generate a first detectable signal when preferentially cut by the one or more CRISPR-Cas systems configured to bind one or more N. gonorrhoeae polynucleotide sequences that identify the presence of N. gonorrhoeae, and
a second detection construct comprising cutting motifs configured to generate one or more second detectable signals when preferentially cut by CRISPR-Cas systems configured to bind N. gonorrhoeae polynucleotide sequences that indicate resistance or susceptibility to an antibiotic,
wherein the antibiotic is not ciprofloxacin.

2. The nucleic acid detection system of claim 1, wherein the N. gonorrhoeae polynucleotide sequences that indicate resistance or susceptibility to an antibiotic identify resistance or susceptibility to cefixime and/or ceftriaxone.

3. The nucleic acid detection system of claim 1, wherein the one or more N. gonorrhoeae polynucleotide sequences that identify the presence of N. gonorrhoeae are located in a porA gene.

4. The nucleic acid detection system of claim 1, wherein the one or more N. gonorrhoeae polynucleotide sequences are located in a nucleotide sequence of SEQ ID NO: 225.

5. The nucleic acid detection system of claim 2, wherein the one or more N. gonorrhoeae polynucleotide sequences that indicate resistance or susceptibility to cefixime or ceftriaxone are located in a penicillin-binding protein 2 (penA) gene.

6. (canceled)

7. The nucleic acid detection system of claim 5, wherein the one or more polynucleotide sequences that indicate resistance to ceftriaxone comprise:
a) single nucleotide polymorphisms (SNPs) in the mosaic allele type 60 of the penA gene; or
b) one or more mutations in codons 311, 316, 483, 501, 512, 516, 542 and 545 of the penA gene, wherein the one or more mutations comprise A311V, V316T/P and T483S.

8. (canceled)

9. The nucleic acid detection system of claim 7, wherein the one or more N. gonorrhoeae polynucleotide sequences that indicate resistance to ceftriaxone further comprise one or more mutations selected from:
a) one or more mutations in codons 120 and 121 of the porB gene; or
b) a mutation in codon 421 of the ponA gene; or
c) both a) and b).

10. (canceled)

11. The nucleic acid detection system of claim 5, wherein the one or more N. gonorrhoeae polynucleotide sequences indicate;
a) resistance to cefixime comprise one or more mutations chosen from A501V, A501P, A501T, N512Y, A516G, G542S, G545S, P551L and P551S in the penA gene; or
b) susceptibility to cefixime comprise a wild type sequence in one or more codons chosen from codons 311, 312, 316, 345, 483, 375, 376, 377, 501, 542 and 551 of the penA gene.

12. (canceled)

13. The nucleic acid detection system of claim 1, further comprising amplification reagents, wherein the amplification reagents comprise one or more primers pairs for amplifying the one or more N. gonorrhoeae polynucleotide sequences that identify the presence of N. gonorrhoeae, the one or more N. gonorrhoeae polynucleotide sequences that indicate antibiotic resistance, or both.

14. (canceled)

15. The nucleic acid detection system of claim 13, wherein the one or more primers pairs for amplifying the one or more N. gonorrhoeae polynucleotide sequences that identify the presence of N. gonorrhoeae are selected from any one of the nucleotide sequences set forth in SEQ ID NO: 210-221.

16. The nucleic acid detection system of claim 1, wherein the first and second CRISPR-Cas systems are independently selected from a Type V or a Type VI CRISPR-Cas system.

17. A nucleic acid detection system for detecting an antibiotic-resistant Neisseria gonorrhoeae pathogen in a patient sample, comprising:
a first CRISPR-Cas system having collateral cleavage activity and configured to bind one or more N. gonorrhoeae polynucleotide sequences that identify the presence of N. gonorrhoeae;
a second CRISPR-Cas system having collateral cleavage activity and configured to bind one or more N. gonorrhoeae polynucleotide sequences that indicate antibiotic resistance;
a first detection construct comprising a cutting motif configured to generate a first detectable signal when preferentially cut by the one or more CRISPR-Cas systems configured to bind one or more N. gonorrhoeae polynucleotide sequences that identify the presence of N. gonorrhoeae, and
a second detection construct comprising cutting motifs configured to generate one or more second detectable signals when preferentially cut by CRISPR-Cas systems configured to bind N. gonorrhoeae polynucleotide sequences that indicate resistance to ciprofloxacin, and either cefixime or ceftriaxone, or
ciprofloxacin, cefixime and ceftriaxone.

18. The nucleic acid detection system of claim 17, wherein the one or more N. gonorrhoeae polynucleotide sequences that identify the presence of N. gonorrhoeae are located in a porA gene.

19. The nucleic acid detection system of claim 17, wherein the one or more N. gonorrhoeae polynucleotide sequences that identify the presence of N. gonorrhoeae are located in a nucleotide sequence set forth in SEQ ID NO: 225.

20. The nucleic acid detection system of claim 17, wherein the one or more N. gonorrhoeae polynucleotide sequences that indicate resistance to cefixime or ceftriaxone are located in a penicillin-binding protein 2 (penA) gene.

21. (canceled)

22. The nucleic acid detection system of claim 17, wherein the one or more polynucleotide sequences that indicate resistance to ceftriaxone comprise:
a) single nucleotide polymorphisms (SNPs) in the mosaic allele type 60 of the penA gene; or
b) one or more mutations in codons 311, 316, 483, 501, 512, 516, 542 and 545 of the penA gene, wherein the one or more mutations comprise A311V, V316T/P and T483S.

23. (canceled)

24. The nucleic acid detection system of claim 17, wherein the one or more N. gonorrhoeae polynucleotide sequences that indicate resistance to ceftriaxone comprise one or more mutations selected from:
a) One or more mutations in codons 120 and 121 of the porB gene;
b) a mutation in codon 421 of the ponA gene; or
c) both a) and b).

25. (canceled)

26. The nucleic acid detection system of claim 17, wherein the one or more N. gonorrhoeae polynucleotide sequences that indicate;
a) resistance to cefixime comprise one or more mutations chosen from A501V, A501P, A501T, N512Y, A516G, G542S, G545S, P551L and P551S in the penA gene; or
b) resistance to cefixime comprise one or more mutations in codons 311, 312, 316, 345, 483, 375, 376, 377, 501, 542 and 551 of the penA gene.

27. (canceled)

28. The nucleic acid detection system of claim 1, further comprising amplification reagents, wherein the amplification reagents comprise one or more primers pairs for amplifying the one or more N. gonorrhoeae polynucleotide sequences that identify the presence of N. gonorrhoeae, the one or more N. gonorrhoeae polynucleotide sequences that indicate antibiotic resistance, or both.

29. (canceled)

30. The nucleic acid detection system of claim 28, wherein the one or more primers pairs for amplifying the one or more N. gonorrhoeae polynucleotide sequences that identify the presence of N. gonorrhoeae are selected from any one of the nucleotide sequences set forth in SEQ ID NO: 210-221.

31. The nucleic acid detection system of claim 17, wherein the first and second CRISPR-Cas systems are independently selected from a Type V or a Type VI CRISPR-Cas system.

32. The nucleic acid detection system of claim 17, wherein the one or more N. gonorrhoeae polynucleotide sequences that indicate resistance to ciprofloxacin are located in a gyrase A (gyrA) gene.

33. The nucleic acid detection system of claim 17, wherein the one or more N. gonorrhoeae polynucleotide sequences that indicate resistance to ciprofloxacin comprise a single nucleotide polymorphism (SNP) in codon 91 of the gyrA gene that is amino acid substitution S91F.

34. (canceled)

35. The nucleic acid detection system of claim 17, wherein the one or more polynucleotide sequences that indicate resistance to ciprofloxacin are selected from any one of the nucleotide sequences set forth in SEQ ID NOs: 230-253.

36. A method for detecting antibiotic-resistant N. gonorrhoeae in a patient sample comprising:
contacting one or more samples with;
a first CRISPR-Cas system having collateral cleavage activity and configured to bind one or more N. gonorrhoeae polynucleotide sequences that identify the presence of N. gonorrhoeae;
a second CRISPR-Cas system having collateral cleavage activity and configured to bind one or more N. gonorrhoeae polynucleotide sequences that indicate antibiotic resistance;
a first detection construct comprising a cutting motif configured to generate a first detectable signal when preferentially cut by the one or more CRISPR-Cas systems configured to bind one or more N. gonorrhoeae polynucleotide sequences that identify the presence of N. gonorrhoeae;
a second detection construct comprising cutting motifs configured to generate one or more second detectable signals when preferentially cut by the one or more CRISPR-Cas systems configured to bind one or more N. gonorrhoeae polynucleotide sequences that indicate resistance to an antibiotic, wherein the antibiotic is not ciprofloxacin; and
detecting the generation of the first detectable signal, the one or more second detectable signals, or both,
wherein the detection of the first detectable signal but not the one or more second detectable signals indicates the presence of an antibiotic-sensitive strain of N. gonorrhoeae in the sample, and
wherein the detection of the first detectable signal and the one or more second detectable signals indicates the presence of a strain of N. gonorrhoeae in the sample that is resistant:
a) an antibiotic other than ciprofloxacin;
b) ciprofloxacin, and either cefixime or ceftriaxone; or
c) ciprofloxacin, cefixime and ceftriaxone.

37. (canceled)

38. The method of claim 36, wherein the one or more N. gonorrhoeae polynucleotide sequences that identify the presence of N. gonorrhoeae are located in a porA gene.

39. The method of claim 36, wherein the one or more N. gonorrhoeae polynucleotide sequences that identify the presence of N. gonorrhoeae are located in the nucleotide sequence set forth in SEQ ID NO: 225.

40. The method of claim 36, wherein the one or more N. gonorrhoeae polynucleotide sequences that indicate resistance to cefixime or ceftriaxone are located in a penicillin-binding protein 2 (penA) gene.

41. (canceled)

42. The method of claim 36, wherein the one or more polynucleotide sequences that indicate resistance to ceftriaxone comprise:
a) single nucleotide polymorphisms (SNPs) in a mosaic allele type 60 of the penA gene; or
b) one or more mutations in codons 311, 316, 483, 501, 512, 516, 542 and 545 of the penA gene, wherein the one or more mutations comprise A311V, V316T/P and T483S.

43. (canceled)

44. The method of claim 36, wherein the one or more N. gonorrhoeae polynucleotide sequences that indicate resistance to ceftriaxone comprise one or more mutations select from:
a) One or more mutations in codons 120 and 121 of the porB gene;
b) a mutation in codon 421 of the ponA gene; or
c) both a) and b).

45. (canceled)

46. The method of claim 36, wherein the one or more N. gonorrhoeae polynucleotide sequences indicate;
a) resistance to cefixime comprise one or more mutations chosen from A501V, A501P, A501T, N512Y, A516G, G542S, G545S, P551L and P551S in the penA gene; or
b) resistance to cefixime comprise one or more mutations in codons 311, 312, 316, 345, 483, 375, 376, 377, 501, 542 and 551 of the penA gene.

47. (canceled)

48. The method of claim 36, further comprising amplification reagents, wherein the amplification reagents comprise one or more primers pairs for amplifying the one or more N. gonorrhoeae polynucleotide sequences that identify the presence of N. gonorrhoeae, the one or more N. gonorrhoeae polynucleotide sequences that indicate antibiotic resistance, or both.

49. (canceled)

50. The method of claim 48, wherein the one or more primers pairs for amplifying the one or more N. gonorrhoeae polynucleotide sequences that identify the presence of N. gonorrhoeae are selected from any one of the nucleotide sequences set forth in SEQ ID NO: 210-221.

51. The method for detecting N. gonorrhoeae of claim 36, wherein:
a) the patient sample is a sample of blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate, a fluid obtained from a joint, or a swab of skin or a mucosal membrane surface; and
b) nucleic acids in the patient sample are extracted by incubating the sample in 0.05%-0.5% Triton-X at about 25-37° C. for about 5 minutes.

52-56. (canceled)

57. The method for detecting N. gonorrhoeae of claim 36,
wherein the one or more N. gonorrhoeae polynucleotide sequences that indicate resistance to ciprofloxacin comprise a single nucleotide polymorphism (SNP) in codon 91 of the gyrA gene that is amino acid substitution S91F.

58-96. (canceled)
